(19) United States
(12) Patent Application Publication
(10) Pub. No.: US 2025/0319032 A1
(43) Pub. Date: Oct. 16, 2025

(54) PHARMACEUTICAL COMPOSITIONS OF RIBOCICLIB

(71) Applicant: Novartis AG, Basel (CH)

(72) Inventors: Jožko Cesar, Ljubljana (SI); Jan Kopriva, Rakovník (CZ); Andreas Karl Georg Kuemmell, Reinach (CH); Bojana Leben, Ljubljana (SI); Bukeyan Miao, Shanghai (CN); Andrej Razpotnik, Ljubljana (SI); Hendrik Schneider, Basel (CH); Jernej Wagger, Kranj (SI)

(21) Appl. No.: 19/176,436

(22) Filed: Apr. 11, 2025

(30) Foreign Application Priority Data
Apr. 15, 2024 (WO) PCT/CN2024/087723
Sep. 10, 2024 (WO) PCT/CN2024/118001

(51) Int. Cl. A61K 9/20; A61J 1/03; A61K 31/519

(52) U.S. Cl. CPC A61K 9/2054; A61J 1/035; A61K 9/2027; A61K 31/519

(57) ABSTRACT

Pharmaceutical compositions of ribociclib or a pharmaceutically acceptable salt thereof are disclosed, wherein the compositions are substantially free of nitrosamines. Pharmaceutical products containing said compositions are also disclosed, as well as methods for preparing and storing said pharmaceutical compositions and pharmaceutical products.

### TECHNICAL FIELD

Pharmaceutical compositions of ribociclib or a pharmaceutically acceptable salt thereof are disclosed, wherein the compositions are substantially free of nitrosamines. Pharmaceutical products containing said compositions are also disclosed, as well as methods for preparing and storing said pharmaceutical compositions and pharmaceutical products.

### BACKGROUND

The term nitrosamine describes a class of compounds having the chemical structure of a nitroso group bonded to an amine (R₁N(—R₂)—N═O), as shown in the figure below. These compounds can form by a nitrosating reaction between amines (secondary, tertiary, or quaternary amines) and nitrous acid (nitrite salts under acidic conditions) (see “Control of Nitrosamine Impurities in Human Drugs”, Guidance for Industry, U.S. Department of Health and Human Services, Food and Drug Administration, February 2021, Revision 1).

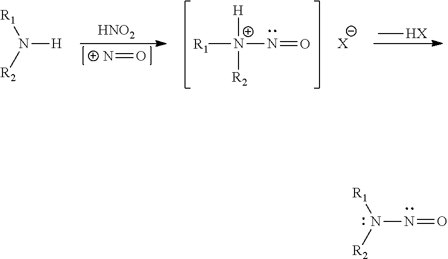

Nitrosamine impurities have been discovered in drugs such as angiotensin II receptor blockers, ranitidine, nizatidine, and metformin. However, nitrosamines are considered by the FDA to be probable or possible human carcinogens and as such, the level of nitrosamines in pharmaceutical compositions or products should be kept at a minimum. In particular, the FDA recommends that the risk of nitrosamines should be assessed in any pharmaceutical product.

Ribociclib is a potent and highly effective inhibitor of cyclin-dependent kinase 4 and cyclin-dependent kinase 6 (CDK4/6) with in vivo activity against hormone receptor-positive, human epidermal growth factor receptor 2-negative (HR+/HER2−) breast cancer and is currently marketed as KISQALI®. It has the following formula (see e.g. WO 2015/022609):

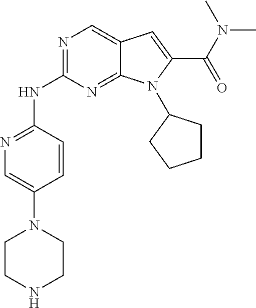 [SMILES: CN(C)C(=O)c1cc2cnc(Nc3ccc(N4CCNCC4)cn3)nc2n1C1CCCC1]

Due to the presence of two secondary amines in the ribociclib structure, there is a potential risk of ribociclib forming nitrosamine impurities if exposed to nitrite salts under acidic reaction conditions. Therefore, there remains a need to provide pharmaceutical compositions and pharmaceutical products of ribociclib or a pharmaceutically acceptable salt thereof which are substantially free of nitrosamines.

### The Invention

The inventors have successfully formulated compositions containing ribociclib or a pharmaceutically acceptable salt thereof which are substantially free of nitrosamines, in particular N-nitroso-ribociclib in free or salt form. The compositions of the invention are intended to be pharmaceutically acceptable (i.e., pharmacologically efficacious and physiologically acceptable).

These compositions are suitable for use in medicine, and in particular for the treatment of cancers, such as breast cancer. The breast cancer may be advanced and/or metastatic breast cancer, or it may be early breast cancer. The breast cancer may be hormone receptor-positive (HR+) breast cancer, for example hormone receptor positive human epidermal growth factor receptor 2-negative (HR+/HER2−) breast cancer.

Accordingly, the invention provides a composition comprising or consisting of ribociclib or a pharmaceutically acceptable salt thereof, wherein said composition is substantially free of nitrosamines, in particular N-nitroso-ribociclib in free or salt form. Thus, in one aspect, the composition comprises ribociclib or a pharmaceutically acceptable salt thereof and is substantially free of nitrosamines, or at least substantially free of N-nitroso-ribociclib in free or salt form. In this aspect, the skilled person would appreciate that the composition comprises little or no nitrosamines, in particular little or no N-nitroso-ribociclib in free or salt form. The composition may further comprise one or more pharmaceutically acceptable excipients. In another aspect, the composition consists of ribociclib or a pharmaceutically acceptable salt thereof. In this aspect, the skilled person would appreciate that the composition comprises no nitrosamines, and therefore no N-nitroso-ribociclib in free or salt form.

The invention further provides a pharmaceutical product comprising a composition of the invention. In particular, the pharmaceutical product may comprise a plurality of oral dosage forms comprising such a composition, e.g., wherein each of the oral dosage forms is a tablet. A pharmaceutical product of the invention may comprise a composition of the invention and documentation, e.g., in the form of packaging or a package insert. For example, a pharmaceutical product may include a document providing instructions to a patient as to how to administer the composition and/or a document which certifies that the composition is substantially free of nitrosamines, or at least substantially free of N-nitroso-ribociclib in free or salt form.

Another aspect of the invention provides methods for preparing ribociclib or a pharmaceutically acceptable salt thereof which is substantially free of nitrosamines, in particular N-nitroso-ribociclib in free or salt form. In particular, the inventors found that limiting the amount of nitrites in the reagents and intermediates that are used to prepare the ribociclib or pharmaceutically acceptable salt thereof provide compositions of ribociclib or pharmaceutically acceptable salts thereof which are substantially free of nitrosamines. The inventors found that this could be achieved by:
Limiting the amount of nitrites in the potassium carbonate that is used in the following deprotection of intermediate B9 to provide ribociclib, B10:

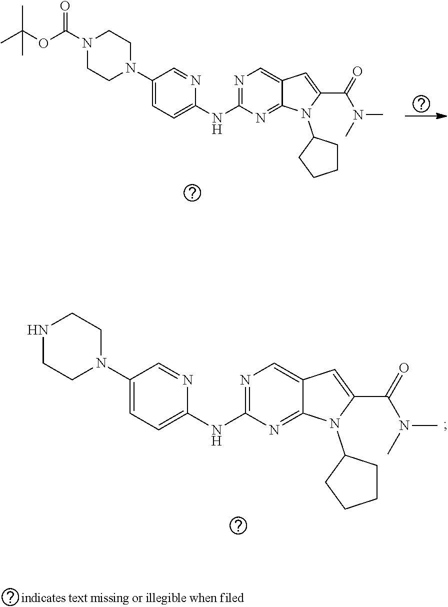 [SMILES: CN(C)C(=O)c1cc2cnc(Nc3ccc(N4CCN(C(=O)OC(C)(C)C)CC4)cn3)nc2n1C1CCCC1 ; CN(C)C(=O)c1cc2cnc(Nc3ccc(N4CCNCC4)cn3)nc2n1C1CCCC1 ; ";"]

and/or
Limiting the amount of nitrites in the organic or inorganic acid (such as succinic acid) that is to be reacted with ribociclib free base so as to form a pharmaceutically acceptable salt of ribociclib.

Another aspect of the invention provides methods for preparing compositions of (i) ribociclib or a pharmaceutically acceptable salt thereof and (ii) one or more pharmaceutically acceptable excipients, wherein said compositions are substantially free of nitrosamines, in particular N-nitroso-ribociclib in free or salt form. The inventors found that limiting the amount of nitrites in any of the excipients and solvents that are used during (i) manufacture of the ribociclib or pharmaceutically acceptable salt thereof or (ii) manufacture of the said compositions, provided compositions which are substantially free of nitrosamines. The inventors found that this could be achieved by:
Limiting the amount of nitrites in any of the excipients that are used during the manufacture of the compositions;
Limiting the exposure of any solvents that are used during the manufacture of the ribociclib or pharmaceutically acceptable salt thereof to oxygen, e.g., by storing the solvents under an atmosphere in which the percentage of oxygen is less than 21% by volume and/or using the solvents shortly after the solvents are unsealed;
Limiting the amount of nitrites in any solvents that are used during the preparation of ribociclib or a pharmaceutically acceptable salt thereof, e.g., by distilling the solvents prior to their use and/or by passing the solvents through an ion exchange resin that is capable of adsorbing nitrites;
If forming a pharmaceutically acceptable salt of ribociclib, wherein ribociclib free base is mixed with an acid in the presence of a solvent, then adding the ribociclib free base last to the mixing vessel;
Filtering any solutions of ribociclib or a pharmaceutically acceptable salt thereof and/or any solutions of the acid that is used to make the salt through a filter which does not comprise a nitrocellulose membrane, e.g., a stainless-steel filter; and/or.
If preparing a crystalline form of ribociclib or a pharmaceutically acceptable salt thereof, then washing the crystalline form with a solvent which is substantially free of nitrites.

Additionally, the inventors found that drying the compositions of ribociclib or a pharmaceutically acceptable salt thereof after mixing the ribociclib or pharmaceutically acceptable salt thereof with one or more excipients provided compositions which were substantially free of nitrosamines despite a period of storage. In particular, when providing the compositions in the form of tablets, the inventors found that drying the tablets during their manufacture (and in particular adding a step of drying the tablets following the compression of the compositions into tablets and the coating of the tablets with a film coating, such as with a moisture barrier film coating) resulted in this reduction in the formation of nitrosamines within the tablets during their subsequent storage. The drying of the tablets until the water activity value of the tablets was less than 0.08, and preferably no more than 0.06, was particularly beneficial. The inventors showed that by conducting this drying step at a relatively low temperature above ambient, and by flowing an atmosphere of low humidity across the tablets at a moderate flow rate, the resulting tablets remained substantially free of nitrosamines despite their long-term storage under ambient conditions, e.g., at 20° C. to 25° C. Temperatures, atmospheres, relative humidities and flow rates for this additional drying step are described elsewhere herein. This use of an additional drying step until a suitable water activity value was reached was found to be a factor in meeting the inventors' objective of producing a pharmaceutical product of ribociclib that avoids the need for cold chain storage at e.g., 2 to 8° C.

Lastly, the inventors found that storing the compositions of ribociclib or a pharmaceutically acceptable salt thereof under certain conditions prevents the formation of nitrosamines such as N-nitroso-ribociclib in free or salt form, meaning that the compositions remained substantially free of nitrosamines. This allows for compositions of ribociclib or pharmaceutically acceptable salts thereof which have a shelf life suitable for pharmaceutical use. In particular, the inventors found that the following storage conditions prevent, retard or reduce the formation of nitrosamines:
Storing the compositions in the presence of a desiccant;
Storing the compositions under an atmosphere in which the percentage of oxygen is less than 21% by volume;
Storing the compositions under an atmosphere in which the humidity is no more than 2.5 g/kg (or a relative humidity of no more than 15%);
Providing the compositions in the form of a plurality of oral dosage forms that are packaged within a blister pack such that each oral dosage form is individually packaged within a substantially moisture and/or gas impermeable blister; and/or
Storing the compositions under refrigerated conditions, e.g., at about 2 to about 8° C.

### FIGURES

FIG. 1 shows the content of nitrite in batches of iPrOH used in the commercial production process.

FIG. 2 shows a chart of N-nitroso-ribociclib content in the ribociclib drug substance by site.

FIG. 3 shows the content of nitrite and NOx in both commercial and distilled batches of/PrOH.

FIG. 4 is a schematic showing the approach taken to ion exchange resin treatment of feed solutions before crystallization of drug substance LEE011 succinate.

FIG. 5 is a schematic showing the approach taken to nitrite remover screening experiments.

FIGS. 6A-6E show the results of nitrite adsorber (ion exchange resin) screens. In the figures, the columns depict “nitrite_change ratio” and the line graph depicts “DMP433_change ratio”, expressed as percentage values.

FIGS. 7A and 7B are shelf life plots of tablet core experiment 011TC and 012TC under conditions of 40° C./75% RH and 50° C./75% RH, respectively.

FIGS. 8A and 8B are shelf life plots of NG4411, NG4412, NG4413 and NH4538 under conditions of 30° C./75% RH and 40° C./75% RH, respectively.

FIGS. 9A, 9B, and 9C are shelf life plots of NK1288 and NK6069 under conditions of 2-8° C., 40° C./75% RH, and 50° C./75% RH, respectively. FIG. 9D is a statistical stability prediction of a standard KISQALI® product across different storage temperatures.

FIG. 10 shows the XRPD diffractogram of Form E of ribociclib succinate recorded in reflection mode. The figure corresponds to FIG. 2 of WO 2020/152629.

FIG. 11 shows the DSC plot of ribociclib succinate Form E at a heating rate of 10 degrees Celsius per minute. This figure corresponds to FIG. 7 of WO 2020/152629.

FIG. 12 shows the thermogravimetric curve of Form E at heating rate at 10 degrees Celsius per minute. This figure corresponds to FIG. 11 of WO 2020/152629.

FIG. 13 is a schematic showing the approach taken to solvent distillation and methods of mixing ribociclib with a solution of acid.

FIG. 14A shows the linearity curve of N-nitroso-ribociclib and FIGS. 14B-14D show the residual plots for Replicates 1-3, respectively.

FIGS. 15A-15E demonstrate a method of detecting the presence and amount of nitrites using the Griess method. FIG. 15A shows the solvent HPLC curve; FIGS. 15B and 15C show the HPLC curves for Blank Solution 1 and Blank Solution 2, respectively; FIG. 15D shows the HPLC curve for the Comparison Solution; and FIG. 15E shows the HPLC curve for a Sample solution.

FIGS. 16A-16F show predictive modelling of accelerated stability data for the drug product. FIG. 16A shows the Arrhenius plot, FIGS. 16B-16E show the shelf-life predictions, and FIG. 16F shows the APS-based predictions versus real-time stability data.

FIGS. 17A-17C show predictive modelling of accelerated stability data for the drug substance. FIG. 17A shows the Arrhenius plot and FIGS. 17B-17C show the shelf-life predictions.

FIG. 18 shows predictive modelling stability profiles.

FIG. 19 shows a validated manufacturing process for ribociclib drug products that are provided in the form of tablets, wherein the process includes blend preparation, roller compaction, compression, and film-coating. As shown in the figure, an additional drying step that uses one of two different drying methods was studied in Example 18 (one method was applied to the film-coated tablets of a first sub-batch A, and one method was applied to the film-coated tablets of a second sub-batch B).

FIG. 20 shows the water activity of the film-coated tablets of sub-batch A (i.e., film-coated tablets that were dried in the Huettlin HKC-400-device) as a function of drying time.

FIG. 21 shows the water activity of the film-coated tablets of sub-batch B (i.e., film-coated tablets that were dried using drying vessels in the PIP drying system) as a function of drying time.

FIG. 22 shows the water activity of the dried ribociclib film-coated tablets of sub-batch A as a function of time, when exposed to environmental conditions with an air humidity of RH NMT 15%.

### DEFINITIONS

The terms “substantially free” or “essentially free” in relation to the presence of a given component within e.g., a composition means that no more than 5% by weight of the composition (e.g., no more than 1% by weight of the composition) is that given component. The word “substantially” does not exclude “completely” e.g. a composition which is “substantially free” from Y may be completely free from Y. Where necessary, the word “substantially” may be omitted from the definition of the invention.

The term “no more than”, when used to refer to an amount of a particular component in a composition, likewise includes the possibility that the composition is completely free of said component.

The use of the articles “a”, “an”, and “the” in both the description and claims are to be construed to cover both the singular and the plural, unless otherwise indicated herein or clearly contradicted by context. The terms “comprising”, “having”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “between” with reference to two values includes those two values e.g., the range “between” 10 mg and 20 mg encompasses e.g., 10, 11, 12, 13, 14, 15, 16, 17, 18, 19 and 20 mg.

As used herein the term “about” is understood by the person of skill in the art to reflect the variability in the numerical value it modifies. Values expressed herein are understood to include a range of variability. To reflect this variability, any numerical value used herein incorporates this variability. As such, numerical values used herein encompass that value stated, as well as the value as modified with “about.” In certain embodiments, the term “about” in relation to a numerical value x is optional and means, for example, x±10%, x±5%, or x±1%.

Any numerical value used herein may be converted and expressed in any equivalent unit. For example, 1 ppm may be expressed as 1000 ppb. Thus, for example, any reference herein to “a composition comprising no more than 1 ppm” of a given component may be expressed as “a composition comprising no more than 1000 ppb” of said component.

“% w/w” means the weight of a component as a percentage of the total weight of a composition, e.g., a dosage form, in which the component is present. For example, a composition comprising “5% w/w of component X” refers to a composition in which the weight of component X is 5% of the total weight of the composition.

The term “pharmaceutically acceptable” with respect to a substance refers to the substance as being generally regarded as safe and suitable for use without undue toxicity, irritation, allergic response, and the like, commensurate with a reasonable benefit/risk ratio.

“Pharmaceutically acceptable salt” refers to a salt of a compound that is pharmaceutically acceptable and that possesses (or can be converted to a form that possesses) the desired pharmacological activity of the parent compound. Such salts can be readily made by a person of ordinary skill in the art, and may include acid addition salts, preferably with organic or inorganic acids. Suitable inorganic acids are, for example, halogen acids, such as hydrochloric acid, sulfuric acid, or phosphoric acid. Suitable organic acids are, e.g., succinic acid, carboxylic acids or sulfonic acid, such as fumaric acid or methanesulfonic acid.

As used herein, the term “salts” includes co-crystals. The term “co-crystal” refers to a crystalline compound comprising two or more molecular components, e.g., wherein proton transfer between the molecular components is partial or incomplete. Accordingly, the term “pharmaceutically acceptable salt” as used herein encompasses salts and co-crystals as defined herein.

The term “solvate” means a molecular complex comprising a compound and one or more pharmaceutically acceptable solvent molecules. Examples of solvent molecules include water and C₁₋₆ alcohols, e.g., ethanol. When the solvate is water, the term “hydrate” may be used. Crystalline forms of ribociclib or a pharmaceutically acceptable salt thereof may be solvates.

“Treating” and “treatment” of a disease include the following:
(1) inhibiting the disease, i.e., arresting or reducing the development of the disease or its clinical symptoms, and
(2) relieving the disease, i.e., causing regression of the disease or its clinical symptoms.

Examples of treatment in the context of early breast cancer include a reduced risk of recurrence of the cancer. An example of early breast cancer is stage II and Ill hormone receptor-positive, human epidermal growth factor receptor 2-negative (HR+/HER2−) early breast cancer.

Examples of treatment in the context of advanced breast cancer include improved progression-free survival (PFS). Tumour growth inhibition, sustained tumour regression and/or delayed tumour regrowth can be observed. An example of advanced breast cancer is hormone receptor-positive, human epidermal growth factor receptor 2-negative (HR+/HER2−) locally advanced or metastatic breast cancer.

The term “effective amount” refers to an amount that may be effective to elicit the desired biological or medical response, including the amount of a compound that, when administered to a subject for treating a disease, is sufficient to effect such treatment for the disease. The effective amount will vary depending on the compound, the disease and its severity, and the age, weight, etc. of the subject to be treated. The effective amount can include a range of amounts.

As used in the context of the present invention, unless otherwise noted, the terms “dose” and “dosage” and “dosage amount” mean the amount of an active compound or pharmaceutical ingredient which is administered to a patient in an individual administration. The term “dosage regimen” (or “dosing regimen”) mean a defined sequence of one or more individual administrations.

The one or more “pharmaceutically acceptable excipients” referred to herein can readily be selected by one of ordinary skill in the art and will be determined by the desired mode of administration. Illustrative examples of modes of administration include oral, nasal, parenteral, topical, transdermal, and rectal. Preferably, the mode of administration is oral. The pharmaceutical compositions of this invention may take any pharmaceutical form recognizable to the skilled person as being suitable. Suitable pharmaceutical forms include solid, semisolid, liquid, or lyophilized formulations, such as tablets, powders, capsules, suppositories, suspensions, liposomes, and aerosols.

A “desiccant” as used herein refers to a hygroscopic material that absorbs moisture from the air and is used to create and maintain dry spaces i.e. to prevent condensation and maintain low humidity levels. Suitable desiccants can readily be selected by a person of ordinary skill in the art. Examples of desiccants include activated alumina, aerogel, benzophenone, bentonite clay, calcium chloride, calcium oxide, calcium sulphate, cobalt (II) chloride, copper (II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. For example, the desiccant may be selected from silica gel, molecular sieve, calcium oxide, and calcium sulphate, e.g., silica gel.

The phrase “stored in the presence of a desiccant” as used herein refers to the inclusion of a desiccant within the packaging of the compositions and pharmaceutical products described. The phrase will be readily understood by a person of ordinary skill in the art who will be readily able to select suitable packaging. Examples of suitable packaging for the compositions and/or pharmaceutical products described herein include amber glass bottles, rigid high-density polyethylene (HDPE) bottles, Alu-bags, or blister packaging, such as rigid high-density polyethylene (HDPE) bottles, Alu-bags, or blister packaging, e.g., plastic (such as Aclar®) blister packaging, laminate (such as Perlalux®) blister packaging, or alu-alu blister packaging, preferably alu-alu blister packaging. The desiccant may be placed in a separate container from the composition or pharmaceutical product, e.g., in a small HDPE canister or kraft paper bag, prior to inclusion in said packaging. The desiccant may form part of the packaging, e.g., the packaging may comprise a layer, a film, and/or a coating which consists of, comprises, or is derived from a desiccant and which is exposed to the void in which the composition or pharmaceutical product is contained. For example, the packaging for a composition or pharmaceutical product as described herein may be a blister package that includes a blister tray that is an aluminium, laminate or thermoformed plastic sheet with a plurality of blister cells or depressions formed therein. Typically, after the compositions or pharmaceutical products described herein are placed in the cells, the compositions or products are retained and protected in the respective cells by securing a backing sheet to the blister tray. One or more of the blister cells or depressions (e.g., each of the blister cells or depressions) and/or the backing sheet may be substantially moisture and/or gas impermeable. Thus, in embodiments wherein the composition is provided as a plurality of oral dosage forms, once the blister package is sealed, each oral dosage form may be individually packaged within a substantially moisture and/or gas impermeable blister. Additionally, one or more of the blister cells or depressions (e.g., each of the blister cells or depressions) may comprise a layer, a film and/or a coating which consists of, comprises, or is derived from a desiccant. Alternatively, or additionally, the backing sheet may comprise a layer, a film and/or a coating which consists, comprises, or is derived from a desiccant. Thus, in these embodiments, once the blister package is sealed, the composition is stored in said sealed blister package which also contains a desiccant as a component comprised within the blister package.

Values of “humidity” may be given either as absolute humidities (as g/kg) or as relative humidities (as a percentage). A person of ordinary skill in the art will readily understand how to convert between values of relative humidity at a given temperature and values of absolute humidity. For example, a relative humidity of 15% at 22.5° C. may correspond to an absolute humidity of 2.5 g/kg.

The term “substantially in accordance with”, with reference to X-ray diffraction peak positions, means that typical peak position and intensity variability are taken into account. For example, one skilled in the art will appreciate that the peak positions (20) will show some inter-apparatus variability, typically as much as 0.2°. Further, one skilled in the art will appreciate that relative peak intensities will show inter-apparatus variability as well as variability due to a degree of crystallinity, preferred orientation, prepared sample surface, and other factors known to those skilled in the art, and they should therefore be taken as a qualitative measure only.

The phrase “substantially moisture and/or gas impermeable” as used herein may, in accordance with the usual definition in the art, be understood to refer to a material (such as a packaging material, e.g., a blister packaging material) which is substantially impermeable to moisture and/or which is substantially impermeable to gas (such as oxygen) transmission. Preferably, the material will be substantially impermeable to moisture, and optionally the material will also be substantially impermeable to gas (such as oxygen) transmission. The phrase will be readily understood by a person of ordinary skill in the art who will be readily able to select suitable packaging, such as any suitable packaging which is known to protect moisture-sensitive and/or oxygen-sensitive pharmaceutical products. For example, a person of ordinary skill in the art will appreciate that suitable packaging may be packaging which has a low water vapor transmission rate (WVTR), for example packaging which has a WVTR of less than 0.1 g/m²/day, preferably less than 0.08 g/m²/day, more preferably less than 0.05 g/m²/day, and most preferably less than 0.01 g/m²/day, optionally wherein the WVTR is measured using ASTM F-1249 at 40° C./75% RH. Additionally, or alternatively, one skilled in the art will appreciate that suitable packaging may be packaging which has a low oxygen transmission rate (OTR), for example packaging which has an OTR of less than 20 cm³/m²/day, preferably less than 15 cm³/m²/day, more preferably less than 5 cm³/m²/day, and most preferably less than 1 cm³/m²/day, optionally wherein the OTR is measured using ASTM D-3985 at 23° C./50% RH. Suitable blister packaging may include aluminium-aluminium (“alu-alu”) blister packaging, plastic blister packaging (e.g., the PCTFE Aclar® blisters which are commercially available through Honeywell, such as Aclar® UltRx 4000), and laminate blister packaging. Suitable laminate blister packaging may include PVC/PE/PVdC blister packaging (e.g., the triplex Perlalux® blisters which are commercially available through Perlen Packaging, such as Perlalux® Tristar ultra) and laminate blister packaging comprising at least one layer of PCTFE (such as PVC/PCTFE blister packaging, e.g., the Pentapharm® Aclar® PA/02 blisters which are commercially available through Klöckner Pentaplast). Preferably, the blister packaging is alu-alu blister packaging. Alu-alu packaging is reported to have a WVTR of less than 0.01 g/m²/day and an OTR of less than 0.5 cm³/m²/day. Aclar® UltRx 4000 (PCTFE) blister packaging is reported to have a WTR of 0.048 g/m²/day and an OTR of 18 cm³/m²/day. Perlalux® Tristar ultra (PVC/PE/PVdC) blister packaging is reported to have a WVTR of 0.06 g/m²/day and an OTR of 0.067 cm³/m²/day.

The term “release of the composition” or “release date” or “date of release” as used herein may, in accordance with the usual definition that is used in the art, refer to the date on which a composition comprising or consisting of ribociclib or a pharmaceutically acceptable salt thereof is placed on the market or made available for purchase by third parties. The shelf life may be calculated as starting from this release date, as described in more detail below. The release date should not exceed 30 days from the date of production of the composition. For a composition comprising ribociclib or a pharmaceutically acceptable salt thereof, the date of production of the composition may be taken as the date that the first step is performed involving combining the ribociclib or pharmaceutically acceptable salt thereof with other ingredients. For a composition consisting of ribociclib or a pharmaceutically acceptable salt thereof, the date of production of the composition may be taken as the initial date on which the ribociclib or pharmaceutically acceptable salt thereof is filled into a container for use as a medicinal product. If the composition is released later than 30 days from the date of production, then the date of production, rather than the release date, should be taken as the start of the shelf life.

“Ribociclib” refers to the CDK4/6 inhibitor known by the IUPAC name 7-cyclopentyl-N, N-dimethyl-2-{[5-(piperazin-1-yl)pyridin-2-yl]amino}-7H-pyrrolo[2,3-d]pyrimidine-6-carboxamide. Ribociclib is currently approved for use in the treatment of breast cancer, in particular hormone receptor-positive, human epidermal growth factor receptor 2-negative (HR+/HER2−) breast cancer and is marketed as KISQALI®. Ribociclib is also known as “LEE011”. Its CAS registry number is 1211441-98-3, its chemical formula is given below (see WO 2015/022609 and WO 2016/166703) and its synthesis is specifically described in WO 2010/020675:

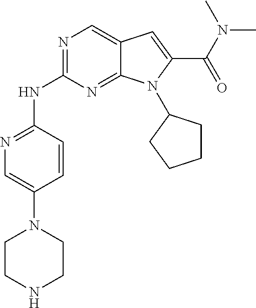 [SMILES: CN(C)C(=O)c1cc2cnc(Nc3ccc(N4CCNCC4)cn3)nc2n1C1CCCC1]

In accordance with the normal definition that is used in the art, the term “drug substance” as used in the context of the invention described herein refers to ribociclib or a pharmaceutically acceptable salt thereof, e.g., ribociclib succinate, which is referred to in the synthetic schemes included herein as “B12” or “LEE011-B12 (IP)”. Similarly, the term “drug product” as used in the context of the invention that is described herein refers to a pharmaceutical composition comprising ribociclib or a pharmaceutically acceptable salt thereof and one or more pharmaceutically acceptable excipients, e.g., pharmaceutical compositions comprising ribociclib succinate and one or more pharmaceutically acceptable excipients, e.g., film-coated tablets comprising ribociclib and one or more pharmaceutically acceptable excipients.

“Nitrosamines” refers to a class of compounds having the chemical structure of a nitroso group bonded to an amine (R₁N(—R₂)—N═O), as shown in the figure below.

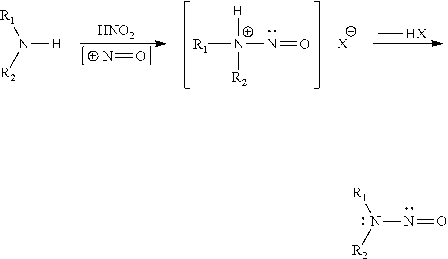

These compounds can form by a nitrosating reaction between amines (secondary, tertiary, or quaternary amines) and nitrous acid (nitrite salts under acidic conditions). Examples of nitrosamine impurities that have been found in pharmaceutical products include N-Nitrosodimethylamine (NDMA), N-Nitrosodiethylamine (NDEA), N-Nitrosomethylphenylamine (NMPA), N-Nitrosodiisopropylamine (NDIPA), N-Nitrosoisopropylethylamine (NIPEA), N-Nitrosodibutylamine (NDBA) and N-Nitroso-N-methyl-4-aminobutyric Acid (NMBA). Nitrosamine drug substance-related impurities (NDSRI) are a class of nitrosamine impurities that share structural similarity to the active pharmaceutical ingredient (API) (i.e., the NDSRI has the API or an API fragment in its chemical structure) and are therefore unique to each API. The nitrosamine that is of particular concern in the present invention, which relates to compositions comprising or consisting of ribociclib, is a nitrosamine of ribociclib, the API. Although ribociclib comprises two secondary amines (one acting as a linker between the aromatic rings and the other in the piperazine moiety of the molecule), it has been found that, even under forced conditions, nitrosation does not occur at the aromatic secondary amine. Therefore, in the context of the present invention, “N-nitroso-ribociclib” (also known as “NVP-DMP433” or “NDSRI DMP433” or “DMP433”) refers to the compound with the chemical name 7-cyclopentyl-N,N-dimethyl-2-((5-(4-nitrosopiperazin-1-yl)pyridin-2-yl)amino)-7H-pyrrolo[2,3-d]pyrimidine-6-carboxamide i.e. the nitrosamine form of ribociclib given by the following structure:

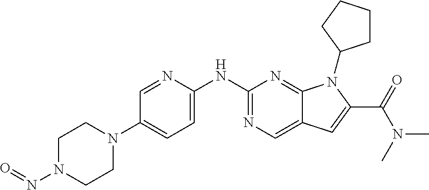 [SMILES: CN(C)C(=O)c1cc2cnc(Nc3ccc(N4CCN(N=O)CC4)cn3)nc2n1C1CCCC1]

Preferably, the amount of DMP433 in the ribociclib drug substance should not exceed 0.4 ppm, in order to ensure that the end-of-shelf-life limit of 1 ppm for the drug product is met with an appropriate level of confidence (e.g., at least 95%) when the drug product is stored under conditions as specified herein. Similarly, the amount of DMP433 in the ribociclib drug product should preferably not exceed 0.6 ppm or 0.7 ppm at release, in order to ensure that the end-of-shelf-life limit of 1 ppm for the drug product is met with an appropriate level of confidence (e.g., at least 95%) when the drug product is stored under conditions as specified herein. A pharmaceutical composition that is to be used in the treatments that are described herein and which is a tablet comprising an amount of ribociclib or a pharmaceutically acceptable salt thereof that corresponds to 200 mg of ribociclib free base should therefore contain no more than 200 ng of DMP433 in free or salt form.

“Palbociclib” refers to the CDK4/6 inhibitor known by the IUPAC name 6-Acetyl-8-cyclopentyl-5-methyl-2-{[5-(1-piperazinyl)-2-pyridinyl]amino}pyrido[2,3-d]pyrimidin-7 (8H)-one. Palbociclib is currently approved for use in the treatment of breast cancer, in particular hormone-receptor positive, human epidermal growth factor receptor 2-negative (HR+/HER2−) breast cancer and is marketed as IBRANCE®. Palbociclib is also known as “PD-0332991”. Its CAS registry number is 571190-30-2 and its chemical structure is shown below:

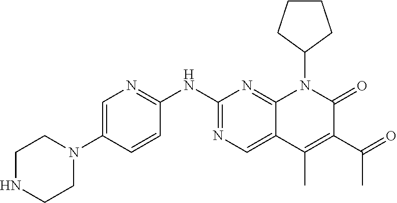 [SMILES: CC(=O)c1c(C)c2cnc(Nc3ccc(N4CCNCC4)cn3)nc2n(C2CCCC2)c1=O]

The method of testing for the presence and/or total amount of nitrites that is used in the methods of the invention may be any method which allows for the detection and/or quantitation of nitrites in a composition. For example, the method may be the Griess test. The Griess test allows for the detection and analysis of nitrite by the formation of a red-pink colour upon treatment of a nitrite-containing sample with the Griess reagent. The Griess reagent consists of two components in an acidic solution: an aniline derivative and a coupling agent. The most common arrangements use sulfanilamide and N-(1-naphthyl)ethylenediamine. For example 0.2% N-(1-naphthyl)ethylenediamine dihydrochloride and 2% sulfanilamide in 5% phosphoric acid may be used as a Griess reagent. Other aniline derivatives that may be used in a Griess reagent include sulfanilic acid, nitroaniline, and p-aminoacetophenone.

The Griess test involves two subsequent reactions, as shown in the scheme below. When the aniline derivative (e.g., sulfanilamide) is added, the nitrite ion reacts with it in the Griess diazotization reaction to form a diazonium salt, which then reacts with N-(1-naphthyl)ethylenediamine in an azo-coupling reaction, forming a pink-red azo-dye. Using a spectrophotometer, the nitrite concentration can then be quantitatively detected.

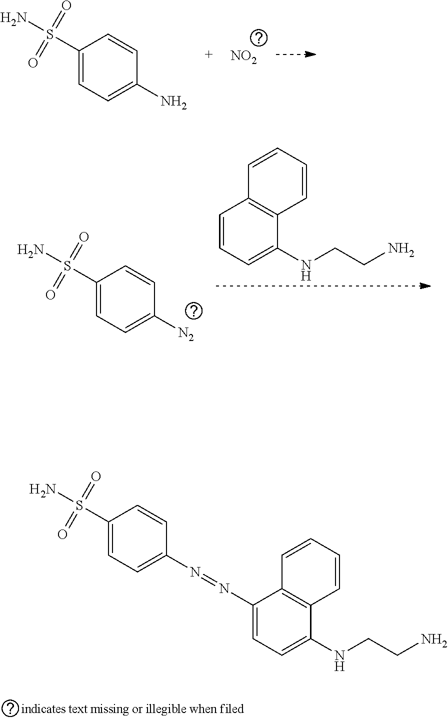

An example of a method of testing for the presence and/or amount of nitrites is provided herein as Example 16.

The method of testing for the presence and/or total amount of nitrosamines (such as the presence and/or total amount of N-nitroso-ribociclib in free or salt form) that is used in the methods of the invention may be any method which allows for the detection of nitrosamines in a composition. For example, the method may involve the Griess test described above. In such a method, the nitrosamines may be first denitrosated (e.g., by photolysis) to form a nitrite, which is then detected in the Griess test. Alternatively, the method may include high performance liquid chromatography (HPLC)- and/or gas chromatography (GC)-mass spectroscopy. An example of a method of testing for the presence and amount of nitrosamines (namely N-nitroso-ribociclib) is provided herein as Example 14.

An aspect of the present invention relates to the use of the Griess test and/or high performance liquid chromatography (HPLC) and/or gas chromatography (GC)-mass spectroscopy to detect the presence and/or amount of an impurity in a composition comprising ribociclib or a pharmaceutically acceptable salt thereof, for example wherein the impurity is N-nitroso-ribociclib in free or salt form. For example, high performance liquid chromatography (HPLC)- and gas chromatography (GC)-mass spectroscopy may be used, such as the high performance liquid chromatography (HPLC)- and gas chromatography (GC)-mass spectroscopy method provided herein as Example 14.

In one aspect, the present invention provides the use of a gradient elution in a high performance liquid chromatography (HPLC) to detect an impurity in a composition comprising ribociclib or a pharmaceutically acceptable salt thereof, for example wherein the impurity is N-nitroso-ribociclib in free or salt form. The gradient elution may be used in a high performance liquid chromatography (HPLC)- and gas chromatography (GC)-mass spectroscopy method. The gradient elution may be carried out with a mobile phase A and a mobile phase B as eluents, wherein mobile phase A may comprise, or consist of, formic acid and water, and wherein mobile phase B may comprise, or consist of, methanol. Mobile phase A may comprise, or consist of, 0.1% v/v formic acid in water. The gradient elution may comprise one or more, and preferably each, of the following stages: (1) a stage that is about 2.5 minutes in duration in which the volume of mobile phase A:mobile phase B is about 60:40, (2) a stage that is about 7 minutes in duration in which the volume of mobile phase A:mobile phase B is about 60:40, (3) a stage that is about 0.5 minutes in duration in which the volume of mobile phase A:mobile phase B is about 5:95, (4) a stage that is about 0.5 minutes in duration in which the volume of mobile phase A:mobile phase B is about 5:95, (5) a stage that is about 3.5 minutes in duration in which the volume of mobile phase A:mobile phase B is about 60:40, and (6) a stage in which the volume of mobile phase A:mobile phase B is about 60:40. Preferably, the gradient elution comprises each of the stages indicated as (1)-(6), preferably in the following order: (1), (2), (3), (4), (5) and (6). The gradient elution may be carried out in a reversed-phase C18 chromatographic column (e.g., an Acquity UPLC BEH column, 1.7 μm×100×2.1 mm), at a column temperature of 40° C., with a flow rate of 0.35 mL/min, and/or with a volume of injection of 10 μL. It will be appreciated that the skilled person would be able to select alternative appropriate columns and/or conditions.

It will be appreciated that references herein to an amount (e.g., the total amount, the maximum amount, or specific numerical amounts) of nitrosamines, or to an amount of nitrosamines and salts thereof, such as a reference to a total amount of N-nitroso-ribociclib and salts thereof, refers to the total amount of said nitrosamine in free or salt form. For example, references to a “total amount of N-nitroso-ribociclib in free or salt form of no more than 1 ppm” or to a “total amount of N-nitroso-ribociclib and salts thereof of no more than 1 ppm” are synonymous expressions which mean that both the amount of N-nitroso-ribociclib in the form of its free base and the amount of N-nitroso-ribociclib in the form of any salt thereof together add up to no more than 1 ppm. The same applies to the references herein to a “total amount of ribociclib in free or salt form” or to a “total amount of ribociclib and salts thereof”, which may each be identified as a reference amount against which any such ppm value is to be considered. As such, a reference to the “total amount of N-nitroso-ribociclib in free or salt form of no more than 1 ppm, relative to the total amount of ribociclib in free or salt form” means that the total amount of N-nitroso-ribociclib, when taking account of both any N-nitroso-ribociclib that is present in the form of its free base and any N-nitroso-ribociclib that is present in the form of a salt, is no more than 1 ppm, relative to the total amount of ribociclib when again taking account of both any ribociclib that is present in the form of its free base and any ribociclib that is present in the form of a salt. References to other ppm values of nitrosamines, e.g., other ppm values of N-nitroso-ribociclib or ppm values of other nitrosamines, should be understood in the same way. Similarly, a composition that is “substantially free of N-nitroso-ribociclib and salts thereof” refers to a composition that is substantially free of N-nitroso-ribociclib in free or salt form.

### DETAILED DESCRIPTION OF THE INVENTION

The following passages describe features of the aspects and embodiments of the invention in more detail. Unless explicitly stated, and/or unless it would not be technically possible to do so, the following passages are to be read together and the features described therein are combinable.

### Compositions of the Invention

An aspect of the present invention relates to a composition comprising or consisting of ribociclib or a pharmaceutically acceptable salt thereof, wherein said composition is substantially free of nitrosamines. The composition may further comprise one or more pharmaceutically acceptable excipients.

The nitrosamines may be N-nitroso-ribociclib in free or salt form. Thus, another aspect of the present invention relates to a composition comprising or consisting of ribociclib or a pharmaceutically acceptable salt thereof, wherein said composition is substantially free of N-nitroso-ribociclib in free or salt form.

The total amount of nitrosamines (such as N-nitroso-ribociclib in free or salt form) in the composition comprising or consisting of ribociclib or a pharmaceutically acceptable salt thereof may be no more than about 5 ppm, for example no more than about 4 ppm, no more than about 3 ppm, no more than about 2 ppm, or no more than about 1 ppm. As appreciated by one of ordinary skill in the art, particularly in the context of the disclosure herein, such ppm values for the total amount of nitrosamines or the total amount of N-nitroso-ribociclib in free or salt form that is present in a composition are relative to the total amount of the API within the composition, i.e., they are relative to the total amount of ribociclib in free or salt form. Preferably, the total amount of nitrosamines (such as N-nitroso-ribociclib in free or salt form) in the composition comprising or consisting of ribociclib or a pharmaceutically acceptable salt thereof may be no more than about 1 ppm. Thus, another aspect of the invention relates to a composition comprising or consisting of ribociclib or a pharmaceutically acceptable salt thereof, wherein the total amount of nitrosamines (such as N-nitroso-ribociclib in free or salt form) in the composition is no more than about 1 ppm. For example, the total amount of nitrosamines (such as N-nitroso-ribociclib in free or salt form) in the composition may be no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, no more than about 0.25 ppm, no more than about 0.2 ppm, no more than about 0.15 ppm, no more than about 0.1 ppm, no more than about 0.05 ppm, or no more than about 0.025 ppm. Preferably, the total amount of nitrosamines (such as N-nitroso-ribociclib in free or salt form) in the composition may be no more than about 0.7 ppm, for example no more than about 0.6 ppm or no more than about 0.4 ppm, relative to the total amount of ribociclib in free or salt form.

The total amount of nitrosamines (such as N-nitroso-ribociclib in free or salt form) in the composition comprising or consisting of ribociclib or a pharmaceutically acceptable salt thereof may be no more than the maximum amount of nitrosamines (such as N-nitroso-ribociclib in free or salt form) that is permitted in such a composition by a regulatory authority. In one aspect of the invention, the total amount of N-nitroso-ribociclib in free or salt form in the composition comprising or consisting of ribociclib or a pharmaceutically acceptable salt thereof may be no more than the maximum amount of N-nitroso-ribociclib that is permitted in such a composition by a regulatory authority. The maximum amount of nitrosamines (such as N-nitroso-ribociclib) that is permitted in such a composition by a regulatory authority may be the amount that is permitted at the time when the composition is prepared, released and/or administered to a patient.

Throughout the present disclosure, a reference to a regulatory authority may be considered to be the regulatory authority that is responsible for the country or jurisdiction in which the composition is, or is intended to be, administered. It may be e.g., the FDA, the EMA, the MHRA, Swissmedic, or the PMDA. Thus, another aspect of the invention relates to a composition comprising or consisting of ribociclib or a pharmaceutically acceptable salt thereof, wherein the total amount of nitrosamines (such as N-nitroso-ribociclib in free or salt form) in the composition is no more than the maximum amount of nitrosamines that is permitted in such a composition, by a regulatory authority. Another aspect of the invention relates to a composition comprising or consisting of ribociclib or a pharmaceutically acceptable salt thereof, wherein the total amount of N-nitroso-ribociclib in free or salt form in the composition is no more than the maximum amount of N-nitroso-ribociclib that is permitted in such a composition, by a regulatory authority. The maximum amount of nitrosamines (such as N-nitroso-ribociclib) that is permitted in such a composition by a regulatory authority may be the amount that is permitted at the time when the composition is prepared, released and/or administered to a patient. In one aspect, the regulatory authority is the US FDA, and the maximum amount of nitrosamines that is permitted in the composition comprising or consisting of ribociclib or a pharmaceutically acceptable salt thereof is 0.6 ppm, relative to the total amount of ribociclib in free or salt form. In another aspect, the regulatory authority is the EMA, and the maximum amount of nitrosamines that is permitted in the composition comprising or consisting of ribociclib or a pharmaceutically acceptable salt thereof is 0.7 ppm, relative to the total amount of ribociclib in free or salt form.

The total amount of nitrosamines (such as N-nitroso-ribociclib in free or salt form) in the composition comprising or consisting of ribociclib or a pharmaceutically acceptable salt thereof may result in no more than 400 ng/day of said nitrosamines being administered to the patient, when the composition is administered according to an approved dosage regimen of ribociclib or pharmaceutically acceptable salt thereof (for example, in the case that two 200 mg ribociclib tablets are administered per day, no more than 200 ng per tablet of said nitrosamines). Thus, another aspect of the invention relates to a composition comprising or consisting of ribociclib or a pharmaceutically acceptable salt thereof wherein nitrosamines (such as N-nitroso-ribociclib in free or salt form), if present, are present in the composition in an amount that results in no more than 400 ng/day of said nitrosamines being administered to the patient, when the composition is administered according to an approved dosage regimen of ribociclib or pharmaceutically acceptable salt thereof.

The composition comprising or consisting of ribociclib or a pharmaceutically acceptable salt thereof may be tested, e.g., may be tested to determine the amount of nitrosamines (such as N-nitroso-ribociclib in free or salt form) in the composition. The composition comprising or consisting of ribociclib or a pharmaceutically acceptable salt thereof may be found in such testing to have a total amount of nitrosamines (such as N-nitroso-ribociclib in free or salt form) of no more than about 5 ppm, for example no more than about 4 ppm, no more than about 3 ppm, no more than about 2 ppm, or no more than about 1 ppm. Preferably, the composition comprising or consisting of ribociclib or a pharmaceutically acceptable salt thereof may be found in such testing to have a total amount of nitrosamines (such as N-nitroso-ribociclib in free or salt form) of no more than about 1 ppm. Thus, another aspect of the invention relates to a composition comprising or consisting of ribociclib or a pharmaceutically acceptable salt thereof which has been tested and found to have a total amount of nitrosamines (such as N-nitroso-ribociclib in free or salt form) of no more than about 1 ppm. For example, the composition comprising or consisting of ribociclib or a pharmaceutically acceptable salt thereof may have been tested and found to have a total amount of nitrosamines (such as N-nitroso-ribociclib in free or salt form) of no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, no more than about 0.25 ppm, no more than about 0.2 ppm, no more than about 0.15 ppm, no more than about 0.1 ppm, no more than about 0.05 ppm, or no more than about 0.025 ppm. Preferably, the composition comprising or consisting of ribociclib or a pharmaceutically acceptable salt thereof may have been tested and found to have a total amount of nitrosamines (such as N-nitroso-ribociclib in free or salt form) of no more than about 0.7 ppm, for example no more than about 0.6 ppm or no more than about 0.4 ppm, relative to the total amount of ribociclib in free or salt form.

The composition comprising or consisting of ribociclib or a pharmaceutically acceptable salt thereof may also be found in such testing to have a total amount of nitrosamines (such as N-nitroso-ribociclib in free or salt form) that is no more than the maximum amount of nitrosamines that is permitted in such a composition, by a regulatory authority. In one aspect, the composition comprising or consisting of ribociclib or a pharmaceutically acceptable salt thereof may be found in such testing to have a total amount of N-nitroso-ribociclib in free or salt form that is no more than the maximum amount of N-nitroso-ribociclib that is permitted in such a composition by a regulatory authority. The maximum amount of nitrosamines (such as N-nitroso-ribociclib) that is permitted in such a composition by a regulatory authority may be the amount that is permitted at the time when the composition is prepared, released and/or administered to a patient. Thus, another aspect of the invention relates to a composition comprising or consisting of ribociclib or a pharmaceutically acceptable salt thereof which has been tested and found to have a total amount of nitrosamines (such as N-nitroso-ribociclib in free or salt form) that is no more than the maximum amount of nitrosamines that is permitted in such a composition by a regulatory authority. Another aspect of the invention relates to a composition comprising or consisting of ribociclib or a pharmaceutically acceptable salt thereof which has been tested and found to have a total amount of N-nitroso-ribociclib in free or salt form that is no more than the maximum amount of N-nitroso-ribociclib that is permitted in such a composition by a regulatory authority. The maximum amount of nitrosamines (such as N-nitroso-ribociclib) that is permitted in such a composition by a regulatory authority may be the amount that is permitted at the time when the composition is prepared, released and/or administered to a patient. In one aspect, the regulatory authority is the US FDA, and the maximum amount of nitrosamines that is permitted in the composition comprising or consisting of ribociclib or pharmaceutically acceptable salt thereof is 0.6 ppm, relative to the total amount of ribociclib in free or salt form. Therefore, this aspect of the invention relates to a composition comprising or consisting of ribociclib or a pharmaceutically acceptable salt thereof which has been tested and found to have a total amount of nitrosamines (such as N-nitroso-ribociclib in free or salt form) that is no more than about 0.6 ppm, as required by the FDA. In another aspect, the regulatory authority is the EMA, and the maximum amount of nitrosamines that is permitted in the composition comprising or consisting of ribociclib or pharmaceutically acceptable salt thereof is 0.7 ppm, relative to the total amount of ribociclib in free or salt form. Therefore, this aspect of the invention relates to a composition comprising or consisting of ribociclib or a pharmaceutically acceptable salt thereof which has been tested and found to have a total amount of nitrosamines (such as N-nitroso-ribociclib in free or salt form) that is no more than about 0.7 ppm, as required by the EMA.

The composition comprising or consisting of ribociclib or a pharmaceutically acceptable salt thereof may contain a total amount of N-nitroso-ribociclib in free or salt form which is no more than about 5 ppm, relative to the total amount of ribociclib in free or salt form, for example no more than about 4 ppm, no more than about 3 ppm, no more than about 2 ppm, or no more than about 1 ppm, relative to the total amount of ribociclib in free or salt form. Preferably, the composition comprising or consisting of ribociclib or a pharmaceutically acceptable salt thereof may contain a total amount of N-nitroso-ribociclib in free or salt form which is no more than about 1 ppm, relative to the total amount of ribociclib in free or salt form. Thus, another aspect of the invention relates to a composition comprising or consisting of ribociclib or a pharmaceutically acceptable salt thereof, wherein the total amount of N-nitroso-ribociclib in free or salt form is no more than about 1 ppm, relative to the total amount of ribociclib in free or salt form. In some embodiments, the total amount of N-nitroso-ribociclib in free or salt form may be no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, no more than about 0.25 ppm, no more than about 0.2 ppm, no more than about 0.15 ppm, no more than about 0.1 ppm, no more than about 0.05 ppm, or no more than about 0.025 ppm, relative to the total amount of ribociclib in free or salt form. Preferably, the invention relates to a composition comprising or consisting of ribociclib or a pharmaceutically acceptable salt thereof, wherein the total amount of N-nitroso-ribociclib in free or salt form is no more than about 0.7 ppm, for example no more than about 0.6 ppm, or no more than about 0.4 ppm, relative to the total amount of ribociclib in free or salt form.

The total amount of N-nitroso-ribociclib in free or salt form in the composition comprising or consisting of ribociclib or a pharmaceutically acceptable salt thereof may result in no more than 400 ng/day of said N-nitroso-ribociclib in free or salt form being administered to the patient, when the composition is administered according to an approved dosage regimen of ribociclib or pharmaceutically acceptable salt thereof (for example, in the case that two 200 mg ribociclib tablets are administered per day, no more than 200 ng per tablet of said nitrosamines). Thus, another aspect of the invention relates to a composition comprising or consisting of ribociclib or a pharmaceutically acceptable salt thereof, wherein N-nitroso-ribociclib in free or salt form, if present, is present in the composition in an amount that results in no more than 400 ng/day of said N-nitroso-ribociclib in free or salt form being administered to the patient, when the composition is administered according to an approved dosage regimen of ribociclib or pharmaceutically acceptable salt thereof (for example, in the case that two 200 mg ribociclib tablets are administered per day, no more than 200 ng per tablet of said nitrosamines).

The composition comprising or consisting of ribociclib or a pharmaceutically acceptable salt thereof may contain an amount of total nitrosamines, including both N-nitroso-ribociclib in free or salt form and other nitrosamines, which is no more than about 5 ppm, for example no more than about 4 ppm, no more than about 3 ppm, no more than about 2 ppm, or no more than about 1 ppm. Preferably, the composition comprising or consisting of ribociclib or a pharmaceutically acceptable salt thereof may contain an amount of total nitrosamines, including both N-nitroso-ribociclib in free or salt form and other nitrosamines, which is no more than about 1 ppm. Thus, another aspect of the invention relates to a composition comprising or consisting of ribociclib or a pharmaceutically acceptable salt thereof, wherein the amount of total nitrosamines, including N-nitroso-ribociclib in free or salt form and other nitrosamines, is no more than about 1 ppm. In some embodiments, the amount of total nitrosamines, including both N-nitroso-ribociclib in free or salt form and other nitrosamines, may be no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, or no more than about 0.25 ppm. Preferably, the invention relates to a composition comprising or consisting of ribociclib or a pharmaceutically acceptable salt thereof, wherein the amount of total nitrosamines, including N-nitroso-ribociclib in free or salt form and other nitrosamines, is no more than about 0.7 ppm, for example no more than about 0.6 ppm or no more than about 0.4 ppm, relative to the total amount of ribociclib in free or salt form.

The amount of total nitrosamines, including both N-nitroso-ribociclib in free or salt form and other nitrosamines, in the composition comprising or consisting of ribociclib or a pharmaceutically acceptable salt thereof may result in no more than 400 ng/day of said total nitrosamines being administered to the patient, when the composition is administered according to an approved dosage regimen of ribociclib or pharmaceutically acceptable salt thereof (for example, in the case that two 200 mg ribociclib tablets are administered per day, no more than 200 ng per tablet of said nitrosamines). Thus, another aspect of the invention relates to a composition comprising or consisting of ribociclib or a pharmaceutically acceptable salt thereof, wherein the amount of total nitrosamines, including both N-nitroso-ribociclib in free or salt form and other nitrosamines, present in the composition results in no more than 400 ng/day of said total nitrosamines being administered to the patient, when the composition is administered according to an approved dosage regimen of ribociclib or pharmaceutically acceptable salt thereof (for example, in the case that two 200 mg ribociclib tablets are administered per day, no more than 200 ng per tablet of said nitrosamines).

The composition comprising or consisting of ribociclib or a pharmaceutically acceptable salt thereof may contain a total amount of NDMA, NDEA, NMPA, NDIPA, NIPEA, NDBA and/or NMBA, which is, in each case, no more than about 100 ppb. For example, the composition comprising or consisting of ribociclib or a pharmaceutically acceptable salt thereof may contain a total amount of N-nitrosodimethylamine (NDMA) which is no more than about 100 ppb. The composition may be substantially free, such as completely free, of NDMA. The composition may be substantially free, such as completely free, of NDMA, NDEA, NMPA, NDIPA, NIPEA, NDBA and NMBA.

As would be understood by one of ordinary skill in the art, when a maximum amount of a component is being specified, such as e.g., an amount or total amount of “no more than” a specified value x, then the reader is being urged not to exceed the specified value in order to increase the benefit that is obtained. One of ordinary skill in the art would therefore understand that in such embodiments, the variability that would otherwise be optionally associated with the given numerical value x, whether it is preceded by the term “about” or not, is preferably absent, i.e., any amount that is greater than x is not permitted. In such cases in which the option of variability is not exercised, the value x might alternatively be expressed as x±0%. Similar considerations apply mutatis mutandis for any reference to an amount which is “less than” a specified value x.

### Ribociclib

Ribociclib (also known as “LEE011” and referred to in the synthetic schemes included herein as “B10” or “LEE011-B10 (IP-1)”) is a potent and highly effective CDK4/6 inhibitor with in vivo activity against hormone receptor-positive, human epidermal growth factor receptor 2-negative (HR+/HER2−) breast cancer.

Ribociclib or its pharmaceutically acceptable salts can be present within the composition in solvated or unsolvated form, and references to “ribociclib or a pharmaceutically acceptable salt thereof” include both of these forms. In particular, references to ribociclib or a pharmaceutically acceptable salt thereof include ribociclib, a pharmaceutically acceptable salt of ribociclib, a pharmaceutically acceptable hydrate or solvate of ribociclib, and a pharmaceutically acceptable hydrate or solvate of a pharmaceutically acceptable salt of ribociclib. Typically, ribociclib is included as its succinate salt (ribociclib succinate, referred to in the synthetic schemes included herein as “B12” or “LEE011-B12 (IP)”), having the formula below:

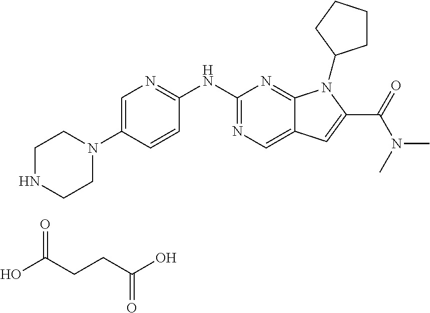

As used herein, and in the absence of a specific reference to a particular pharmaceutically acceptable salt and/or solvate of the compound of ribociclib (e.g., ribociclib succinate), any dosages, whether expressed in e.g., milligrams or as a % by weight, should be taken as referring to the amount of ribociclib free base, i.e., the amount of:

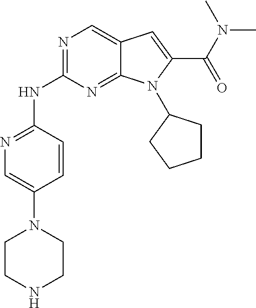 [SMILES: CN(C)C(=O)c1cc2cnc(Nc3ccc(N4CCNCC4)cn3)nc2n1C1CCCC1]

For example, therefore, a reference to “400 mg of ribociclib or a pharmaceutically acceptable salt thereof” means an amount of ribociclib or a pharmaceutically acceptable salt thereof which provides the same amount of ribociclib as 400 mg of ribociclib free base, i.e., 508.7 mg of ribociclib succinate. Similarly, a reference to a “200 mg ribociclib tablet” means a tablet which includes an amount of ribociclib or a pharmaceutically acceptable salt thereof which provides the same amount of ribociclib as 200 mg of ribociclib free base, i.e., 254.4 mg of ribociclib succinate.

The total amount of ribociclib or a pharmaceutically acceptable salt thereof included in the compositions of the invention may provide the same amount of ribociclib as about 50-1000 mg of ribociclib free base, e.g., about 100-1000 mg of ribociclib free base. The total amount of ribociclib or a pharmaceutically acceptable salt thereof included in the compositions of the invention may provide the same amount of ribociclib as about 50 mg, 100 mg, 200 mg, 400 mg, or 600 mg of ribociclib free base. The total amount of ribociclib or a pharmaceutically acceptable salt thereof included in the compositions of the invention may provide the same amount of ribociclib as about 200 mg of ribociclib free base. The total amount of ribociclib or a pharmaceutically acceptable salt thereof included in the compositions of the invention may provide the same amount of ribociclib as about 400 mg of ribociclib free base. The total amount of ribociclib or a pharmaceutically acceptable salt thereof included in the compositions of the invention may provide the same amount of ribociclib as about 600 mg ribociclib free base. Preferably, the total amount of ribociclib or a pharmaceutically acceptable salt thereof in the compositions of the invention provides the same amount of ribociclib as about 200 mg of ribociclib free base.

The ribociclib or pharmaceutically acceptable salt thereof included in the compositions of the invention may be in the solid form. The ribociclib or pharmaceutically acceptable salt thereof included in the compositions of the invention may be of any solid form. For example, the ribociclib or pharmaceutically acceptable salt thereof may be in amorphous or crystalline form. Examples of suitable crystalline forms of ribociclib and pharmaceutically acceptable salts thereof are provided in WO 2020/152629 and in U.S. Pat. No. 9,868,739, which are incorporated by reference in their entirety.

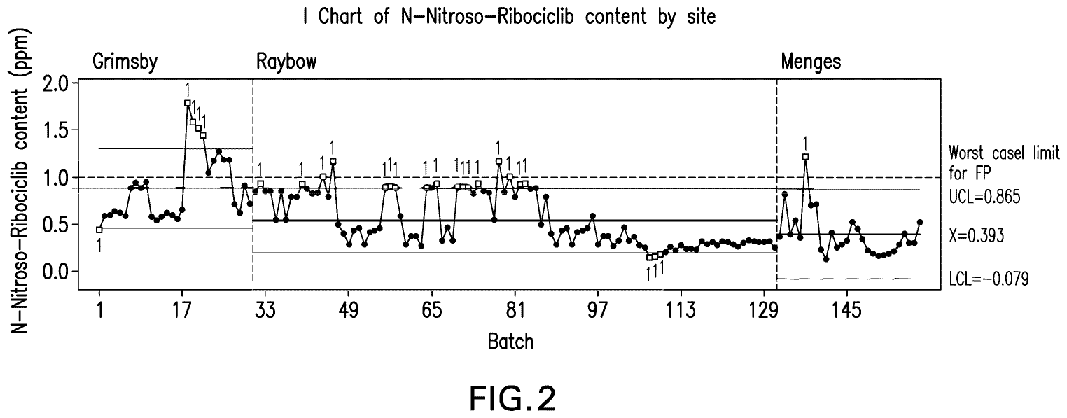

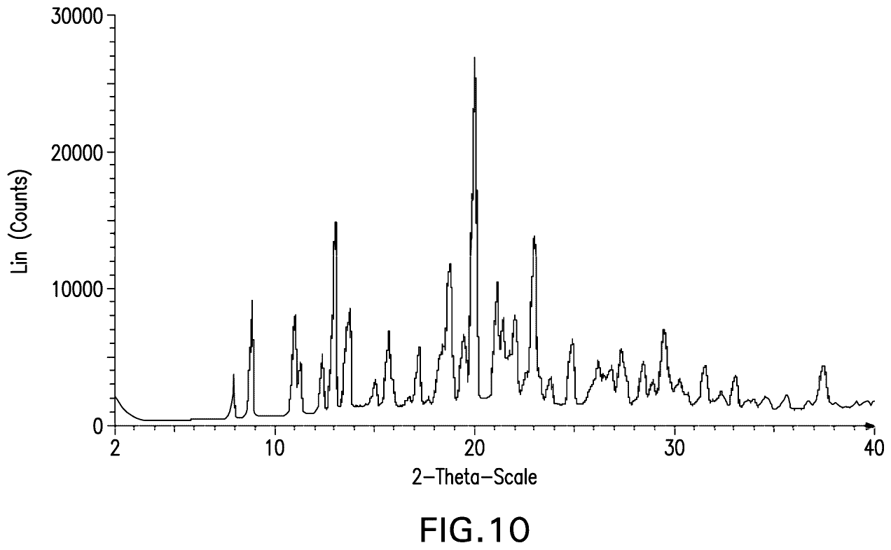

For example, the ribociclib or pharmaceutically acceptable salt thereof may be a crystalline form of ribociclib succinate. The crystalline form of ribociclib succinate may be Form E as described in WO 2020/152629, which is incorporated by reference in its entirety. In particular, Examples 2, 6, 12, 16, and 19 of WO 2020/152629 describe the formation and characterization of Form E of ribociclib succinate, and these examples are incorporated by reference. Thus, the ribociclib or pharmaceutically acceptable salt thereof included in the compositions of the invention may be characterized by an X-ray powder diffraction pattern having characteristic peaks expressed in degrees 2-Theta at angles of 11.0°+/−0.2°, 13.0+/−0.2°, and 17.2°+/−0.2°. The ribociclib or pharmaceutically acceptable salt thereof included in the compositions of the invention may be characterized by an X-ray powder diffraction patten having at least four characteristic peaks expressed in degrees 2-Theta selected from 11.0°+/−0.2°, 13.0+/−0.2°, 17.2°+/−0.2°, 20.0+/−0.2°, and 23.0+/−0.2°. The ribociclib or pharmaceutically acceptable salt thereof included in the compositions of the invention may be characterized by an X-ray powder diffraction patten having at least five characteristic peaks expressed in degrees 2-Theta selected from 8.8°+/−0.2°, 11.0°+/−0.2°, 13.0+/−0.2°, 13.7+/−0.2°, 15.7+/−0.2°, 17.2+/−0.2°, 18.7+/−0.2°, 20.0+/−0.2°, 21.1+/−0.2°, 23.0+/−0.2°, and 24.9°+/−0.2°. The ribociclib or pharmaceutically acceptable salt thereof included in the compositions of the invention may be characterized by an X-ray powder diffraction patten having at least seven characteristic peaks expressed in degrees 2-Theta selected from 7.9°+/−0.2°, 8.8°+/−0.2°, 11.0°+/−0.2°, 12.4°+/−0.2°, 13.0+/−0.2°, 13.7+/−0.2°, 15.7+/−0.2°, 17.2+/−0.2°, 18.7+/−0.2°, 20.0+/−0.2°, 21.1+/−0.2°, 23.0+/−0.2°, and 24.9°+/−0.2°. The ribociclib or pharmaceutically acceptable salt thereof included in the compositions of the invention may exhibit an X-ray powder diffraction pattern substantially in accordance with FIG. 10 (corresponding to FIG. 2 of WO 2020/152629) or the following table (corresponding to Table 4 of WO 2020/152629).

XRPD peak table for Form E of ribociclib succinate

| Angle (°2Θ) | Intensity (qualitative) |
|---|---|
| 7.9 | Low |
| 8.8 | Medium |
| 11.0 | Medium |
| 12.4 | Low |
| 13.0 | High |
| 13.7 | Medium |
| 15.7 | Medium |
| 17.4 | Low |
| 18.7 | Medium |
| 20.0 | High |
| 21.1 | Medium |
| 23.0 | High |
| 24.9 | Medium |

(High intensity > 50%; 50% > Medium > 20%; Low < 20%)
The value of each of the 2Θ values is accurate within ±0.2.

### Dosage Forms of the Invention

The compositions of the invention may be administered by any mode recognizable by the skilled person as being suitable. Illustrative examples of modes of administration include oral, nasal, parenteral, topical, transdermal, and rectal. Preferably, the compositions of the invention are orally administrable.

The pharmaceutical compositions of this invention may take any pharmaceutical form recognizable to the skilled person as being suitable. Suitable pharmaceutical forms include solid, semisolid, liquid, or lyophilized formulations, such as tablets, powders, capsules, suppositories, suspensions, liposomes, and aerosols. Preferably, the compositions of the invention may be administered in the form of a solid oral dosage form, for example a tablet, capsule, pill, or a patch. The composition of the invention may be administered in the form of a capsule, e.g. a biodegradable capsule. Alternatively, the composition of the invention may be administered in a solid oral dosage form which is not a capsule. Preferably, the composition of the invention may be administered in the form of a tablet.

Alternatively, the compositions of the invention may be in the form of a liquid dosage form, for example a solution or a suspension.

The compositions of the invention may be present in a sealed container. The compositions of the invention may further be under an atmosphere in which the humidity is no more than 2.5 g/kg (or the relative humidity is no more than 15%) and/or the percentage of oxygen is less than 21% by volume. The atmosphere may be air, or it may be an inert gas such as helium, nitrogen, argon, or neon. Preferably, the inert atmosphere is nitrogen.

In one embodiment, the composition may be provided as a plurality of oral dosage forms (e.g., the composition may be divided into a plurality of oral dosage forms) that are packaged within a blister pack such that each oral dosage form is individually packaged. Preferably the blister pack is substantially moisture and/or gas impermeable, more preferably such that each oral dosage form is individually packaged within a substantially moisture and/or gas impermeable blister. Materials for packaging that are substantially moisture and/or gas impermeable, e.g., materials that may be used to provide blister packs, are described elsewhere herein. Preferably, the substantially moisture and/or gas impermeable blister is an alu-alu blister.

The composition may be present in a sealed container which also contains a desiccant, or the composition may be present in a sealed pharmaceutical package which also contains a desiccant in a separate container. The composition may be stored in the presence of a desiccant. The desiccant may be selected from the following list: activated alumina, aerogel, benzophenone, bentonite clay, calcium chloride, calcium oxide, calcium sulphate, cobalt(II) chloride, copper(II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. Preferably, the desiccant may be selected from silica gel, molecular sieve, calcium oxide, and calcium sulphate. More preferably, the desiccant may be silica gel.

The compositions of the invention may be administered in the form of a solid oral dosage form, such as a tablet. For example, a pharmaceutical product may be provided which comprises a plurality of oral dosage forms, e.g., a plurality of tablets. The pharmaceutical product may comprise at least 5, at least 10, at least 15, at least 20, at least 25, at least 30, at least 35, at least 40, at least 45, at least 50 or at least 60 of said oral dosage forms, such as 7, 14, 21, 28, 35, 42, 49, 56, 63 or 70 of said oral dosage forms.

Each of the oral dosage forms in the pharmaceutical product may satisfy a requirement as specified herein for the total amount of nitrosamines and/or the total amount of N-nitroso-ribociclib in free or salt form that is present in a composition comprising ribociclib or a pharmaceutically acceptable salt thereof. Each of the oral dosage forms may satisfy the same requirement.

Each of the oral dosage forms, e.g., each of the tablets, may comprise or consist of ribociclib or a pharmaceutically acceptable salt thereof, wherein:
(i) each of the oral dosage forms is substantially free of N-nitroso-ribociclib in free or salt form,
(ii) the total amount of N-nitroso-ribociclib in free or salt form in each of the oral dosage forms is no more than 1 ppm, relative to the total amount of ribociclib in free or salt form, preferably wherein the total amount of N-nitroso-ribociclib in free or salt form in each of the oral dosage forms is no more than 0.7 ppm, more preferably no more than 0.6 ppm, relative to the total amount of ribociclib in free or salt form, or
(iii) the total amount of N-nitroso-ribociclib in free or salt form in each of the oral dosage forms is no more than the maximum amount of nitrosamines that is permitted in such a composition, by a regulatory authority, for example wherein the total amount of N-nitroso-ribociclib in free or salt form in each of the oral dosage forms is no more than the maximum amount of N-nitroso-ribociclib that is permitted in such a composition, by a regulatory authority.

As would be understood by one of ordinary skill in the art in the absence of any explicit statement to the contrary, each of the oral dosage forms within a pharmaceutical product will have the same composition, save for the fact that there may be some degree of variation between these oral dosage forms in terms of the total amount of nitrosamines and/or the total amount of N-nitroso-ribociclib in free or salt form. For example, each of these oral dosage forms may satisfy the same requirement for the total amount of nitrosamines and/or the total amount of N-nitroso-ribociclib (e.g., the total amount of N-nitroso-ribociclib in free or salt form in each of the oral dosage forms may be no more than 1 ppm, preferably no more than 0.7 ppm, and more preferably no more than 0.6 ppm, relative to the total amount of ribociclib in free or salt form in each of the oral dosage forms), but the exact total amount(s) in each of these oral dosage forms may vary between them (e.g., one or more of the oral dosage forms may be completely free of N-nitroso-ribociclib in free or salt form, and/or one or more of the oral dosage forms may comprise about 0.1 ppm of N-nitroso-ribociclib in free or salt form, and/or one or more of the oral dosage forms may comprise about 0.2 ppm of N-nitroso-ribociclib in free or salt form, etc.). As a person of ordinary skill in the art will readily appreciate, such variations may occur as a result of e.g., the different individual oral dosage forms within a particular pharmaceutical product being manufactured using different batches of ribociclib or of a pharmaceutical acceptable salt thereof and/or using different batches or sources of excipients.

### Pharmaceutical Products of the Invention

Another aspect of the invention relates to a pharmaceutical product comprising a composition of the invention and a document providing instructions to a patient as to how to administer the composition. The document may provide the instructions either directly or via a link to an electronic database.

The pharmaceutical product of the invention may comprise a document which certifies, either directly or via a link to an electronic database, that the total amount of nitrosamines (such as N-nitroso-ribociclib in free or salt form) in the composition is no more than about 5 ppm, for example no more than about 4 ppm, no more than about 3 ppm, no more than about 2 ppm, or no more than about 1 ppm. Preferably, the document may certify, either directly or via a link to an electronic database, that the total amount of nitrosamines (such as N-nitroso-ribociclib in free or salt form) in the composition is no more than about 1 ppm. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines (such as N-nitroso-ribociclib in free or salt form) in the composition is no more than about 1 ppm. For example, the document may certify, either directly or via a link to an electronic database, that the total amount of nitrosamines (such as N-nitroso-ribociclib in free or salt form) may be no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, no more than about 0.25 ppm. Preferably, an aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines (such as N-nitroso-ribociclib in free or salt form) in the composition is no more than about 0.7 ppm, for example no more than about 0.6 ppm.

The pharmaceutical product of the invention may comprise a document which certifies, either directly or via a link to an electronic database, that the total amount of nitrosamines (such as N-nitroso-ribociclib in free or salt form) in the composition results in no more than 400 ng/day of said nitrosamines being administered to the patient, when the composition is administered according to an approved dosage regimen of ribociclib or pharmaceutically acceptable salt thereof. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines (such as N-nitroso-ribociclib in free or salt form) in the composition results in no more than 400 ng/day of said nitrosamines being administered to the patient, when the composition is administered according to an approved dosage regimen of ribociclib or pharmaceutically acceptable salt thereof.

The pharmaceutical product of the invention may also comprise a document which certifies, either directly or via a link to an electronic database, that the total amount of nitrosamines in the composition is no more than the maximum amount of nitrosamines that is permitted in such a composition by a regulatory authority. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines in the composition is no more than the maximum amount of nitrosamines that is permitted in such a composition by a regulatory authority. In one of the above aspects, the document may certify, either directly or via a link to an electronic database, that the total amount of N-nitroso-ribociclib in free or salt form in the composition is no more than the maximum amount of N-nitroso-ribociclib that is permitted in such a composition by a regulatory authority. The maximum amount of nitrosamines (such as N-nitroso-ribociclib) that is permitted in such a composition by a regulatory authority may be the amount that is permitted at the time when the composition is prepared, released and/or administered to a patient. In one aspect, the regulatory authority is the US FDA, and the maximum amount of nitrosamines that is permitted in the composition comprising or consisting of ribociclib or pharmaceutically acceptable salt thereof is 0.6 ppm. Thus, such an aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines in the composition is no more than about 0.6 ppm, relative to the total amount of ribociclib in free or salt form. In another aspect, the regulatory authority is the EMA, and the maximum amount of nitrosamines that is permitted in the composition comprising or consisting of ribociclib or pharmaceutically acceptable salt thereof is 0.7 ppm. Thus, such an aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of nitrosamines in the composition is no more than about 0.7 ppm, relative to the total amount of ribociclib in free or salt form.

The pharmaceutical product of the invention may also comprise a document which certifies, either directly or via a link to an electronic database, that the total amount of N-nitroso-ribociclib in free or salt form is no more than about 5 ppm, relative to the total amount of ribociclib in free or salt form, for example no more than about 4 ppm, no more than about 3 ppm, no more than about 2 ppm, or no more than about 1 ppm, relative to the total amount of ribociclib in free or salt form. Preferably, the document may certify, either directly or via a link to an electronic database, that the total amount of N-nitroso-ribociclib in free or salt form is no more than about 1 ppm, relative to the total amount of ribociclib in free or salt form. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib in free or salt form is no more than about 1 ppm, relative to the total amount of ribociclib in free or salt form. The document may certify, either directly or via a link to an electronic database, that the total amount of N-nitroso-ribociclib in free or salt form is no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm, relative to the total amount of ribociclib in free or salt form. Preferably, an aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib in free or salt form is no more than about 0.7 ppm, for example no more than about 0.6 ppm, relative to the total amount of ribociclib in free or salt form.

The pharmaceutical product of the invention may comprise a document which certifies, either directly or via a link to an electronic database, that the total amount of N-nitroso-ribociclib in free or salt form in the composition results in no more than 400 ng/day of said N-nitroso-ribociclib in free or salt form being administered to the patient, when the composition is administered according to an approved dosage regimen of ribociclib or pharmaceutically acceptable salt thereof. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib in free or salt form is present in the composition results in no more than 400 ng/day of said N-nitroso-ribociclib in free or salt form being administered to the patient, when the composition is administered according to an approved dosage regimen of ribociclib or pharmaceutically acceptable salt thereof.

The pharmaceutical product of the invention may also comprise a document which certifies, either directly or via a link to an electronic database, that the amount of total nitrosamines, including both N-nitroso-ribociclib in free or salt form and other nitrosamines, is no more than about 5 ppm, for example no more than about 4 ppm, no more than about 3 ppm, no more than about 2 ppm, or no more than about 1 ppm. Preferably, the document may certify, either directly or via a link to an electronic database, that the amount of total nitrosamines, including both N-nitroso-ribociclib in free or salt form and other nitrosamines, is no more than about 1 ppm. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the amount of total nitrosamines, including both N-nitroso-ribociclib in free or salt form and other nitrosamines, is no more than about 1 ppm. The document may certify, either directly or via a link to an electronic database, that the amount of total nitrosamines, including both N-nitroso-ribociclib in free or salt form and other nitrosamines, is no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. Preferably, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the amount of total nitrosamines, including both N-nitroso-ribociclib in free or salt form and other nitrosamines, is no more than about 0.7 ppm, for example no more than about 0.6 ppm, relative to the total amount of ribociclib in free or salt form.

The pharmaceutical product of the invention may comprise a document which certifies, either directly or via a link to an electronic database, that the amount of total nitrosamines, including both N-nitroso-ribociclib in free or salt form and other nitrosamines, in the composition results in no more than 400 ng/day of said total nitrosamines being administered to the patient, when the composition is administered according to an approved dosage regimen of ribociclib or pharmaceutically acceptable salt thereof. Thus, another aspect of the invention relates to a pharmaceutical product containing: (a) a pharmaceutical composition comprising ribociclib or a pharmaceutically acceptable salt thereof; and (b) a document which certifies, either directly or via a link to an electronic database, that the amount of total nitrosamines, including both N-nitroso-ribociclib in free or salt form and other nitrosamines, present in the composition results in no more than 400 ng/day of total nitrosamines being administered to the patient, when the composition is administered according to an approved dosage regimen of ribociclib or pharmaceutically acceptable salt thereof.

The pharmaceutical product of the invention may comprise a document providing, either directly or via a link to an electronic database, instructions which specify how the composition should be stored, wherein if the composition is stored in accordance with the instructions, then the composition will be substantially free of N-nitroso-ribociclib in free or salt form.

The pharmaceutical product of the invention may comprise a document providing, either directly or via a link to an electronic database, instructions which specify how the composition should be stored, wherein if the composition is stored in accordance with the instructions, then the total amount of N-nitroso-ribociclib in free or salt form in the composition will be no more than the maximum amount of nitrosamines that is permitted in such a composition, by a regulatory authority, optionally wherein the total amount of N-nitroso-ribociclib in free or salt form in the composition will be no more than the maximum amount of N-nitroso-ribociclib that is permitted in such a composition, by a regulatory authority.

The pharmaceutical product of the invention may comprise a document providing, either directly or via a link to an electronic database, instructions which specify how the composition should be stored, wherein if the composition is stored in accordance with the instructions, then the total amount of N-nitroso-ribociclib in free or salt form in the composition will be no more than 5 ppm, relative to the total amount of ribociclib in free or salt form, for example wherein the total amount of N-nitroso-ribociclib in free or salt form in the composition is or will be no more than about 4 ppm, no more than about 3 ppm, no more than about 2 ppm, no more than about 1 ppm, no more than about 0.9 ppm, no more than about 0.8 ppm, no more than about 0.7 ppm, no more than about 0.65 ppm, no more than about 0.6 ppm, no more than about 0.55 ppm, no more than about 0.5 ppm, no more than about 0.45 ppm, no more than about 0.4 ppm, no more than about 0.35 ppm, no more than about 0.3 ppm, or no more than about 0.25 ppm, relative to the total amount of ribociclib in free or salt form. Preferably, if the composition is stored in accordance with the instructions, then the total amount of N-nitroso-ribociclib in free or salt form is or will be no more than about 1 ppm, no more than about 0.7 ppm, or no more than about 0.6 ppm, relative to the total amount of ribociclib in free or salt form.

The document which provides the instructions which specify how the composition should be stored may specify a set of storage conditions comprising a storage time of no more than 60 months, optionally no more than 54, no more than 48, no more than 42, no more than 36, no more than 30, no more than 24, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 months and a temperature of no more than 25° C., optionally wherein the specified temperature is about 2 to about 8° C., or about 20° C. to about 25° C. Optionally, the instructions specify: (i) a first set of storage conditions; and (ii) a second set of storage conditions for use after the first set of storage conditions. In this embodiment, the first set of storage conditions may comprise a storage time of no more than 60 months, optionally no more than 54, no more than 48, no more than 42, no more than 36, no more than 30, no more than 24, no more than 18, no more than 17, no more than 16, no more than 15, no more than 14, no more than 13, no more than 12, or no more than 11 months, and a temperature of about 2 to about 8° C., and/or the second set of storage conditions may comprise a storage time of no more than 10 months, optionally no more than 9, no more than 8, no more than 7, no more than 6, no more than 5, no more than 4, no more than 3, no more than 2, or no more than 1 months, and a temperature of no more than 25° C.

The pharmaceutical products of the invention may comprise a document providing instructions to a patient as to how to administer the composition.

The document which provides the certification as herein described may also be the document which provides the instructions to the patient. Alternatively, the document which provides the certification as described herein may be a separate document from that which provides the instructions. The document which provides the certification may be a component of the packaging of the composition, such as a label, a blister pack, a box, or a carton. A certification via a link to an external database may be e.g., a numerical or alphanumerical identifier, a barcode, or a QR code. An expiry date of the composition, e.g., the end of the shelf life of the composition, may be presented on the document that provides the certification.

A plurality of the pharmaceutical products as herein described may be provided, optionally wherein the pharmaceutical products are packaged together. The plurality may comprise at least 5, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900 or at least 1000 of said pharmaceutical products. One or more (e.g., each) of said pharmaceutical products may comprise one or any combination of:
a. a document providing, either directly or via a link to an electronic database, instructions to a patient as to how to administer the composition. The document may be as described herein.
b. a document providing, either directly or via a link to an electronic database, instructions which specify how the composition should be stored. The document may be as described herein.
c. a document which certifies, either directly or via a link to an electronic database, that the total amount of N-nitroso-ribociclib in free or salt form in the composition is no more than 1 ppm, relative to the total amount of ribociclib in free or salt form. The document may be as described herein.
d. a document which indicates that, provided that the composition is stored in accordance with a set of instructions, the total amount of N-nitroso-ribociclib in free or salt form in the composition will be no more than 1 ppm, relative to the total amount of ribociclib in free or salt form. The document may be as described herein.

The pharmaceutical products as herein described may further comprise means to prevent, retard or reduce the formation of N-nitroso-ribociclib in free or salt form in the composition. The means may be one or any combination of the means described herein. For example, the means may be one or any combination of:
a. the composition is present in a sealed container under an atmosphere in which the percentage of oxygen is less than 21% by volume, optionally wherein the atmosphere is nitrogen;
b. the composition is present in a sealed container which also contains a desiccant; or the composition is present in a sealed pharmaceutical package which also contains a desiccant in a separate container;
c. the composition comprises one or more pharmaceutically acceptable additives which prevent, retard or reduce the formation of N-nitroso-ribociclib in free or salt form, optionally wherein each of the one or more additives is selected from the group consisting of pH adjusters, antioxidants, radical scavenging agents, and peroxide-quenching agents;
d. the composition is present in a sealed container under an atmosphere in which the relative humidity is less than 75%, optionally wherein the relative humidity is less than 60%, and preferably wherein the relative humidity is no more than 15%, e.g., no more than 2.5 g/kg.

For example, in the case of a pharmaceutical product comprising a plurality of oral dosage forms (e.g., a plurality of tablets), then one or any combination of the following may apply:
a. the oral dosage forms are each individually or together present in a sealed container under an atmosphere in which the percentage of oxygen is less than 21% by volume, optionally wherein the atmosphere is nitrogen;
b. the oral dosage forms are each individually or together present in a sealed container which also contains a desiccant; or the oral dosage forms are together present in a sealed pharmaceutical package which also contains a desiccant in a separate container;
c. the oral dosage forms each further comprise one or more pharmaceutically acceptable additives which prevent, retard or reduce the formation of N-nitroso-ribociclib in free or salt form, optionally wherein each of the one or more additives is selected from the group consisting of pH adjusters, antioxidants, radical scavenging agents, and peroxide-quenching agents.

Additionally, or alternatively, and preferably in the case of a pharmaceutical product comprising a plurality of oral dosage forms (e.g., a plurality of tablets), the oral dosage forms are packaged within a blister pack such that each oral dosage form is individually packaged within a substantially moisture and/or gas impermeable blister, preferably wherein the humidity of the atmosphere within each blister is no more than 2.5 g/kg. Materials that are substantially moisture and/or gas impermeable and that may be used to provide blister packs are described elsewhere herein. Preferably, the blister pack may be an alu-alu blister pack.

### Medical Uses

An aspect of the invention provides compositions or pharmaceutical products of the invention for use in methods of therapy. The compositions or pharmaceutical products of the invention may be for use in the treatment of cancer, in particular breast cancer. The methods may comprise administering a therapeutically effective amount of ribociclib or a pharmaceutically acceptable salt thereof in a composition or pharmaceutical product of the invention to a patient in need thereof.

The breast cancer may be locally advanced and/or metastatic breast cancer. Metastatic breast cancer (also known as stage 4 breast cancer) refers to breast cancer which has metastasized to other organs in the body. Thus, the compositions and pharmaceutical products of the invention may be for use in the treatment of advanced and/or metastatic breast cancer.

Alternatively, the breast cancer may be early breast cancer (EBC). Early breast cancer refers to breast cancer that is contained within the breast, and which may or may not have spread to the lymph nodes in the armpit. Early breast cancer includes stage I, stage II and stage III breast cancer. The compositions and pharmaceutical products of the invention may be particularly useful in the treatment of early breast cancer.

The breast cancer may be hormone receptor-positive (HR+) and/or human epidermal growth factor receptor 2-negative (HER2−). The breast cancer may be HR+/HER2− breast cancer. Thus, the compositions and pharmaceutical products of the invention may be for use in treating HR+/HER2− breast cancer, optionally HR+/HER2− advanced and/or metastatic breast cancer or HR+/HER2− early breast cancer. The compositions and pharmaceutical products of the invention may be particularly useful for treating HR+/HER2− early breast cancer.

An example of advanced breast cancer in the context of this invention is therefore hormone receptor-positive, human epidermal growth factor receptor 2-negative (HR+/HER2−) locally advanced or metastatic breast cancer.

An example of early breast cancer that is particularly relevant in the context of this invention is stage II or III hormone receptor-positive, human epidermal growth factor receptor 2-negative (HR+/HER2−) early breast cancer.

It will be understood by a person skilled in the art that the invention also encompasses a method of treatment, including a method of treating cancer, in particular breast cancer as described herein, by administering to a patient in need thereof a therapeutically effective amount of ribociclib or a pharmaceutically acceptable salt thereof in a composition or pharmaceutical product of the invention. The invention also encompasses a pharmaceutical composition or pharmaceutical product comprising ribociclib or a pharmaceutically acceptable salt thereof for use in therapy, including for use in the treatment of cancer, in particular breast cancer as described herein. The invention also encompasses the use of a composition or pharmaceutical product of the invention for the manufacture of a medicament for the treatment of a disease, including for the treatment of cancer, in particular breast cancer as described herein. The invention also encompasses the use of a composition or pharmaceutical product of the invention for therapy, including for treating cancer, in particular breast cancer as described herein.

Such methods of the invention may be for treating advanced and/or metastatic breast cancer. Alternatively, such methods of the invention may be for treating early breast cancer. Methods of the invention for treating early breast cancer may be particularly useful.

The breast cancer may be hormone receptor-positive (HR+) and/or human epidermal growth factor receptor 2-negative (HER2−). The breast cancer may be HR+ and HER2− breast cancer. Thus, the methods of the invention may be for treating HR+/HER2− breast cancer, optionally HR+/HER2− advanced and/or metastatic breast cancer or HR+/HER2− early breast cancer. Methods of the invention for treating HR+/HER2− early breast cancer may be particularly useful.

The patient in need of treatment with the compositions or pharmaceutical products of the invention may be a woman, for example a woman with breast cancer. The patient may be a woman with locally advanced and/or metastatic breast cancer. Alternatively, the patient may be a woman with early breast cancer. The patient may be a woman with HR+/HER2− breast cancer, for example HR+/HER2− advanced and/or metastatic breast cancer or HR+/HER2− early breast cancer.

The patient may also be receiving initial endocrine-based therapy, e.g., an aromatase inhibitor or fulvestrant. For example, the compositions or pharmaceutical products of the invention may be administered in combination with an aromatase inhibitor or fulvestrant as initial endocrine-based therapy. Alternatively, the patient may have received prior endocrine therapy. Suitable aromatase inhibitors are letrozole, exemestane or anastrozole. In particular in the context of early breast cancer, the patient may receive ribociclib or a pharmaceutically acceptable salt thereof in combination with endocrine therapy. Examples of suitable combination therapies are provided in WO 2015/022609 A1, which is incorporated by reference in its entirety.

The patient may be a woman who is pre-or peri-menopausal. In such patients, the initial endocrine-based therapy may be combined with a luteinising hormone-releasing hormone (LHRH) agonist.

In the case of oral administration, the daily dose of ribociclib or a pharmaceutically acceptable salt thereof may provide the same amount of ribociclib as a daily dose of about 200 mg to about 600 mg of ribociclib free base. For example, the daily dose of ribociclib or a pharmaceutically acceptable salt thereof may provide the same amount of ribociclib as a daily dose of about 200 mg, about 400 mg, or about 600 mg of ribociclib free base. Preferably, the daily dose of ribociclib or a pharmaceutically acceptable salt thereof may provide the same amount of ribociclib as about 400 mg or about 200 mg, e.g., about 400 mg, of ribociclib free base, in particular when used in the treatment of early breast cancer as described herein. Alternatively, the daily dose of ribociclib or a pharmaceutically acceptable salt thereof may provide the same amount of ribociclib as about 600 mg, about 400 mg, or about 200 mg, e.g., about 600 mg, of ribociclib free base, in particular when used in the treatment of advanced or metastatic breast cancer as described herein. The mg dose amounts refer to the total daily dose.

The total daily dose may be administrated once daily for about 21 consecutive days followed by about 7 days off treatment, resulting in a complete cycle of about 28 days. The treatment may be continued as long as the patient is deriving clinical benefit from therapy or until unacceptable toxicity occurs. The dose may be reduced when toxicity occurs. For example, a dose of 600 mg may be reduced to 400 mg or 200 mg. A dose of 400 mg may be reduced to 200 mg.

In the case of oral administration, the compositions of the invention may, as a result of the administration, provide an amount of nitrosamines (such as N-nitroso-ribociclib in free or salt form) to the patient that corresponds to an amount which is no more than about 1500 ng/day of nitrosamine free base (such as N-nitroso-ribociclib free base), for example no more than 400 ng/day, no more than about 300 ng/day, no more than about 200 ng/day, no more than about 100 ng/day, or no more than about 26.5 ng/day. The compositions of the invention may, as a result of their administration, provide an amount of N-nitroso-ribociclib in free or salt form to the patient that corresponds to an amount which is no more than about 400 ng/day of N-nitroso-ribociclib free base, for example no more than about 300 ng/day, no more than about 200 ng/day, no more than about 100 ng/day, or no more than about 26.5 ng/day. The compositions of the invention may, as a result of their administration, provide an amount of total nitrosamines, including both N-nitroso-ribociclib in free or salt form and any other nitrosamines, to the patient that corresponds to an amount which is no more than about 400 ng/day of nitrosamine free base, for example no more than about 300 ng/day, no more than about 200 ng/day, no more than about 100 ng/day, or no more than about 26.5 ng/day. In particular, the compositions of the invention may, as a result of their administration, provide an amount of N-nitroso-ribociclib in free or salt form to the patient that corresponds to an amount which is no more than about 200 ng/day of N-nitroso-ribociclib free base.

The patient to be treated may receive, as a result of the administration of the composition in accordance with the treatment, a daily total amount of nitrosamines (such as N-nitroso-ribociclib in free or salt form) that corresponds to an amount of no more than about 400 ng of nitrosamine free base (such as N-nitroso-ribociclib free base), for example of no more than about 300 ng, no more than about 200 ng, no more than about 100 ng, or no more than about 26.5 ng. Thus, the patient to be treated may receive, as a result of the administration of the composition in accordance with the treatment, a daily total amount of N-nitroso-ribociclib in free or salt form that corresponds to an amount of no more than about 400 ng of N-nitroso-ribociclib free base. The patient to be treated may also receive, as a result of the administration of the composition in accordance with the treatment, a daily total amount of total nitrosamines, including both N-nitroso-ribociclib in free or salt form and any other nitrosamines, that corresponds to an amount of no more than about 400 ng of nitrosamine free base, for example of no more than about 300 ng, no more than about 200 ng, no more than about 100 ng, or no more than about 26.5 ng.

### Manufacture of the Drug Substance

The invention further provides methods of preparing ribociclib or a pharmaceutically acceptable salt thereof which is substantially free of nitrosamines, particularly N-nitroso-ribociclib in free or salt form. While the methods are described separately below, it will be readily apparent to a person of ordinary skill in the art that two or more of these methods may also be used in combination. Thus, the present invention also provides these combined methods of preparing ribociclib or a pharmaceutically acceptable salt thereof. The ribociclib or pharmaceutically acceptable salt thereof produced by these methods may be used in the compositions and pharmaceutical products of the invention as described herein.

An aspect of the invention relates to a method wherein (i) the reagents used to prepare the ribociclib or a pharmaceutically acceptable salt thereof and/or (ii) the intermediates used to prepare the ribociclib or a pharmaceutically acceptable salt thereof are independently substantially free of nitrites.

An aspect of the invention relates to a method comprising the following deprotection step in which the amount of nitrites in the potassium carbonate is no more than about 5 ppm, for example no more than about 4 ppm, no more than about 3 ppm, no more than about 2 ppm, or no more than about 1 ppm. Preferably, the amount of nitrites in the potassium carbonate is no more than about 1 ppm. The amount of nitrites in the potassium carbonate may optionally be no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm.

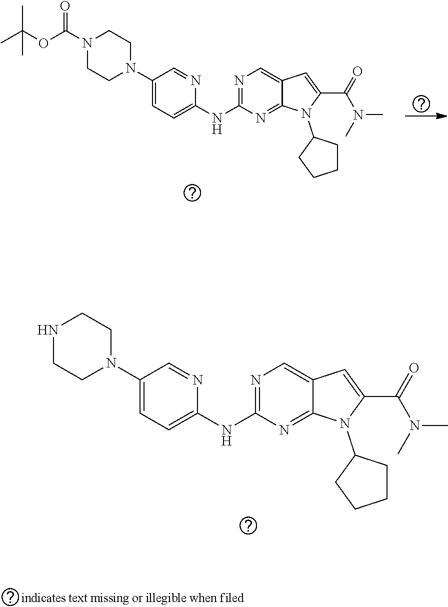 [SMILES: CN(C)C(=O)c1cc2cnc(Nc3ccc(N4CCN(C(=O)OC(C)(C)C)CC4)cn3)nc2n1C1CCCC1 ; CN(C)C(=O)c1cc2cnc(Nc3ccc(N4CCNCC4)cn3)nc2n1C1CCCC1]

### Manufacture of a Pharmaceutically Acceptable Salt of Ribociclib

An aspect of the invention relates to a method of preparing a pharmaceutically acceptable salt of ribociclib which comprises using a ribociclib free base which is substantially free of nitrites. For example, the amount of nitrites in the ribociclib free base may be no more than about 5 ppm, for example no more than about 4 ppm, no more than about 3 ppm, no more than about 2 ppm, no more than about 1 ppm, no more than about 0.5 ppm, no more than about 0.25 ppm, no more than about 0.1 ppm, less than about 0.1 ppm, less than about 0.05 ppm, or less than about 0.025 ppm. Preferably, the amount of nitrites in the ribociclib free base is less than about 0.1 ppm.

The method may comprise forming the salt under acidic conditions. For example, organic or inorganic acids may be reacted with the ribociclib base. Suitable inorganic acids are, for example, halogen acids, such as hydrochloric acid, sulfuric acid, or phosphoric acid. Suitable organic acids are, e.g., succinic acid, carboxylic acids or sulfonic acid, such as fumaric acid or methanesulfonic acid. The organic or inorganic acid used may be substantially free of nitrites. For example, the amount of nitrites in the organic or inorganic acid (such as succinic acid) may be no more than about 5 ppm, for example no more than about 4 ppm, no more than about 3 ppm, no more than about 2 ppm, or no more than about 1 ppm. Preferably, the amount of nitrites in the organic or inorganic acid (such as succinic acid) is no more than about 1 ppm. The amount of nitrites in the organic or inorganic acid (such as succinic acid) may be no more than about 0.5 ppm, no more than about 0.25 ppm, no more than about 0.1 ppm, less than about 0.1 ppm, less than about 0.05 ppm, or less than about 0.025 ppm.

In certain embodiments, the amount of nitrites in the ribociclib free base and the amount of nitrites in the organic or inorganic acid (such as succinic acid) may each be no more than about 5 ppm, for example no more than about 4 ppm, no more than about 3 ppm, no more than about 2 ppm, no more than about 1 ppm, no more than about 0.5 ppm, no more than about 0.25 ppm, no more than about 0.1 ppm, less than about 0.1 ppm, less than about 0.05 ppm, or less than about 0.025 ppm.

As disclosed elsewhere herein, the amount of nitrites in a composition, e.g., the amount of nitrites in the ribociclib free base and/or the amount of nitrites in the organic or inorganic acid (such as succinic acid) may be determined using the Griess test. Accordingly, the invention provides a method of preparing ribociclib or a pharmaceutically acceptable salt thereof, wherein the method comprises the use of the Griess test to determine the amount of nitrites in a composition comprising ribociclib free base and/or the amount of nitrites in an organic or inorganic acid (such as succinic acid).

### Solvent Oxygen Content

An aspect of the invention relates to a method of preparing ribociclib or a pharmaceutically acceptable salt thereof with an acid, wherein the method comprises the use of one or more solvents which, immediately prior to their use in the method, are stored under an atmosphere in which the percentage of oxygen is less than 21% by volume.

The method may comprise the use of said one or more solvents for dissolving ribociclib and/or said acid. Furthermore, the method may be for preparing a pharmaceutically acceptable salt of ribociclib with an acid, wherein the method comprises the use of said one or more solvents for dissolving ribociclib and said acid, respectively, prior to their combination to form the pharmaceutically acceptable salt of ribociclib. Thus, an aspect of the invention relates to a method of preparing ribociclib or a pharmaceutically acceptable salt thereof with an acid, wherein the method comprises: (a) obtaining one or more solvents, where the one or more solvents are contained in a sealed container under an atmosphere in which the percentage of oxygen is less than 21% by volume; and (b) using the one or more solvents to dissolve ribociclib and/or said acid. Preferably, the one or more solvents are used shortly after, such as immediately after, the sealed container is unsealed, e.g., within 12 hours, within 9 hours, within 6 hours, or within 3 hours.

The atmosphere may be an inert gas such as helium, nitrogen, argon, or neon. Preferably, the atmosphere is nitrogen.

The one or more solvents may be substantially free of nitrites. The total amount of nitrites in each of the one or more solvents may be no more than about 5 ppb, relative to the amount of the respective solvent, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the amount of the respective solvent. Preferably, the total amount of nitrites in each of the one or more solvents may be no more than about 5 ppb, no more than about 3.5 ppb, or no more than about 1 ppb, relative to the amount of the respective solvent. More preferably, the total amount of nitrites in each of the one or more solvents may be no more than about 1 ppb, relative to the amount of the respective solvent.

Where there is more than one solvent, the combined total amount of nitrites in the solvents may be no more than about 5 ppb, relative to the combined amount of the solvents, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the combined amount of the solvents. Preferably, the combined total amount of nitrites in the solvents may be no more than about 5 ppb, no more than about 3.5 ppb, or no more than about 1 ppb, relative to the combined amount of the solvents. More preferably, the combined total amount of nitrites in the solvents may be no more than about 1 ppb, relative to the combined amount of the solvents.

As disclosed elsewhere herein, the amount of nitrites in a composition, e.g., the amount of nitrites in each of the one or more solvents, may be determined using the Griess test. Accordingly, the invention provides a method of preparing a composition comprising ribociclib or a pharmaceutically acceptable salt thereof, wherein the method comprises the use of the Griess test to determine the amount of nitrites in each of one or more solvents that are used in the method.

The one or more solvents may be isopropanol, n-propanol, methyl tert-butyl ether, ethyl acetate, isopropyl acetate, or ethanol. The one or more solvents may be or may comprise isopropanol, optionally wherein the isopropanol is substantially free of isopropyl nitrite, e.g., the amount of isopropyl nitrite in the isopropanol is no more than one of the individual values presented above for the total amount of nitrites in each of the one or more solvents, e.g., preferably less than about 1 ppb. By way of example, the one or more solvents may be or may comprise isopropanol and water, for example the one or more solvents may be or may comprise a solvent mixture of isopropanol and water with at least 10%, at least 20 vol %, at least 30 vol %, at least 40 vol %, at least 50 vol %, at least 60 vol %, and preferably at least 70 vol % isopropanol. Preferably, the solvent consists of isopropanol. Alternatively, the one or more solvents may not be or may not comprise isopropanol.

### Solvent Distillation

Another aspect of the invention relates to a method of preparing ribociclib or a pharmaceutically acceptable salt thereof, wherein the method comprises the use of one or more solvents, and wherein the method comprises distilling said one or more solvents to reduce their content of nitrites, prior to their use. Accordingly, the invention also provides a method of preparing ribociclib or a pharmaceutically acceptable salt thereof, wherein the method comprises distilling one or more solvents to reduce their content of nitrites.

The ribociclib or a pharmaceutically acceptable salt thereof may be prepared using an acid. The method of preparation may comprise the use of one or more solvents for dissolving ribociclib and/or said acid, and the method may comprise distilling the one or more solvents to reduce their content of nitrites, prior to said use. Thus, an aspect of the invention relates to a method of preparing ribociclib or a pharmaceutically acceptable salt thereof with an acid, wherein the method comprises the use of one or more solvents for dissolving ribociclib and/or said acid, and wherein the method comprises distilling the one or more solvents to reduce their content of nitrites, prior to said use.

The ribociclib or a pharmaceutically acceptable salt thereof may be prepared using an acid and the method of preparation may comprise: (a) obtaining one or more solvents, (b) distilling said one or more solvents to reduce their content of nitrites; and (c) using the one or more distilled solvents to dissolve ribociclib and/or said acid. Thus, another aspect of the invention relates to a method of preparing ribociclib or a pharmaceutically acceptable salt thereof with an acid, wherein the method comprises: (a) obtaining one or more solvents; (b) distilling said one or more solvents to reduce their content of nitrites; and (c) using the one or more distilled solvents to dissolve ribociclib and/or said acid.

The one or more solvents, prior to and/or after distillation, may be contained in a sealed container under an atmosphere in which the percentage of oxygen is less than 21% by volume. The atmosphere may be an inert gas such as helium, nitrogen, argon, or neon. Preferably, the atmosphere is nitrogen.

The total amount of nitrites in the or each solvent, following the distillation, may be no more than about 5 ppb, relative to the amount of the respective solvent, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the amount of the respective solvent. Preferably, the total amount of nitrites in the or each solvent, following the distillation, may be no more than about 5 ppb, no more than about 3.5 ppb, no more than about 1 ppb, or less than about 1 ppb, relative to the amount of the respective solvent. More preferably, the total amount of nitrites in the or each solvent, following the distillation, may be no more than about 1 ppb, relative to the amount of the respective solvent.

Where there is more than one solvent, the combined total amount of nitrites in the solvents, following the distillation, may be no more than about 5 ppb, relative to the combined amount of the solvents, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the combined amount of the solvents. Preferably, the combined total amount of nitrites in the solvents, following the distillation, may be no more than about 5 ppb, no more than about 3.5 ppb, or no more than about 1 ppb, relative to the combined amount of the solvents. More preferably, the combined total amount of nitrites in the solvents, following the distillation, may be no more than about 1 ppb, relative to the combined amount of the solvents.

The distillation may reduce the total amount of nitrites in the one or more solvents, e.g., in each of the solvents that are distilled, by at least 5%, at least 10%, at least 15%, at least 20%, at least 30%, at least 40%, at least 50%, at least 60%, or at least 70%, relative to the total amount of nitrites in the solvent(s) prior to the distillation. Where there is more than one solvent, the distillation may reduce the combined total amount of nitrites in the solvents that are distilled by at least 5%, at least 10%, at least 15%, at least 20%, at least 30%, at least 40%, at least 50%, at least 60%, or at least 70%, relative to the combined total amount of nitrites in the solvents prior to the distillation.

The one or more solvents, following the distillation, may be substantially free of nitrites.

The total amount of nitrites in each of the one or more solvents may be no more than about 5 ppb, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb. Preferably, the total amount of nitrites in each of the one or more solvents may be no more than about 5 ppb, no more than about 3.5 ppb, no more than about 1 ppb, or less than about 1 ppb. More preferably, the total amount of nitrites in each of the one or more solvents may be less than about 1 ppb.

Where there is more than one solvent, the combined total amount of nitrites in the solvents may be no more than about 5 ppb, relative to the combined amount of the solvents, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb. Preferably, the combined total amount of nitrites in the solvents may be no more than about 5 ppb, no more than about 3.5 ppb, or no more than about 1 ppb. More preferably, the combined total amount of nitrites in the solvents may be no more than about 1 ppb.

As disclosed elsewhere herein, the amount of nitrites in a composition, e.g., the amount of nitrites in a solvent prior to and/or following its distillation, may be determined using the Griess test. Accordingly, the invention provides a method of preparing a composition comprising ribociclib or a pharmaceutically acceptable salt thereof, wherein the method comprises the use of the Griess test to determine the amount of nitrites in each of one or more solvents that are used in the method, prior to and/or following their distillation.

The one or more solvents may be isopropanol, n-propanol, methyl tert-butyl ether, ethyl acetate, isopropyl acetate, or ethanol. The one or more solvents may be or may comprise isopropanol, optionally wherein the isopropanol is (i.e., becomes as a result of the distillation, or having fulfilled the criterion before the distillation is also compliant with the criterion after the distillation) substantially free of isopropyl nitrite, e.g., the amount of isopropyl nitrite in the isopropanol is no more than one of the individual values presented above for the total amount of nitrites in the one or more solvents, e.g., preferably no more than 1 ppb. By way of example, the one or more solvents may be or may comprise isopropanol and water, for example the one or more solvents may be or may comprise a solvent mixture of isopropanol and water with at least 10%, at least 20 vol %, at least 30 vol %, at least 40 vol %, at least 50 vol %, at least 60 vol %, and preferably at least 70 vol % isopropanol. Preferably, the solvent consists of isopropanol. Alternatively, the one or more solvents may not be or may not comprise isopropanol.

The distillation may be over and/or through ascorbic acid, preferably over ascorbic acid. Ascorbic acid 10% may be used.

Following the distillation, the method may further comprise: (a) testing different fractions of the distillate for the presence and/or total amount of nitrites; (b) identifying the fraction(s) with the lowest amount of nitrites; and (c) using said fraction(s) with the lowest amount of nitrites as the one or more distilled solvents in the step of dissolving the ribociclib and/or said acid.

Dissolving ribociclib and/or said acid using the one or more distilled solvents may be performed by mixing the ribociclib and/or said acid with the one or more distilled solvents. The mixing may be performed by adding the components to a mixing vessel. The (i) ribociclib and/or said acid and the (ii) one or more distilled solvents may be added to the mixing vessel simultaneously, or they may be added subsequently, in any order. The ribociclib and/or said acid may be added last to the mixing vessel. The one or more distilled solvents may be added first to the mixing vessel, and the ribociclib and/or said acid may be added subsequently to the mixing vessel.

### Ion Exchange Resins

Another aspect of the invention relates to a method of preparing ribociclib or a pharmaceutically acceptable salt thereof, wherein the method comprises the use of one or more solvents, and wherein the method comprises passing said one or more solvents through an ion exchange resin that is capable of adsorbing nitrites. Accordingly, the invention also provides a method of preparing ribociclib or a pharmaceutically acceptable salt thereof, wherein the method comprises passing one or more solvents through an ion exchange resin that is capable of adsorbing nitrites.

Another aspect of the invention relates to a method of preparing a pharmaceutically acceptable salt of ribociclib with an acid, wherein the method comprises mixing a first solution of ribociclib with a second solution of said acid. The mixing of the first and second solutions may be performed by adding the two solutions to a mixing vessel. The first and second solutions may be added to the mixing vessel simultaneously, or may be added subsequently, in any order. Preferably, the first solution (i.e., of ribociclib) may be added last to the mixing vessel. Thus, in some aspects, the second solution (i.e., of the acid) is added first to the mixing vessel, and the first solution (i.e., of ribociclib) is added subsequently to the mixing vessel.

The method may comprise one or more steps of reducing the content of nitrites in said first and/or second solutions, prior to their combination. The one or more steps may involve passing the first and/or second solutions through an ion exchange resin that is capable of adsorbing nitrites.

The preparation method may comprise the use of one or more solvents for dissolving ribociclib and/or dissolving an acid that will be used to make a pharmaceutically acceptable salt of ribociclib. The method may further comprise passing the one or more solvents through an ion exchange resin which is capable of adsorbing nitrites, prior to said use. Thus, an aspect of the invention relates to a method of preparing ribociclib or a pharmaceutically acceptable salt thereof with an acid, wherein the method comprises the use of one or more solvents for dissolving ribociclib and/or said acid, and wherein the method comprises passing the one or more solvents through an ion exchange resin which is capable of adsorbing nitrites, prior to said use.

The method may further comprise: (a) distilling the one or more solvents to reduce their content of nitrites; and (b) passing the one or more distilled solvents through said ion exchange resin. Alternatively, the method may further comprise: (a) passing the one or more solvents through said ion exchange resin; and (b) distilling the one or more solvents to reduce their content of nitrites. Accordingly, the step of passing the one or more solvents through an ion exchange resin may occur before, after, or both before and after the step of distillation.

The method of preparation may comprise a step of passing a solution comprising or consisting of: (a) ribociclib or a pharmaceutically acceptable salt thereof; and (b) a solvent, through an ion exchange resin that is capable of adsorbing nitrites from the solution. Thus, another aspect of the invention relates to a method of preparing ribociclib or a pharmaceutically acceptable salt thereof with an acid, wherein the method comprises a step of passing a solution comprising or consisting of: (a) ribociclib or a pharmaceutically acceptable salt thereof; and (b) a solvent, through an ion exchange resin that is capable of adsorbing nitrites from the solution.

A pharmaceutically acceptable salt of ribociclib may be prepared with an acid and the method of preparation may comprise a step of passing a solution comprising or consisting of: (a) said acid; and (b) a solvent, through an ion exchange resin that is capable of adsorbing nitrites from the solution. Thus, an aspect of the invention relates to a method of preparing a pharmaceutically acceptable salt of ribociclib with an acid, wherein the method comprises a step of passing a solution comprising or consisting of: (a) said acid; and (b) a solvent, through an ion exchange resin that is capable of adsorbing nitrites from the solution.

The method may further comprise one or more of the following steps: (a) obtaining one or more solvents; (b) distilling the one or more solvents to reduce their content of nitrites; and (c) using the one or more distilled solvents to dissolve ribociclib and/or said acid. The distillation may be over and/or through ascorbic acid, preferably over ascorbic acid. Preferably, the method comprises all of steps (a)-(c). The method may further comprise: (d) passing the solution of ribociclib and/or the solution of said acid through an ion exchange resin that is capable of adsorbing nitrites from a solution.

Alternatively, the method may further comprise one or more of the following steps: (a) obtaining one or more solvents; (b) passing the one or more solvents through an ion exchange resin that is capable of adsorbing nitrites from a solution, and (c) using the one or more solvents to dissolve ribociclib and/or said acid. The method may further comprise (d) distilling the one or more solvents to reduce their content of nitrites, either before or after step (b). The distillation may be over and/or through ascorbic acid, preferably over ascorbic acid. Preferably, the method comprises all of steps (a)-(d), for example in the order (a), (b), (d) and (c), or in the order (a), (d), (b) and (c).

The ion exchange resin that is capable of adsorbing nitrites for use in the methods described herein may adsorb no more than 5% by weight of the total amount of ribociclib in free or salt form. The ion exchange resin may adsorb no more than 4%, no more than 3%, no more than 2%, or no more than 1% by weight of said ribociclib in free or salt form.

The ion exchange resin that is capable of adsorbing nitrites for use in the methods described herein may be selected from the group consisting of an activated charcoal resin, a silica gel resin, a celite resin, or an aluminium oxide resin. The ion exchange resin that is capable of adsorbing nitrites for use in the methods described herein may be a silica gel resin, a celite resin, or an aluminium oxide resin. In one embodiment, the functional group of the ion exchange resin is an organic acid with ammonium ion, e.g., sulfonic acid with ammonium ion, e.g., AmberLite™ MB. In one embodiment, the ion exchange resin is AmberLite™ IRA-402. In one embodiment, the ion exchange resin is an activated charcoal resin, optionally having the composition of any one of activated charcoal resins 1-47 in Example 3 below.

The ion exchange resin is preferably a basic resin or a mixed bed resin containing both cationic and ion exchange functional groups on its surface. Preferably, the ion exchange resin contains a quaternary ammonium, as shown in the structure below in the case of the ion exchange resin AmberLite™ IRA-402:

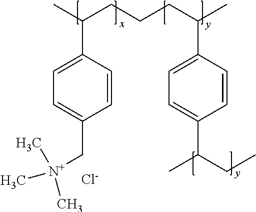

In such resins, the counterion associated with the quaternary ammonium (Cl— in the above resin) will be replaced by nitrite in the solution that is being passed through the ion exchange resin.

Following passage through the ion exchange resin, the one or more solvents may be substantially free of nitrites. The total amount of nitrites in the or each solvent may be no more than about 5 ppb, relative to the amount of the respective solvent, for example no more than about 4 ppb, no more than about 3.5 ppb, no more than about 3 ppb, no more than about 2.5 ppb, no more than about 2 ppb, no more than about 1.5 ppb, no more than about 1 ppb, less than about 1 ppb, or no more than about 0.5 ppb, relative to the amount of the respective solvent. Preferably, the total amount of nitrites in the or each solvent may be no more than about 5 ppb, no more than about 3.5 ppb, or no more than about 1 ppb, relative to the amount of the respective solvent. More preferably, the total amount of nitrites in the or each solvent may be no more than about 1 ppb, relative to the amount of the respective solvent.

In one aspect, in which the one or more solvents is more than one solvent, the combined total amount of nitrites in the solvents, following passage through the ion exchange resin, may be no more than about 5 ppb, relative to the combined amount of the solvents, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the combined amount of the solvents. Preferably, the combined total amount of nitrites in the solvents, following passage through the ion exchange resin, may be no more than about 5 ppb, no more than about 3.5 ppb, or no more than about 1 ppb, relative to the combined amount of the solvents. More preferably, the combined total amount of nitrites in the solvents, following passage through the ion exchange resin may be no more than about 1 ppb, relative to the combined amount of the solvents.

As disclosed elsewhere herein, the amount of nitrites in a composition, e.g., the amount of nitrites in a solvent prior to and/or following its passage through an ion exchange resin that is capable of absorbing nitrites, may be determined using the Griess test. Thus, the invention provides a method of preparing a composition comprising ribociclib or a pharmaceutically acceptable salt thereof, wherein the method comprises the use of the Griess test to determine the amount of nitrites in each of one or more solvents that are used in the method, prior to and/or following their passage through an ion exchange resin that is capable of absorbing nitrites.

The one or more solvents may be isopropanol, n-propanol, methyl tert-butyl ether, ethyl acetate, isopropyl acetate, and/or ethanol. The one or more solvents may be or may comprise isopropanol, optionally wherein the isopropanol is (i.e., becomes as a result of its passage through the ion exchange resin, or having fulfilled the criterion before its passage through the ion exchange resin is also compliant with the criterion after said passage) substantially free of isopropyl nitrite, e.g., the amount of isopropyl nitrite in the isopropanol is no more than one of the individual values presented above for the total amount of nitrites in the or each solvent, e.g., preferably no more than 1 ppb. By way of example, the one or more solvents may be or may comprise isopropanol and water, for example the one or more solvents may be or may comprise a solvent mixture of isopropanol and water with at least 10%, at least 20 vol %, at least 30 vol %, at least 40 vol %, at least 50 vol %, at least 60 vol %, and preferably at least 70 vol % isopropanol. Preferably, the solvent consists of isopropanol. Alternatively, the one or more solvents may not be or may not comprise isopropanol.

The one or more solvents that are used in the methods described herein may, prior to and/or after passage through the ion exchange resin, be contained in a sealed container under an atmosphere in which the percentage of oxygen is less than 21% by volume. The atmosphere may be an inert gas such as helium, nitrogen, argon, or neon. Preferably, the atmosphere is nitrogen. The distillation of the one or more solvents may be over and/or through ascorbic acid, preferably over ascorbic acid.

### Use of Filters

Another aspect of the invention relates to a method of preparing ribociclib or a pharmaceutically acceptable salt thereof with an acid, wherein the method comprises the use of one or more filters which do not comprise a nitrocellulose membrane. In some aspects, none of the filters that are used in the method comprises a nitrocellulose membrane. The one or more filters may be used to filter a solution of ribociclib and/or a solution of said acid.

For example, an aspect of the invention relates to a method of preparing ribociclib or a pharmaceutically acceptable salt thereof with an acid, wherein the method comprises: (a) obtaining a solution of ribociclib and/or a solution of said acid; and (b) filtering the or each solution with a filter that does not comprise a nitrocellulose membrane.

The or each filter may be a metal filter, preferably a stainless steel filter.

The method may comprise the use of one or more carbon filters and one or more polishing filters, wherein the filters do not comprise a nitrocellulose membrane. In some aspects of the method, none of the carbon or polishing filters comprises a nitrocellulose membrane. The or each polishing filter may be a metal filter, preferably a stainless steel filter. For example, the method of preparing a pharmaceutically acceptable salt of ribociclib may comprise a salt formation step, wherein the salt formation step comprises the use of one carbon filter and two polishing filters, wherein the filters do not comprise a nitrocellulose membrane, optionally wherein none of the carbon or polishing filters comprises a nitrocellulose membrane.

If a carbon filter is used, a charcoal treatment step may be implemented prior to use of the carbon filter.

The filtration step(s) may each be carried out at a temperature of about 60° C. to about 90° C., or about 70 to about 80° C., or about 75° C.

The method may comprise filtering a solution of acid (such as succinic acid) with a filter of about 10 to 50 inches, such as about 30 inches. The method may comprise filtering a solution of acid (such as succinic acid) with a filter having a mesh size of about 0.1 to 1.0 μm, such as about 0.5 μm.

### Washing of the Crystalline Salt

Another aspect of the invention relates to a method of preparing a crystalline form of ribociclib or a pharmaceutically acceptable salt thereof, wherein the method comprises washing the crystalline form with a solvent. The pharmaceutically acceptable salt of ribociclib may be ribociclib succinate. Thus, an aspect of the invention relates to a method of preparing ribociclib succinate, wherein the method comprises: (a) preparing a crystalline form of ribociclib succinate; and (b) washing the crystalline form with a solvent. The washing has the effect of reducing the total amount of nitrosamines (such as the total amount of N-nitroso-ribociclib in free or salt form) within the crystalline form.

The solvent may be substantially free of nitrites. The total amount of nitrites in the solvent may be no more than about 5 ppb, relative to the amount of the solvent, for example no more than about 4 ppb, no more than about 3.5 ppb, no more than about 3 ppb, no more than about 2.5 ppb, no more than about 2 ppb, no more than about 1.5 ppb, no more than about 1 ppb, or no more than about 0.5 ppb, relative to the amount of the respective solvent. Preferably, the total amount of nitrites in the solvent may be no more than about 5 ppb, no more than about 3.5 ppb, or no more than about 1 ppb, relative to the amount of the solvent. More preferably, the total amount of nitrites in the solvent may be no more than about 1 ppb, relative to the amount of the solvent. The solvent may be isopropanol, n-propanol, methyl tert-butyl ether, ethyl acetate, isopropyl acetate, or ethanol. The solvent may be isopropanol or ethanol, optionally wherein the isopropanol is substantially free of isopropyl nitrite, e.g., the amount of isopropyl nitrite in the isopropanol is no more than one of the individual values presented above for the total amount of nitrites in the solvent, e.g., preferably no more than 1 ppb. By way of example, the one or more solvents may be or may comprise isopropanol and water, for example the one or more solvents may be or may comprise a solvent mixture of isopropanol and water with at least 10%, at least 20 vol %, at least 30 vol %, at least 40 vol %, at least 50 vol %, at least 60 vol %, and preferably at least 70 vol % isopropanol. The solvent may consist of isopropanol. Preferably, the solvent is ethanol.

The washing step (b) may be carried out at a temperature of less than about 30° C., or less than about 25° C., or less than about 20° C., or less than about 15° C., such as about 10° C.

The washing step (b) may be repeated multiple times, such as two times or three times.

### Manufacture of the Drug Product

The invention further provides methods of preparing a pharmaceutical composition comprising (i) ribociclib or a pharmaceutically acceptable salt thereof and (ii) one or more pharmaceutically acceptable excipients, wherein the pharmaceutical composition is substantially free of nitrosamines, particularly N-nitroso-ribociclib in free or salt form. While methods of preparing such pharmaceutical compositions are described separately below, it will be readily apparent to a person of ordinary skill in the art that two or more of these methods may also be used in combination. Thus, the present invention also provides the combined methods of preparing a pharmaceutical composition comprising (i) ribociclib or a pharmaceutically acceptable salt thereof and (ii) one or more pharmaceutically acceptable excipients, wherein the pharmaceutical composition is substantially free of nitrosamines, particularly N-nitroso-ribociclib in free or salt form. The pharmaceutical compositions produced by these methods may be used in the pharmaceutical products described herein.

Thus, an aspect of the invention relates to a method of preparing a pharmaceutical composition comprising mixing ribociclib or a pharmaceutically acceptable salt thereof with one or more pharmaceutically acceptable excipients, wherein the one or more pharmaceutically acceptable excipients, and optionally each of the one or more pharmaceutically acceptable excipients, has a content of nitrites of no more than 5 ppm, for example no more than 4 ppm, no more than 3 ppm, no more than 2 ppm, no more than 1 ppm, or less than about 0.5 ppm. The one or more pharmaceutically acceptable excipients, e.g., each of the one or more pharmaceutically acceptable excipients, may have a content of nitrites of less than about 0.4 ppm, less than about 0.3 ppm, less than about 0.2 ppm, or less than about 0.1 ppm. It will be appreciated by a person of ordinary skill in the art that references herein to mixing two or more components may alternatively be expressed as combining said two or more components.

Accordingly, the invention provides a method of preparing a pharmaceutical composition comprising mixing ribociclib or a pharmaceutically acceptable salt thereof with one or more pharmaceutically acceptable excipients, wherein each of the one or more pharmaceutically acceptable excipients has a content of nitrites of no more than 5 ppm, for example no more than 4 ppm, no more than 3 ppm, no more than 2 ppm, no more than 1 ppm, or less than about 0.5 ppm, relative to the amount of the respective excipient. Each of the one or more pharmaceutically acceptable excipients may have a content of nitrites of less than about 0.4 ppm, less than about 0.3 ppm, less than about 0.2 ppm, or less than about 0.1 ppm, relative to the amount of the respective excipient.

Another aspect of the invention relates to a method of preparing a pharmaceutical composition comprising mixing ribociclib or a pharmaceutically acceptable salt thereof with more than one pharmaceutically acceptable excipient, wherein the combined excipients have a total content of nitrites of no more than 5 ppm, for example no more than 4 ppm, no more than 3 ppm, no more than 2 ppm, no more than 1 ppm, or less than about 0.5 ppm, relative to the combined amount of the excipients. The combined excipients may have a total content of nitrites of less than about 0.4 ppm, less than about 0.3 ppm, less than about 0.2 ppm, or less than about 0.1 ppm, relative to the combined amount of the excipients.

Any of the pharmaceutical compositions as described herein which comprise or consist of (i) ribociclib or a pharmaceutically acceptable salt thereof and (ii) one or more pharmaceutically acceptable excipients, regardless of the method by which those compositions are prepared, may have a total content of nitrites as described herein, for example a total content of nitrites of no more than 5 ppm, no more than 4 ppm, no more than 3 ppm, no more than 2 ppm, no more than 1 ppm, or no more than 0.5 ppm, preferably wherein the total content of nitrites is no more than 0.4 ppm, no more than 0.3 ppm, no more than 0.2 ppm, no more than 0.1 ppm, no more than 0.05 ppm, or no more than 0.025 ppm. Such a pharmaceutical composition may be an oral dosage form, such as a tablet, e.g., a film-coated tablet. As would be understood by a person of ordinary skill in the art, these ppm values for the content of nitrites in a pharmaceutical composition should take account of all of the components of the composition. Pharmaceutical compositions of the invention having reduced concentrations of nitrites are further described elsewhere herein. They may be included within the pharmaceutical products of the invention, e.g., they may be provided as a plurality of oral dosage forms, e.g., a plurality of tablets, optionally wherein the tablets are packaged in a blister pack as described elsewhere herein, more preferably such that each tablet is individually packaged within a substantially moisture and/or gas impermeable blister, such as within a substantially moisture and/or gas impermeable blister as described elsewhere herein, preferably an alu-alu blister.

The method of preparing a pharmaceutical composition comprising ribociclib or a pharmaceutically acceptable salt thereof, and one or more pharmaceutically acceptable excipients, may comprise a step of mixing ribociclib or said pharmaceutically acceptable salt thereof with the one or more pharmaceutically acceptable excipients. The method may further comprise a step of drying the resulting mixture. Thus, an aspect of the invention relates to a method of preparing a pharmaceutical composition comprising (i) ribociclib or a pharmaceutically acceptable salt thereof; and (ii) one or more pharmaceutically acceptable excipients, wherein the method comprises: (a) mixing ribociclib or said pharmaceutically acceptable salt thereof with the one or more excipients; and (b) drying the resulting mixture. The drying step may be carried out until the water activity value becomes less than 0.12, less than 0.10, less than 0.09 or less than 0.08, preferably wherein the water activity level becomes no more than 0.06, and more preferably no more than 0.05. The method may further comprise, either before or after the step of drying, a step of processing the composition into a solid oral dosage form. For example, the method may comprise a further step of compressing the composition into a tablet. Preferably, the step of compressing the composition into a tablet is before the step of drying. Thus, the invention provides a method of preparing a pharmaceutical composition comprising: (i) ribociclib or a pharmaceutically acceptable salt thereof; and (ii) one or more pharmaceutically acceptable excipients, wherein the method comprises: (a) providing the composition; (b) compressing the composition into one or more tablets; and (c) drying the tablet(s) until the water activity value of the tablet(s) is less than 0.08, preferably wherein the water activity value is no more than 0.06, and more preferably no more than 0.05.

Alternatively, the method may comprise a further step of filling the composition into a capsule.

The method may comprise, after the step of drying, a further step of storing the composition in the presence of a desiccant, as described elsewhere herein, e.g., storing the composition in a sealed container which also contains a desiccant, or storing the composition in a sealed pharmaceutical package which also contains a desiccant in a separate container.

The one or more pharmaceutically acceptable excipients may be selected from the group consisting of magnesium stearate, microcrystalline cellulose, crospovidone, hydroxypropyl cellulose and silica. Preferably, the pharmaceutical composition comprises more than one pharmaceutically acceptable excipient. Preferably, the pharmaceutical composition comprises magnesium stearate, microcrystalline cellulose, crospovidone, hydroxypropyl cellulose and silica.

In the case where the pharmaceutical product is a tablet, the tablet may be coated or uncoated. In an uncoated tablet, the tablet core is the whole tablet. In a coated tablet, the tablet core is the portion of the tablet excluding the coating.

In the case where the pharmaceutical product is a tablet, the tablet core may comprise or consist of (i) ribociclib or a pharmaceutically acceptable salt thereof, and (ii) one or more pharmaceutically acceptable excipients. The tablet core may comprise or consist of (i) ribociclib or a pharmaceutically acceptable salt thereof, and (ii) one or more pharmaceutically acceptable excipients selected from the group consisting of magnesium stearate, microcrystalline cellulose, crospovidone, hydroxypropyl cellulose and silica. Preferably, the tablet core comprises or consists of ribociclib succinate, magnesium stearate, microcrystalline cellulose, crospovidone, hydroxypropyl cellulose and silica.

A tablet comprising ribociclib succinate may have a drug load of at least 20%, at least, 25%, at least 30%, at least 35%, at least 40%, at least 45%, at least 50% or at least 55% when measured in w/w percentage of the ribociclib succinate of the tablet core. The % of ribociclib succinate (w/w) may be at least 40%, at least 50% or at least 55% of the tablet core. The % of ribociclib succinate (w/w) may be from about 55% to about 65% of the tablet core. The % of ribociclib succinate (w/w) may be about 59% of the tablet core.

A tablet comprising ribociclib free base may have a drug load of at least 32%, at least 40%, or at least 44%, when measured in w/w percentage of the ribociclib free base of the tablet core. The % of ribociclib free base (w/w) may be at least 32%, at least 40%, at least 44%, at least 47% or at least 52% of the tablet core. The % of ribociclib free base (w/w) may be from about 44% to about 52% of the tablet core. The % of ribociclib free base (w/w) may be about 47% of the tablet core.

The tablet core may comprise or consist of from about 59 wt % of ribociclib succinate, about 4 wt % magnesium stearate, about 16 wt % microcrystalline cellulose, about 10 wt % crospovidone, about 11 wt % hydroxypropyl cellulose and about 1 wt % silica, wherein the wt % are each defined relative to the weight of the tablet core. In the case where the pharmaceutical product is a 200 mg ribociclib tablet, the tablet core may comprise or consist of about 254 mg of ribociclib succinate, about 15 mg magnesium stearate, about 67 mg microcrystalline cellulose, about 42 mg crospovidone, about 48 mg hydroxypropyl cellulose and about 3 mg silica. Preferably, the tablet core may comprise or consist of about 254.40 mg of ribociclib succinate, about 14.82 mg magnesium stearate, about 67.44 mg microcrystalline cellulose, about 42.04 mg crospovidone, about 48.12 mg hydroxypropyl cellulose and about 3.18 mg silica.

The total tablet core weight may be from about 94% to about 99%, preferably from about 95% to about 98%, preferably from about 96% to about 97%, preferably from about 96.05% to about 96.25%, preferably about 96.15% by weight of the total tablet weight. In the case where the tablet is a 200 mg ribociclib tablet, the total coated tablet weight may be about 447.20 mg and the total tablet core weight may be from about 425 mg to about 435 mg, preferably about 430.00 mg.

In the case where the pharmaceutical product is a 200 mg ribociclib tablet, the tablet core may comprise or consist of the following:

| | Amount (mg) per 200 | |
|---|---|---|
| Ingredient | mg ribociclib tablet | Function |
| Ribociclib succinate | 254.40 | Drug substance |
| Microcrystalline cellulose | 67.44 | Diluent |
| Hydroxypropyl cellulose | 48.12 | Binder |
| Crospovidone | 42.04 | Disintegrant |
| Magnesium stearate | 14.82 | Lubricant |
| Silica | 3.18 | Glidant |

In the case where the pharmaceutical product is a tablet, the tablet may comprise a film coating. The film coating may be a moisture barrier film coating. The film coating may be an aqueous moisture barrier (“amb”) film coating. Suitable amb coatings are known in the art, such as the Opadry® amb and Opadry® amb II film coatings which are commercially available through Colorcon, Inc. Opadry® amb film coatings are preferred.

The film coating (such as the preferred amb film coating) may comprise iron oxide black (E172), iron oxide red (E172), soya lecithin (E322), polyvinyl alcohol (partially hydrolysed), talc, titanium dioxide (E171), and/or xanthan gum. The film coating may comprise or consist of about 0.5 wt % iron oxide black (E172), about 0.3 wt % iron oxide red (E172), about 2.0 wt % soya lecithin (E322), about 45.5 wt % polyvinyl alcohol (partially hydrolysed), about 20.0 wt % talc, about 31.3 wt % titanium dioxide (E171), and about 0.5 wt % xanthan gum, wherein the wt % are each defined relative to the weight of the film coating. Preferably, the film coating comprises or consists of about 0.47 wt % iron oxide black (E172), about 0.27 wt % iron oxide red (E172), about 2.00 wt % soya lecithin (E322), about 45.51 wt % polyvinyl alcohol (partially hydrolysed), about 20.00 wt % talc, about 31.26 wt % titanium dioxide (E171), and about 0.48 wt % xanthan gum, wherein the wt % are each defined relative to the weight of the film coating. In the case where the pharmaceutical product is a 200 mg ribociclib tablet, the film coating may comprise or consist of about 0.1 mg iron oxide black (E172), about 0.05 mg iron oxide red (E172), about 0.3 mg soya lecithin (E322), about 7.8 mg polyvinyl alcohol (partially hydrolysed), about 3.4 mg talc, about 5.4 mg titanium dioxide (E171), and about 0.1 mg xanthan gum. Preferably, the film coating may comprise or consist of about 0.08 mg iron oxide black (E172), about 0.05 mg iron oxide red (E172), about 0.34 mg soya lecithin (E322), about 7.83 mg polyvinyl alcohol (partially hydrolysed), about 3.44 mg talc, about 5.38 mg titanium dioxide (E171), and about 0.08 mg xanthan gum.

The total film coating weight may be from about 1% to about 6%, preferably from about 2% to about 5%, more preferably from about 3% to about 4%, even more preferably from about 3.75% to about 3.95%, and most preferably about 3.84% by weight of the total tablet weight. In the case where the tablet is a 200 mg ribociclib tablet, the total coated tablet weight may be about 447.20 mg and the total coating weight may be from about 15 mg to about 20 mg, preferably about 17.20 mg.

In the case where the pharmaceutical product is a 200 mg ribociclib tablet, the film coating may comprise or consist of the following premix ingredients:

| | | |
|---|---|---|
| | Quantity (mg) per | |
| | 200 mg ribociclib | |
| Ingredient | tablet | Function |

Basic coating premix white

| | | |
|---|---|---|
| Polyvinyl alcohol | 7.649 | Film-forming agent |
| (partially hydrolysed) | | |
| Titanium dioxide (E171) | 5.377 | Opacifier |
| Talc | 3.361 | Anti-tacking agent |
| Lecithin (soya) (E322) | 0.336 | Emulsifying agent |
| Xanthan gum | 0.081 | Film-forming agent |

Basic coating premix black

| | | |
|---|---|---|
| Polyvinyl alcohol | 0.113 | Film-forming agent |
| (partially hydrolysed) | | |
| Iron oxide black (E172)/ | 0.080 | Colorant |
| ferrosoferric | | |
| oxide | | |
| Talc | 0.050 | Anti-tacking agent |
| Lecithin (soya) (E322) | 0.005 | Emulsifying agent |
| Xanthan gum | 0.001 | Film-forming agent |

Basic coating premix red

| | | |
|---|---|---|
| Polyvinyl alcohol | 0.066 | Film-forming agent |
| (partially hydrolysed) | | |
| Iron oxide red (E172) | 0.047 | Colorant |
| Talc | 0.029 | Anti-tacking agent |
| Lecithin (soya) (E322) | 0.003 | Emulsifying agent |
| Xanthan gum | 0.001 | Film-forming agent |

The one or more pharmaceutically acceptable excipients may be or may comprise magnesium stearate. The content of nitrites in the magnesium stearate may be no more than about 7 ppm, optionally no more than about 5 ppm, no more than about 4 ppm, no more than about 3.5 ppm.

The one or more pharmaceutically acceptable excipients may be or may comprise microcrystalline cellulose. The content of nitrites in the microcrystalline cellulose may be no more than about 500 ppb, no more than about 400 ppb, no more than about 300 ppb, no more than about 200 ppb, no more than about 100 ppb, less than about 100 ppb, no more than about 90 ppb, no more than about 80 ppb, no more than about 70 ppb, no more than about 60 ppb, no more than about 50 ppb, no more than about 40 ppb, no more than about 30 ppb, no more than about 20 ppb, or no more than about 10 ppb. Preferably, the content of nitrites in the microcrystalline cellulose may be no more than about 100 ppb or less than about 100 ppb.

Suitable microcrystalline cellulose excipients are known to those skilled in the art and include types MCC PH102 and MCC PH105. The microcrystalline cellulose may be MCC PH102. The microcrystalline cellulose may have a bulk density of about 0.28 to about 0.33 g/mL.

The one or more pharmaceutically acceptable excipients may be or may comprise cross-linked polyvinylpyrrolidone (also known as crospovidone). The content of nitrites in the crospovidone is preferably no more than or less than about 100 ppb, optionally no more than about 90 ppb, no more than about 80 ppb, no more than about 70 ppb, no more than about 60 ppb, no more than about 50 ppb, no more than about 40 ppb, no more than about 30 ppb, no more than about 20 ppb, or no more than about 10 ppb.

Suitable cross-linked polyvinylpyrrolidone (also known as crospovidone) excipients are known to those skilled in the art. The cross-linked polyvinylpyrrolidone may be a Type A cross-linked polyvinylpyrrolidone.

The one or more pharmaceutically acceptable excipients may be or may comprise hydroxypropyl cellulose. The content of nitrites in the hydroxypropyl cellulose may be no more than about 500 ppb, no more than about 400 ppb, no more than about 300 ppb, no more than about 200 ppb, no more than about 100 ppb, no more than about 90 ppb, no more than about 80 ppb, no more than about 70 ppb, no more than about 60 ppb, no more than about 50 ppb, no more than about 40 ppb, no more than about 30 ppb, no more than about 20 ppb, or no more than about 10 ppb. Preferably, the content of nitrites in the hydroxypropyl cellulose may be no more than about 100 ppb.

Suitable hydroxypropyl cellulose excipients are known to those skilled in the art. The hydroxypropyl cellulose may be a low substituted hydroxypropyl cellulose.

The one or more pharmaceutically acceptable excipients may be or may comprise silica. The content of nitrites in the silica may be no more than about 500 ppb, no more than about 400 ppb, no more than about 300 ppb, no more than about 200 ppb, or no more than about 100 ppb.

As disclosed elsewhere herein, the amount of nitrites in a composition, e.g., the amount of nitrites in a pharmaceutically acceptable excipient, may be determined using the Griess test. Thus, the invention provides a method of preparing a composition comprising (i) ribociclib or a pharmaceutically acceptable salt thereof and (ii) one or more pharmaceutically acceptable excipients, wherein the method comprises the use of the Griess test to determine the amount of nitrites in one, a plurality, or each of the one or more excipients, optionally wherein the one or more excipients in which the amount of nitrites is determined is selected from the group consisting of magnesium stearate, microcrystalline cellulose, crospovidone, hydroxypropyl cellulose and silica. In some embodiments, the one or more excipients in which the amount of nitrites is determined is or comprises crospovidone. Additionally, or alternatively, the one or more excipients in which the amount of nitrites is determined is or comprises microcrystalline cellulose.

It will be understood that the preferred pharmaceutical compositions discussed hereinabove (such as the preferred tablet core and/or the preferred tablet coating compositions) may be used in or as, or be applicable to, each of the compositions, embodiments, clauses, claims and statements of invention that are described elsewhere herein.

The ribociclib or pharmaceutically acceptable salt thereof that is used in the methods of preparing a pharmaceutical composition may be prepared by the methods of preparing ribociclib or a pharmaceutically acceptable salt thereof that are described elsewhere herein.

The invention further provides methods of preparing a pharmaceutical composition comprising (i) ribociclib or a pharmaceutically acceptable salt thereof and (ii) one or more additives, wherein the pharmaceutical composition is substantially free of nitrosamines, particularly N-nitroso-ribociclib in free or salt form. The one or more additives prevent, retard or reduce the formation of nitrosamines, particularly N-nitroso-ribociclib in free or salt form, during the method of preparation of the pharmaceutical composition and/or during the subsequent storage of the composition. For example, the one or more additives is selected from the group consisting of pH adjusters, antioxidants, radical scavenging agents, and peroxide-quenching agents. The method of preparing a pharmaceutical composition comprising ribociclib or a pharmaceutically acceptable salt thereof, and one or more additives, may comprise a step of mixing ribociclib or said pharmaceutically acceptable salt thereof with the one or more additives. Accordingly, one aspect of the present invention provides a method of preparing a pharmaceutical composition comprising (i) ribociclib or a pharmaceutically acceptable salt thereof and (ii) one or more additives, wherein the method comprises mixing the ribociclib or pharmaceutically acceptable salt thereof with the one or more additives, wherein the one or more additives prevent, retard, or reduce the formation of N-nitroso-ribociclib in free or salt form during storage of the composition, optionally wherein the one or more additives are each selected from the group consisting of pH adjusters, antioxidants, radical scavenging agents, and peroxide-quenching agents.

### Testing of the Drug Substance/Drug Product

The invention further provides methods of testing the ribociclib or pharmaceutically acceptable salt thereof, as well as methods of testing the pharmaceutical compositions comprising (i) ribociclib or a pharmaceutically acceptable salt thereof and (ii) one or more pharmaceutically acceptable excipients, for the presence and/or amount of nitrosamines, in particular N-nitroso-ribociclib in free or salt form. Thus, an aspect of the invention relates to a method of evaluating a composition comprising or consisting of ribociclib or a pharmaceutically acceptable salt thereof, the method comprising testing the composition for the presence and/or amount of nitrosamines, in particular N-nitroso-ribociclib in free or salt form.

The method may be used to validate a process for the production of a composition comprising or consisting of ribociclib or a pharmaceutically acceptable salt thereof. Thus, an aspect of the invention relates to a method of validating a process for the production of a composition comprising or consisting of ribociclib or a pharmaceutically acceptable salt thereof, the method comprising testing the composition produced by said process for the presence and/or amount of nitrosamines, in particular N-nitroso-ribociclib in free or salt form.

Another aspect of the invention relates to a method of obtaining regulatory approval for a pharmaceutical composition which comprises or consists of ribociclib or a pharmaceutically acceptable salt thereof, wherein the method comprises (i) testing the composition for the presence and/or amount of nitrosamines, in particular N-nitroso-ribociclib in free or salt form, and (ii) submitting the results of said testing to a regulatory authority. Suitable regulatory authorities to which the results may be submitted are described elsewhere herein, e.g., the FDA, the EMA, the MHRA, Swissmedic, or the PMDA.

In some aspects of the methods of the invention, a batch of the composition is tested to determine the presence and/or amount of nitrosamines in said batch, in particular the presence and/or total amount of N-nitroso-ribociclib in free or salt form. In particular, a sample of the batch is tested. The batch testing may be used to determine whether to prepare a pharmaceutical product from said batch. For example, a pharmaceutical product may be prepared from the batch only if the batch is determined to have a total amount of nitrosamines of no more than about 5 ppm, for example no more than about 4 ppm, no more than about 3 ppm, no more than about 2 ppm, no more than about 1 ppm, no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.25 ppm, no more than about 0.2 ppm, no more than about 0.15 ppm, or no more than about 0.1 ppm. Preferably, the pharmaceutical product may be prepared from the batch only if the batch is determined to have a total amount of nitrosamines of no more than about 1 ppm.

Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the total amount of nitrosamines in said batch, in particular by testing a sample of the batch; and
c. preparing the pharmaceutical product from the batch only if the batch is determined to have a total amount of nitrosamines of no more than about 1 ppm. In some aspects, the pharmaceutical product is prepared from the batch only if the batch is determined to have a total amount of nitrosamines of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. In a preferred aspect, the pharmaceutical product is prepared from the batch only if the batch is determined to have a total amount of nitrosamines of no more than about 0.7 ppm, for example no more than about 0.6 ppm or no more than about 0.4 ppm.

In some aspects, a pharmaceutical product may be prepared from the batch only if the batch is determined to have a total amount of nitrosamines (such as N-nitroso-ribociclib in free or salt form) which would result in an amount of said nitrosamines (such as N-nitroso-ribociclib in free or salt form) that corresponds to an amount of no more than 400 ng/day of nitrosamine free base (such as no more than 400 ng/day of N-nitroso-ribociclib free base) being administered to the patient when the composition is administered according to an approved dosage regimen of the ribociclib or pharmaceutically acceptable salt thereof. Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the total amount of nitrosamines (such as N-nitroso-ribociclib in free or salt form) in said batch, in particular by testing a sample of the batch; and
c. preparing the pharmaceutical product from the batch only if the batch is determined to have a total amount of nitrosamines (such as N-nitroso-ribociclib in free or salt form) which would result in an amount of said nitrosamines (such as N-nitroso-ribociclib in free or salt form) that corresponds to an amount of no more than 400 ng/day of nitrosamine free base (such as no more than 400 ng/day of N-nitroso-ribociclib free base) being administered to the patient when the composition is administered according to an approved dosage regimen of the ribociclib or pharmaceutically acceptable salt thereof.

In some aspects, a batch of the composition is tested to determine the total amount of N-nitroso-ribociclib in free or salt form in said batch. In particular, a sample of the batch is tested. This batch testing may be used to determine whether to prepare a pharmaceutical product from said batch. For example, a pharmaceutical product may be prepared from the batch only if the batch is determined to have a total amount of N-nitroso-ribociclib in free or salt form of no more than about 5 ppm, relative to the total amount of ribociclib in free or salt form, for example no more than about 4 ppm, no more than about 3 ppm, no more than about 2 ppm, or no more than about 1 ppm, relative to the total amount of ribociclib in free or salt form. Preferably, a pharmaceutical product may be prepared from the batch only if the batch is determined to have a total amount of N-nitroso-ribociclib in free or salt form of no more than about 1 ppm, relative to the total amount of ribociclib in free or salt form. Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the total amount of N-nitroso-ribociclib in free or salt form in said batch, in particular by testing a sample of the batch; and
c. preparing the pharmaceutical product from the batch only if the batch is determined to have a total amount of N-nitroso-ribociclib in free or salt form of no more than about 1 ppm, relative to the total amount of ribociclib in free or salt form. In some aspects, the pharmaceutical product is prepared from the batch only if the batch is determined to have a total amount of N-nitroso-ribociclib in free or salt form of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm, relative to the total amount of ribociclib in free or salt form. In a preferred aspect, the pharmaceutical product is prepared from the batch only if the batch is determined to have a total amount of N-nitroso-ribociclib in free or salt form of no more than about 0.7 ppm, for example no more than about 0.6 ppm or no more than about 0.4 ppm.

In some aspects, a pharmaceutical product may be prepared from the batch only if the batch is determined to have a total amount of N-nitroso-ribociclib in free or salt form which would result in an amount of said N-nitroso-ribociclib in free or salt form that corresponds to an amount of no more than 400 ng/day of said N-nitroso-ribociclib free base being administered to the patient when the composition is administered according to an approved dosage regimen of the ribociclib or pharmaceutically acceptable salt thereof. Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the total amount of N-nitroso-ribociclib in free or salt form in said batch, in particular by testing a sample of the batch; and
c. preparing the pharmaceutical product from the batch only if the batch is determined to have a total amount of N-nitroso-ribociclib in free or salt form which would result in an amount of said N-nitroso-ribociclib in free or salt form that corresponds to an amount of no more than 400 ng/day of N-nitroso-ribociclib free base being administered to the patient when the composition is administered according to an approved dosage regimen of the ribociclib or pharmaceutically acceptable salt thereof.

In some aspects, a batch of the composition is tested to determine the amount of total nitrosamines, including both N-nitroso-ribociclib in free or salt form and other nitrosamines, in said batch. In particular, a sample of the batch is tested. This batch testing may be used to determine whether to prepare a pharmaceutical product from said batch. For example, a pharmaceutical product may be prepared from the batch only if the batch is determined to have an amount of total nitrosamines, including N-nitroso-ribociclib in free or salt form and other nitrosamines, of no more than about 5 ppm, for example no more than about 4 ppm, no more than about 3 ppm, no more than about 2 ppm, or no more than about 1 ppm. Preferably, a pharmaceutical product may be prepared from the batch only if the batch is determined to have an amount of total nitrosamines, including both N-nitroso-ribociclib in free or salt form and other nitrosamines, of no more than about 1 ppm. Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the amount of total nitrosamines, including both N-nitroso-ribociclib in free or salt form and other nitrosamines, in said batch, in particular by testing a sample of the batch; and
c. preparing the pharmaceutical product from the batch only if the batch is determined to have an amount of total nitrosamines, including both N-nitroso-ribociclib in free or salt form and other nitrosamines, of no more than about 1 ppm. In some aspects, the pharmaceutical product is prepared from the batch only if the batch is determined to have an amount of total nitrosamines, including both N-nitroso-ribociclib in free or salt form and other nitrosamines, of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. In some aspects, the pharmaceutical product is prepared from the batch only if the batch is determined to have an amount of total nitrosamines, including both N-nitroso-ribociclib in free or salt form and other nitrosamines, of no more than about 0.7 ppm, for example no more than about 0.6 ppm or no more than about 0.4 ppm.

In some aspects, a pharmaceutical product may be prepared from the batch only if the batch is determined to have an amount of total nitrosamines, including both N-nitroso-ribociclib in free or salt form and other nitrosamines, which would result in an amount of said total nitrosamines that corresponds to an amount of no more than 400 ng/day of nitrosamine free base being administered to the patient when the composition is administered according to an approved dosage regimen of ribociclib or pharmaceutically acceptable salt thereof. Thus, an aspect of the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the amount of total nitrosamines, including both N-nitroso-ribociclib in free or salt form and other nitrosamines, in said batch, in particular by testing a sample of the batch; and
c. preparing the pharmaceutical product from the batch only if the batch is determined to have an amount of total nitrosamines, including both N-nitroso-ribociclib in free or salt form and other nitrosamines, which would result in an amount of said total nitrosamines that corresponds to an amount of no more than 400 ng/day of nitrosamine free base being administered to the patient when the composition is administered according to an approved dosage regimen of ribociclib or pharmaceutically acceptable salt thereof.

In each of the above aspects of the invention, the batch of ribociclib or of a pharmaceutically acceptable salt thereof that is obtained in step a. of the process may be a batch of the ribociclib drug substance (i.e., a batch of the ribociclib free base or a batch of a pharmaceutically acceptable salt of ribociclib), or it may be a batch of a ribociclib drug product, (i.e., a batch in which the ribociclib or pharmaceutically acceptable salt thereof is combined with one or more pharmaceutically acceptable excipients).

In some aspects, stability testing may be performed using a sample of a batch of the composition. Following this stability testing, the sample of the batch may be tested for the total amount of nitrosamines (such as N-nitroso-ribociclib in free or salt form). This testing of the sample of the batch may be used to determine whether the batch is suitable for distribution and/or administration to a patient. For example, the batch may be determined to be suitable for distribution only if the sample of the batch after stability testing is determined to have a total amount of nitrosamines (such as N-nitroso-ribociclib in free or salt form) of no more than about 5 ppm, for example no more than about 4 ppm, no more than about 3 ppm, no more than about 2 ppm, or no more than about 1 ppm. Preferably, the batch may be determined to be suitable for distribution only if the sample of the batch after stability testing is determined to have a total amount of nitrosamines (such as N-nitroso-ribociclib in free or salt form) of no more than about 1 ppm. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of nitrosamines (such as N-nitroso-ribociclib in free or salt form) in the sample of the batch after stability testing; and
d. validating the batch for distribution only if the sample of the batch after stability testing is determined to have a total amount of nitrosamines (such as N-nitroso-ribociclib in free or salt form) of no more than about 1 ppm. In some aspects, the batch is validated for distribution only if the sample of the batch after stability testing is determined to have a total amount of nitrosamines (such as N-nitroso-ribociclib in free or salt form) of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. In a preferred aspect, the batch is validated for distribution only if the sample of the batch after stability testing is determined to have a total amount of nitrosamines (such as N-nitroso-ribociclib in free or salt form) of no more than about 0.7 ppm, for example no more than 0.6 ppm or no more than about 0.4 ppm.

In some aspects, the batch may be determined to be suitable for distribution only if the sample of the batch after stability testing is determined to have a total amount of nitrosamines (such as N-nitroso-ribociclib in free or salt form) which would result in an amount of said nitrosamines (such as N-nitroso-ribociclib in free or salt form) that corresponds to an amount of no more than 400 ng/day of nitrosamine free base (such as no more than 400 ng/day of N-nitroso-ribociclib free base) being administered to the patient when the composition is administered according to an approved dosage regimen of ribociclib or pharmaceutically acceptable salt thereof. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of nitrosamines (such as N-nitroso-ribociclib in free or salt form) in the sample of the batch after stability testing; and
d. validating the batch for distribution only if the sample of the batch after stability testing is determined to have a total amount of nitrosamines (such as N-nitroso-ribociclib in free or salt form) which would result in an amount of said nitrosamines (such as N-nitroso-ribociclib in free or salt form) that corresponds to an amount of no more than 400 ng/day of nitrosamine free base (such as no more than 400 ng/day of N-nitroso-ribociclib free base) being administered to the patient when the composition is administered according to an approved dosage regimen of ribociclib or pharmaceutically acceptable salt thereof.

In some aspects, stability testing may be performed using a sample of a batch of the composition and the sample of the batch may be tested for the total amount of N-nitroso-ribociclib in free or salt form in said sample of the batch after stability testing. This testing of the sample of the batch may be used to determine whether the batch is suitable for distribution and/or administration to a patient. For example, the batch may be determined to be suitable for distribution only if the sample of the batch after stability testing is determined to have a total amount of N-nitroso-ribociclib in free or salt form of no more than about 5 ppm, relative to the total amount of ribociclib in free or salt form, for example no more than about 4 ppm, no more than about 3 ppm, no more than about 2 ppm, or no more than about 1 ppm, relative to the total amount of ribociclib in free or salt form. Preferably, the batch may be determined to be suitable for distribution only if the sample of the batch after stability testing is determined to have a total amount of N-nitroso-ribociclib in free or salt form of no more than about 1 ppm, relative to the total amount of ribociclib in free or salt form. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of N-nitroso-ribociclib in free or salt form in said sample of said batch after stability testing; and
d. validating the batch for distribution only if the sample of the batch after stability testing is determined to have a total amount of N-nitroso-ribociclib in free or salt form of no more than about 1 ppm, relative to the total amount of ribociclib in free or salt form. In some aspects, the batch is validated for distribution only if the sample of the batch after stability testing is determined to have a total amount of N-nitroso-ribociclib in free or salt form of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm, relative to the total amount of ribociclib in free or salt form. In a preferred aspect, the batch is validated for distribution only if the sample of the batch after stability testing is determined to have a total amount of N-nitroso-ribociclib in free or salt form of no more than about 0.7 ppm, for example no more than 0.6 ppm or no more than about 0.4 ppm.

In some aspects, the batch may be determined to be suitable for distribution only if the sample of the batch after stability testing is determined to have a total amount of N-nitroso-ribociclib in free or salt form which would result in an amount of said N-nitroso-ribociclib in free or salt form that corresponds to an amount of no more than 400 ng/day of N-nitroso-ribociclib free base being administered to the patient when the composition is administered according to an approved dosage regimen of ribociclib or pharmaceutically acceptable salt thereof. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of N-nitroso-ribociclib in free or salt form in the sample of the batch after stability testing; and
d. validating the batch for distribution only if the sample of the batch after stability testing is determined to have a total amount of N-nitroso-ribociclib in free or salt form which would result in an amount of said N-nitroso-ribociclib in free or salt form that corresponds to an amount of no more than 400 ng/day of N-nitroso-ribociclib free base being administered to the patient when the composition is administered according to an approved dosage regimen of ribociclib or pharmaceutically acceptable salt thereof.

In some aspects, stability testing may be performed using a sample of a batch of the composition and the sample of the batch may be tested for the amount of total nitrosamines, including both N-nitroso-ribociclib in free or salt form and other nitrosamines, in said batch after stability testing. This testing of the sample of the batch may be used to determine whether the batch is suitable for distribution and/or administration to a patient. For example, the batch may be determined to be suitable for distribution only if the sample of the batch after stability testing is determined to have an amount of total nitrosamines, including both N-nitroso-ribociclib in free or salt form and other nitrosamines, of no more than about 5 ppm, for example no more than about 4 ppm, no more than about 3 ppm, no more than about 2 ppm, or no more than about 1 ppm. Preferably, the batch may be determined to be suitable for distribution only if the sample of the batch after stability testing is determined to have an amount of total nitrosamines, including both N-nitroso-ribociclib in free or salt form and other nitrosamines, of no more than about 1 ppm. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total nitrosamines, including both N-nitroso-ribociclib in free or salt form and other nitrosamines, in said batch after stability testing; and
d. validating the batch for distribution only if the sample of the batch after stability testing is determined to have an amount of total nitrosamines, including both N-nitroso-ribociclib in free or salt form and other nitrosamines, of no more than about 1 ppm. In some aspects, the batch is validated for distribution only if the sample of the batch after stability testing is determined to have an amount of total nitrosamines, including both N-nitroso-ribociclib in free or salt form and other nitrosamines, of no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. In a preferred aspect, the batch is validated for distribution only if the sample of the batch after stability testing is determined to have an amount of total nitrosamines, including both N-nitroso-ribociclib in free or salt form and other nitrosamines, of no more than about 0.7 ppm, for example no more than 0.6 ppm or no more than about 0.4 ppm.

In some aspects, the batch may be determined to be suitable for distribution only if the sample of the batch after stability testing is determined to have an amount of total nitrosamines, including both N-nitroso-ribociclib in free or salt form and other nitrosamines, which would result in an amount of said total nitrosamines which corresponds to an amount of no more than 400 ng/day of nitrosamine free base being administered to the patient when the composition is administered according to an approved dosage regimen of ribociclib or pharmaceutically acceptable salt thereof. Thus, an aspect of the invention provides a process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total nitrosamines, including both N-nitroso-ribociclib in free or salt form and other nitrosamines, in the sample of the batch after stability testing; and
d. validating the batch for distribution only if the sample of the batch after stability testing is determined to have an amount of total nitrosamines, including both N-nitroso-ribociclib in free or salt form and other nitrosamines, which would result in an amount of said total nitrosamines which corresponds to an amount of no more than 400 ng/day of nitrosamine free base being administered to the patient when the composition is administered according to an approved dosage regimen of ribociclib or pharmaceutically acceptable salt thereof.

In any of these methods, the step of testing for the presence and/or amount of nitrosamines may be performed using the Griess test. Likewise, the step of determining the presence and/or total amount of N-nitroso-ribociclib in free or salt form may be performed using the Griess test.

Alternatively, in any of these methods, the step of testing for the presence and/or amount of nitrosamines may be performed using high performance liquid chromatography (HPLC)- and/or gas chromatography (GC)-mass spectroscopy. Likewise, the step of determining the total amount of N-nitroso-ribociclib in free or salt form may be performed using high performance liquid chromatography (HPLC)- and/or gas chromatography (GC)-mass spectroscopy. For example, the HPLC-MS method performed may be the method provided in Example 14.

The invention also provides for the use of N-nitroso-ribociclib or a salt thereof as a reference standard to detect an impurity in a composition comprising ribociclib or a pharmaceutically acceptable salt thereof. The composition may comprise ribociclib or a pharmaceutically acceptable salt thereof and one or more pharmaceutically acceptable excipients. The impurity may be N-nitroso-ribociclib in free or salt form. The detection of the impurity may be performed using the Griess test. Alternatively, the detection of the impurity may be performed using high performance liquid chromatography (HPLC)- and/or gas chromatography (GC)-mass spectroscopy.

The invention further provides a process for preparing a pharmaceutical product comprising a composition that comprises: (i) ribociclib or a pharmaceutically acceptable salt thereof; and (ii) one or more pharmaceutically acceptable excipients, the process comprising:
a. preparing a first batch of a composition comprising or consisting of ribociclib or a pharmaceutically acceptable salt thereof;
b. determining the total amount of N-nitroso-ribociclib in free or salt form in a sample of said first batch;
c. discarding the first batch if the first batch is determined to have a total amount of N-nitroso-ribociclib in free or salt form of more than 1 ppm, relative to the total amount of ribociclib in free or salt form; and
d. preparing a second batch of a composition comprising or consisting of ribociclib or a pharmaceutically acceptable salt thereof;
wherein the reagents and/or conditions that are used in step d are adjusted relative to the reagents and/or conditions that are used in step a so as to prevent, retard or reduce the formation of N-nitroso-ribociclib in free or salt form.

The first batch may be discarded if the first batch is determined to have a total amount of N-nitroso-ribociclib in free or salt form of more than 0.7 ppm, relative to the total amount of ribociclib in free or salt form. More preferably, the first batch is discarded if the first batch is determined to have a total amount of N-nitroso-ribociclib in free or salt form of more than 0.6 ppm, relative to the total amount of ribociclib in free or salt form.

As a person of ordinary skill in the art will readily appreciate, “discarding the first batch” includes, for example, rejecting the first batch for further use, throwing away the first batch, destroying the first batch, repurposing the first batch, and/or recycling one or more components from the first batch.

The step of determining the total amount of N-nitroso-ribociclib in free or salt form may be performed by a method which includes the Griess test and/or by high performance liquid chromatography (HPLC)-mass spectroscopy and/or gas chromatography (GC)-mass spectroscopy.

The reagents and/or conditions that are adjusted in the process may include any one or any combination of the following:
the ribociclib or pharmaceutically acceptable salt thereof, optionally wherein the pharmaceutically acceptable salt is ribociclib succinate;
the one or more pharmaceutically acceptable excipients, optionally wherein the one or more pharmaceutically acceptable excipients are selected from the group consisting of magnesium stearate, microcrystalline cellulose, crospovidone, hydroxypropyl cellulose, and silica; and
one or more solvents that are used in preparing the composition, optionally wherein the one or more solvents is or comprises isopropanol.

The invention further provides a corresponding process in which the amount of total nitrosamines, including both N-nitroso-ribociclib in free or salt form and other nitrosamines, is determined in step b, and in step c, the first batch is discarded if it is determined to have an amount of total nitrosamines, including both N-nitroso-ribociclib in free or salt form and other nitrosamines, which exceeds the relevant threshold (e.g., 1 ppm, 0.7 ppm, or 0.6 ppm). In step d, the reagents and/or conditions are adjusted to prevent, retard or reduce the formation of total nitrosamines, or at least to prevent, retard or reduce the formation of N-nitroso-ribociclib in free or salt form.

In a related aspect, the invention provides a process for preparing a pharmaceutical product comprising a composition that comprises: (i) ribociclib or a pharmaceutically acceptable salt thereof; and (ii) one or more pharmaceutically acceptable excipients, the process comprising:
a. preparing a first batch of a composition comprising or consisting of ribociclib or a pharmaceutically acceptable salt thereof;
b. determining the total amount of N-nitroso-ribociclib in free or salt form in a sample of said first batch; and
c. adjusting the process (e.g., the reagents and/or conditions) in order to reduce the total amount of N-nitroso-ribociclib in free or salt form in the composition relative to the total amount of N-nitroso-ribociclib in free or salt form as determined for the first batch.

The process may be adjusted such that or until the total amount of N-nitroso-ribociclib in free or salt form satisfies a requirement as specified herein for the total amount of N-nitroso-ribociclib in free or salt form, e.g., a requirement as to the maximum ppm relative to the total amount of ribociclib in free or salt form (such as the requirement that the total amount of N-nitroso-ribociclib in free or salt form is no more than 1 ppm, preferably no more than 0.7 ppm, and more preferably no more than 0.6 ppm, relative to the total amount of ribociclib in free or salt form).

The invention further provides a corresponding process in which it is the amount of total nitrosamines, including both N-nitroso-ribociclib in free or salt form and other nitrosamines, that is determined in step b, and in which, in step c, the process is adjusted in order to reduce the amount of total nitrosamines, including both N-nitroso-ribociclib in free or salt form and other nitrosamines, in the composition. The process may be adjusted such that or until the amount of total nitrosamines, including both N-nitroso-ribociclib in free or salt form and other nitrosamines, satisfies a requirement as specified herein for the amount of total nitrosamines that is permitted in a composition (e.g., a requirement that there should be no more than 1 ppm, preferably no more than 0.7 ppm, and more preferably no more than 0.6 ppm, relative to the total amount of ribociclib in free or salt form).

Optionally, the process of this related aspect of the invention may comprise a further step d of preparing a second batch of a composition comprising or consisting of ribociclib or a pharmaceutically acceptable salt thereof using the process as adjusted in step c.

Adjusting the reagents in any process of the invention may be performed as described herein, e.g., by reducing the amount of nitrites in the potassium carbonate that may be used in any step in which intermediate B9 is deprotected to provide ribociclib B10 (such as by substituting a first batch or source of the potassium carbonate with a second batch or source of the potassium carbonate having a lower content of nitrites), by reducing the amount of nitrites in the organic or inorganic acid (such as succinic acid) that may be reacted with ribociclib free base in any step of forming a pharmaceutically acceptable salt of ribociclib (such as by substituting a first batch or source of the acid with a second batch or source of the acid having a lower content of nitrites and/or by passing the acid through an ion exchange resin as described elsewhere herein), by reducing the amount of nitrites in any pharmaceutically acceptable excipient or combination of pharmaceutically acceptable excipients that may be used in the process (such as by substituting a first batch or source of the excipient(s) with a second batch or source of the excipient(s) having a lower content of nitrites), by reducing the amount of nitrites in any of the solvents that may be used in the process (such as by substituting a first batch or source of the solvent with a second batch or source of the solvent having a lower content of nitrites, by distilling the solvent as described elsewhere herein, and/or by passing the solvent through an ion exchange resin as described elsewhere herein), and/or by reducing the exposure to oxygen of any of the solvents that may be used in the process (such as by substituting a first batch or source of a solvent with a second batch or source of the solvent having a lower content of dissolved oxygen or a lower content of oxygen in the headspace of its container and/or by sparging the solvent with an inert gas—examples of such gases being identified herein). Adjusting the reagents in any process of the invention for preparing a drug product may also be performed by substituting a first batch or source of ribociclib or a pharmaceutically acceptable salt thereof with a second batch or source of ribociclib or a pharmaceutically acceptable salt thereof having a lower content of N-nitroso-ribociclib and/or a lower content of total nitrosamines.

Adjusting the process conditions in any process of the invention may also be performed as described elsewhere herein, e.g., by adding the solution of ribociclib free base last to the mixing vessel in any step of the process in which a solution of the free base is mixed with a solution of an acid in order to form a pharmaceutically acceptable salt of ribociclib, by filtering any solution of ribociclib and/or any solution of a pharmaceutically acceptable salt of ribociclib and/or any solution of an acid that is used to make a pharmaceutically acceptable salt of ribociclib through a filter which does not comprise a nitrocellulose membrane, by washing any crystalline form of ribociclib or any crystalline form of a pharmaceutically acceptable salt of ribociclib with a solvent that is substantially free of nitrites, by storing any composition of ribociclib or any composition of a pharmaceutically acceptable salt of ribociclib in the presence of a desiccant, by storing any composition of ribociclib or any composition of a pharmaceutically acceptable salt of ribociclib under an atmosphere in which the percentage of oxygen is less than 21% by volume, by storing any composition of ribociclib or any composition of a pharmaceutically acceptable salt of ribociclib under an atmosphere in which the humidity is no more than 2.5 g/kg (e.g., in which the relative humidity is no more than 15%), and/or by storing any composition of ribociclib or any composition of a pharmaceutically acceptable salt of ribociclib under refrigerated conditions.

### Manufacture of the Pharmaceutical Product

The invention further provides methods of preparing a pharmaceutical product as described herein, and in particular a pharmaceutical product comprising a plurality of oral dosage forms as described herein. While the methods are described separately below, it will be readily apparent to a person of ordinary skill in the art that two or more of these methods may also be used in combination. Thus, the present invention also provides these combined methods of preparing a pharmaceutical product as described herein, e.g., a pharmaceutical product comprising a plurality of oral dosage forms as described herein.

One such aspect of the invention provides a method of preparing a pharmaceutical product comprising a plurality of oral dosage forms, wherein each of the oral dosage forms is a tablet, and wherein said oral dosage forms comprise a composition comprising: (i) ribociclib or a pharmaceutically acceptable salt thereof; and (ii) one or more pharmaceutically acceptable excipients, wherein the method comprises: (a) providing the composition and (b) compressing the composition into tablets.

Preferably, the method comprises a further step of (c) drying the tablets.

The drying of the tablets is preferably carried out until the water activity value of the tablets is less than 0.2, for example wherein the water activity value of the tablets is no more than 0.18, no more than 0.16, no more than 0.14, no more than 0.12, less than 0.12, no more than 0.10, less than 0.10, no more than 0.08, less than 0.08, no more than 0.075, no more than 0.07, no more than 0.065, no more than 0.06, no more than 0.055, no more than 0.05, or no more than 0.045. More preferably, the drying of the tablets is carried out until the water activity value of the tablets is less than 0.08, more preferably until the water activity value of the tablets is no more than 0.06, most preferably no more than 0.05.

Methods of measuring water activity are commonly known in the art and would be readily available to the skilled person. For example, the method used to measure the water activity may be USP <922> of the United States Pharmacopeia, e.g., as published in May 2021. Where the tablet is round, the tablet may be cut in half perpendicular to the diameter of the tablet, with the water activity measurement being performed on the said cut tablet. Optionally, the water activity measurement may be performed on a plurality of tablets (e.g., eight tablets, such as eight film-coated tablets).

In some embodiments, the method further comprises a step of applying a coating to each of the tablets before the step of drying the tablets. The coating may be a film coating. Optionally the coating is a moisture barrier film coating, as described elsewhere herein.

The step of drying the tablets may be conducted at a temperature of about 25° C. to about 50° C., e.g., at a temperature of about 25° C. to about 40° C., about 25° C. to about 35° C., about 28° C. to about 32° C., or about 30° C. Preferably, the step of drying the tablets is conducted at a temperature of about 28° C. to about 32° C., and more preferably at a temperature of about 30° C.

The step of drying the tablets may be conducted for a time period of at least 44 hours, at least 48 hours, at least 52 hours, at least 56 hours, at least 60 hours, at least 64 hours, at least 68 hours, or at least 72 hours, preferably wherein the step of drying the tablets is conducted for a time period of at least 56 hours. The time period may a time period within the range of 44 hours to 72 hours, 48 hours to 68 hours, 52 hours to 64 hours, 56 hours to 64 hours, or 56 hours to 60 hours, preferably wherein the time period is at least 56 hours.

The step of drying the tablets may be conducted at a temperature of about 25° C. to about 50° C., preferably at a temperature of about 28° C. to about 32° C., and more preferably at a temperature of about 30° C., for a time period of at least 44 hours, at least 48 hours, at least 52 hours, at least 56 hours, at least 60 hours, at least 64 hours, at least 68 hours, or at least 72 hours, preferably wherein the step of drying the tablets is conducted for a time period of at least 56 hours. In one embodiment, the step of drying the tablets may be conducted at a temperature of about 28° C. to about 32° C. for a time period of at least 44 hours, and preferably at least 56 hours. In another embodiment, the step of drying the tablets may be conducted at a temperature of about 30° C. for a time period of at least 44 hours, and preferably at least 56 hours.

The step of drying the tablets may be conducted by flowing an atmosphere over the tablets, wherein the humidity of the atmosphere is no more than 0.5 g/kg, preferably no more than 0.1 g/kg, and more preferably no more than 0.01 g/kg. In some embodiments, the relative humidity of the atmosphere is about 0%. As will be appreciated by a person of ordinary skill in the art, the humidity and relative humidity of the atmosphere that is flowed over the tablets as described herein in the context of the step of drying the tablets preferably define the inlet air humidity and inlet relative air humidity, respectively.

Any technique for drying the tablets by flowing such a low humidity atmosphere over the tablets may be used in the methods of the present invention. The selected technique and apparatus used therein should preferably allow the humidity, temperature and/or flow rate of the atmosphere to be controlled. One such technique involves fluid bed drying, in which case, a Huettlin fluid bed dryer (otherwise known as a Hüttlin device) may be used. Alternatively, a drying technique other than fluid bed drying is used, such as a technique in which the flow rate of the atmosphere is less than the flow rate that is typically used in fluid bed drying.

The flow rate of the atmosphere may be less than 3000 m³/h, e.g., less than 2500 m³/h, less than 2000 m³/h, less than 1500 m³/h, less than 1000 m³/h, or less than 500 m³/h, preferably wherein the flow rate is less than 100 m³/h. For example, the flow rate may be about 20 to about 100 m³/h, about 20 to about 75 m³/h, about 20 to about 50 m³/h, about 25 to about 40 m³/h, about 26 to about 35 m³/h, or about 28 to about 30 m³/h, preferably wherein the flow rate of the atmosphere is about 28 to about 30 m³/h. The use of lower atmospheric flow rates means that the tablets are less exposed to any NOx species in the atmosphere. It is believed that this will result in a reduced formation of nitrosamines in the tablets, e.g., a reduced formation of N-nitroso-ribociclib in free or salt form in the tablets, during their subsequent storage. As will be appreciated by a person of ordinary skill in the art, if the drying technique and apparatus used in the drying technique comprises multiple drying vessels that are used in parallel, the flow rates described herein represent the normed flow rate (i.e., the flow rate per individual vessel). For example, if the drying apparatus used comprises four parallel drying vessels and the flow rate of the atmosphere is specified as 30 m³/h, then the total flow rate would be 120 m³/h, and this total flow rate would be distributed across the four vessels.

Preferably, in the embodiment in which the step of drying the tablets is conducted by flowing an atmosphere over the tablets, the atmosphere is air. Prior to being flowed over the tablets, the air may be treated to reduce its content of NOx species, for example by subjecting the air to an NOx absorption pretreatment device such as a NOx scrubber, by subjecting the air to selective catalytic reduction, by subjecting the air to electron beam flue gas treatment, and/or by using scavengers to remove NOx from the air.

Alternatively, the step of drying the tablets may be conducted by vacuum drying, such as by HSM vacuum drying.

In some embodiments of the methods of the invention which comprise a step of drying the tablets, the total amount of nitrosamines in each of the tablets, or at least the total amount of N-nitroso-ribociclib in free or salt form in each of the tablets, is no more than 0.7 ppm, and preferably no more than 0.6 ppm, relative to the total amount of ribociclib in free or salt form, both before and after the step of drying the tablets.

In these methods of the invention of preparing a pharmaceutical product comprising a plurality of tablets, the one or more pharmaceutically acceptable excipients which are present in the composition, including the amounts of those excipients, may be as described elsewhere herein. In particular, the one or more pharmaceutically acceptable excipients may be selected from the group consisting of magnesium stearate, microcrystalline cellulose, crospovidone, hydroxypropyl cellulose, and silica. The one or more pharmaceutically acceptable excipients may comprise each of magnesium stearate, microcrystalline cellulose, crospovidone, hydroxypropyl cellulose, and silica.

The content of nitrites in the one or more excipients that are used in the preparation of the composition that is provided in step (a) and/or the content of nitrites in each of the tablets after the drying step (c) may be as described elsewhere herein. For example, the composition may comprise microcrystalline cellulose and crospovidone and (1) the content of nitrites in the microcrystalline cellulose may be no more than 100 ppb and/or (2) the content of nitrites in the crospovidone may be no more than 100 ppb. Additionally, or alternatively, the tablets after the drying step may have a total content of nitrites of no more than 5 ppm, no more than 4 ppm, no more than 3 ppm, no more than 2 ppm, no more than 1 ppm, or no more than 0.5 ppm, preferably wherein the content of nitrites is no more than 0.4 ppm, no more than 0.3 ppm, no more than 0.25 ppm, or no more than 0.2 ppm.

The indicated step (a) of providing the composition may comprise a method of preparing ribociclib or pharmaceutically acceptable salt thereof as described elsewhere herein. For example, it may comprise a method of preparing ribociclib or pharmaceutically acceptable salt thereof that uses one or more solvents and (i) the total amount of nitrites in each of the one or more solvents is no more than 5 ppb, preferably no more than 3.5 ppb, and more preferably no more than 1 ppb, relative to the amount of the respective solvent; and/or (ii) the combined total amount of nitrites in the solvents is no more than 5 ppb, preferably no more than 3.5 ppb, and more preferably no more than 1 ppb, relative to the combined amount of the solvents.

Following the step of drying the tablets, the tablets may be stored and/or packaged in a sealed container or blister pack as described elsewhere herein, optionally under an atmosphere in which the humidity is no more than 2.5 g/kg (or no more than 15% RH at 22.5° C.). Preferably, the tablets may be stored and/or packaged in a substantially moisture and/or gas impermeable blister pack, for example such that each tablet is individually packaged within a substantially moisture and/or gas impermeable blister. Examples of substantially moisture and/or gas impermeable blister packs are provided elsewhere herein. Preferably, the substantially moisture and/or gas impermeable blister pack is an alu-alu blister pack.

The tablets that result from the drying step, and in particular those that are subsequently stored and/or packaged in a sealed container or blister pack as described herein, such as those that are subsequently stored and/or packaged in a substantially moisture and/or gas impermeable blister pack, such as in a substantially moisture and/or gas impermeable blister pack as described herein, e.g., in an alu-alu blister pack, may have a shelf life as described herein. Preferably, they avoid the need for cold chain storage. More preferably, they have a shelf life of at least 18 months, and more preferably a shelf life of at least 24 months, when stored at a temperature of 20° C. to 25° C.

Another aspect of the invention provides a method of identifying a process for drying a pharmaceutical product comprising a plurality of oral dosage forms, wherein each of the oral dosage forms is a tablet, wherein said oral dosage forms comprise a composition comprising: (i) ribociclib or a pharmaceutically acceptable salt thereof; and (ii) one or more pharmaceutically acceptable excipients, wherein the process of drying comprises flowing an atmosphere (e.g., air) over the tablets, and wherein any one or any combination of the following parameters is adjusted until the process produces a product in which both:
(1) the water activity value of the tablets is less than 0.2, for example wherein the water activity value of the tablets is no more than 0.18, no more than 0.16, no more than 0.14, no more than 0.12, less than 0.12, no more than 0.10, less than 0.10, no more than 0.08, less than 0.08, no more than 0.075, no more than 0.07, no more than 0.065, no more than 0.06, no more than 0.055, no more than 0.05, or no more than 0.045, preferably wherein the water activity is less than 0.08, more preferably no more than 0.06, and most preferably no more than 0.05; and
(2) the total amount of nitrosamines in each of the tablets, or at least the total amount of N-nitroso-ribociclib in free or salt form in each of the tablets, is no more than 1 ppm, preferably no more than 0.7 ppm, and more preferably no more than 0.6 ppm, relative to the total amount of ribociclib in free or salt form, the said parameters being:
   a. the temperature at which the drying is conducted, optionally wherein the temperature is adjusted within the range of about 25° C. to about 50° C., about 25° C. to about 40° C., about 25° C. to about 35° C., or about 28° C. to about 32° C., preferably wherein the temperature is adjusted within the range of about 25° C. to about 35° C., more preferably within the range of about 28° C. to about 32° C.;
   b. the time period over which the drying is conducted, optionally wherein the time period is adjusted within the range of about 44 hours to about 72 hours, and preferably within the range of about 52 hours to about 64 hours, e.g., within the range of about 52 hours to about 60 hours, or within the range of about 54 hours to about 58 hours;
   c. the humidity of the atmosphere, optionally wherein the humidity is adjusted within the range of no more than 0.5 g/kg, preferably no more than 0.1 g/kg, and more preferably no more than 0.01 g/kg;
   d. the content of NOx species in the atmosphere, preferably wherein the content of NOx species in the atmosphere is reduced relative to the content of NOx species in the atmosphere before the adjustment; and
   e. the flow rate of the atmosphere, optionally wherein the flow rate is adjusted within the range of less than 3000 m³/h, e.g., less than 2500 m³/h, less than 2000 m³/h, less than 1500 m³/h, less than 1000 m³/h, or less than 500 m³/h, preferably wherein the flow rate of the atmosphere is less than 100 m³/h. For example, the flow rate of the atmosphere may be about 20 to about 100 m³/h, about 20 to about 75 m³/h, about 20 to about 50 m³/h, about 25 to about 40 m³/h, about 26 to about 35 m³/h, or about 28 to about 30 m³/h, preferably wherein the flow rate of the atmosphere is about 28 to about 30 m³/h.

The invention further provides a tablet comprising (i) ribociclib or a pharmaceutically acceptable salt thereof; and (ii) one or more pharmaceutically acceptable excipients, wherein the tablet has a water activity value of less than 0.08, preferably no more than 0.06, and more preferably no more than 0.05. The total amount of nitrosamines in the tablet, or at least the total amount of N-nitroso-ribociclib in free or salt form in the tablet, may be no more than 1 ppm, preferably no more than 0.7 ppm, and more preferably no more than 0.6 ppm, relative to the total amount of ribociclib in free or salt form. Examples of suitable excipients, combinations of excipients, amounts of excipients, amounts of nitrites within the excipients, and amounts of nitrites within the tablet are described elsewhere herein.

An aspect of the invention relates to a method of preparing a pharmaceutical product comprising a plurality of oral dosage forms as described herein, wherein the method comprises sealing the oral dosage forms individually or together in a container under an atmosphere in which the percentage of oxygen is less than 21% by volume. The atmosphere may be an inert gas such as helium, nitrogen, argon, or neon. Preferably, the atmosphere is nitrogen.

An aspect of the invention relates to a method of preparing a pharmaceutical product comprising a plurality of oral dosage forms as described herein, wherein the method comprises sealing the oral dosage forms individually or together in a container which also contains a desiccant, or sealing the oral dosage forms together in a pharmaceutical package which also contains a desiccant in a separate container. The desiccant may be selected from the following list: activated alumina, aerogel, benzophenone, bentonite clay, calcium chloride, calcium oxide, calcium sulphate, cobalt (II) chloride, copper (II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. Preferably, the desiccant is selected from the group consisting of silica gel, molecular sieve, calcium oxide, and calcium sulphate. More preferably, the desiccant is silica gel.

An aspect of the invention relates to a method of preparing a pharmaceutical product comprising a plurality of oral dosage forms as described herein, wherein the method comprises incorporating in each of the oral dosage forms one or more pharmaceutically acceptable additives which prevent, retard or reduce the formation of N-nitroso-ribociclib in free or salt form. Each of the one or more additives may be selected from the group consisting of pH adjusters, antioxidants, radical scavenging agents, and peroxide-quenching agents.

An aspect of the invention relates to a method of preparing a pharmaceutical product comprising a plurality of oral dosage forms as described herein, wherein the method comprises sealing the oral dosage forms individually or together in a container under an atmosphere in which the humidity is no more than 2.5 g/kg (or a relative humidity of no more than 15%). The oral dosage forms may be sealed within a blister pack such that each oral dosage form is individually sealed within a substantially moisture and/or gas impermeable blister, e.g., they may be sealed within a substantially moisture and/or gas impermeable blister pack as described herein, preferably within an alu-alu blister pack.

In any of these aspects, which may be used in any combination, the plurality of oral dosage forms may be a plurality of tablets.

The invention further provides a process for preparing a composition comprising or consisting of ribociclib or a pharmaceutically acceptable salt thereof, wherein the process has been determined to consistently produce a composition which satisfies a requirement as specified herein for the amount of total nitrosamines in such a composition and/or the total amount of N-nitroso-ribociclib in free or salt form in such a composition. For example, the process may have been determined to consistently produce a composition in which the total amount of N-nitroso-ribociclib in free or salt form and/or the amount of total nitrosamines (including both N-nitroso-ribociclib in free or salt form and other nitrosamines) is no more than 1 ppm, preferably no more than 0.7 ppm, more preferably no more than 0.6 ppm, or even more preferably no more than 0.4 ppm, relative to the total amount of ribociclib in free or salt form. The composition may further comprise one or more pharmaceutically acceptable excipients. Examples of suitable excipients, combinations of excipients, and amounts of excipients are as described elsewhere herein.

The invention further provides a process for preparing a pharmaceutical product comprising a composition that comprises: (i) ribociclib or a pharmaceutically acceptable salt thereof; and (ii) one or more pharmaceutically acceptable excipients, wherein the process has been determined to consistently produce a composition which satisfies a requirement as specified herein for the amount of total nitrosamines in such a composition and/or the total amount of N-nitroso-ribociclib in free or salt form in such a composition. For example, the process may have been determined to consistently produce a composition in which the total amount of N-nitroso-ribociclib in free or salt form and/or the amount of total nitrosamines (including both N-nitroso-ribociclib in free or salt form and other nitrosamines) is no more than 1 ppm, preferably no more than 0.7 ppm, more preferably no more than 0.6 ppm, or even more preferably no more than 0.4 ppm, relative to the total amount of ribociclib in free or salt form. As with the above process, examples of suitable excipients, combinations of excipients, and amounts of excipients are as described elsewhere herein.

These processes may be used in the preparation of a pharmaceutical product comprising a plurality of oral dosage forms as described herein, e.g., a plurality of tablets, wherein the processes may have been determined to consistently produce a plurality of oral dosage forms in which the total amount of N-nitroso-ribociclib in free or salt form and/or the amount of total nitrosamines (including both N-nitroso-ribociclib in free or salt form and other nitrosamines) in each of the oral dosage forms is no more than 1 ppm, preferably no more than 0.7 ppm, more preferably no more than 0.6 ppm, or even more preferably no more than 0.4 ppm, relative to the total amount of ribociclib in free or salt form.

Corresponding processes which have been determined to consistently produce a composition (e.g., a plurality of oral dosage forms) which, in addition to satisfying a requirement as specified herein for the amount of total nitrosamines and/or the total amount of N-nitroso-ribociclib in free or salt form, also satisfies a requirement as specified herein for the total amount of nitrites, are also provided.

The determination that a particular process consistently produces a composition which satisfies a requirement as specified herein for the amount of total nitrosamines and/or the total amount of N-nitroso-ribociclib in free or salt form in such a composition may be reflected in the fact that the composition that is obtainable by the process has been approved by a regulatory authority, such as the FDA, the EMA, the MHRA, Swissmedic, or the PMDA. As such, the above-mentioned references to a process which has been determined to consistently produce a composition which satisfies a requirement as specified herein for the amount of total nitrosamines and/or the total amount of N-nitroso-ribociclib in free or salt form in such a composition can alternatively be expressed as a process for preparing a composition (or a pharmaceutical product comprising a composition) which comprises or consists of ribociclib or a pharmaceutically acceptable salt thereof and, optionally, one or more pharmaceutically acceptable excipients, wherein said composition: (a) satisfies a requirement as specified herein for the amount of total nitrosamines and/or the total amount of N-nitroso-ribociclib in free or salt form in such a composition; and (b) is a composition that is approved by a regulatory authority, such as the FDA, the EMA, the MHRA, Swissmedic, or the PMDA. Any such approval may be one that takes account of the amount of total nitrosamines and/or the total amount of N-nitroso-ribociclib in free or salt form in such a composition. The approval may be one issued subsequently to the publication of the FDA document entitled Control of Nitrosamine Impurities in Human Drugs, Guidance for Industry, U.S. Department of Health and Human Services, Food and Drug Administration, February 2021, Revision 1 and/or the EMA document entitled European Medicines Regulatory Network approach for the implementation of the CHMP Opinion pursuant to Article 5 (3) of Regulation (EC) No 726/2004 for nitrosamine impurities in human medicines, EMA/425645/2020, 22 Feb. 2021.

### Storage of the Drug Substance/Drug Product

The invention further provides methods of storing the ribociclib or pharmaceutically acceptable salt thereof, as well as methods of storing the pharmaceutical compositions comprising (i) ribociclib or a pharmaceutically acceptable salt thereof and (ii) one or more pharmaceutically acceptable excipients. These methods may be used to prevent, retard or reduce the formation of nitrosamines, in particular N-nitroso-ribociclib in free or salt form.

### Storage of the Drug Substance

An aspect of the invention relates to a method of storing a composition comprising or consisting of ribociclib or a pharmaceutically acceptable salt thereof, wherein the method comprises storing the composition in the presence of a desiccant, e.g., storing the composition in a sealed container which also contains a desiccant, or storing the composition in a sealed pharmaceutical package which also contains a desiccant, optionally in a separate container. The method may further comprise testing the composition for the presence and/or amount of nitrosamines (such as the presence and/or total amount of N-nitroso-ribociclib in free or salt form). The method may comprise testing the composition for the presence and/or amount of nitrites.

The desiccant may be used to prevent, retard or reduce the formation of nitrosamines (such as N-nitroso-ribociclib and/or salts thereof) in a composition comprising or consisting of ribociclib or a pharmaceutically acceptable salt thereof. For example, the composition comprising or consisting of ribociclib may be stored in the presence of a desiccant, e.g., the composition may be stored in a sealed container which also contains a desiccant, or the composition may be stored in a sealed pharmaceutical package which also contains a desiccant, optionally in a separate container. Thus, an aspect of the invention relates to the use of a desiccant for preventing, retarding or reducing the formation of nitrosamines (such as N-nitroso-ribociclib or salts thereof) in a composition comprising or consisting of ribociclib or a pharmaceutically acceptable salt thereof, wherein the use comprises storing the composition in the presence of the desiccant, e.g., storing the composition in a sealed container which also contains a desiccant, or storing the composition in a sealed pharmaceutical package which also contains a desiccant, optionally in a separate container.

The desiccant may be selected from the following list: activated alumina, aerogel, benzophenone, bentonite clay, calcium chloride, calcium oxide, calcium sulphate, cobalt (II) chloride, copper (II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. Preferably, the desiccant may be selected from silica gel, molecular sieve, calcium oxide, and calcium sulphate. More preferably, the desiccant may be silica gel.

Examples of how the desiccant may be included in or incorporated into the sealed container or sealed pharmaceutical package are described elsewhere herein.

Another aspect of the invention relates to a method of storing a composition comprising or consisting of ribociclib or a pharmaceutically acceptable salt thereof, wherein the method comprises storing the composition under an atmosphere in which the percentage of oxygen is less than 21% by volume. Thus, an aspect of the invention is the use of an atmosphere in which the percentage of oxygen is less than 21% by volume for preventing, retarding or reducing the formation of nitrosamines (such as N-nitroso-ribociclib in free or salt form) in a composition comprising or consisting of ribociclib or a pharmaceutically acceptable salt thereof, wherein the composition is stored under the atmosphere. The atmosphere may be an inert gas such as helium, nitrogen, argon, or neon. Preferably, the atmosphere is nitrogen.

Once again, these methods, like any of the methods of storage that are described herein, may further comprise testing the composition comprising or consisting of ribociclib or a pharmaceutically acceptable salt thereof for the presence and/or amount of nitrosamines (such as the presence and/or total amount of N-nitroso-ribociclib in free or salt form). The method of testing for the presence and/or amount of nitrosamines (such as the presence and/or total amount of N-nitroso-ribociclib in free or salt form) as used in the methods of the invention may be the Griess test. Alternatively, the method of testing for the presence and/or total amount of nitrosamines (such as the presence and/or total amount of N-nitroso-ribociclib in free or salt form) as used in the methods of the invention may be high performance liquid chromatography (HPLC)- and/or gas chromatography (GC)-mass spectroscopy.

Testing for the presence and/or amount of nitrosamines (such as the presence and/or total amount of N-nitroso-ribociclib in free or salt form) may have particular utility in the context of a method of evaluating the stability of a composition comprising ribociclib or a pharmaceutically acceptable salt thereof, such as a pharmaceutical composition comprising (i) ribociclib or a pharmaceutically acceptable salt thereof and (ii) one or more pharmaceutically acceptable excipients. Such a method of evaluating the stability of a composition comprising ribociclib or a pharmaceutically acceptable salt thereof provides a further aspect of the invention. In such methods, the impact during storage of the composition on the formation of nitrosamines, or at least on the formation of N-nitroso-ribociclib in free or salt form, of one or more parameters may be evaluated. Such parameters may be selected from (a) storage conditions such as temperature, humidity, atmosphere, the presence of a desiccant, and/or the duration of storage or (b) the presence of one or more additives or pharmaceutically acceptable excipients in the composition, e.g., excipients which have been selected for their low concentration of nitrites. Various parameters which have been shown herein to have an impact on the formation of nitrosamines (such as N-nitroso-ribociclib in free or salt form) in a composition comprising ribociclib or a pharmaceutically acceptable salt thereof are described elsewhere herein. The testing for the presence and/or amount of nitrosamines (such as the presence and/or total amount of N-nitroso-ribociclib in free or salt form) may occur at the start of the storage, at the end of the storage, or both at the start and at the end of the storage. The results of such a method of evaluating the stability of a composition comprising ribociclib or a pharmaceutically acceptable salt thereof may be submitted to a regulatory authority.

Another aspect of the invention relates to a method of storing a composition comprising or consisting of ribociclib or a pharmaceutically acceptable salt thereof, wherein the method comprises storing the composition at a temperature of less than 40° C., or less than 30° C., or less than 25° C., or less than 20° C., or between about 1 to about 10° C. In some aspects, the composition is stored at about 2 to about 8° C. In some aspects, the composition is stored at about 4 to about 6° C. In some aspects, the composition is stored at about 5° C. In other aspects, the composition is stored at about 20° C. to about 25° C. The method may further comprise storing the composition under relative humidity conditions of less than 75%, less than 60%, or no more than 15%. The method may further comprise testing the composition for the presence and/or amount of nitrosamines, such as the presence and/or total amount of N-nitroso-ribociclib in free or salt form.

The method may comprise storing the composition at a temperature of between about 1 to about 10° C. (such as at about 2 to about 8° C., at about 4 to about 6° C., or at about 5° C.) or at room temperature (such as at about 20° C. to about 35° C., e.g., at about 20° C. to about 25° C.).

### Storage of the Drug Product

Another aspect of the invention relates to a method of storing a composition comprising (i) ribociclib or a pharmaceutically acceptable salt thereof and (ii) one or more pharmaceutically acceptable excipients, wherein the method comprises storing the composition at a temperature of about 1 to about 10° C. In some aspects, the composition is stored at about 2 to about 8° C. In some aspects, the composition is stored at about 4 to about 6° C. In some aspects, the composition is stored at about 5° C. The method may further comprise testing the composition for the presence and/or amount of nitrosamines, such as the presence and/or total amount of N-nitroso-ribociclib in free or salt form.

The method may further comprise storing the composition under relative humidity conditions of less than 75%, or less than 60%, preferably wherein the relative humidity is no more than 15%.

The method may comprise storing the composition at a temperature of between about 1 to about 10° C. (such as at about 2 to about 8° C., at about 4 to about 6° C., or at about 5° C.).

The method may comprise storing the composition at a first temperature, followed by storing the composition at a second temperature. For example, the composition may be stored at a first temperature when it, or any other composition that is present in the same container or with which it is packaged, is not in use, followed by storage at a second temperature once the composition, or the respective other composition, is in use (e.g., when packaging containing the drug product, such as a sealed pharmaceutical package containing a plurality of dosage forms, such as a plurality of orally administrable tablets, has been opened). The first temperature may be between about 1 to about 10° C. (such as at about 2 to about 8° C., at about 4 to about 6° C., or at about 5° C.). The second temperature may be at room temperature (such as at about 20° C. to about 25° C.). Therefore, the composition may be stored at about 2 to about 8° C. when it or said any other composition is not in use, and then stored at about 20° C. to about 25° C. once it or the respective other composition is in use (i.e., when the packaging containing the drug product has been opened).

An alternative aspect of the invention relates to a method of storing a composition comprising (i) ribociclib or a pharmaceutically acceptable salt thereof and (ii) one or more pharmaceutically acceptable excipients, wherein the method comprises storing the composition at a temperature of about 20° C. to about 25° C. The method may further comprise testing the composition for the presence and/or amount of nitrosamines, such as the presence and/or total amount of N-nitroso-ribociclib in free or salt form.

The method may further comprise storing the composition under relative humidity conditions of less than 75%, or less than 60%, preferably wherein the relative humidity is no more than 15%.

The composition comprising (i) ribociclib or a pharmaceutically acceptable salt thereof and (ii) one or more pharmaceutically acceptable excipients may be stored under an atmosphere in which the percentage of oxygen is less than 21% by volume. The atmosphere may be an inert gas such as helium, nitrogen, argon, or neon. Preferably, the atmosphere is nitrogen.

The composition comprising (i) ribociclib or a pharmaceutically acceptable salt thereof and (ii) one or more pharmaceutically acceptable excipients may be stored in the presence of a desiccant, e.g., the composition may be stored in a sealed container which also contains a desiccant, or the composition may be stored in a sealed pharmaceutical package which also contains a desiccant, optionally in a separate container. The desiccant may be selected from the following list: activated alumina, aerogel, benzophenone, bentonite clay, calcium chloride, calcium oxide, calcium sulphate, cobalt (II) chloride, copper (II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. Preferably, the desiccant may be selected from silica gel, molecular sieve, calcium oxide, and calcium sulphate. More preferably, the desiccant may be silica gel.

Another aspect of the invention relates to a method of evaluating the storage stability of a composition comprising (i) ribociclib or a pharmaceutically acceptable salt thereof and (ii) one or more pharmaceutically acceptable excipients, wherein the method comprises storing the composition for a predetermined period of time under a predetermined set of conditions. The method may further comprise testing the composition for the presence and/or amount of nitrosamines, such as the presence and/or total amount of N-nitroso-ribociclib in free or salt form. The testing may occur at the start of the predetermined period of time, at the end of the predetermined period of time, or both at the start and end of the predetermined period of time. It may also occur at one or more intermediate time points during the predetermined period of time.

The predetermined period of time may be 2 weeks, 1 month, 2 months, 3 months, 5 months, 6 months, 9 months, 12 months, 18 months, 24 months, or 36 months. The predetermined period of time may be 1 month, 2 months, 3 months, 5 months, or 6 months, for example 1 month, 3 months, or 6 months. The predetermined set of conditions may be (i) a temperature of 25° C., 30° C., 40° C., or 50° C. and (ii) RH of 60%. Alternatively, the predetermined set of conditions may be (i) a temperature of 25° C., 30° C., 40° C., or 50° C. and (ii) RH of 75%. The predetermined set of conditions may be 25° C./60% RH, 30° C./75% RH, 40° C./75% RH, or 50° C./75% RH, for example 30° C./75% RH, 40° C./75% RH or 50° C./75% RH. For example, the predetermined period of time and set of conditions may be:
(i) 2 weeks at 30° C./75%;
(ii) 1 month at 30° C./75%;
(iii) 3 months at 30° C./75%;
(iv) 6 months at 30° C./75%;
(v) 2 weeks at 40° C./75%;
(vi) 1 month at 40° C./75%;
(vii) 3 months at 40° C./75%;
(viii) 6 months at 40° C./75%;
(ix) 2 weeks at 50° C./75%;
(x) 1 month at 50° C./75%;
(xi) 3 months at 50° C./75%; and/or
(xii) 6 months at 50° C./75%;

The composition comprising (i) ribociclib or a pharmaceutically acceptable salt thereof and (ii) one or more pharmaceutically acceptable excipients may be present in a pharmaceutical product which further comprises a document providing instructions to a patient as to how to administer the composition.

Another aspect of the invention relates to a method of storing over a period of time a composition comprising or consisting of ribociclib or a pharmaceutically acceptable salt thereof, wherein the total amount of N-nitroso-ribociclib in free or salt form in the composition at the start of the period of time is no more than a first threshold amount, and wherein the total amount of N-nitroso-ribociclib in free or salt form in the composition at the end of the period of time is no more than a second threshold amount, wherein the storing of the composition incorporates means to prevent, retard or reduce the formation of N-nitroso-ribociclib in free or salt form. The means to prevent, retard or reduce the formation of N-nitroso-ribociclib in free or salt form may comprise or consist of one or any combination of the following:
a. storing the composition in a sealed container under an atmosphere in which the percentage of oxygen is less than 21% by volume. The atmosphere may be an inert gas such as helium, nitrogen, argon, or neon. Preferably, the atmosphere is nitrogen;
b. storing the composition in a sealed container which also contains a desiccant, or storing the composition in a sealed pharmaceutical package which also contains a desiccant in a separate container. The desiccant may be selected from the following list: activated alumina, aerogel, benzophenone, bentonite clay, calcium chloride, calcium oxide, calcium sulphate, cobalt (II) chloride, copper (II) sulphate, lithium chloride, lithium bromide, magnesium chloride hexahydrate, magnesium sulphate, magnesium perchlorate, molecular sieve, phosphorus pentoxide, potassium carbonate, potassium hydroxide, rice, silica gel, sodium, sodium chlorate, sodium chloride, sodium hydroxide, sodium sulphate, sucrose, sulfuric acid, triethylene glycol, and zeolite. Preferably, the desiccant may be selected from silica gel, molecular sieve, calcium oxide, and calcium sulphate. More preferably, the desiccant may be silica gel;
c. storing the composition under an atmosphere in which the relative humidity is less than 75%, optionally wherein the relative humidity is less than 60%, preferably wherein the relative humidity is no more than 15%;
d. storing the composition at a temperature of less than about 25° C. The composition may be stored at a temperature of between about 1 to about 10° C. (such as at about 2 to about 8° C., at about 4 to about 6° C., or at about 5° C.) or it may be stored at room temperature (such as at about 20° C. to less than about 25° C.);
e. storing the composition in a sealed container under an atmosphere in which the humidity is no more than 2.5 g/kg, optionally wherein the composition is provided as a plurality of oral dosage forms that are packaged within a blister pack such that each oral dosage form is individually packaged within a substantially moisture and/or gas impermeable blister;
f. incorporating in the composition one or more pharmaceutically acceptable additives which prevent, retard or reduce the formation of N-nitroso-ribociclib in free or salt form, optionally wherein each of the one or more additives is selected from the group consisting of pH adjusters, antioxidants, radical scavenging agents, and peroxide-quenching agents.

The period of time may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months, preferably wherein the period of time is at least 12 months, more preferably wherein the period of time is at least 18 months, most preferably wherein the period of time is at least 24 months, for example at least 36 months, at least 48 months, or at least 60 months.

The first threshold amount may be about 5 ppm, for example about 4 ppm, about 3 ppm, about 2 ppm, or about 1 ppm. For example, the first threshold amount may be about 0.9 ppm, about 0.8 ppm, about 0.7 ppm, about 0.65 ppm, about 0.6 ppm, about 0.55 ppm, about 0.5 ppm, about 0.45 ppm, about 0.4 ppm, about 0.35 ppm, about 0.3 ppm, about 0.25 ppm, about 0.2 ppm, about 0.15 ppm, about 0.1 ppm, about 0.05 ppm, or about 0.025 ppm. Preferably, the first threshold amount may be about 0.7 ppm, for example about 0.6 ppm, or about 0.4 ppm.

The second threshold amount may be about 5 ppm, for example about 4 ppm, about 3 ppm, about 2 ppm, or about 1 ppm. Preferably, the second threshold amount may be about 1 ppm. For example, the second threshold amount may be about 0.9 ppm, about 0.8 ppm, about 0.7 ppm, about 0.65 ppm, about 0.6 ppm, about 0.55 ppm, about 0.5 ppm, about 0.45 ppm, about 0.4 ppm, about 0.35 ppm, about 0.3 ppm, about 0.25 ppm, about 0.2 ppm, about 0.15 ppm, about 0.1 ppm, about 0.05 ppm, or about 0.025 ppm.

The second threshold amount may be no more than 1.5 ppm greater than the first threshold amount, for example no more than 1, no more than 0.9, no more than 0.8, no more than 0.7, no more than 0.6, no more than 0.5 or no more than 0.4 ppm greater than the first threshold amount. The second threshold amount may no more than 200% of the first threshold amount, for example no more than 190, no more than 180, no more than 170, no more than 160, no more than 150, no more than 140, no more than 130, no more than 120 or no more than 110% of the first threshold amount. Optionally, the first and second threshold amounts are the same.

In such methods involving storing or evaluating the stability of a composition comprising ribociclib or a pharmaceutically acceptable salt thereof, the composition may be a pharmaceutical composition as described herein. For example, the composition may comprise one or more pharmaceutically acceptable excipients, wherein the one or more pharmaceutically acceptable excipients may be as described herein.

The composition may be an oral dosage form. For example, the composition may be a tablet. The composition may be part of a pharmaceutical product as described herein, such as a pharmaceutical product comprising a plurality of oral dosage forms. The composition may be part of a pharmaceutical product which further comprises one or more of:
a. a document providing, either directly or via a link to an electronic database, instructions to a patient as to how to administer the composition;
b. a document providing, either directly or via a link to an electronic database, instructions which specify how the composition should be stored;
c. a document which, either directly or via a link to an electronic database, indicates that, provided that the composition is stored in accordance with specified instructions, the total amount of N-nitroso-ribociclib in free or salt form in the composition will be no more than the second threshold amount.

The method may comprise a step of determining the total amount of N-nitroso-ribociclib in free or salt form in the composition. The step of determining the total amount of N-nitroso-ribociclib in free or salt form in the composition may be performed by a method which includes the Griess test and/or high performance liquid chromatography (HPLC)-mass spectroscopy and/or gas chromatography (GC)-mass spectroscopy, as described elsewhere herein.

A related aspect of the invention relates to corresponding methods of storing over a period of time a composition comprising or consisting of ribociclib or a pharmaceutically acceptable salt thereof, wherein the total amount of nitrosamines in the composition at the start of the period of time is no more than the first threshold amount, and wherein the total amount of nitrosamines in the composition at the end of the period of time is no more than the second threshold amount, i.e., the references to N-nitroso-ribociclib in free or salt form should each be read as a reference to nitrosamines.

### Shelf Life

Another aspect of the invention relates to a composition comprising ribociclib or a pharmaceutically acceptable salt thereof, wherein the composition has a shelf life of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months when stored at a temperature of no more than 40° C. Preferably the shelf life is at least 18 months, and more preferably at least 24 months.

The shelf life may be under storage at a temperature of about 2 to about 8° C., for example at a temperature of about 4 to about 6° C. or at about 5° C. The shelf life may be under storage at a temperature of no more than about 30° C., optionally at a temperature of no more than about 25° C., for example at a temperature of no more than about 20° C. to about 25° C., such as a temperature of no more than about 20° C., no more than about 21° C., no more than about 22° C., no more than about 23° C., no more than about 24° C. or no more than about 25° C. The shelf life may be at least 24 months, for example at least 36 months, at least 48 months, or at least 60 months. The shelf life may be at least 12 months under storage at a temperature of about 2 to about 8° C., for example at a temperature of about 4 to about 6° C. or at about 5° C. For example, the shelf life may be at least 18 months under storage of about 2 to about 8° C., for example at a temperature of about 4 to about 6° C. or at about 5° C. Alternatively, the shelf life may be at least 24 months under storage at a temperature of no more than about 30° C., for example at least 36 months under storage at a temperature of no more than about 20° C. to about 25° C. Preferably, the shelf life is at least 18 months, more preferably at least 24 months, under storage at a temperature of no more than about 20° C. to about 25° C.

The shelf life storage conditions may differ depending on the stage of drug usage, for example depending on whether the drug product packaging has been opened and is in use, as described elsewhere herein. In one aspect, a first shelf life is provided for storage of the composition comprising ribociclib or a pharmaceutically acceptable salt thereof when it, or any other composition that is present in the same container or with which it is packaged, is not in use, followed by a second shelf life for storage of the composition when it, or the respective other composition, is in use. The first shelf life may be under storage at a temperature of about 2 to about 8° C., for example at a temperature of about 4 to about 6° C. or at about 5° C. The first shelf life may be at least 10 months, e.g., at least 11, at least 12, at least 13, at least 14, at least 15, at least 16, at least 17, at least 18, at least 24, at least 30, at least 36, at least 42, at least 48, at least 54 or at least 60 months. The second shelf life may be under storage at a temperature of no more than about 30° C., optionally at a temperature of no more than about 25° C., for example at a temperature of no more than about 20° C. to about 25° C. The second shelf life may be least 1 month, e.g., at least 2, at least 3, at least 4, at least 5, at least 6, at least 7, at least 8, or at least 9 months. Preferably, the second shelf life is at least 2 months. In a preferred aspect, the first shelf life is at least 10 months under storage at a temperature of about 2 to about 8° C., and the second shelf life is at least 2 months under storage at a temperature of no more than about 20° C. to about 25° C. In preferred embodiments, the second shelf life is at least 18 months, and more preferably at least 24 months, under storage at a temperature of 20° C. to about 25° C.

The shelf life may be the period of time in which the total amount of nitrosamines (such as N-nitroso-ribociclib in free or salt form) in the composition remains no more than about 5 ppm, for example no more than about 4 ppm, no more than about 3 ppm, no more than about 2 ppm, or no more than about 1 ppm. Preferably, the shelf life may be the period of time in which the total amount of nitrosamines (such as N-nitroso-ribociclib in free or salt form) in the composition remains no more than about 1 ppm. In some aspects, the shelf life may be the period of time in which the total amount of nitrosamines (such as N-nitroso-ribociclib in free or salt form) in the composition remains no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. Preferably, the shelf life may be the period of time in which the total amount of nitrosamines (such as N-nitroso-ribociclib in free or salt form) in the composition remains no more than about 0.7 ppm, for example no more than 0.6 ppm. This period of time may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months. For example, the total amount of nitrosamines (such as N-nitroso-ribociclib in free or salt form) in the composition may remain no more than about 1 ppm for at least 9 months, for example no more than 0.7 ppm or no more than 0.6 ppm for at least 9 months.

Preferably, the total amount of nitrosamines (such as N-nitroso-ribociclib in free or salt form) in the composition may remain no more than about 1 ppm for at least 9 months (for example no more than 0.7 ppm or no more than 0.6 ppm for at least 9 months) when the composition is stored at a temperature of no more than 40° C., or no more than about 25° C., or between about 2 to about 8° C. In one aspect, the total amount of nitrosamines (such as N-nitroso-ribociclib in free or salt form) in the composition may remain no more than about 1 ppm (for example no more than 0.7 ppm or no more than 0.6 ppm) for at least 10 months when the composition is stored at a temperature of between about 2 to about 8° C. In another aspect, the total amount of nitrosamines (such as N-nitroso-ribociclib in free or salt form) in the composition may remain no more than about 1 ppm (for example no more than 0.7 ppm or no more than 0.6 ppm) for at least 2 months when the composition is stored at a temperature of between about 20° C. to about 25° C. In a preferred aspect, the total amount of nitrosamines (such as N-nitroso-ribociclib in free or salt form) in the composition may remain no more than about 1 ppm (for example no more than 0.7 ppm or no more than 0.6 ppm) when stored for at least 10 months at a temperature of between about 2 to about 8° C., followed by at least 2 months when stored at a temperature of between about 20° C. to about 25° C.

In highly preferred embodiments, the total amount of nitrosamines (or at least the total amount of N-nitroso-ribociclib in free or salt form) in the composition remains no more than about 1 ppm for at least 18 months (for example no more than 0.7 ppm, or no more than 0.6 ppm for at least 18 months) when the composition is stored at a temperature of no more than about 25° C. In even more highly preferred embodiments, the total amount of nitrosamines (or at least the total amount of N-nitroso-ribociclib in free or salt form) in the composition remains no more than about 1 ppm (for example no more than 0.7 ppm, or no more than 0.6 ppm) for at least 24 months when the composition is stored at a temperature of between about 20 to about 25° C.

The shelf life may be the period of time in which the total amount of nitrosamines (such as N-nitroso-ribociclib in free or salt form) in the composition remains no more than an amount which would result in an amount of said nitrosamines (such as N-nitroso-ribociclib in free or salt form) that corresponds to an amount of 400 ng/day of nitrosamine free base (such as N-nitroso-ribociclib free base) being administered to the patient when the composition is administered according to an approved dosage regimen of ribociclib or pharmaceutically acceptable salt thereof. This period of time may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months, for example at least 9 months or at least 10 months, preferably at least 18 months, and more preferably at least 24 months.

The shelf life may be the period of time in which the total amount of N-nitroso-ribociclib in free or salt form in the composition remains no more than about 5 ppm, relative to the total amount of ribociclib in free or salt form, for example no more than about 4 ppm, no more than about 3 ppm, no more than about 2 ppm, or no more than about 1 ppm, relative to the total amount of ribociclib in free or salt form. Preferably, the shelf life may be the period of time in which the total amount of N-nitroso-ribociclib in free or salt form in the composition remains no more than about 1 ppm, relative to the total amount of ribociclib in free or salt form. In some aspects, the shelf life may be the period of time in which the total amount of N-nitroso-ribociclib in free or salt form in the composition remains no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm, relative to the total amount of ribociclib in free or salt form. In a preferred aspect, the shelf life may be the period of time in which the total amount of N-nitroso-ribociclib in free or salt form in the composition remains no more than about 0.7 ppm, for example no more than 0.6 ppm, relative to the total amount of ribociclib in free or salt form. This period of time may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months. Preferably, this period of time is at least 18 months, more preferably at least 24 months. In one example, the total amount of N-nitroso-ribociclib in free or salt form in the composition may remain no more than about 1 ppm, relative to the total amount of ribociclib in free or salt form, for at least 9 months, e.g., for at least 10 months, preferably for at least 18 months, and more preferably for at least 24 months. In another example, the total amount of N-nitroso-ribociclib in free or salt form in the composition may remain no more than about 0.7 ppm, relative to the total amount of ribociclib in free or salt form, for at least 9 months, e.g., for at least 10 months, preferably for at least 18 months, and more preferably for at least 24 months. In a third example, the total amount of N-nitroso-ribociclib in free or salt form in the composition may remain no more than about 0.6 ppm, relative to the total amount of ribociclib in free or salt form, for at least 9 months, e.g., for at least 10 months, preferably for at least 18 months, and more preferably for at least 24 months.

The shelf life may be the period of time in which the total amount of N-nitroso-ribociclib in free or salt form in the composition remains no more than an amount which would result in an amount of said N-nitroso-ribociclib in free or salt form that corresponds to an amount of 400 ng/day of N-nitroso-ribociclib free base being administered to the patient when the composition is administered according to an approved dosage regimen of ribociclib or pharmaceutically acceptable salt thereof. This period of time may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 24, at least 30, at least 36, at least 42, at least 48, at least 54 or at least 60 months, for example at least 9 months or at least 10 months, preferably at least 18 months, and more preferably at least 24 months.

The invention also provides a composition comprising ribociclib or a pharmaceutically acceptable salt thereof, wherein the amount of nitrosamines (such as N-nitroso-ribociclib in free or salt form) in the composition is no more than about 5 ppm when measured after 9 months or 10 months, more preferably after 18 or 24 months, for example wherein the amount of nitrosamines (such as N-nitroso-ribociclib in free or salt form) in the composition is no more than about 4 ppm, no more than about 3 ppm, no more than about 2 ppm, or no more than about 1 ppm when measured after 9 months or 10 months, more preferably after 18 or 24 months. Preferably, the amount of nitrosamines (such as N-nitroso-ribociclib in free or salt form) in the composition is no more than about 1 ppm when measured after 9 months or 10 months, more preferably after 18 or 24 months. In some aspects, the amount of nitrosamines (such as N-nitroso-ribociclib in free or salt form) in the composition is no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm when measured after 9 or 10 months, more preferably after 18 or 24 months. In a preferred aspect, the amount of nitrosamines (such as N-nitroso-ribociclib in free or salt form) in the composition is no more than about 0.7 ppm, for example no more than about 0.6 ppm, when measured after 9 or 10 months, more preferably after 18 or 24 months. For example, the measurement may be taken 9 months or 10 months, more preferably 18 months or 24 months, after the date of release of the composition.

The invention also provides a composition comprising ribociclib or a pharmaceutically acceptable salt thereof, wherein the amount of nitrosamines (such as N-nitroso-ribociclib in free or salt form) in the composition when measured after 9 months or 10 months, more preferably after 18 or 24 months, is no more than an amount which would result in an amount of said nitrosamines (such as N-nitroso-ribociclib in free or salt form) that corresponds to an amount of 400 ng/day of nitrosamine free base (such as N-nitroso-ribociclib free base) being administered to the patient when the composition is administered according to an approved dosage regimen of ribociclib or pharmaceutically acceptable salt thereof. For example, the measurement may be taken 9 months or 10 months, more preferably 18 months or 24 months, after the date of release of the composition.

The invention also provides a composition comprising ribociclib or a pharmaceutically acceptable salt thereof, wherein the total amount of N-nitroso-ribociclib in free or salt form in the composition is no more than about 5 ppm, relative to the total amount of ribociclib in free or salt form, when measured after 9 months or 10 months, more preferably after 18 or 24 months, for example wherein the total amount of N-nitroso-ribociclib in free or salt form in the composition is no more than about 4 ppm, no more than about 3 ppm, no more than about 2 ppm, or no more than about 1 ppm, relative to the total amount of ribociclib in free or salt form, when measured after 9 months or 10 months, more preferably after 18 or 24 months. Preferably, the total amount of N-nitroso-ribociclib in free or salt form in the composition is no more than about 1 ppm, relative to the total amount of ribociclib in free or salt form, when measured after 9 months or 10 months, more preferably after 18 or 24 months. In some aspects, the total amount of N-nitroso-ribociclib in free or salt form in the composition is no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than 0.2 ppm, no more than about 0.1 ppm, no more than about 0.05 ppm, or no more than about 0.025 ppm, relative to the total amount of ribociclib in free or salt form, when measured after 9 months or 10 months, more preferably after 18 or 24 months. In a preferred aspect, the total amount of N-nitroso-ribociclib in free or salt form in the composition is no more than about 0.7 ppm, for example no more than about 0.6 ppm, relative to the total amount of ribociclib in free or salt form, when measured after 9 months or 10 months, more preferably after 18 or 24 months. For example, the measurement may be taken 9 months or 10 months, more preferably 18 months or 24 months, after the date of release of the composition.

The invention also provides a composition comprising ribociclib or a pharmaceutically acceptable salt thereof, wherein the amount of N-nitroso-ribociclib in free or salt form in the composition when measured after 9 months or 10 months, more preferably after 18 or 24 months, is no more than an amount which would result in an amount of said N-nitroso-ribociclib in free or salt form that corresponds to an amount of 400 ng/day of N-nitroso-ribociclib free base being administered to the patient when the composition is administered according to an approved dosage regimen of ribociclib or pharmaceutically acceptable salt thereof. For example, the measurement may be taken 9 months or 10 months, more preferably 18 months or 24 months, after the date of release of the composition.

The invention also provides a composition comprising ribociclib or a pharmaceutically acceptable salt thereof, wherein the amount of total nitrosamines, including both N-nitroso-ribociclib in free or salt form and other nitrosamines, in the composition is no more than about 5 ppm when measured after 9 months or 10 months, more preferably after 18 or 24 months, for example wherein the total amount of the amount of total nitrosamines, including both N-nitroso-ribociclib in free or salt form and other nitrosamines, in the composition is no more than about 4 ppm, no more than about 3 ppm, no more than about 2 ppm, or no more than about 1 ppm when measured after 9 months or 10 months, more preferably after 18 or 24 months. Preferably, the amount of total nitrosamines, including both N-nitroso-ribociclib in free or salt form and other nitrosamines, in the composition is no more than about 1 ppm when measured after 9 months or 10 months, more preferably after 18 or 24 months. In some aspects, the amount of total nitrosamines, including both N-nitroso-ribociclib in free or salt form and other nitrosamines, in the composition is no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm when measured after 9 months or 10 months, more preferably after 18 or 24 months. In a preferred aspect, the amount of total nitrosamines, including both N-nitroso-ribociclib in free or salt form and other nitrosamines, in the composition is no more than about 0.7 ppm, for example no more than about 0.6 ppm, when measured after 9 months or 10 months, more preferably after 18 or 24 months. For example, the measurement may be taken 9 months or 10 months, more preferably 18 months or 24 months, after the date of release of the composition.

The invention also provides a composition comprising ribociclib or a pharmaceutically acceptable salt thereof, wherein the amount of total nitrosamines, including both N-nitroso-ribociclib in free or salt form and other nitrosamines, in the composition, when measured after 9 months or 10 months, more preferably after 18 or 24 months, is no more than an amount which would result in an amount of said total nitrosamines that corresponds to an amount of 400 ng/day of total nitrosamine free base being administered to the patient when the composition is administered according to an approved dosage regimen of ribociclib or pharmaceutically acceptable salt thereof. For example, the measurement may be taken 9 months or 10 months, more preferably 18 months or 24 months, after the date of release of the composition.

The start of shelf life may be determined in accordance with EU guidance CPMP/QWP/072/96 (transmitted to CPMP in June 1996 and coming into operation in December 2001), e.g., it is no later than the date on which the ribociclib or pharmaceutically acceptable salt thereof is first combined with an excipient. In particular, for a composition comprising ribociclib or a pharmaceutically acceptable salt thereof, the date of production of the composition may be taken as the date that the first step is performed involving combining the ribociclib or pharmaceutically acceptable salt thereof with other ingredients. For a composition consisting of ribociclib or a pharmaceutically acceptable salt thereof, the date of production of the composition may be taken as the date on which the ribociclib or pharmaceutically acceptable salt thereof is filled into a container for use as a medicinal product. The date of release of the composition may be taken as the start of shelf life. However, if the composition is released later than 30 days from the date of production, then the date of production, rather than the date of release, may be taken as the start of the shelf life.

### Manufacture of N-Nitroso-Ribociclib

The invention further provides a method of producing N-nitroso-ribociclib or a salt thereof, wherein the method comprises reacting ribociclib or a salt thereof with nitrous acid or a derivative thereof so as to produce said compound. The derivative of nitrous acid may be a nitrite, for example sodium nitrite.

The invention also provides a method of manufacturing a solution for use as a reference standard in a method of detecting an impurity in a composition comprising ribociclib or a pharmaceutically acceptable salt thereof. Preferably, the impurity is N-nitroso-ribociclib or a salt thereof. The method may comprise the steps of: (a) obtaining an amount of N-nitroso-ribociclib or a salt thereof; and (b) dissolving the N-nitroso-ribociclib or salt thereof in an amount of a solvent, thereby to produce a solution of said N-nitroso-ribociclib or salt thereof at a predetermined concentration. The method may further comprise the step of (c) diluting the solution in the solvent to provide one or more further solutions having different predetermined concentrations of said N-nitroso-ribociclib or salt thereof. A further aspect of the invention is a set of solutions comprising N-nitroso-ribociclib or a salt thereof at different concentrations.

Compositions with Reduced Concentration of Nitrites

The invention further provides a composition comprising or consisting of ribociclib or a pharmaceutically acceptable salt thereof, wherein said composition is substantially free of nitrites.

The total amount of nitrites in the composition may be no more than 5 ppm, for example no more than about 4 ppm, no more than about 3 ppm, no more than about 2 ppm, no more than about 1 ppm, no more than about 0.5 ppm, no more than about 0.4 ppm, no more than about 0.3 ppm, no more than about 0.25 ppm, or no more than about 0.2 ppm. Preferably, the total amount of nitrites in the composition is no more than 1 ppm. When crospovidone is used as an excipient in the composition, preferably, the nitrite contributed by the crospovidone is less than 0.1 ppm. When microcrystalline cellulose is used as an excipient in the composition, preferably, the nitrite contributed by the microcrystalline cellulose is less than 0.1 ppm. Thus, an aspect of the invention relates to a composition comprising or consisting of ribociclib or a pharmaceutically acceptable salt thereof, wherein the total amount of nitrites in the composition is no more than 1 ppm. For example, the total amount of nitrites in the composition may be no more than about 0.9 ppm, no more than about 0.8 ppm, no more than about 0.7 ppm, no more than about 0.6 ppm, no more than about 0.5 ppm, no more than about 0.4 ppm, no more than about 0.3 ppm, no more than about 0.2 ppm, no more than about 0.1 ppm, no more than about 0.05 ppm, or no more than about 0.025 ppm. Preferably, the total amount of nitrites in the composition may be no more than about 0.7 ppm, for example no more than about 0.6 ppm.

The composition of ribociclib or a pharmaceutically acceptable salt thereof may be in the form of a solution. For example, the composition may be a solution of ribociclib or a pharmaceutically acceptable salt thereof in a solvent. The solvent may be or comprise isopropanol, n-propanol, methyl tert-butyl ether, ethyl acetate, isopropyl acetate, or ethanol. In some aspects, the solvent may be isopropanol. Alternatively, the one or more solvents may not be or may not comprise isopropanol. The solvent may comprise isopropanol and water, for example it may be or may comprise a solvent mixture of isopropanol and water with at least 10%, at least 20 vol %, at least 30 vol %, at least 40 vol %, at least 50 vol %, at least 60 vol %, and preferably at least 70 vol % isopropanol.

The composition of the invention may be administered in the form of a tablet. Examples of suitable tablets and suitable methods for making the tablets are provided in WO 2016/166703 and in U.S. Pat. No. 10,799,506 which are incorporated by reference in their entirety. In particular, Example 3 of U.S. Pat. No. 10,799,506 describes coated tablets, and this example is incorporated by reference.

The tablets may be coated with an immediate release coating material. Coating materials such as Opadry®, Opadry® 200, Opadry® amb II, Opadry®, Opadry® 200, Opadry® amb II, Opadry® fx™, Opadry® II, and Opalux® are all commercially available through Colorcon, Inc. Coating materials such as Opadry White, Opadry Red and Opadry Black are likewise all commercially available through Colorcon, Inc. In some embodiments in which the composition of the invention is in the form of a coated tablet, the tablet coating may comprise or consist of iron oxide black (E172), iron oxide red (E172), soya lecithin (E322), polyvinyl alcohol (partially hydrolysed), talc, titanium dioxide (E171), and/or xanthan gum.

In some embodiments in which the composition of the invention is in the form of a coated tablet, the tablet coating may comprise or consist of about 0.5 wt % iron oxide black (E172), about 0.3 wt % iron oxide red (E172), about 2.0 wt % soya lecithin (E322), about 45.5 wt % polyvinyl alcohol (partially hydrolysed), about 20.0 wt % talc, about 31.3 wt % titanium dioxide (E171), and about 0.5 wt % xanthan gum, wherein the wt % are each defined relative to the weight of the tablet coating. Preferably, the tablet coating comprises or consists of about 0.47 wt % iron oxide black (E172), about 0.27 wt % iron oxide red (E172), about 2.00 wt % soya lecithin (E322), about 45.51 wt % polyvinyl alcohol (partially hydrolysed), about 20.00 wt % talc, about 31.26 wt % titanium dioxide (E171), and about 0.48 wt % xanthan gum, wherein the wt % are each defined relative to the weight of the tablet coating. In some embodiments in which the composition of the invention is in the form of a 200 mg ribociclib tablet which is a coated tablet, the tablet coating may comprise or consist of about 0.1 mg iron oxide black (E172), about 0.05 mg iron oxide red (E172), about 0.3 mg soya lecithin (E322), about 7.8 mg polyvinyl alcohol (partially hydrolysed), about 3.4 mg talc, about 5.4 mg titanium dioxide (E171), and about 0.1 mg xanthan gum. In some embodiments in which the composition of the invention is in the form of a 200 mg ribociclib tablet which is a coated tablet, the tablet coating may comprise or consist of about 0.08 mg iron oxide black (E172), about 0.05 mg iron oxide red (E172), about 0.34 mg soya lecithin (E322), about 7.83 mg polyvinyl alcohol (partially hydrolysed), about 3.44 mg talc, about 5.38 mg titanium dioxide (E171), and about 0.08 mg xanthan gum.

In some embodiments in which the composition of the invention is in the form of a coated tablet, the total coating weight may be from about 1% to about 6%, preferably from about 2% to about 5%, more preferably from about 3% to about 4%, even more preferably from about 3.75% to about 3.95%, and most preferably about 3.84% by weight of the total coated tablet weight.

In some embodiments in which the composition of the invention is in the form of a 200 mg ribociclib tablet which is a coated tablet, the total coated tablet weight may be about 447.20 mg and the total coating weight may be from about 15 mg to about 20 mg, preferably about 17.20 mg.

In some embodiments in which the composition of the invention is in the form of a 200 mg ribociclib tablet which is a coated tablet, the tablet coating may comprise or consist of the following premix ingredients:

| | |
|---|---|
| | Quantity (mg) per 200 mg ribociclib |
| Ingredient | tablet |

Basic coating premix white

| | |
|---|---|
| Polyvinyl alcohol | 7.649 |
| (partially hydrolysed) | |
| Titanium dioxide (E171) | 5.377 |
| Talc | 3.361 |
| Lecithin (soya) (E322) | 0.336 |
| Xanthan gum | 0.081 |

Basic coating premix black

| | |
|---|---|
| Polyvinyl alcohol | 0.113 |
| (partially hydrolysed) | |
| Iron oxide black (E172)/ | 0.080 |
| ferrosoferric | |
| oxide | |
| Talc | 0.050 |
| Lecithin (soya) (E322) | 0.005 |
| Xanthan gum | 0.001 |

Basic coating premix red

| | |
|---|---|
| Polyvinyl alcohol | 0.066 |
| (partially hydrolysed) | |
| Iron oxide red (E172) | 0.047 |
| Talc | 0.029 |
| Lecithin (soya) (E322) | 0.003 |
| Xanthan gum | 0.001 |

The content of nitrites in the or each coating material, or in the coating as a whole, may be no more than about 2 ppm, no more than about 1 ppm, no more than about 0.5 ppm, no more than about 0.2 ppm, or no more than about 0.1 ppm.

### Palbociclib Embodiments

Another CDK4/6 inhibitor that has a similar structure to ribociclib and which is likewise used in the treatment of breast cancer is palbociclib. Palbociclib also contains a piperazine ring that is a potential target for nitrosamine formation. Thus, the findings presented herein in relation to ribociclib are expected to be applicable mutatis mutandis to palbociclib. As such, the disclosure herein of aspects, embodiments, clauses, claims and statements of invention that are presented in relation to ribociclib give rise to corresponding aspects, embodiments, clauses, claims and statements of invention in which ribociclib and N-nitroso-ribociclib are substituted for palbociclib and N-nitroso-palbociclib, respectively. Such aspects, embodiments, clauses, claims and statements of invention relating to palbociclib are within the scope of the present disclosure, including the present invention.

### Examples

The present examples summarize the investigations that were performed by the present inventors in order to identify the root cause of the formation of N-nitroso-ribociclib (“DMP433”) in the commercial process for manufacturing ribociclib succinate. The examples also describe the development and evaluation of measures that were aimed at minimizing the formation and content of nitrosamines, and in particular N-nitroso-ribociclib in free or salt form, in a preparation comprising ribociclib or a pharmaceutically acceptable salt thereof, such as a preparation of ribociclib succinate.

### Example 1—Manufacturing Process

The synthetic route and the manufacturing process for the synthesis of ribociclib succinate from the starting materials are presented below:

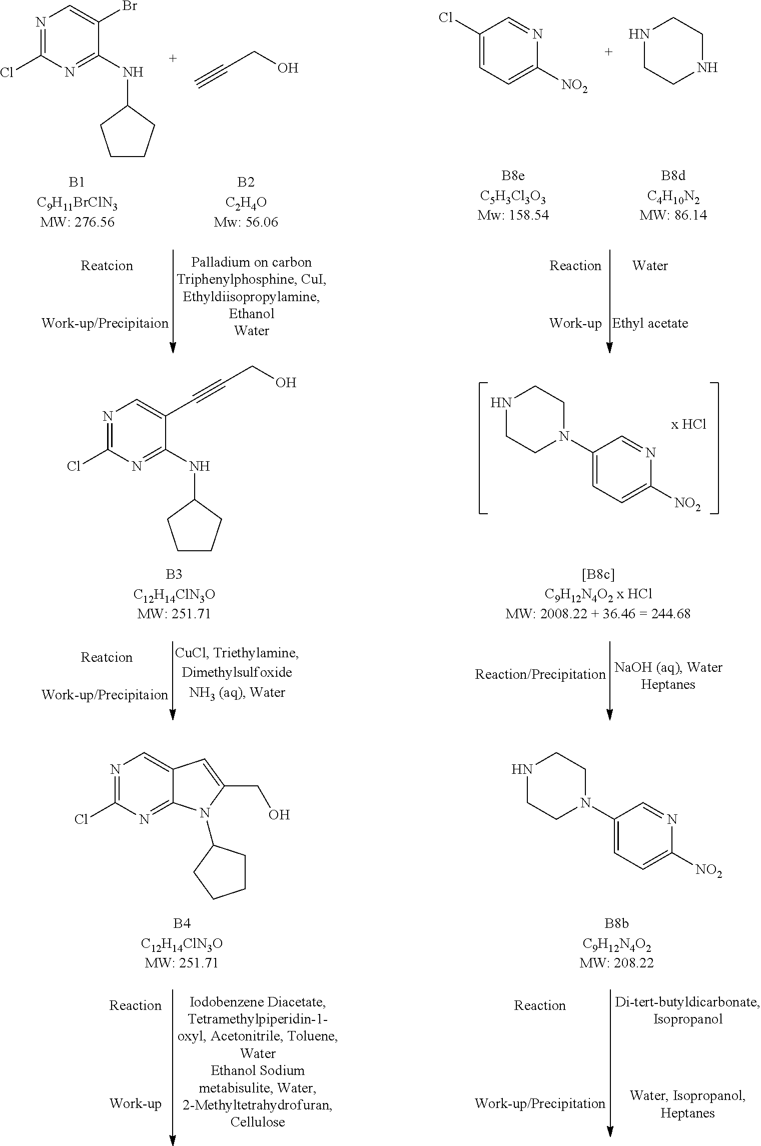

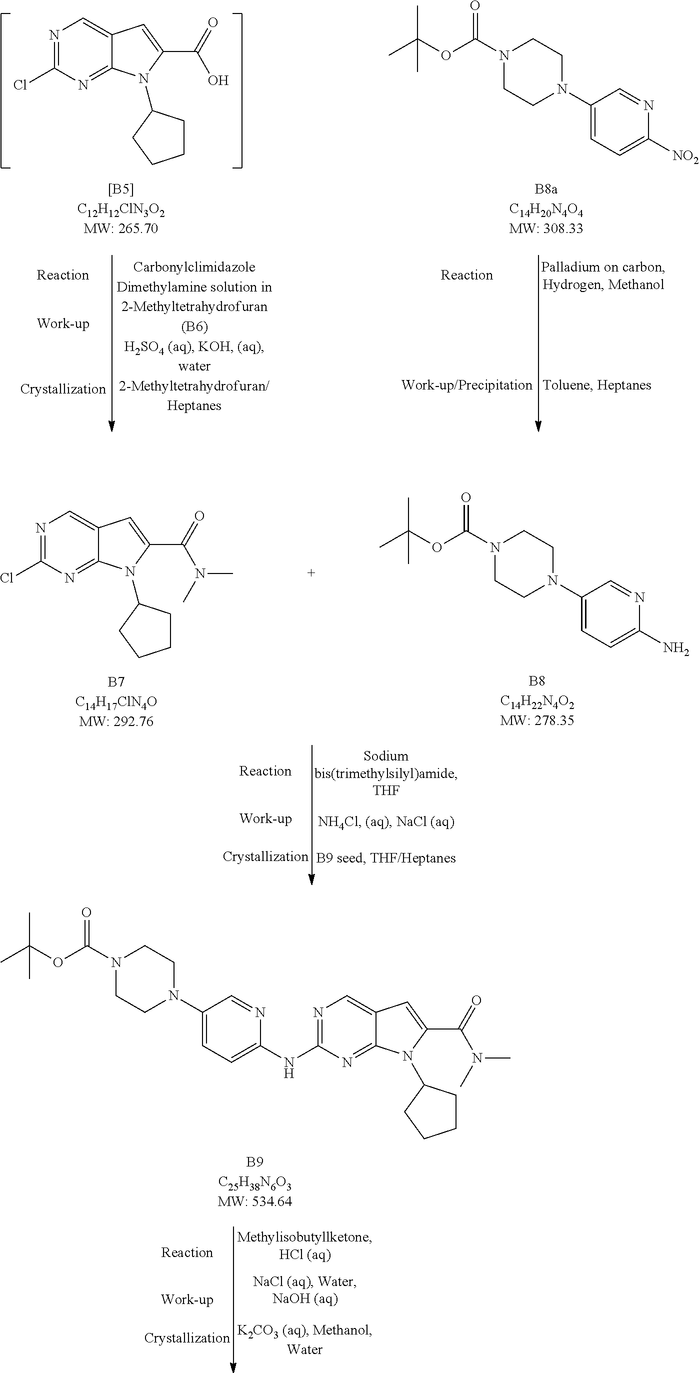

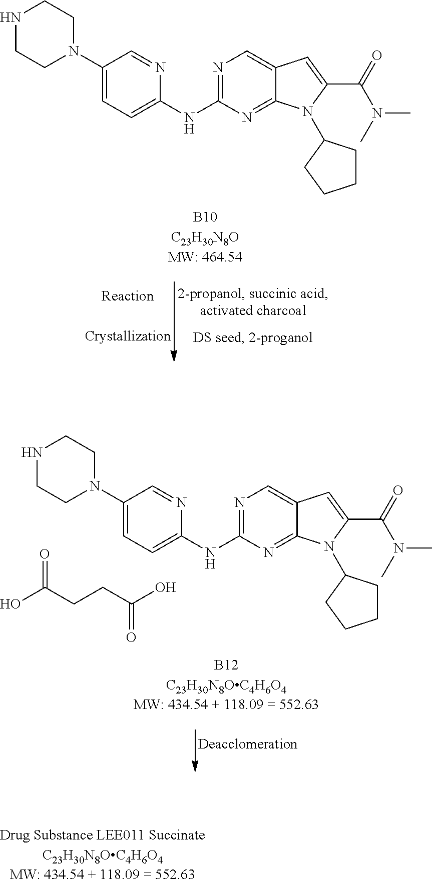

For the formation of the nitrosamine drug substance-related impurity (NDSRI) DMP433, besides the free secondary amine, a nitrite source is thought to be required. For this reason, the final two chemical steps in the above scheme, starting from compound B9, were the focus of an investigation: after cleavage of the Boc protecting group, a vulnerable secondary amine is generated. An examination of the synthesis scheme revealed no obvious source of nitrite.

### Example 2—Root Cause Investigation

The investigation into the root cause of the production of DMP433 focused on an evaluation of the manufacturing equipment, as well as the reagents, solvents and intermediates used in the final two manufacturing steps. In particular, their potential to cause the formation of DMP433 was examined.

### Manufacturing Equipment

For the manufacturing of ribociclib succinate, standard manufacturing equipment is used. In the salt formation step B10 to B12, one active carbon filter as well as two polishing filters are used. These filters were evaluated for the presence of nitrite.

### Carbon Filter:

The carbon filter in step B12 is used to remove any residual Pd. Pd is used in step B3 and to avoid a coloration of the isolated drug substance, ribociclib succinate.

The carbon filter material was tested for the presence of nitrite. In all investigated samples, the amount of nitrite found was found to be <1 ppm.

Additional experiments were performed in which the reaction from B10 to B12 was performed using different carbon filter parts, and the formation of DMP433 was monitored (two different carbon filterers from Pall and 3M were tested; Experiments “LEE011-cusakal1-050” and “LEE011-cusakal1-048”). In parallel, a control experiment without the use of carbon filter parts was also performed (Experiment “LEE011-cusakal1-049”). Since there was no significant difference between the amounts of DMP433 formed in the control experiment and in the experiments with the carbon filters, it was concluded that carbon filters do not contribute significantly to the formation of DMP433.

Conclusion: due to the very low amount of nitrite on the active carbon filter, the filter is not regarded as a significant contributor to DMP433 formation.

The investigated filters (3M, Pall) and the results of the testing for the presence of nitrites are presented in the following table:

### Nitrite Found in Active Carbon Filter

| | Content of Nitrite in | |
|---|---|---|
| Sample - batch No. | Activated carbon filter | Production site |
| 22162A061 (3M) | <1 ppm | Raybow |
| 922160A012(Pall) | <1 ppm | Raybow |
| 922162A046(3M) | <1 ppm | Raybow |
| 2-ABD/018 | <1 ppm | Mengeš |

### Polishing Filter:

Different types of polishing filters are used in the commercial manufacturing process.

It was evaluated if there is a correlation between the type of polishing filter used and the amount of DMP433 that is found in the drug substance (“DS”). The results are presented in the following table:

Dmp433 Found when Different Polishing Filters are Used

| | | | DMP433 |
|---|---|---|---|
| Description | Batch No. | Vendor | (ppm) |
| 1st pre.val.batch | B692472 | Hangzhou Cobetter | 0.364 |
| | | Filtration | |
| 1st.val.batch | B695104 | EMD Millipore Corporation | 76.702 |
| 2nd.val.batch | B695105 | EMD Millipore Corporation | 71.498 |
| 3rd.val.batch | B695106 | Hangzhou Cobetter | 0.811 |
| | | Filtration | |
| 5th post val. Batch | B696587 | Hangzhou Cobetter | 0.383 |
| | | Filtration | |
| 6th post val. Batch | B696588 | Hangzhou Cobetter | 0.537 |
| | | Filtration | |
| 7th post val. Batch | B696589 | Hangzhou Cobetter | 0.363 |
| | | Filtration | |
| 8th post val. Batch | B696590 | EMD Millipore Corporation | 58.916 |
| 9th post val. Batch | B698293 | EMD Millipore Corporation | 59.598 |
| 10th post val. Batch | B698294 | EMD Millipore Corporation | 95.528 |
| 11th post val. Batch | B698295 | EMD Millipore Corporation | 86.595 |
| 12th post val. Batch | B698296 | EMD Millipore Corporation | 87.001 |
| 13th post val. Batch | B698297 | Hangzhou Cobetter | 1.211 |
| | | Filtration | |
| 14th post val. Batch | B698298 | Pall Ilfracombe | 0.694 |
| 15th post val. Batch | B699003 | Pall Ilfracombe | 0.705 |

A clear correlation between use of the EMD Millipore filter and high levels of DMP433 in the B12 step were observed. In the laboratory, tests were performed to confirm these high DMP433 values: individual parts of the dismantled filter including the housing, the membrane and the O-ring were added to the reaction mixture. A clear correlation between the filter membrane and the formation of DMP433 was demonstrated.

Results of the laboratory use tests are shown in the following table:

Dmp433 Found when Testing Parts of the EMD Millipore Filter

| No. | Experiments | DMP433 [ppm] | Nitrite [ppm] | Comment | LEE-B10 |
|---|---|---|---|---|---|
| 1 | LEE011- | 3949.394 | app. 1.24 | w. Millipore filter | Batch: |
| | cusakal1-019 | (AS&T LJ LC-MS/MS) | (QLS LJ LC-FLD/DAN) | (Mengeš) | B685591* |
| | I32942112W | | | | |
| 2 | LEE011- | 2.105 LJ | app. 0.40 ppm | Control experiment | |
| | cusakal1-020 | (AS&T LJ LC-MS/MS) | (QLS LJ LC-FLD/DAN) | | |
| | I32942212W | | | | |
| 3 | LEE011- | 1.682 LJ | app. 0.26 ppm | w. Cobetter filter | |
| | cusakal1-021 | (AS&T LJ LC-MS/MS) | (QLS LJ LC-FLD/DAN) | (Mengeš) | |
| | I32943312W | | | | |
| 4 | LEE011- | 1.687 LJ | app. 0.32 ppm | Control experiment | |
| | cusakal1-022 | (AS&T LJ LC-MS/MS) | (QLS LJ LC-FLD/DAN) | | |
| | I32943412W | | | | |
| 5 | LEE011- | 2.823 | app. 0.3 ppm | w. Pall Fluorodyne II | |
| | cusakal1-030 | (AS&T LJ LC-MS/MS) | (QLS LJ LC-FLD/DAN) | filter (Mengeš) | |
| | I72904501A | | | | |
| 6 | LEE011- | 1.755 | app. 0.3 ppm | w. 3M Betafine filter | |
| | cusakal1-031 | (AS&T LJ LC-MS/MS) | (QLS LJ LC-FLD/DAN) | cartridge (Raybow) | |
| | I72904601A | | | | |
| 7 | LEE011- | 1.487 | app. 0.3 ppm | w. Amazon Filter | |
| | cusakal1-032 | (AS&T LJ LC-MS/MS) | (QLS LJ LC-FLD/DAN) | (Raybow) | |
| | I72904701A | | | | |
| 8 | LEE011- | 1.272 | app. 0.3 ppm | w. 3M Life ASSURE | |
| | cusakal1-033 | (AS&T LJ LC-MS/MS) | (QLS LJ LC-FLD/DAN) | PFS Series Filter | |
| | I72904801A | | | Cartridge, | |
| | | | | Lot: M33316-002 | |
| | | | | (Raybow) | |
| 9 | LEE011- | 1.789 | app. 0.2 ppm | w. 3M LifeASSURE | Batch: |
| | cusakal1-034 | (AS&T LJ LC-MS/MS) | (QLS LJ LC-FLD/DAN) | PFS Series Filter | B705468** |
| | I72903401A | | | Cartridge, | |
| | | | | Lot: M28621004 | |
| | | | | (Raybow) | |
| 10 | LEE011- | 1.661 | app. 0.2 ppm | Control experiment | |
| | cusakal1-035 | (AS&T LJ LC-MS/MS) | (QLS LJ LC-FLD/DAN) | | |
| | I72903501A | | | | |
| 11 | LEE011- | 2.100 | app. 0.3 ppm | w. Advanced | |
| | cusakal1-036 | (AS&T LJ LC-MS/MS) | (QLS LJ LC-FLD/DAN) | Microdevices PVT. Ltd. | |
| | I72903601A | | | (Divis) | |
| 12 | LEE011- | 1.932 | app. 0.3 ppm | w. Divi's Laboratories | |
| | cusakal1-037 | (AS&T LJ LC-MS/MS) | (QLS LJ LC-FLD/DAN) | Ltd. Membrane multi | |
| | I72903701A | | | filament Defender | |
| | | | | (Divis) | |
| 13 | LEE011- | 0.876 | app. 0.2 ppm | w. 3M carbon filter | Batch: |
| | cusakal1-048 | (AS&T LJ LC-MS/MS) | (QLS LJ LC-FLD/DAN) | (Mengeš) | B705468** |
| | I72908502A | | | | |
| 14 | LEE011- | 0.530 | app. 0.6 ppm | w. Pall carbon filter | |
| | cusakan1-050 | (AS&T LJ LC-MS/MS) | (QLS LJ LC-FLD/DAN) | (Supradisc) (Mengeš) | |
| | I72909402A | | | | |
| 15 | LEE011- | 0.399 | app. 0.4 ppm | Control experiment | |
| | cusakan1-049 | (AS&T LJ LC-MS/MS) | (QLS LJ LC-FLD/DAN) | | |
| | I72908602A | | | | |

*LEE011-B10 SM (B685591): DMP433 0.008 ppm; nitrite 1.4 ppm.
**LEE011-B10 SM (B705468): DMP433 0.03 ppm; nitrite < 0.1 ppm.

Upon request, EMD Millipore confirmed that their filter membrane contains nitrocellulose, which is a significant nitrite source in the process. The Hangzhou Cobetter filter Pall Ilfracombe, 3M and Amazon filters were also investigated, but did not show an impact on the amount of DMP433 that is formed in the process.

Conclusion: the EMD Millipore filter has been identified as a significant source of nitrite, which in combination with LEE011 B10 results in the formation of high amounts of DMP433.

The stainless steel 316L (SS316L) filter was tested at two sites. It was confirmed in both experiments that no corrosion occurred (no metal ions in the sample) when succinic acid was contacted with the stainless steel. Thus, it was confirmed that there was no metal release with the metal filter construction.

### Raw Materials Used in the Synthesis of Ribociclib Succinate Manufacturing of Ribocliclib Free Base (LEE011 B10)

The Boc protecting group is cleaved under acidic conditions by HCl. In cases where nitrite is present, the reaction conditions would be optimal to form the NDSRI DMP433. Potassium carbonate was identified as a potential source of nitrite.

### Potassium Carbonate

Several batches of potassium carbonate were investigated with respect to their nitrite content. The results of this investigation are provided in the following table:

### Nitrite Content of Potassium Carbonate Batches

| Vendor/User | Code | Nitrite (ppm) | Testing site |
|---|---|---|---|
| Raybow | 801720A109 | 13 | Solvias |
| Raybow | 801720A107 | 14 | Solvias |
| Raybow | 801720A108 | 13 | Solvias |
| Menges | 32384211 | 21 | Solvias |
| Menges | 32504795 | 26 | Solvias |
| Kronox | SEZ-ACG/001 | 1.9 | QLS Ljubljana |
| Kronox | SEZ-ACJ/003 | 1.0 | QLS Ljubljana |
| Kronox | DSN-ACK/004 | 0.9 | QLS Ljubljana |
| Kronox | DSN-ACG/003 | 2.5 | QLS Ljubljana |
| Andrasugar | 20231220 | 4.6 | QLS Ljubljana |
| Andrasugar | 20231219 | 1.8 | QLS Ljubljana |
| Andrasugar | 20231218 | 1.7 | QLS Ljubljana |
| Menges * | 32384211 | 24.5 | QLS Ljubljana |

The data show that there is a significant range of nitrite levels found in potassium carbonate. However, a major impact of the content of nitrite in potassium carbonate on the formation of DMP433 in LEE011 B10 could not be demonstrated, as shown in the following table:

Nitrite Content in Potassium Carbonate Vs. DMP433 in B10 Batches on Commercial Scale

| | | Nitrite level in | | | | DMP433 |
|---|---|---|---|---|---|---|
| | In use | Potassium carbonate | Potassium | | Nitrite | (ppm) |
| No. | number | (ppm) | carbonate vendor | Amount | (ppm) | in B10 |
| | LEE011-B10 | 0.6 | Sinopharm | ~2 g | 0.5 | 0.09 |
| | 24001 | | Chemical Reagent | | | |
| | | | Co., Ltd | | | |
| 2 | LEE011-810 | 0.6 | Sinopharm | ~2 g | 0.1 | 0.05 |
| | 24002 | | Chemical Reagent | | | |
| | | | Co., Ltd | | | |
| 3 | LEE011-B10 | 23 | Zhejiang Dayang | ~2 g | 0.1 | 0.05 |
| | 24003 | | Biotech Group Co., | | | |
| | | | Ltd | | | |

Conclusion: potassium carbonate did not appear to be a significant contributor of nitrite into the process.

Further experiments were carried out at a laboratory scale. 2×30g experiments (with Sinopharm as the supplier of potassium carbonate), up to B10, were completed, including a 1×30g experiment with another supplier for comparison. The batches of potassium carbonate were investigated with respect to their nitrite content. The formation of DMP433 in LEE011 B10 was also investigated. The results of this investigation are provided in the following table:

| LEE011-B10 Batch | Potassium carbonate source/Nitrite | Nitrite | DMP433 (LOQ: 30 |
|---|---|---|---|
| number | content(ppm) | (LOQ: 0.1 ppm) | ppb) |
| 805193A095 | Dayang(801720A111)/29.0 ppm | <0.1 (0.08) | 55.8 ppb |
| | | ppm | |
| 805193A096 | | <0.1 (0.05) | 70.5 ppb |
| | | ppm | |
| 805193A097 | | <0.1(0.07) | 73.9 ppb |
| | | ppm | |
| 805193A098 | Sinopharm(923048A002)/4.8 ppm | <0.1(0.07) | 78.8 ppb |
| | | ppm | |

The data shows that the content of nitrite in potassium carbonate does not play the most important role in the formation of DMP433. Sinopharm supplied potassium carbonate with a lower amount of nitrite (4.8 ppm) compared to Raybow. The conclusions from the laboratory scale have been confirmed on a production scale. Despite the low, or no, impact of the nitrite content in potassium carbonate on the B10 quality, it is proposed to use potassium carbonate with a low nitrite content, such as the potassium carbonate from a supplier such as Sinopharm and/or Andra Sugar.

Further experiments were carried out to identify potassium carbonate sources with low nitrite. Potassium carbonate from AndraSugar (4 batches), with improved sample preparation, resulted at around 1 ppm. Evonik (2×200g) were tested and the results were high. Kronox (India) samples were tested and are low on nitrites.

Manufacturing of Ribociclib succinate (LEE011 B12)

Due to the nature of succinic acid, the salt formation is performed under acidic conditions, so there is a possibility that any nitrite that is present in that step of the process will lead to the formation of DMP433. Ribociclib base (LEE011 B10), succinic acid (LEE011 B11) and i-propanol were investigated as potential sources of nitrite.

### Ribociclib Base

Batches of ribociclib base (LEE011 B10) were tested for the presence of nitrites by ion chromatography (LOQ app. 3 ppm, LOD app. 1 ppm) and of DMP433 (LOQ app. 0.0625 ppm). The results are presented in the following table:

### Nitrite Content in Ribociclib Base (LEE011 B10)

| | | | DMP433 [ppm] |
|---|---|---|---|
| | | Nitrite [ppm] | LOQ |
| Sample | Batch | LOD (1 ppm) | (0.0625 ppm) |
| LEE011-B10, Raybow | 805193A074 | <LOD | <LOQ* (0.029) |
| LEE011-B10, Raybow | 923037A005 | <LOD | <LOQ* (0.048) |
| LEE011-B10, Raybow | 923037A002 | <LOD | <LOQ* (0.047) |
| LEE011-B10, Raybow | 805193A073 | <LOD | <LOQ* (0.030) |
| LEE011-B10, Raybow | 805193A072 | <LOD | <LOQ* (0.060) |
| LEE011-B10, Raybow | 923037A005 | <LOD | <LOQ* (0.048) |
| LEE011-B10, Mengeš | B687416 | <LOD | — |
| LEE011-B10, Mengeš | B685591 | <LOD | — |
| LEE011-B10, Mengeš | B687418 | <LOD | — |
| LEE011-B10, Mengeš | B689736 | <LOD | — |
| LEE011-B10, Mengeš | B704193 | — | <LOQ* (0.006) |
| LEE011-B10, Mengeš | B704194 | — | <LOQ* (0.005) |
| LEE011-B10, Mengeš | B704196 | — | <LOQ* (0.019) |
| LEE011-B10, Mengeš | B705465 | — | <LOQ* (0.037) |
| LEE011-B10, Mengeš | B0705466 | — | <LOQ* (0.043) |
| LEE011-B10, Mengeš | B0705467 | — | <LOQ* (0.035) |
| LEE011-B10, Mengeš | B0705468 | — | <LOQ* (0.030) |

*All of the tested DMP433 results in ribociclib base (B10) were below LOQ < 0.0625 ppm. Results in brackets represent an extrapolation and are only an estimate of actual values.

Additional batches of ribociclib base (LEE011 B10 (IP-1)) were tested for the presence of nitrites and DMP433. The results are presented in the following table:

| | | | Nitrite |
|---|---|---|---|
| | | | [ppm] |
| | | | LOD |
| Sample | Batch | DMP433 [ppb] | (1 ppm) |
| LEE011-B10 (IP-1), Raybow | 805193A077 | 51 | <LOD |
| LEE011-B10 (IP-1), Raybow | 805193A078 | 78 | <LOD |
| LEE011-B10 (IP-1), Mengeš | B704193 | 57 | <LOD |
| LEE011-B10 (IP-1), Mengeš | B705468 | 130 | <LOD |

### Succinic Acid

Several commercial batches of succinic acid were tested for their nitrite content. In all cases only very low nitrite content was found. The results are presented in the following table:

### Nitrite Content in Succinic Acid

| Material | Supplier | Batch No. | Nitrite (ppm) |
|---|---|---|---|
| Succinic | Merck | K54521481303 | 0.1 |
| acid | Merck | K54510281303 | 0.1 |
| | Merck | 32498370 | 0.1 |
| | Merck | 802851A019 | 0.1 |
| | Merck | 802851A018 | <0.1 |
| | Merck | 802851A020 | <0.1 |
| | Raybow | 802851A017 | 0.013 |

Taking the amount of succinic acid into consideration and based on the assumption that all of the nitrite in succinic acid would result in formation of DMP433, there could be a maximum 0.345 ppm of DMP433 out of the nitrite in succinic acid.

Conclusion: Succinic acid is not a major contributor to DMP433 formation in the process.

The theoretical contributions of nitrite in the ribociclib base and i-propanol to the formation of DMP433 were also calculated. The results are presented in the following table, based on the assumption that there is 5 ppb of nitrite in the i-propanol:

| | | | | | | | Theoretical DMP433 |
|---|---|---|---|---|---|---|---|
| | | mass [g], | | DMP433 | Nitrite | | contribution in B12 |
| No. | SM/RM used | (w/w %) | | [ppm] | [ppm] | Nitrite [g] | [ppm] |
| 1 | LEE-B10 (B705468) | 2 | (1.6) | 0.03 | <0.1 | n.a. | 0.024 (0.8%) carry |
| | | | | | | | over from B10 |
| 2 | iPrOH (BCCG9775) | 124.19 | (97.7) | n.a. | 0.005 | 0.000012419 | 2.500 (87.0%) |
| 3 | Succinic acid | 0.87 | (0.7) | n.a. | 0.1 | 0.000000087 | 0.345 (12.2%) |
| | (K54521481 303) | | | | | | |

Further samples of succinic acid were tested by Menges (three samples) and the nitrite testing gave results of 0.1 ppm.

Isopropanol (iPrOH)

Commercial batches of isopropanol (iPrOH), including those under nitrogen (N₂), were tested for their content of nitrites. Very low levels of nitrite <5 ppb (LOD) were found in all cases. However, since the amount of iPrOH that is used in the process is around 80 times the amount of LEE011 B10, the total amount of nitrite in iPrOH could potentially have a significant impact on the formation of DMP433. Based on a worst-case calculation, 1 ppb of nitrite in/PrOH could lead to the formation of approx. 510 ppb of DMP433 in ribociclib succinate.

It has been demonstrated on a laboratory scale that/PrOH can take up NOx to form i-propyl nitrite, which has been confirmed by HS-GC/MS. i-propyl nitrite is known as a nitrosating species and it could therefore result in the formation of DMP433.

On a laboratory scale, iPrOH batches containing different amounts of nitrites were used in the salt formation step. As shown in the following table, the amount of nitrite in/PrOH has a direct correlation to the amount of DMP433 in LEE011 B12. It is also important to mention that, on a laboratory scale, no carbon or polishing filters were used.

Impact of Nitrite in iPrOH on DMP433 Formation

| | Nitrite in iPrOH | | | |
|---|---|---|---|---|
| LEE011-B10 | (LOQ <5 ppb) | DMP433 in LEE011 B12 | | Nitrite in LEE011 B12 |
| Batch 805193A078 | <5 ppb | 995 | ppb | 448 ppb |
| | (1.3 ppb | | | |
| | extrapolated) | | | |
| | <5 ppb | 1513 | ppb | 326 ppb |
| | (3.1 ppb | | | |
| | extrapolated) | | | |
| | 119.6 ppb | 14238 | ppb | Test on-going |
| | 162.7 ppb | 18011 | ppb | Test on-going |

Conclusion: Isopropanol is a significant contributor DMP433 formation.

Content of Nitrite in Batches of iPrOH Used in the Commercial Production Process

| | | | | Nitrite |
|---|---|---|---|---|
| | | | Nitrite | average |
| Material | Storage | Sample | [ppb] | [ppb] |
| Isopropanol | Truck tanker | I72914503A | <5 ppb | |
| Menges | | | | |
| Isopropanol | Truck tanker | I72914503A | <5 ppb | <5 ppb |
| Menges | | | | |
| Isopropanol | Truck tanker | I72914603A | <5 ppb | |
| Menges | | | | |
| Isopropanol | Truck tanker | I72914603A | <5 ppb | <5 ppb |
| Menges | | | | |
| Isopropanol | Farm tank | I72914803A | <5 ppb | |
| Menges | (delivery Feb. 2, 2024) | | | |
| Isopropanol | Farm tank | I72914803A | <5 ppb | <5 ppb |
| Menges | (delivery Feb. 2, 2024) | | | |
| Isopropanol | Farm tank | I72914903A | <5 ppb | |
| Menges | (delivery Feb. 2, 2024) | | | |
| Isopropanol | Farm tank | I72914903A | <5 ppb | <5 ppb |
| Menges | (delivery Feb. 2, 2024) | | | |
| Isopropanol | Farm tank under N₂ | I72915003A | <5 ppb | |
| Menges | (delivery Feb. 2, 2024) | | | |
| Isopropanol | Farm tank under N₂ | I72915003A | <5 ppb | <5 ppb |
| Menges | (delivery Feb. 2, 2024) | | | |
| Isopropanol | Farm tank under N₂ | I72915103A | <5 ppb | |
| Menges | (delivery Feb. 2, 2024) | | | |
| Isopropanol | Farm tank under N₂ | I72915103A | <5 ppb | <5 ppb |
| Menges | (delivery Feb. 2, 2024) | | | |

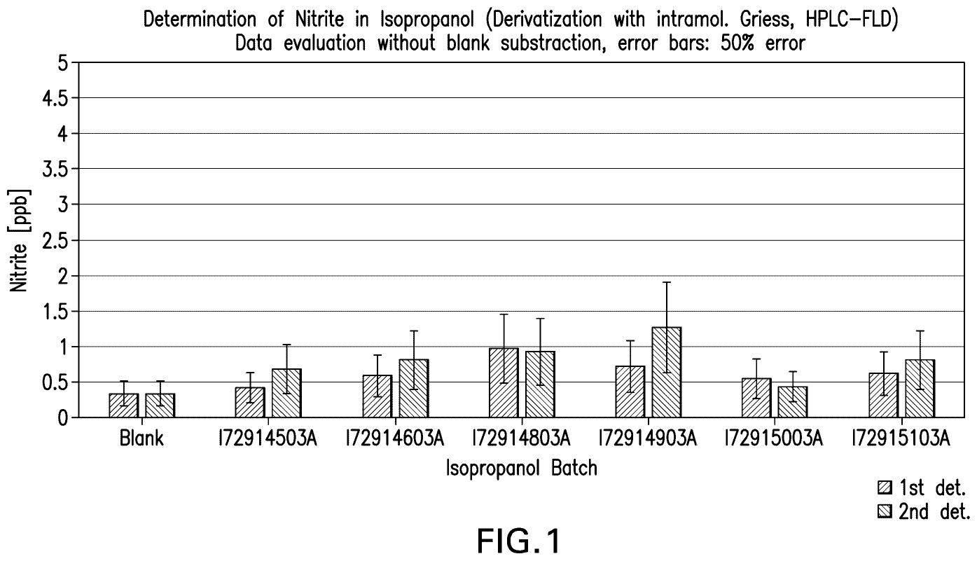

Detailed results are shown in FIG. 1. The results could indicate poorer quality of iPrOH from the not inertised farm tank.

Impact of Water Content on Nitrite in iPrOH

The impact of water content in iPrOH/H₂O solvent mixtures on nitrite content was investigated. The results showed a correlation between water content and nitrite content, indicating that the nitrite content is generated from the hydrolysis of isopropyl nitrite.

Nitrite testing results by IC (Theoretical con. 183.3 ng/mL)

| Solvents of different | | | |
|---|---|---|---|
| ratios of | | nitrite con. T0 | nitrite con. T1_12 h |
| IPA and water | IPA % (v/v) | (ng/ml) | (ng/ml) |
| IPA | 100% | 7.0 | 7.1 |
| IPA/Water = 70/30 | 70% | 6.2 | 7.8 |
| IPA/Water = 50/50 | 50% | 18.7 | 54.9 |
| IPA/Water = 40/60 | 40% | 63.5 | 127.8 |
| IPA/Water = 30/70 | 30% | 161.5 | 181.1 |
| IPA/Water = 20/80 | 20% | 182.8 | 174.1 |
| IPA/Water = 10/90 | 10% | 170.0 | 162.8 |
| Water | 0% | 148.1 | 143.5 |

Further experiments were carried out to determine the active nitrosating species in isopropanol. Isopropyl nitrite was identified.

Storage of Isopropanol (iPrOH)

In order to evaluate the impact of exposing the solvent to air, the nitrite content in batches of iPrOH was measured (i) in fresh commercial batches, i.e., in newly opened bottles (except where indicated otherwise in the table below), and (ii) following exposure to air for two days. For (ii), the cap bottle was opened and the bottle was placed in the hood over the weekend. The results are shown in the following table.

| | | | | Nitrite [ppb] following exposure to |
|---|---|---|---|---|
| Brand | Batch number | Nitrite [ppb] | | air for 2 days |
| SCRC (Lab) | 20230612 | 3.5 | ppb (newly opened) | 155.6 ppb (opened for 2 days) |
| | 20230719 | 3.5 | ppb (newly opened) | 134.6 ppb (opened for 2 days) |
| | 20230823 | 3.9 | ppb (newly opened) | 178.7 ppb (opened for 2 days) |
| | 20231103 | 4.2 | ppb (newly opened) | 199.5 ppb (opened for 2 days) |
| | — | 1.3 | ppb (newly opened) | |
| | 20230612 | 21.0 | ppb (used bottle) | |
| LSC-CN | 1010058236 | 2.1 | ppb | |

The data shows that the average nitrite level in fresh commercial iPrOH batches is about 1-5 ppb. However, the nitrite level increased rapidly if the iPrOH was left exposed to air. It is believed that this may be due to NOx absorption.

### Additional Washing of Isolated Ribociclib Succinate

In order to evaluate if DMP433 is incorporated in the ribociclib succinate crystal or on the surface of the crystal, additional washing was evaluated on a laboratory scale.

| eLN number | Conditions | DMP433 in LEE011-B12 | |
|---|---|---|---|
| LEE011-haenepa1-012 | Current crystallization process with 2 × washes in | 746 | ppb |
| | iPrOH | | |
| LEE011-SUIJQ-002 | Current crystallization process with 2 × washes in | 727 | ppb |
| | iPrOH | | |
| LEE011-haenepa1-013 | Re-slurry of LEE011-B12 (B698294 - 87 ppm of | 64 | ppm |
| | DMP443) in iPrOH at 10° C. | | |
| LEE011-haenepa1-034 | Current crystallization process with 3 × washes | 484 | ppb |

with iPrOH at 25° C.

The experiment using 2 washes of isopropanol showed a level of 746 ppb of DMP433 whereas a material which was washed with 3 washes of isopropanol showed a level of 484 ppb of DMP433 in the final dried cake. Thus, it was demonstrated that the amount of DMP433 in the ribociclib succinate (B12) crystal could be depleted by about 38% as a result of the additional washing.

Depletion of DMP433 may be carried out using ethanol as the purging solvent.

Conclusion from Evaluation

| Evaluated parameter | Impact on DMP433 formation | Implementation in process |
|---|---|---|
| Potassium carbonate | Based on available data, no major | Supplier with low nitrite content will |
| | impact on DMP433 content in B12 | be used as preventive measure |
| Carbon filter | Based on available data, no major | No change planned |
| | impact on DMP433 content in B12 | |
| Polishing filter | EMD Millipore filter showed high | Millipore filter planned to be |
| | impact on DMP433 content in B12 | replaced by a nitrocellulose free |
| | | filter, for example a metal |
| | | (stainless steel) filter, a Pall |
| | | Ilfracombe filter, a Hangzhou |
| | | Cobetter Filtration filter |
| | | A stainless steel (SS 316L polish |
| | | filter) will be introduced for filtration |
| | | of B10 and succinic acid to prevent |
| | | foreign matter contamination |
| | | during salt formation step (IP step). |
| | | The implementation of stainless |
| | | steel filter may involve increasing |
| | | the filter from 10 to 30 inches to |
| | | accelerate addition of succinic |
| | | acid. The stainless steel filter may |
| | | be made with a mesh size of 0.5 |
| | | μm. |
| Succinic acid | Based on available data, no major | Different suppliers of succinic acid |
| | impact on DMP433 content in B12 | were evaluated, but based on |
| | | current analytical methods, no |
| | | significant difference in nitrite |
| | | content could be observed |
| Additional washing of B12 | Based on laboratory data, | Further tests have been performed |
| | additional washing resulted in | (see infra) |
| | depletion of DMP433 in B12 | |
| Isopropanol (iPrOH) | Based on laboratory data, a | Further tests have been performed |
| | distillation of iPrOH showed a | (see infra) |
| | reduction on DMP433 content in | |
| | B12 by approx. 48% | |

### Technical Trials

The impact of a distillation of iPrOH on the content of DMP433 in B12 was investigated. The impact of additional washing of the isolated product on the reduction of DMP433 content in the crystalline drug substance was also evaluated.

Two 5 kg technical batches were manufactured in the pilot plant. Equipment and process were designed to be as close as possible to the those of the commercial setup, although to a smaller scale (compromises with respect to the reactor volume were made). The starting material B10, the succinic acid, and the filter that were used in the process were ordered from the commercial supply pipeline at Raybow. The iPrOH was freshly ordered.

The first technical batch was performed using iPrOH without distillation. The NOx content was <5 ppb, which is the LOQ of the analytical method applied. Extrapolation of the test results gave approx. 3.5 ppb of nitrite in the undistilled/PrOH.

After salt formation, the drug substance was isolated on a filter, and was washed with iPrOH according to the amounts corresponding to those used in the commercial process. After taking a sample, the remaining filter cake was washed with additional/PrOH. After drying, the product was sieved.

The same process was repeated with/PrOH that had been distilled before use in the process and for washing. The NOx content was <5 ppb, which is the LOQ of the analytical method applied. Extrapolation of the test results gave a value of approx. 1.4 ppb of nitrite in the distilled/PrOH.

The obtained products were tested for DMP433, PSD and polymorphic form. The results are summarized below:

| | Technical batch 1 | | Technical batch 2 | | |
|---|---|---|---|---|---|
| | non-distilled i-PrOH | | distilled i-PrOH | | |
| | normal wash | additional wash | normal wash | additional wash | |
| DMP433 in B10 | 55.8 ppb | | 55.8 ppb | | z |
| NOx in i-PrOH | <5 ppb = LOQ (extrapolated 3.5 ppb) | | <5 ppb = LOQ (extrapolated 1.4 ppb) | | |
| Polymorphic form | A | A | A | A | |
| PSD (after sieving) | X50: 339 um | X50: 285 um | X50: 207 um | X50: 194 um | Coarser compared |
| | X90: 782 um | X90: 622 um | X90: 411 um | X90: 383 um | to commercial DS |
| DMP433 | 443 ppb | 404 ppb | 203 ppb | 192 ppb | |

The above results demonstrate that the use of distilled iPrOH reduces the amount of DMP433 that is formed in the process by approx. 50% (compare the results obtained with technical batch 2, distilled/PrOH, with the results obtained for technical batch 1 with non-distilled iPrOH).

For the product prepared using distilled iPrOH, additional washing of the isolated product did not make a major contribution to the depletion of DMP433.

To evaluate any possible impact of the batch size and differences in the equipment, a repeat of these experiments on a commercial scale is planned.

Distillation to Reduce Nitrite Content in/PrOH

The impact of distillation on the content of nitrite in several batches of PrOH was investigated in a further experiment.

| Nitrite in | Nitrite in iPrOH | Nitrite in |
|---|---|---|
| iPrOH before | after distillation | distillation residue |
| distillation [ppb] | [ppb] | [ppb] |
| 21.0 | 3.4 | — |
| 162.7 | 119.6 | 1657.2 |
| 162.7 (repeat) | 73.8 | 1373.4 |

The above results demonstrate that distilling the/PrOH solvent reduced the amount of nitrites in the solvent. Although the distillation could partially remove nitrite from the PrOH, the nitrite was not entirely removed and this is likely caused by the equilibrium of NOx and NO₂⁻ in the system.

Distilation is an efficient means of intervention to reduce nitrite content in/PrOH. The reduced nitrite content after distillation is expected to result in less DMP433 formed.

### Drug Substance Stability

### Stability Data for DMP433 for the Drug Substance (“DS”) Manufactured at Raybow and Grimsby (Standard Process)

| DS | | Storage | Content of DMP433 (ppm) | | | | | | | | |
|---|---|---|---|---|---|---|---|---|---|---|---|
| Manufacturer | Batch no | condition | 0 | 3 M | 6 M | 9 M | 12 M | 18 M | 24 M | 36 M | 48 M |
| Raybow | 833268A092 | 30° C./75% RH | NP | NP | NP | NP | NP | NP | NP | 0.45 | X |
| | 833268A116 | 30° C./75% RH | NP | NP | NP | NP | NP | NP | 0.26 | X | X |
| | 833268A117 | 30° C./75% RH | NP | NP | NP | NP | NP | NP | 0.22 | X | X |
| | 833268A118 | 30° C./75% RH | NP | NP | NP | NP | NP | NP | 0.22 | X | X |
| | 833268A126 | 30° C./75% RH | NP | NP | NP | NP | NP | NP | 0.24 | X | X |
| | 833268A143 | 30° C./75% RH | NP | NP | NP | NP | 0.27 | X | X | X | X |
| | 833268A145 | 30° C./75% RH | NP | NP | NP | NP | 0.21 | X | X | X | X |
| | 834325A007 | 40° C./75% RH | NP | NP | NP | 0.24 | — | — | — | — | — |
| | | 30° C./75% RH | NP | NP | NP | 0.19 | X | X | X | X | X |
| | 834325A001 | 40° C./75% RH | NP | NP | NP | 0.28 | — | — | — | — | — |
| | | 30° C./75% RH | NP | NP | NP | 0.24 | X | X | X | X | X |
| | 834325A004 | 40° C./75% RH | NP | NP | NP | 0.21 | — | — | — | — | — |
| | | 30° C./75% RH | NP | NP | NP | 0.19 | X | X | X | X | X |
| | 834325A010 | 40° C./75% RH | NP | NP | NP | 0.28 | — | — | — | — | — |
| | | 30° C./75% RH | NP | NP | NP | 0.23 | X | X | X | X | X |
| Grimsby | XC0002 | 30° C./75% RH | NP | NP | NP | NP | NP | NP | NP | NP | 0.54 |
| | XC0003 | 30° C./75% RH | NP | NP | NP | NP | NP | NP | NP | NP | 0.63 |
| | XC0004 | 25° C./60% RH | NP | NP | NP | NP | NP | NP | NP | NP | 0.57 |
| | XC0004 | 30° C./75% RH | NP | NP | NP | NP | NP | NP | NP | NP | 0.64 |
| | XC0005 | 30° C./75% RH | NP | NP | NP | NP | NP | NP | NP | NP | 0.61 |
| | XC0006 | 30° C./75% RH | NP | NP | NP | NP | NP | NP | NP | NP | 0.59 |
| | XC0007 | 30° C./75% RH | NP | NP | NP | NP | NP | NP | NP | NP | 0.63 |
| Menges | B692472 | 30/75 | 0.37 | 0.25 | 0.68 | | | | | | |
| | B692472 | 40/75 | 0.37 | 0.26 | 1.06 | | | | | | |
| | B695104 | 30/75 | 76.702 | 66.52 | 60.53 | | | | | | |
| | B695104 | 40/75 | 76.702 | 65.86 | 56.15 | | | | | | |
| | B695105 | 30/75 | 71.498 | 61.87 | 57.22 | | | | | | |
| | B695105 | 40/75 | 71.498 | 63.26 | 60.27 | | | | | | |
| | B695106 | 30/75 | 0.82 | 0.63 | 0.89 | | | | | | |
| | B695106 | 40/75 | 0.82 | 0.68 | 1.17 | | | | | | |
| | B696587 | 30/75 | 0.383 | 0.36 | | | | | | | |
| | B696587 | 40/75 | 0.383 | 0.46 | | | | | | | |
| | B711867 | 30/75 | 0.3 | | | | | | | | |
| | B711867 | 40/75 | 0.3 | | | | | | | | |

NP: Not performed
X: Planned testing
— Not planned

### Stability Data for DMP433 for the Drug Substance (“DS”) Manufactured at Menges

| DS | Batch | Year of | Storage | Nitrite content T0 | Content of DMP433 (ppm) | | | | | | | | |
|---|---|---|---|---|---|---|---|---|---|---|---|---|---|
| Manufacturer | no | manufacture | condition | (enhanced method) | 0 | 3 M | 6 M | 9 M | 12 M | 18 M | 24 M | 36 M | 48 M |
| Menges (DS06) | B692472 | 2023 | 30/75 | 0.1 | 0.37 | 0.25 | 0.68 | | | | | | |
| | B692472 | 2023 | 40/75 | 0.1 | 0.37 | 0.26 | 1.06 | | | | | | |
| | B695104 | 2023 | 30/75 | 0.5 | 76.702 | 66.52 | 60.53 | | | | | | |
| | B695104 | 2023 | 40/75 | 0.5 | 76.702 | 65.86 | 56.15 | | | | | | |
| | B695105 | 2023 | 30/75 | 0.3 | 71.498 | 61.87 | 57.22 | | | | | | |
| | B695105 | 2023 | 40/75 | 0.3 | 71.498 | 63.26 | 60.27 | | | | | | |
| | B695106 | 2023 | 30/75 | 0.1 | 0.82 | 0.63 | 0.89 | | | | | | |
| | B695106 | 2023 | 40/75 | 0.1 | 0.82 | 0.68 | 1.17 | | | | | | |
| | B696587 | 2023 | 30/75 | <0.1 | 0.383 | 0.36 | | | | | | | |
| | B696587 | 2023 | 40/75 | <0.1 | 0.383 | 0.46 | | | | | | | |
| | B711867 | 2023 | 30/75 | <0.1 | 0.3 | | | | | | | | |
| | B711867 | 2023 | 40/75 | <0.1 | 0.3 | | | | | | | | |

### Quality Assessment of Drug Substance (DS) Batches at Release

| | | | DMP433 | Nitrite | Nitrite NAT |
|---|---|---|---|---|---|
| | | | (LC- | NAP | enhanced |
| | | | HRMS) | method | sample prep. |
| Material | Manufacturer | Control Nr. | (ppm) | (ppm) | (ppm) |
| RIBOCICLIB | Menges - DS06 | B692472 | 0.364 | 0.2 | 0.1 |
| SUCCINATE | | | | | |
| RIBOCICLIB | Menges - DS06 | B695104 | 76.702 | 0.3 | 0.5 |
| SUCCINATE | | | | | |
| RIBOCICLIB | Menges - DS06 | B695105 | 71.498 | 0.3 | 0.3 |
| SUCCINATE | | | | | |
| RIBOCICLIB | Menges - DS06 | B695106 | 0.82 | 0.2 | 0.1 |
| SUCCINATE | | | | | |
| RIBOCICLIB | Menges - DS06 | B696587 | 0.383 | 0.2 | <0.1 |
| SUCCINATE | | | | | |
| RIBOCICLIB | Menges - DS06 | B696588 | 0.537 | 0.2 | 0.1 |
| SUCCINATE | | | | | |
| RIBOCICLIB | Menges - DS06 | B696589 | 0.363 | 0.2 | <0.1 |
| SUCCINATE | | | | | |
| RIBOCICLIB | Menges - DS06 | B696590 | 58.916 | 0.3 | 0.4 |
| SUCCINATE | | | | | |
| RIBOCICLIB | Menges - DS06 | B698293 | 59.598 | 0.3 | 0.4 |
| SUCCINATE | | | | | |
| RIBOCICLIB | Menges - DS06 | B698294 | 95.528 | 0.2 | 0.2 |
| SUCCINATE | | | | | |
| RIBOCICLIB | Menges - DS06 | B698295 | 86.595 | 0.3 | 0.4 |
| SUCCINATE | | | | | |
| RIBOCICLIB | Menges - DS06 | B698296 | 87.001 | 0.4 | 0.3 |
| SUCCINATE | | | | | |
| RIBOCICLIB | Menges - DS06 | B698297 | 1.211 | 0.2 | 0.1 |
| SUCCINATE | | | | | |
| RIBOCICLIB | Menges - DS06 | B698298 | 0.694 | 0.2 | <0.1 |
| SUCCINATE | | | | | |
| RIBOCICLIB | Menges - DS06 | B699003 | 0.71 | 0.1 | <0.1 |
| SUCCINATE | | | | | |
| RIBOCICLIB | Menges - DS06 | B707657 | 0.41 | 0.2 | 0.1 |
| SUCCINATE | | | | | |
| RIBOCICLIB | Menges - DS06 | B708687 | 0.25 | 0.2 | <0.1 |
| SUCCINATE | | | | | |
| RIBOCICLIB | Menges - DS06 | B708688 | 0.23 | 0.2 | <0.1 |
| SUCCINATE | | | | | |
| RIBOCICLIB | Menges - DS06 | B709049 | 0.13 | 0.2 | <0.1 |
| SUCCINATE | | | | | |
| RIBOCICLIB | Menges - DS06 | B709050 | 0.29 | 0.2 | 0.1 |
| SUCCINATE | | | | | |
| RIBOCICLIB | Menges - DS06 | B709051 | 0.33 | 0.2 | <0.1 |
| SUCCINATE | | | | | |
| RIBOCICLIB | Menges - DS06 | B709938 | 0.52 | 0.2 | <0.1 |
| SUCCINATE | | | | | |
| RIBOCICLIB | Menges - DS06 | B709939 | 0.45 | 0.2 | <0.1 |
| SUCCINATE | | | | | |
| RIBOCICLIB | Menges - DS06 | B709940 | 0.34 | 0.2 | <0.1 |
| SUCCINATE | | | | | |
| RIBOCICLIB | Menges - DS06 | B709941 | 0.16 | 0.1 | 0.1 |
| SUCCINATE | | | | | |
| RIBOCICLIB | Menges - DS06 | B710665 | 0.17 | 0.2 | 1.6 |
| SUCCINATE | | | | | |
| RIBOCICLIB | Menges - DS06 | B710666 | 0.19 | 0.5 | <0.1 |
| SUCCINATE | | | | | |
| RIBOCICLIB | Menges - DS06 | B710667 | 0.21 | / | / |
| SUCCINATE | | | | | |
| RIBOCICLIB | Menges - DS06 | B710668 | 0.29 | 0.2 | 0.1 |
| SUCCINATE | | | | | |
| RIBOCICLIB | Menges - DS06 | B710669 | 0.4 | 0.1 | <0.1 |
| SUCCINATE | | | | | |
| RIBOCICLIB | Menges - DS06 | B711439 | 0.22 | 0.1 | <0.1 |
| SUCCINATE | | | | | |
| RIBOCICLIB | Menges - DS06 | B711440 | 0.19 | 0.1 | 0.9 |
| SUCCINATE | | | | | |
| RIBOCICLIB | Menges - DS06 | B711866 | 0.3 | 0.2 | <0.1 |
| SUCCINATE | | | | | |
| RIBOCICLIB | Menges - DS06 | B711867 | 0.3 | 0.1 | 0.1 |
| SUCCINATE | | | | | |
| RIBOCICLIB | Menges - DS06 | B711868 | 0.52 | / | <0.1 |
| SUCCINATE | | | | | |

Stability data (see infra) for low nitrosamine batches showed that DMP433 increased more under accelerated conditions (40° C./75% RH). However, a slight increase was also observed under regular conditions, indicating that some nitrosating agents may be present in the DS. For high nitrosamine batches, a slight decrease under both conditions was observed.

| | | | | Nitrite NAP | Nitrite NAT |
|---|---|---|---|---|---|
| | | | DMP433 (LC- | method | enhanced sample |
| Material | Manufacturer | Control Nr. | HRMS) (ppm) | (ppm) | prep. (ppm) |
| RIBOCICLIB | Raybow - DS04 | 833268A037 | 0.90 | / | / |
| SUCCINATE | | | | | |
| RIBOCICLIB | Raybow - DS04 | 833268A039 | 0.89 | / | / |
| SUCCINATE | | | | | |
| RIBOCICLIB | Raybow - DS04 | 833268A039 | 0.89 | / | / |
| SUCCINATE | | | | | |
| RIBOCICLIB | Raybow - DS04 | 833268A045 | 0.82 | / | / |
| SUCCINATE | | | | | |
| RIBOCICLIB | Raybow - DS04 | 833268A049 | 0.94 | / | / |
| SUCCINATE | | | | | |
| RIBOCICLIB | Raybow - DS04 | 833268A050 | 0.85 | / | / |
| SUCCINATE | | | | | |
| RIBOCICLIB | Raybow - DS04 | 833268A051 | 0.83 | / | / |
| SUCCINATE | | | | | |
| RIBOCICLIB | Raybow - DS04 | 833268A055 | 0.55 | / | / |
| SUCCINATE | | | | | |
| RIBOCICLIB | Raybow - DS04 | 833268A060 | 1.17 | / | / |
| SUCCINATE | | | | | |
| RIBOCICLIB | Raybow - DS04 | 833268A061 | 0.84 | / | / |
| SUCCINATE | | | | | |
| RIBOCICLIB | Raybow - DS04 | 833268A064 | 1.00 | / | / |
| SUCCINATE | | | | | |
| RIBOCICLIB | Raybow - DS04 | 833268A068 | 0.79 | / | / |
| SUCCINATE | | | | | |
| RIBOCICLIB | Raybow - DS04 | 833268A069 | 0.93 | / | / |
| SUCCINATE | | | | | |
| RIBOCICLIB | Raybow - DS04 | 833268A069 | 0.93 | / | / |
| SUCCINATE | | | | | |
| RIBOCICLIB | Raybow - DS04 | 833268A070 | 0.88 | / | / |
| SUCCINATE | | | | | |
| RIBOCICLIB | Raybow - DS04 | 833268A070 | 0.88 | / | / |
| SUCCINATE | | | | | |
| RIBOCICLIB | Raybow - DS04 | 833268A084 | 0.50 | / | / |
| SUCCINATE | | | | | |
| RIBOCICLIB | Raybow - DS04 | 833268A085 | 0.80 | / | / |
| SUCCINATE | | | | | |
| RIBOCICLIB | Raybow - DS04 | 833268A092 | 0.40 | / | / |
| SUCCINATE | | | | | |
| RIBOCICLIB | Raybow - DS04 | 833268A096 | 0.29 | / | / |
| SUCCINATE | | | | | |
| RIBOCICLIB | Raybow - DS04 | 833268A110 | 0.43 | / | / |
| SUCCINATE | | | | | |
| RIBOCICLIB | Raybow - DS04 | 833268A111 | 0.46 | / | / |
| SUCCINATE | | | | | |
| RIBOCICLIB | Raybow - DS04 | 833268A131 | 0.29 | / | / |
| SUCCINATE | | | | | |
| RIBOCICLIB | Raybow - DS04 | 833268A132 | 0.42 | / | / |
| SUCCINATE | | | | | |
| RIBOCICLIB | Raybow - DS04 | 833268A133 | 0.44 | / | / |
| SUCCINATE | | | | | |
| RIBOCICLIB | Raybow - DS04 | 833268A136 | 0.46 | / | / |
| SUCCINATE | | | | | |
| RIBOCICLIB | Raybow - DS04 | 833268A139 | 0.59 | / | / |
| SUCCINATE | | | | | |
| RIBOCICLIB | Raybow - DS04 | 833268A144 | 0.23 | / | <0.1 |
| SUCCINATE | | | | | |
| RIBOCICLIB | Raybow - DS04 | 833268A146 | 0.29 | / | / |
| SUCCINATE | | | | | |
| RIBOCICLIB | Raybow - DS04 | 833268A148 | 0.38 | / | / |
| SUCCINATE | | | | | |
| RIBOCICLIB | Raybow - DS04 | 833268A151 | 0.37 | / | / |
| SUCCINATE | | | | | |
| RIBOCICLIB | Raybow - DS04 | 833268A152 | 0.27 | / | / |
| SUCCINATE | | | | | |
| RIBOCICLIB | Raybow - DS04 | 833268A153 | 0.33 | / | / |
| SUCCINATE | | | | | |
| RIBOCICLIB | Raybow - DS04 | 833268A156 | 0.47 | / | / |
| SUCCINATE | | | | | |
| RIBOCICLIB | Raybow - DS04 | 833268A167 | 0.33 | / | / |
| SUCCINATE | | | | | |
| RIBOCICLIB | Raybow - DS04 | 833268A193 | 0.33 | / | 0.1 |
| SUCCINATE | | | | | |
| RIBOCICLIB | Raybow - DS04 | 833268A196 | 0.31 | / | 1.1 |
| SUCCINATE | | | | | |
| RIBOCICLIB | Raybow - DS04 | 833268A198 | 0.29 | / | 0.1 |
| SUCCINATE | | | | | |
| RIBOCICLIB | Raybow - DS04 | 833268A201 | 0.35 | / | 0.1 |
| SUCCINATE | | | | | |
| RIBOCICLIB | Raybow - DS04 | 833268A202 | 0.38 | / | 0.1 |
| SUCCINATE | | | | | |
| RIBOCICLIB | Raybow - DS04 | 833268A207 | 0.43 | / | 0.1 |
| SUCCINATE | | | | | |
| RIBOCICLIB | Raybow - DS04 | 833268A211 | 0.29 | / | <0.1 |
| SUCCINATE | | | | | |
| RIBOCICLIB | Raybow - DS04 | 833268A212 | 0.27 | / | <0.1 |
| SUCCINATE | | | | | |
| RIBOCICLIB | Raybow - DS04 | 833268A252 | 0.37 | / | / |
| SUCCINATE | | | | | |
| RIBOCICLIB | Raybow - DS04 | 833268A253 | 0.28 | / | / |
| SUCCINATE | | | | | |
| RIBOCICLIB | Raybow - DS04 | 833268A254 | 0.25 | / | / |
| SUCCINATE | | | | | |
| RIBOCICLIB | Raybow - DS05 | 834325A003 | 0.15 | / | / |
| SUCCINATE | | | | | |
| RIBOCICLIB | Raybow - DS05 | 834325A006 | 0.16 | / | / |
| SUCCINATE | | | | | |
| RIBOCICLIB | Raybow - DS05 | 834325A009 | 0.18 | / | / |
| SUCCINATE | | | | | |
| RIBOCICLIB | Raybow - DS05 | 834325A012 | 0.21 | / | / |
| SUCCINATE | | | | | |
| RIBOCICLIB | Raybow - DS05 | 834325A015 | 0.33 | / | 0.1 |
| SUCCINATE | | | | | |
| RIBOCICLIB | Raybow - DS05 | 834325A016 | 0.28 | / | 0.1 |
| SUCCINATE | | | | | |
| RIBOCICLIB | Raybow - DS05 | 834325A023 | 0.26 | / | / |
| SUCCINATE | | | | | |
| RIBOCICLIB | Raybow - DS05 | 834325A024 | 0.22 | / | / |
| SUCCINATE | | | | | |
| RIBOCICLIB | Raybow - DS05 | 834325A025 | 0.28 | / | / |
| SUCCINATE | | | | | |
| RIBOCICLIB | Raybow - DS05 | 834325A026 | 0.24 | / | / |
| SUCCINATE | | | | | |
| RIBOCICLIB | Raybow - DS05 | 834325A027 | 0.24 | / | / |
| SUCCINATE | | | | | |
| RIBOCICLIB | Raybow - DS05 | 834325A028 | 0.31 | / | / |
| SUCCINATE | | | | | |
| RIBOCICLIB | Raybow - DS05 | 834325A029 | 0.28 | / | / |
| SUCCINATE | | | | | |
| RIBOCICLIB | Raybow - DS05 | 834325A030 | 0.32 | / | / |
| SUCCINATE | | | | | |
| RIBOCICLIB | Raybow - DS05 | 834325A031 | 0.31 | / | / |
| SUCCINATE | | | | | |
| RIBOCICLIB | Raybow - DS05 | 834325A032 | 0.29 | / | / |
| SUCCINATE | | | | | |
| RIBOCICLIB | Raybow - DS05 | 834325A033 | 0.26 | / | / |
| SUCCINATE | | | | | |
| RIBOCICLIB | Raybow - DS05 | 834325A034 | 0.30 | / | / |
| SUCCINATE | | | | | |
| RIBOCICLIB | Raybow - DS05 | 834325A035 | 0.33 | / | / |
| SUCCINATE | | | | | |
| RIBOCICLIB | Raybow - DS05 | 834325A036 | 0.32 | / | / |
| SUCCINATE | | | | | |
| RIBOCICLIB | Raybow - DS05 | 834325A015 | 0.26 | / | 0.1 |
| SUCCINATE | | | | | |
| RIBOCICLIB | Raybow - DS05 | 834325A016 | 0.32 | / | 0.1 |
| SUCCINATE | | | | | |
| RIBOCICLIB | Raybow - DS05 | 833268A196 | 0.31 | / | 0.1 |
| SUCCINATE | | | | | |
| RIBOCICLIB | Raybow - DS05 | 833268A144 | 0.31 | / | <0.1 |
| SUCCINATE | | | | | |
| RIBOCICLIB | Raybow - DS05 | 833268A193 | 0.33 | / | 0.1 |
| SUCCINATE | | | | | |
| RIBOCICLIB | Raybow - DS05 | 833268A198 | 0.28 | / | 0.1 |
| SUCCINATE | | | | | |
| RIBOCICLIB | Raybow - DS05 | 833268A201 | 0.26 | / | 0.1 |
| SUCCINATE | | | | | |
| RIBOCICLIB | Raybow - DS05 | 833268A202 | 0.29 | / | 0.1 |
| SUCCINATE | | | | | |
| RIBOCICLIB | Raybow - DS05 | 833268A207 | 0.25 | / | 0.1 |
| SUCCINATE | | | | | |
| RIBOCICLIB | Raybow - DS05 | 833268A255 | 0.26 | / | |
| SUCCINATE | | | | | |
| RIBOCICLIB | Raybow - DS05 | 833268A256 | 0.32 | / | |
| SUCCINATE | | | | | |
| RIBOCICLIB | Raybow - DS05 | 833268A257 | 0.31 | / | |
| SUCCINATE | | | | | |
| RIBOCICLIB | Raybow - DS05 | 833268A258 | 0.31 | / | |
| SUCCINATE | | | | | |
| RIBOCICLIB | Raybow - DS05 | 833268A259 | 0.33 | / | |
| SUCCINATE | | | | | |
| RIBOCICLIB | Raybow - DS05 | 833268A260 | 0.28 | / | |
| SUCCINATE | | | | | |
| RIBOCICLIB | Raybow - DS05 | 833268A261 | 0.26 | / | |
| SUCCINATE | | | | | |
| RIBOCICLIB | Raybow - DS05 | 833268R017 | 0.29 | / | |
| SUCCINATE | | | | | |
| RIBOCICLIB | Raybow - DS05 | 833268R018 | 0.25 | / | |
| SUCCINATE | | | | | |
| RIBOCICLIB | Raybow - DS05 | 833268A262 | 0.13 | / | |
| SUCCINATE | | | | | |
| RIBOCICLIB | Raybow - DS05 | 833268A263 | 0.16 | / | |
| SUCCINATE | | | | | |
| RIBOCICLIB | Raybow - DS05 | 833268A264 | 0.19 | / | |
| SUCCINATE | | | | | |
| RIBOCICLIB | Raybow - DS05 | 833268A265 | 0.13 | / | |
| SUCCINATE | | | | | |
| RIBOCICLIB | Raybow - DS05 | 833268A266 | 0.17 | / | |
| SUCCINATE | | | | | |
| RIBOCICLIB | Raybow - DS05 | 833268A267 | 0.21 | / | |
| SUCCINATE | | | | | |
| RIBOCICLIB | Raybow - DS05 | 833268A268 | 0.19 | / | |
| SUCCINATE | | | | | |
| RIBOCICLIB | Raybow - DS05 | 833268A269 | 0.18 | / | |
| SUCCINATE | | | | | |
| RIBOCICLIB | Raybow - DS05 | 833268A270 | 0.2 | / | |
| SUCCINATE | | | | | |
| RIBOCICLIB | Raybow - DS05 | 833268A271 | 0.22 | / | |
| SUCCINATE | | | | | |
| RIBOCICLIB | Raybow - DS04 | 833268R001 | 0.23 | / | / |
| SUCCINATE | | | | | |
| RIBOCICLIB | Raybow - DS04 | 833268R002 | 0.32 | / | / |
| SUCCINATE | | | | | |
| RIBOCICLIB | Raybow - DS04 | 833268R003 | 0.29 | / | / |
| SUCCINATE | | | | | |
| RIBOCICLIB | Raybow - DS04 | 833268R004 | 0.31 | / | / |
| SUCCINATE | | | | | |
| RIBOCICLIB | Raybow - DS04 | 833268R005 | 0.31 | / | / |
| SUCCINATE | | | | | |
| RIBOCICLIB | Raybow - DS04 | 833268R006 | 0.32 | / | / |
| SUCCINATE | | | | | |
| RIBOCICLIB | Raybow - DS04 | 833268R007 | 0.25 | / | / |
| SUCCINATE | | | | | |
| RIBOCICLIB | Raybow - DS04 | 833268R008 | 0.11 | / | 0.3 |
| SUCCINATE | | | | | |
| RIBOCICLIB | Raybow - DS04 | 833268R009 | 0.10 | / | <0.1 |
| SUCCINATE | | | | | |
| RIBOCICLIB | Raybow - DS04 | 833268R010 | 0.13 | / | <0.1 |
| SUCCINATE | | | | | |
| RIBOCICLIB | Raybow - DS04 | 833268R011 | 0.14 | / | <0.1 |
| SUCCINATE | | | | | |
| RIBOCICLIB | Raybow - DS04 | 833268R012 | 0.12 | / | <0.1 |
| SUCCINATE | | | | | |
| RIBOCICLIB | Raybow - DS04 | 833268R013 | 0.15 | / | <0.1 |
| SUCCINATE | | | | | |
| RIBOCICLIB | Raybow - DS04 | 833268R014 | 0.14 | / | <0.1 |
| SUCCINATE | | | | | |
| RIBOCICLIB | Raybow - DS04 | 833268R015 | 0.15 | / | <0.1 |
| SUCCINATE | | | | | |
| RIBOCICLIB | Raybow - DS04 | 833268R016 | 0.12 | / | <0.1 |
| SUCCINATE | | | | | |

### Further Nitrite Testing Gave the Following Results:

| | | | DMP433 | Nitrite |
|---|---|---|---|---|
| Material | Manufacturer | Control Nr. | (ppb) | (ppb) |
| LEE011-DS | Raybow | 833268A258 | 380 | 369 |
| | Raybow | 833268A259 | 395 | 284 |

An overview of the release results of DMP433 in the drug substance (DS) is provided in FIG. 2.

### Further Evaluation of Parameters Discussed Hereinabove

A further experiment was carried out to evaluate the nitrite content in the reagents and filters, and their potential impact on the DMP433 formation. The following synthesis steps were considered:

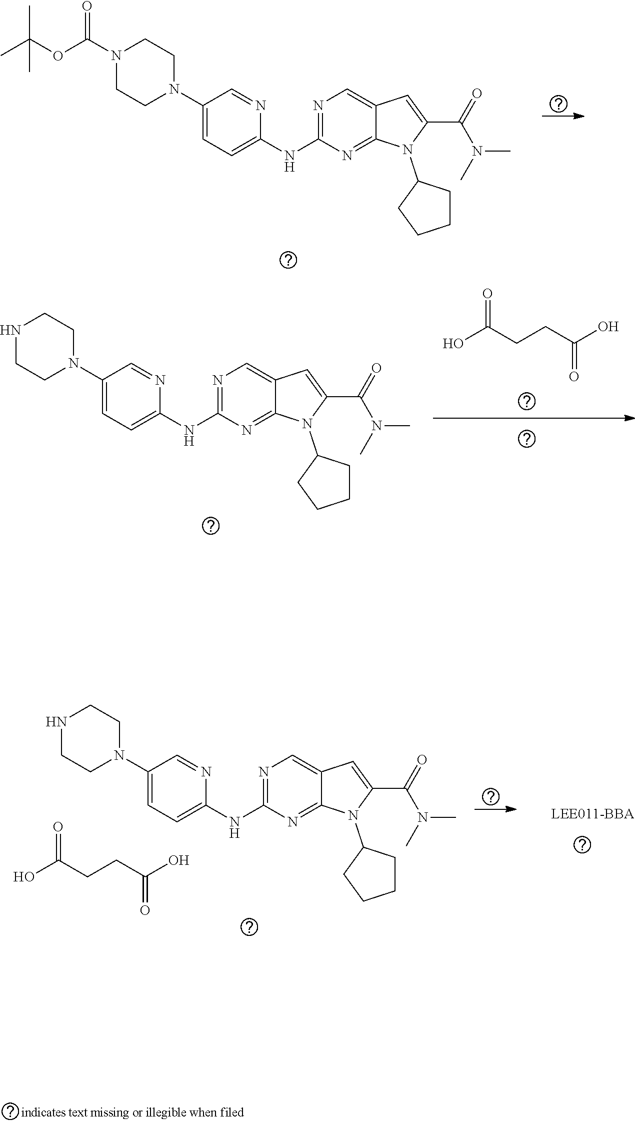

The results of the evaluation are provided in the tables below.

Step B9 to B10

| | NO₂⁻ in B10 | |
|---|---|---|
| NO₂⁻ in K₂CO₃ [ppm] | LOD (1 ppm) | DMP433 [ppm] in B10 |
| 0.5-34 ppm | <LOD | Raybow: 0.03-0.06 |
| | | Menges: 0.008-0.037 |
| Observation: There | Observation: the nitrite | The low DMP433 levels in B10 suggest |
| appears to be no | levels in B10 did not appear | that DMP433 formation would primarily |
| correlation to the | to contribute significantly to | take place in the salt formation step |
| DMP levels observed | any DMP433 formation in | |
| in the DS | the next step | |

Step B10 to DS

| | | | DMP433 |
|---|---|---|---|
| | | | [ppm] in |
| NO₂⁻ in Succinic acid | NO₂⁻ on Carbon filter | Polish filter | DS |
| <0.1 ppm | <1 ppm | EMD Milliporefilter: | 58-95 |
| | | Cobetter filter <LOQ: | ppm |
| | | PALL filter <LOQ | 0.15-0.5 |
| | | | ppm |
| Observation: Low nitrite | Observation: Low nitrite | Observation: There appears to | |
| content was found. Thus, | content was found. Thus, this | be a clear correlation between | |
| this does not appear to be a | parameter does not appear to | the use of Millipore filter | |
| root cause for any increase | be a root cause for any | (nitrocellulose membrane) and | |
| of DMP433 in the DS | increase of DMP433 in DS. | DMP433 generation, as | |
| | | confirmed by a leaching | |
| | | experiment. | |
| | | Leaching experiments for other | |
| | | filter suppliers were carried out, | |
| | | and an increase of DMP was | |
| | | not observed. | |

Distillation of Isopropanol (iPrOH) Over Ascorbic Acid

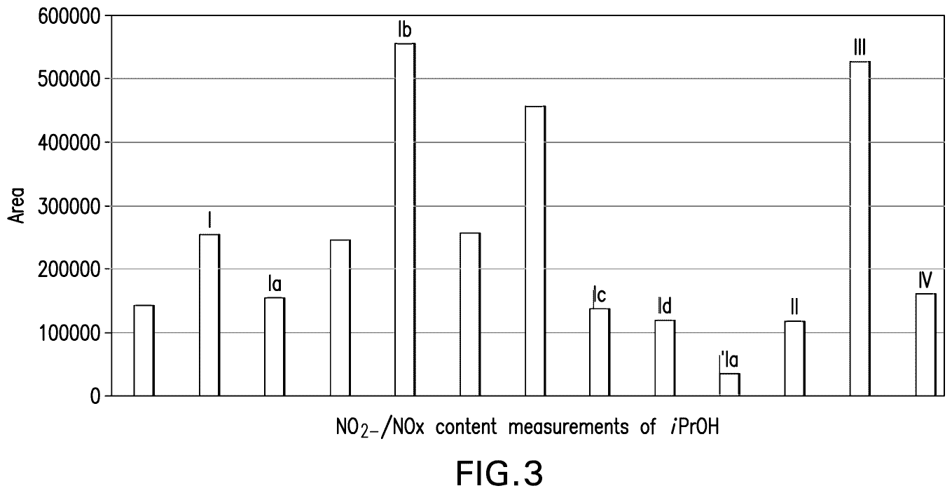

Batches of isopropanol (iPrOH) were tested for their content of nitrite and NOx. The batches tested included untreated “from the shelf” iPrOH (see I, II, III and IV below) where: I-III were lab samples and IV was a production sample used in a commercial campaign. Ia was a distilled sample of I. Ib was a sample of I purged with compressed air. Ic was a distilled sample of I after four days under an argon atmosphere. Id was a sample of I distilled over ascorbic acid. IIa was a sample of II distilled over and through ascorbic acid. The results are presented in FIG. 3.

The results suggest that the quality of the PrOH, with respect to the content of the nitrite/NOx, can vary from batch to batch. Treatment of the/PrOH with ascorbic acid was shown to reduce the nitrite/NOx levels.

Further discussion of the impact of distilling/PrOH over ascorbic acid can be found in Example 13 below.

### Nitrite Content

In many of the examples herein, the nitrite content in the system is evaluated. Spiking experiments confirmed that nitrite is a major cause for the generation of DMP433 (at around the hundreds ppb level) in the ribociclib succinate drug substance (LEE011-IP/DS). It was observed that nitrate (NO₃⁻) did not cause DMP433 generation. Thus, the spiking experiments confirmed that nitrite levels are a root cause of DMP433 generation.

| | DMP433 [ppb] | Nitrite [ppm] |
|---|---|---|
| | in LEE011-B12 | in LEE011-B12 |
| LEE011-B10 sample | (IP) | (IP) |
| B10 (batch 805193A078) control | 995 | 448 |
| experiment | | |
| B10 (batch 805193A078) spiked | 66100 | 335 |
| with ~20 ppm NaNO₂* | | |
| B10 (batch 805193A078) spiked | 888 | 368 |
| with ~20 ppm NaNO₃ | | |

*20 ppm of NaNO₂ in B10 equals 310 ppb of nitrite in B10/IPA solution

### Example 3—Ion Exchange Resins

More than 100 heterogeneous nitrite adsorbers (resin, active charcoal, functionalized silica gel, celites, Al₂O₃, etc.) were screened. These experiments showed that some of the ion exchange resins successfully reduced the nitrite amount in B10/IPA (IPA=isopropanol) solution.

There were two ways to carry out the ion exchange procedure, namely to carry out ion exchange using IPA solvent only (see option 1 process below) or on both IPA solvent and B10/IPA solution (see option 2 process below). In option 2, nitrite carried over from B10 can also be removed.

### Option 1

Step 1: Distil IPA
Step 2: Use resin to treat the distilled IPA
Step 3: Filtration to remove resin
Step 4: The obtained purified IPA to be used in LEE011-B12 step

### Option 2

In the B12 process, there are two portions of solution (B10 in IPA and succinic acid in IPA) that were prepared and mixed in a vessel to obtain LEE011-B12.
Step 1: B10 dissolved in distilled IPA to afford B10/IPA solution
Step 2: Use resin to treat the obtained B10/IPA solution
Step 3: Succinic acid dissolved in distilled and resin treated IPA to afford succinic acid/IPA solution (Note: Succinic acid/IPA solution could not be treated with resin since succinic acid will also be partially removed by resin)
Step 4: Follow the current process to obtain LEE011-B12

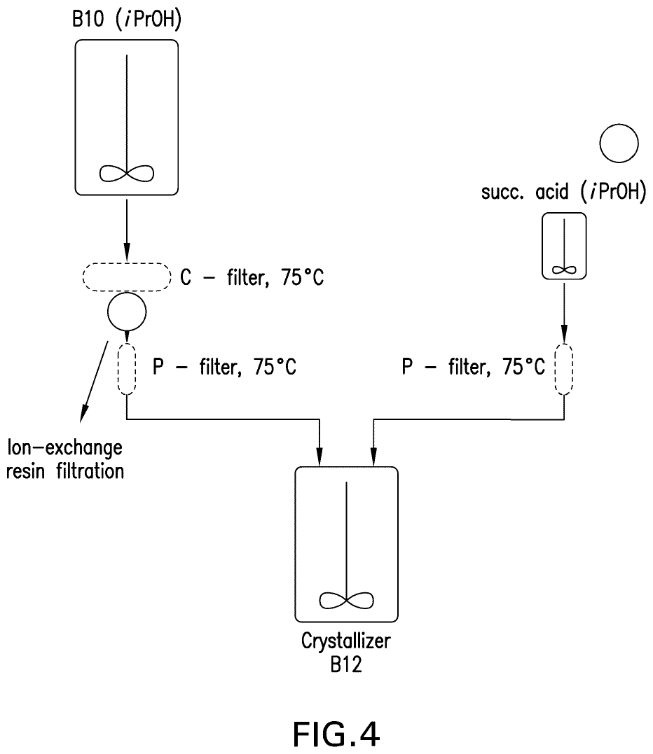

According to option 2, treatment of feed solutions prior to crystallization of drug substance was carried out according to the schematic shown in FIG. 4.

The following Table shows the results of studies carried out to investigate the impact of ion exchange resin and distillation on the amount of DMP433 in LEE011-IP in a set of cross control experiments. The aim of these experiments was to directly compare the impact of distillation and/or resin treatment on reducing the level of DMP433 in LEE011-IP.

| [symbol] | [symbol] | [symbol] | [symbol] | [symbol] | [symbol] | [symbol] | [symbol] |
|---|---|---|---|---|---|---|---|
| 1 | Raybow | 162.7 | No | No | 18011 ppb | on-going | — |
| 2 | (Batch | ppb | Yes* | No | 14238 ppb | on-going | — |
| 3 | 805193A078) | | No | Yes | 1002 ppb | on-going | <100 ppb (n.d.) |
| 4 | DMP433: 92 ppb | 1.3-1.6 | Yes* | Yes | 377 ppb | on-going | <100 ppb (n.d.) |
| 5 | (tested on | ppb | No | No | 995 ppb | 448 ppb | — |
| 6 | Feb 4^{th}) | | Yes | Yes | 220 ppb | on-going | <100 ppb (n.d.) |
| | Nitrite: 257 ppb | | | | | | |

*Nitrite reduced from 162.7 ppb to 73.8 ppb after distillation
** Treated with Amberlite MB resin (commercially available from DuPont)
For entries 1 and 5: the original B12 process was followed with IPA neither distilled nor treated with resin, as control experiments
For entry 2: option 1 process
For entries 4 and 6: option 2 process
For entry 3: in this experiment, the practice was similar to the option 2 process except that the IPA was not distilled [symbol]
indicates data missing or illegible when filed

The yield for the commercial LEE011-IP step is ˜90%. Isolated yield for all gram-scale drug substance reactions with resin treatment varies from 85% to 90%. B10 loss in resin is less than 5%. No new impurity above the identification threshold was observed with cross-checked HPLC methods.

Studies were also carried out to assess the impact of residual nitrite in B10. The results are shown in the Table below.

| | | | DMP433 in | Nitrite in |
|---|---|---|---|---|
| | Nitrite | | LEE011- | LEE011- |
| LEE011-B10 | in IPA | Resin treatment | B12 (IP) | B12 (IP) |
| Raybow (Batch | 1.6 ppb | Only IPA (nitrite | 528 ppb | on-going |
| 805193A078) | | carryover from | | |
| | | B10 remained) | | |
| DMP433: 92 ppb | | B10/IPA solution | 220 ppb | on-going |
| Nitrite 257 ppb | | | | |

DMP433 and nitrite levels in LEE011-B10 could be further reduced by using purified potassium carbonate (with very low [NO₂⁻]) in IP-1 step.

Experiments were also conducted (according to the option 2 process described above) to investigate the impact on DMP433 levels of changing the loading of the ion exchange resin. Increasing resin loading failed to further reduce DMP433 level, very likely caused by equilibrium of adsorption and desorption of nitrite. An ion exchange resin with stronger nitrite depletion is required to achieve lower DMP433 levels in LEE011-IP/DS. The results are shown in the following table.

| | | | Resin | DMP433 in | Nitrite in | NDMA in |
|---|---|---|---|---|---|---|
| | | Nitrite | Loading | LEE011-B12 | LEE011-B12 | LEE011-B12 |
| Entry | LEE011-B10 | in IPA | vs B10 | (IP) | (IP) | (IP) |
| 1 | Raybow (Batch | 1.6 | 20 w/w % | 220 ppb | on-going | <100 ppb |
| | 805193A078) | ppb | | | | (n.d.) |
| 2 | DMP433: | | 50 w/w % | 211 ppb | on-going | <100 ppb |
| | 92 ppb | | | (wet cake) | | (n.d.) |
| 3 | Nitrite | | 100 w/w % | 238 ppb | on-going | <100 ppb |
| | 257 ppb | | | (wet cake) | | (n.d.) |

Nb. for a resin loading of 20 w/w %, if B10 is 100 g then 20 g of resin will be added.

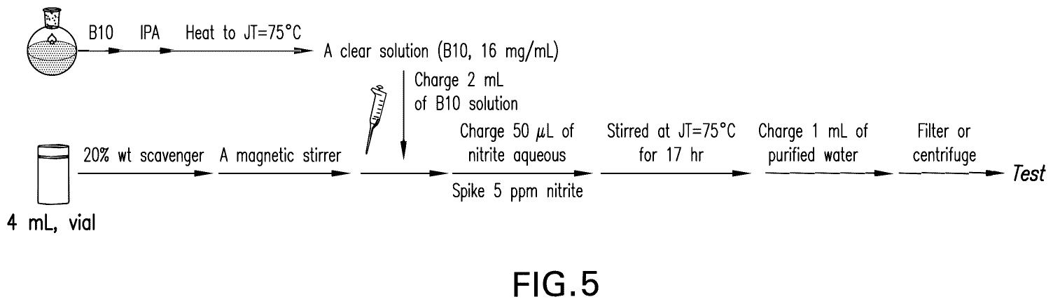

Experiments were also conducted to screen and select the top nitrite remover candidates, which could then be further studied according to the processes of options 1 and 2. In these experiments, B10 was dissolved in IPA, and then the B10/IPA solution was spiked with nitrite. The desired nitrite remover should be able to reduce the nitrite content without inducing an increase in DMP433 level. Under certain conditions, especially where the reaction mixture was acidic, nitrite could react with B10 to form DMP433. This is the reason why, in various cases, the nitrite level decreased but the DMP433 level increased, meaning that nitrites were not purged but instead were transferred to DMP433. These experiments did not focus on the absolute nitrite or DMP433 levels but instead considered the relative changes in nitrite and DMP433 levels as compared with control group, in which no heterogeneous nitrite remover was added. These screening experiments were conducted according to the process shown in FIG. 5.

The results of these experiments as shown in the Table below are stated as percentages (level of nitrite or DMP433 in the experiment vs in the control).

| | | | Nitrite content | DMP433 content |
|---|---|---|---|---|
| Entry | Brand/Series | Product Code | vs control | vs control |
| 1 | AmberChrom ™ | 1 × 2 100-200 mesh | 25.0% | 142.8% |
| 2 | | 1 × 4 Cl, 50-100 mesh | 13.1% | 178.5% |
| 3 | | 1 × 4 Cl, 100-200 mesh | 14.0% | 130.8% |
| 4 | | 1 × 8 Cl, 50-100 mesh | 7.7% | 168.8% |
| 5 | | 1 × 8 Cl, 100-200 mesh | [symbol] .7% | 299.2% |
| 6 | | 1 × 8 Cl, 200-400 mesh | 7.7% | 262.1% |
| 7 | Amberlite ™ | XAD-2 | 157.6% | 157.0% |
| 8 | | XAD-4, 20-60 mesh | 111.3% | 148.4% |
| 9 | | XAD-7HP, 20-60 mesh | 71.7% | 135.3% |
| 10 | | XAD-16N, 20-60 mesh | 94.6% | 140.7% |
| 11 | | HPR650 H | 97.7% | 673.0% |
| 12 | | HPR4100 Cl | [symbol] 0.5% | 10 [symbol] .0% |
| 13 | | IR120 Na | 74.8% | 94.1% |
| 14 | | IR120 H | 21.3% | 764.1% |
| 15 | | IRN78 | 39.8% | 80.5% |
| 16 | | IRN78 OH | [symbol] 0.8% | 181.1% |
| 17 | | IRN150 | 6.3% | 149.1% |
| 18 | | IRA410 Cl, 20-25 mesh | 7. [symbol] % | 125.9% |
| 19 | | IRA743 free base | 28.8% | 140.9% |
| 20 | | IRA900 Cl | 11.8% | 115.9% |
| 21 | | CG50 H | 24.1% | 1415. [symbol] % |
| 22 | | MAC-3 H | 50.6% | 1069.0% |
| 23 | | FPA- [symbol] free base | 15.8% | 112.6% |
| 24 | | MB | 9.0% | 100.8% |
| 25 | | MB20 | 8.0% | 86.3% |
| 26 | Amberlyst ™ | 15 H | 32.7% | 8 [symbol] .7% |
| 27 | | A21 | 16.9% | 93.3% |
| 28 | | A26 OH | [symbol] .5% | 120.1% |
| 29 | | A25 | 9. [symbol] % | 74.3% |
| 30 | AmberSep ™ | Retardion 11A8, 50-100 mesh | 93.2% | 111.4% |
| 31 | | 900 OH, strongly basic | 27.2% | 126.5% |
| 32 | | [symbol] | 8.1% | 68.1% |
| 33 | Diaion | WA30 free base | 12.9% | 104.0% |
| 34 | Lewatit | MP- [symbol] 2 | 71.3% | 232.8% |
| 35 | Sephadex | LH-20 | 97.3% | 117.3% |
| 36 | | A-25 Ct | 84.1% | 783.5% |

[symbol]

indicates data missing or illegible when filed

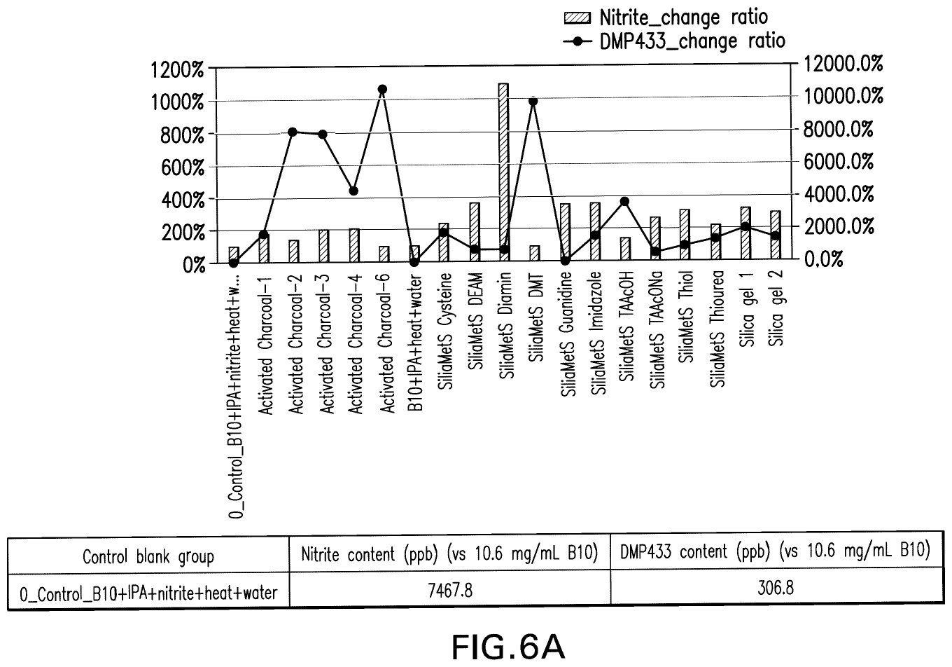

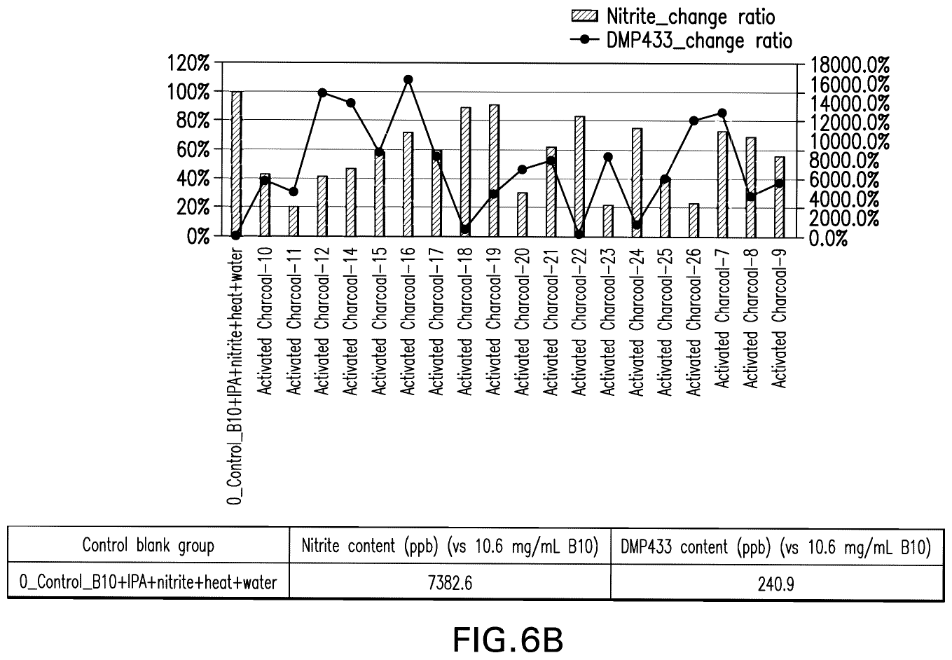

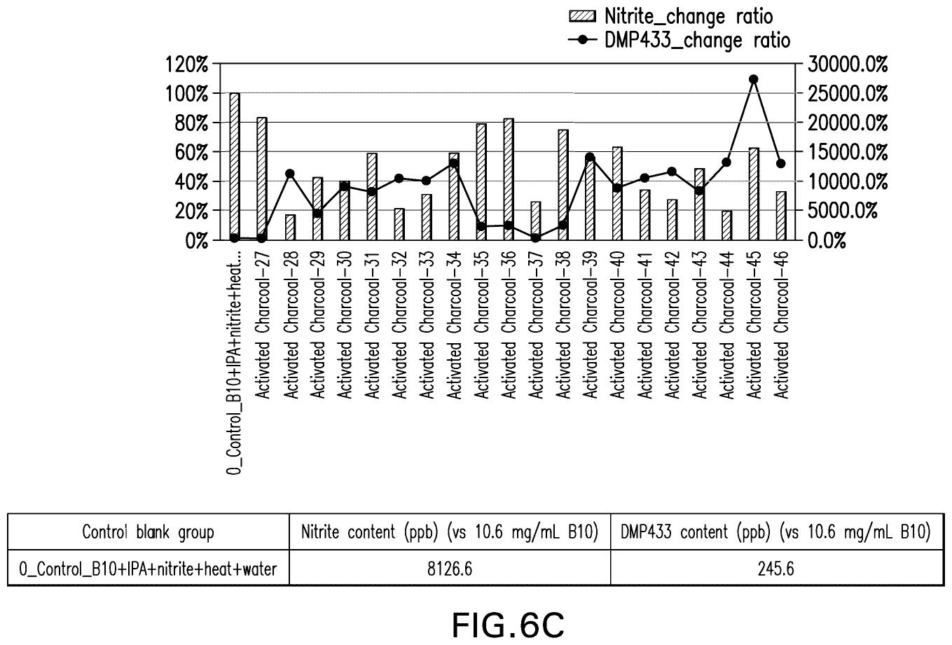

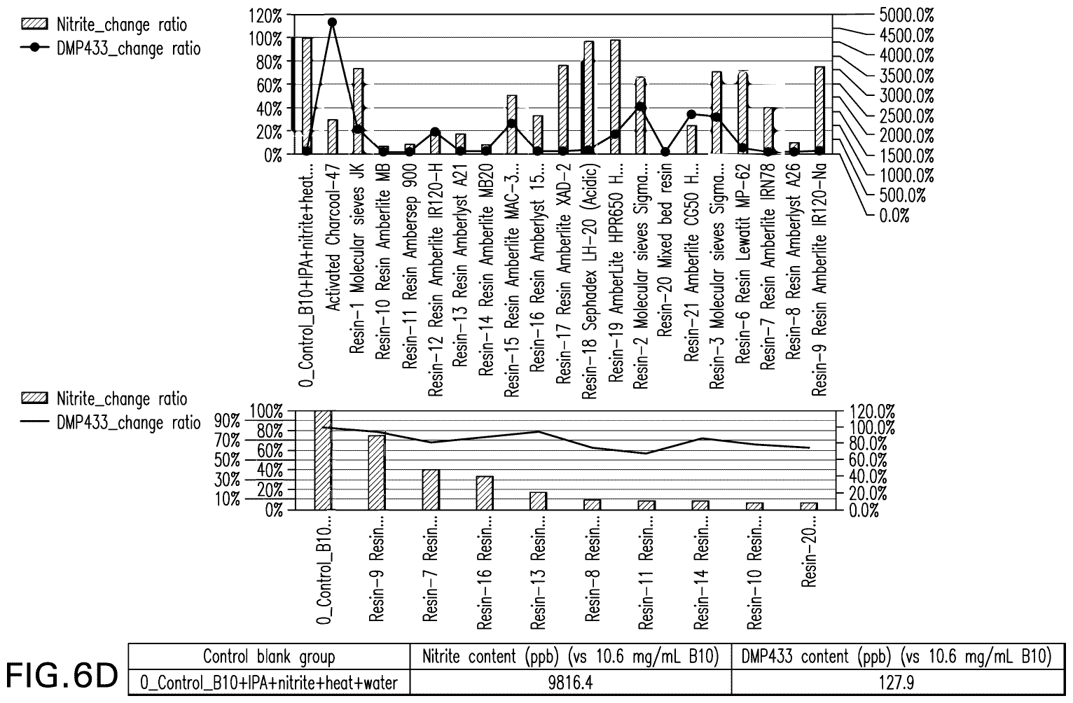

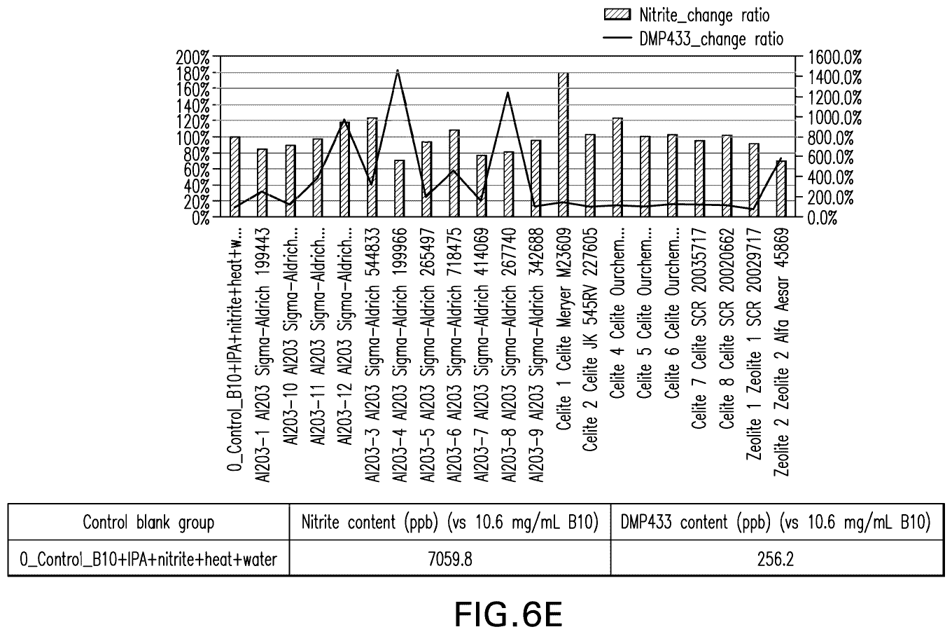

Further results of such studies are shown in the tables below and in FIGS. 6A-6E. In these figures, the columns depict “nitrite_change ratio” and the line graph depicts “DMP433_change ratio”, again expressed as percentage values. The brand, catalogue number and batch number of commercial activated charcoals nos. 1-47 are shown in the following table.

| AC No. | Vendor | Catalogue No | Batch No |
|---|---|---|---|
| 1 | SCR | 4007560 | 20220829 |
| 2 | SCR | 10006719 | 20220328 |
| 3 | SCR | 10006619 | 20230626 |
| 4 | Thermo scientific | 134342500 | A0443812 |
| 5 | TCI | C2194 | CQ3KE-IC |
| 6 | J&K | 972329 | LK60X58 |
| 7 | Meryer | J01094-100G | 230203100068 |
| 8 | Liyang Desheng | 610 | |
| 9 | Liyang Desheng | 615 | |
| 10 | Liyang Desheng | 720 | |
| 11 | Liyang Desheng | 767 | |
| 12 | Liyang Desheng | 777 | |
| 13 | Shaanxi Rock | UC05N | ZT10033-230430 |
| 14 | Shaanxi Rock | T1 | Y2309001 |
| 15 | Shaanxi Rock | T5 | Y2309003 |
| 16 | Shaanxi Rock | TE | Y2309002 |
| 17 | Sigma-Aldrich | 53663 | BCCH6157 |
| 18 | Sigma-Aldrich | 22874 | BCCH5863 |
| 19 | Sigma-Aldrich | 80485 | BCCF4393 |
| 20 | Sigma-Aldrich | C4386 | BCCJ4175 |
| 21 | Sigma-Aldrich | 05105 | BCCJ8333 |
| 22 | Sigma-Aldrich | C2764 | BCCJ1366 |
| 23 | Sigma-Aldrich | C9157 | BCCJ1475 |
| 24 | Sigma-Aldrich | 05112 | BCCD3050 |
| 25 | Sigma-Aldrich | 329428 | MKCJ3374 |
| 26 | Sigma-Aldrich | C5510 | SHBM4994 |
| 27 | Sigma-Aldrich | 29204 | BCCD7702 |
| 28 | Sigma-Aldrich | 242276 | SHBN4374 |
| 29 | Sigma-Aldrich | C3014 | MKCH5018 |
| 30 | Sigma-Aldrich | 242241 | SHBL1409 |
| 31 | Sigma-Aldrich | 242233 | SHBN7922 |
| 32 | Sigma-Aldrich | 161551 | SHBN0379 |
| 33 | Sigma-Aldrich | 242268 | MKCH6016 |
| 34 | Grave technologies | ECOSORB C-941 | SR-4227 |
| 35 | Grave technologies | ECOSORB C-902 | NB2196-190 |
| 36 | Grave technologies | ECOSORB C-905 | SR-5728 |
| 37 | Grave technologies | ECOSORB C-981 | SR-5727 |
| 38 | Grave technologies | ECOSORB C-906 | SR-5629 |
| 39 | SiliCycle | EPAK C-947 | 14389A |
| 40 | SiliCycle | EPAK C-941 | 14359A |
| 41 | NORIT | NORIT SX2 | |
| 42 | NORIT | NORIT CASPF | |
| 43 | NORIT | NORIT DARCO | |
| 44 | NORIT | NORIT SX | |
| 45 | Suzhou Hemera | Enopc | |
| 46 | Suzhou Hemera | DCL320 | |
| 47 | Suzhou Hemera | CPG | |

### Screening Results (i)

| | Nitrite_change | DMP433_change |
|---|---|---|
| Scavenger [symbol] | ratio | ratio |
| 0_Control_B10 + IPA + | 100% | 100.0% |
| nitrite + heat + water | | |
| Activated Charcoal-1 | 214% | 1747.8% |
| Activated Charcoal-2 | 141% | 8008.1% |
| Activated Charcoal-3 | 200% | 7827.2% |
| Activated Charcoal-4 | 204% | 4392.5% |
| Activated Charcoal-6 | 96% | 10602.2% |
| B10 + IPA + heat + water | 100% | 100.0% |
| SiliaMetS Cysteine | 238% | 1887.5% |
| SiliaMetS DEAM | 360% | 818.9% |
| SiliaMetS Diamin | 1085% | 784.9% |
| SiliaMetS DMT | 91% | 9856.5% |
| SiliaMetS Guanidine | 350% | 113.8% |
| SiliaMetS Imidazole | 353% | 1596.4% |
| SiliaMetS TAAcOH | 141% | 3621.4% |
| SiliaMetS TAAcONa | 266% | 553.9% |
| SiliaMetS Thiol | 310% | 985.3% |
| SiliaMetS Thiourea | 215% | 1395.7% |
| Silica gel 1 | 323% | 2098.3% |
| Silica gel 2 | 298% | 1469.9% |

### Screening Results (ii)

| | Nitrite_change | DMP433_change |
|---|---|---|
| Scavenger [symbol] | ratio | ratio |
| 0_Control_B10 + IPA + | 100% | 100.0% |
| nitrite + heat + water | | |
| Activated Charcoal-10 | 43% | 5706.8% |
| Activated Charcoal-11 | 21% | 4483.6% |
| Activated Charcoal-12 | 42% | 14848.1% |
| Activated Charcoal-14 | 47% | 13799.6% |
| Activated Charcoal-15 | 63% | 8688.4% |
| Activated Charcoal-16 | 72% | 16179.8% |
| Activated Charcoal-17 | 60% | 8241.7% |
| Activated Charcoal-18 | 89% | 775.3% |
| Activated Charcoal-19 | 91% | 4405.0% |
| Activated Charcoal-20 | 31% | 6883.4% |
| Activated Charcoal-21 | 62% | 7911.3% |
| Activated Charcoal-22 | 83% | 230.4% |
| Activated Charcoal-23 | 22% | 8262.2% |
| Activated Charcoal-24 | 76% | 1246.7% |
| Activated Charcoal-25 | 44% | 5975.7% |
| Activated Charcoal-26 | 23% | 12038.5% |
| Activated Charcoal-7 | 73% | 12836.5% |
| Activated Charcoal-8 | 70% | 4239.3% |
| Activated Charcoal-9 | 56% | 5589.5% |

Screening Results (iii)

| | Nitrite_change | DMP433_change |
|---|---|---|
| Scavenger [symbol] | ratio | ratio |
| 0_Control_B10 + IPA + | 100% | 100.0% |
| nitrite + heat + water | | |
| Activated Charcoal-27 | 84% | 198.8% |
| Activated Charcoal-28 | 17% | 11286.6% |
| Activated Charcoal-29 | 42% | 4560.6% |
| Activated Charcoal-30 | 40% | 8958.2% |
| Activated Charcoal-31 | 59% | 8219.3% |
| Activated Charcoal-32 | 21% | 10344.6% |
| Activated Charcoal-33 | 31% | 10023.0% |
| Activated Charcoal-34 | 59% | 13033.9% |
| Activated Charcoal-35 | 79% | 2205.6% |
| Activated Charcoal-36 | 83% | 2358.8% |
| Activated Charcoal-37 | 26% | 335.3% |
| Activated Charcoal-38 | 75% | 2446.3% |
| Activated Charcoal-39 | 58% | 14012.8% |
| Activated Charcoal-40 | 64% | 8746.5% |
| Activated Charcoal-41 | 34% | 10537.5% |
| Activated Charcoal-42 | 27% | 11354.8% |
| Activated Charcoal-43 | 49% | 8330.3% |
| Activated Charcoal-44 | 20% | 13069.4% |
| Activated Charcoal-45 | 62% | 27339.3% |
| Activated Charcoal-46 | 33% | 12956.5% |

### Screening Results (iv)

| Scavenger [symbol] | Nitrite_change ratio | DMP433_change ratio |
|---|---|---|
| 0_Control_B10 + IPA + nitrite + heat + water | 100% | 100.0% |
| Al2O3-1 Al2O3 Sigma-Aldrich 199443 | 84% | 254.0% |
| Al2O3-10 Al2O3 Sigma-Aldrich 342653 | 89% | 128.8% |
| Al2O3-11 Al2O3 Sigma-Aldrich 551643 | 97% | 378.6% |
| Al2O3-12 Al2O3 Sigma-Aldrich 517747 | 114% | 967.3% |
| Al2O3-3 Al2O3 Sigma-Aldrich 544833 | 123% | 323.1% |
| Al2O3-4 Al2O3 Sigma-Aldrich 199966 | 71% | 1441.8% |
| Al2O3-5 Al2O3 Sigma-Aldrich 265497 | 94% | 200.7% |
| Al2O3-6 Al2O3 Sigma-Aldrich 718475 | 109% | 459.3% |
| Al2O3-7 Al2O3 Sigma-Aldrich 414069 | 76% | 172.4% |
| Al2O3-8 Al2O3 Sigma-Aldrich 267740 | 81% | 1229.6% |
| Al2O3-9 Al2O3 Sigma-Aldrich 342688 | 94% | 108.9% |
| Celite 1 Celite Meryer M23609 | 181% | 154.3% |
| Celite 2 Celite JK 545RV 227605 | 103% | 110.3% |
| Celite 4 Celite Ourchem XW688555496 | 124% | 117.1% |
| Celite 5 Celite Ourchem XW688555495 | 100% | 106.6% |
| Celite 6 Celite Ourchem XW688555498 | 103% | 123.8% |
| Celite 7 Celite SCR 20035717 | 95% | 123.9% |
| Celite 8 Celite SCR 20020662 | 101% | 120.3% |
| Zeolite 1 Zeolite 1 SCR 20029717 | 91% | 69.0% |
| Zeolite 2 Zeolite 2 Alfa Aesar 45869 | 69% | 578.1% |

### Screening Results (v)

| | Nitrite_change | DMP433_change |
|---|---|---|
| Scavenger [symbol] | ratio | ratio |
| 0_Control_B10 + IPA + nitrite + | 100% | 100.0% |
| heat + water | | |
| Resin-9 Resin Amberlite IR120-Na | 75% | 94.1% |
| Resin-7 Resin Amberlite IRN78 | 40% | 80.5% |
| Resin-16 Resin Amberlyst 15 | 33% | 86.7% |
| (Acidic) | | |
| Resin-13 Resin Amberlyst A21 | 17% | 93.3% |
| Resin-8 Resin Amberlyst A26 | 9% | 74.3% |
| Resin-11 Resin Ambersep 900 | 8% | 68.1% |
| Resin-14 Resin Amberlite MB20 | 8% | 86.3% |
| Resin-10 Resin Amberlite MB | 7% | 80.4% |
| Resin-20 Mixed bed resin | 6% | 76.1% |

### Resin 8, 10, 11, 14, 20-Repeat and Check Studies

| | | Reaction Time | Nitrite_change | DMP433_change |
|---|---|---|---|---|
| Scavenger [symbol] | Filter or not [symbol] | (h) [symbol] | ratio | ratio |
| [symbol] Resin-10 Resin Amberlite MB | [symbol] Centrifuge | 6 | 7.6% | 98.2% |
| | | 17 | 4.9% | 101.2% |
| | | 89 | 10.3% | 111.0% |
| | [symbol] Filter | 6 | 7.1% | 95.9% |
| | | 17 | 6.4% | 131.5% |
| | | 21 | 6.6% | 80.4% |
| | | 89 | 12.7% | 113.9% |
| [symbol] Resin-11 Resin Ambersep 900 | [symbol] Centrifuge | 6 | 5.3% | 98.5% |
| | | 17 | 6.6% | 85.6% |
| | | 89 | 11.0% | 57.8% |
| | [symbol] Filter | 6 | 12.5% | 85.2% |
| | | 17 | 8.8% | 90.8% |
| | | 21 | 8.1% | 68.1% |
| | | 89 | 10.3% | 65.9% |
| [symbol] Resin-14 Resin Amberlite MB20 | [symbol] Centrifuge | 6 | 4.8% | 98.1% |
| | | 17 | 5.5% | 96.9% |
| | | 89 | 23.3% | 115.8% |
| | [symbol] Filter | 6 | 9.2% | 95.6% |
| | | 17 | 5.9% | 136.7% |
| | | 21 | 8.0% | 86.3% |
| | | 89 | 41.6% | 108.9% |
| [symbol] Resin-20 Mixed bed resin | [symbol] Centrifuge | 6 | 27.7% | 123.1% |
| | | 17 | 13.6% | 86.7% |
| | | 89 | 33.5% | 124.2% |
| | [symbol] Filter | 6 | 22.2% | 96.3% |
| | | 17 | 20.6% | 139.0% |
| | | 21 | 6.2% | 76.1% |
| | | 89 | 19.6% | 102.3% |
| [symbol] Resin-8 Resin Amberlyst A26 | [symbol] Centrifuge | 6 | 7.9% | 88.1% |
| | | 17 | 7.1% | 78.6% |
| | | 89 | 13.0% | 76.7% |
| | [symbol] Filter | 6 | 12.4% | 90.5% |
| | | 17 | 8.8% | 86.9% |
| | | 21 | 9.0% | 74.3% |
| | | 89 | 11.7% | 85.4% |

Control blank group

| Filter or | Reaction | Nitrite | DMP433 |
|---|---|---|---|
| Centrifuge | Time (h) | content (ppb) | content (ppb) |
| no Filter | 6 | 6033.1 | 245.4 |
| no Filter | 17 | 5595.42 | 201.1 |
| no Filter | 89 | 5669.92 | 239.6 |
| Filter | 6 | 7059.8 | 256.2 |
| Filter | 17 | 5577.28 | 175.1 |
| Filter | 89 | 5850.78 | 224.5 |
| Centrifuge | 6 | 6342.1 | 282.3 |
| Centrifuge | 17 | 5513.80 | 185.9 |
| Centrifuge | 89 | 5615.66 | 254.4 |

### Example 4—Impact of Low Nitrite Excipients

Tablet core experiments were prepared with low nitrite excipients (“011TC”) or standard excipients (“012TC”). By tablet core, it will be understood that a tablet coating was not used.

The standard microcrystalline cellulose (MCC102) and crospovidone used in 012TC were exchanged with low nitrite MCC102 (with a nitrite level of below 100 ppb) and low nitrite crospovidone (with a nitrite level of about 20 ppb) respectively to form 011TC. Final blends were prepared by homogenization of the tablet core excipients in MS&T lab and compressing them on Fette 2090i on 1 punch. The drug substance DS06 (manufactured at Menges, batch no. B699003AA) was used for the preparation of the tablet core experiments, and it had a DMP433 content of 0.655 ppm (corrected value) at release.

The experiments were packed in aluminium bags. The compositions were stored for 2 weeks, 1 month and 2 months under storage conditions of 40 C/75% RH or 50 C/75% RH. The compositions were tested for the presence and amount of DMP433 (ppm), and the results are shown in the Table below.

| | | T 2 w | T 1 m | T 2 m | T 2 w | T 1 m | T 2 m |
|---|---|---|---|---|---|---|---|
| | | (40° C./ | (40° C./ | (40° C./ | (50° C./ | (50° C./ | (50° C./ |
| Tablet core: | T0 | 75% RH) | 75% RH) | 75% RH) | 75% RH) | 75% RH) | 75% RH) |
| 011TC (low | 0.5 ppm | 0.6 ppm | 0.5 ppm | 0.6 ppm | 0.7 ppm | 0.6 ppm | 0.8 ppm |
| nitrite | | | | | | | |
| excipients) - | | | | | | | |
| cores | | | | | | | |
| 011TC (low | 0.6 ppm | 0.6 ppm | 0.5 ppm | 0.7 ppm | 0.7 ppm | 0.7 ppm | 0.8 ppm |
| nitrite | | | | | | | |
| excipients) - | | | | | | | |
| blend | | | | | | | |
| 012TC | 0.5 ppm | 1.2 ppm | 1.8 ppm | 2.9 ppm | 2.9 ppm | 4.3 ppm | 6.2 ppm |
| (standard | | | | | | | |
| excipients) - | | | | | | | |
| cores | | | | | | | |
| 012TC | 0.6 ppm | 0.9 ppm | 1.3 ppm | 2.5 ppm | 2.2 ppm | 3.4 ppm | 5.6 ppm |
| (standard | | | | | | | |
| excipients) - | | | | | | | |
| blend | | | | | | | |

The 2 month results from testing show that there was no significant DMP433 increase with the low nitrite excipients (for both the blend and core), and a significant increase with the standard excipients (blend and core). Thus, these experiments show that methods of preparing a composition comprising excipients with a low nitrite content may be advantageous.

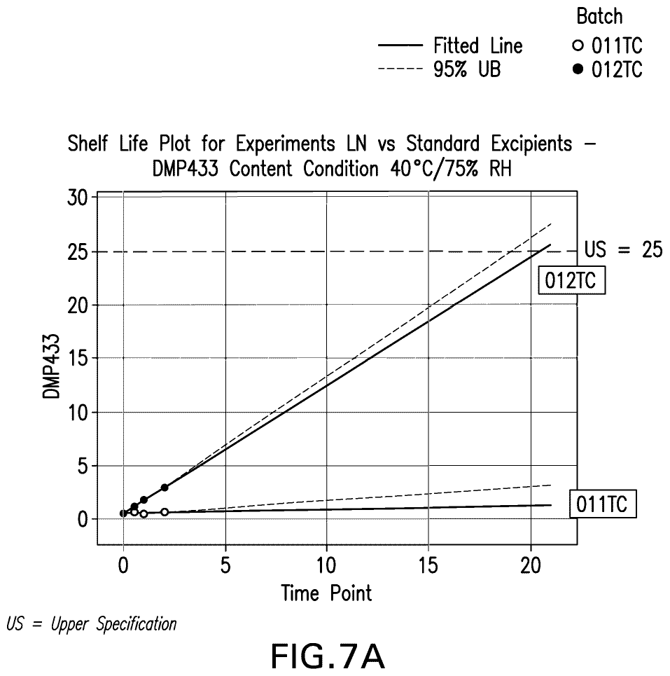

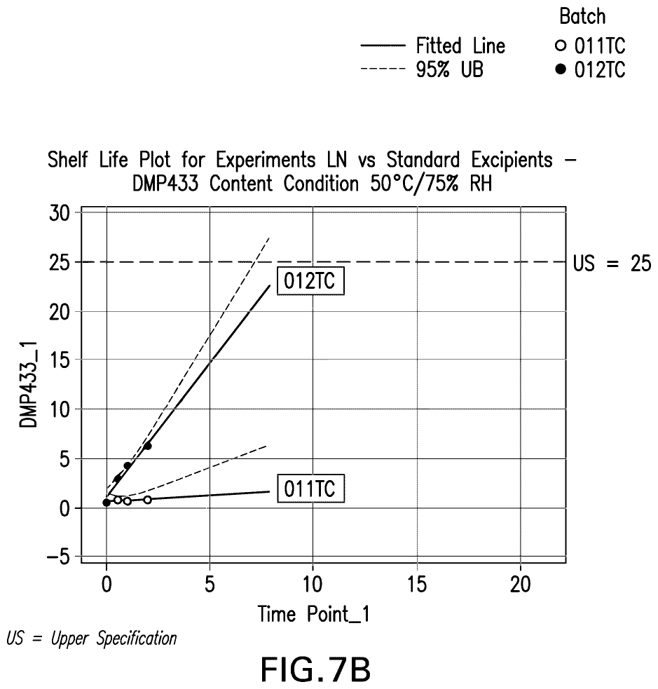

From the shelf life plots (see FIGS. 7A and 7B) for conditions 40 C/75% RH and 50 C/75% RH significant differences in slopes of the experiments with standard excipients (012TC) and low nitrite excipients (011TC) can be observed. This indicates a significant difference in the rate of conversion of DMP at higher temperatures.

Further Studies of Ribociclib Succinate with Excipients

The excipients with the highest relative ratio in the ribociclib film-coated tablet composition (namely microcrystalline cellulose (15.1% of unit composition), low substituted hydroxypropyl cellulose (L-HPC) (10.8% of unit composition) and crospovidone (Type A) (9.4% of unit composition)) were investigated.

Samples of crospovidone (Type A) with standard quality (nitrite levels up to 7 ppm) were compared to samples of the excipient with a nitrite level of ≤100 ppb in binary mixtures of ribociclib succinate drug substance with excipients and in ribociclib 200 mg tablet cores and film-coated tablets. The experiments showed that crospovidone (Type A) is a major contributor to the presence of residual nitrites in ribociclib film-coated tablet and to the increase of DMP433 in the drug product.

Samples of microcrystalline cellulose with standard quality (nitrite levels up to 0.2 ppm) were compared to samples of the excipient with a nitrite level of ≤100 ppb in binary mixtures of ribociclib succinate drug substance with excipients and in ribociclib 200 mg tablet cores and film-coated tablets. Although in the experiments, microcrystalline cellulose did not show significant impact on the increase of DMP433 levels in the drug product, it was decided to use microcrystalline cellulose with levels of nitrites level of ≤100 ppb.

Low substituted hydroxypropyl cellulose (L-HPC) was also evaluated in binary mixtures with ribociclib succinate drug substance and did not show a significant impact on the increase of DMP433 levels in the drug product. L-HPC is available with nitrite levels up to 0.1 ppm.

The results of these technical experiments are provided hereinbelow.

### Binary Studies

Blends and compressed (compacted) mixtures of drug substance with the excipients crospovidone (Type A) and microcrystalline cellulose with standard and low nitrite content, and low substituted hydroxypropyl cellulose (L-HPC) from the drug product composition were subjected to accelerated storage conditions (40° C. and 50° C.) for periods up to 1 month. Results are provided in the table below and suggested a significant increase in DMP433 content in binary mixtures of ribociclib succinate drug substance with compacted crospovidone (Type A) as compared to binary mixtures of the drug substance with compacted low nitrite crospovidone (Type A). The increased formation of DMP433 in the other binary mixtures was less significant.

| | | 40° C. | | 50° C. | |
|---|---|---|---|---|---|
| Sample | T0 | T 2 Weeks | T 1 Month | T 2 Weeks | T 1 Month |
| DS (B699003AA) compact [symbol] | 0.7 ppm | 0.9 ppm | — | 0.8 ppm | 1.1 ppm |
| DS + MCC blend | 0.6 ppm | 0.7 ppm | 0.9 ppm | 0.7 ppm | 1.0 ppm |
| DS + MCC compact | 0.7 ppm | 1.0 ppm | — | 1.0 ppm | 1.1 ppm |
| DS + LN MCC blend | 0.6 ppm | 0.6 ppm | 0.8 ppm | 0.7 ppm | 0.9 ppm |
| DS + LN MCC compact | 0.6 ppm | 0.8 ppm | — | 0.8 ppm | 0.9 ppm |
| DS + L-HPC blend | 0.5 ppm | 0.6 ppm | 0.8 ppm | 0.8 ppm | 1.0 ppm |
| DS + L-HPC compact | 0.6 ppm | 1.0 ppm | — | 1.0 ppm | 1.3 ppm |
| DS + Crospovidone (Type A) blend | 0.6 ppm | 0.6 ppm | 0.9 ppm | 0.9 ppm | 1.6 ppm |
| DS + Crospovidone (Type A) compact | 0.7 ppm | 2.4 ppm | — | 5.7 ppm | 7.5 ppm |
| DS + LN Crospovidone (Type A) blend | 0.6 ppm | 0.5 ppm | 0.7 ppm | 0.5 ppm | 0.7 ppm |
| DS + LN Crospovidone (Type A) | 0.5 ppm | 0.5 ppm | — | 0.6 ppm | 0.7 ppm |
| compact | | | | | |

MCC: Microcrystalline cellulose
L-HPC: Low substituted hydroxypropylcellulose
LN: Low Nitrite
—: not tested [symbol]
Compact: packed in closed glass vial. Blend: packed in glass vial and welded in alu bag. [symbol]
indicates data missing or illegible when filed

DMP433 Content in Tablet Cores Manufactured with Standard Quality and Low Nitrite Excipients on Stability

DMP433 levels in laboratory samples of tablet cores manufactured with either standard quality or low nitrite crospovidone (Type A) and microcrystalline cellulose were analysed after storage for 1 week at stress conditions (60° C., 70° C. and 80° C.). Results are provided in the table below and demonstrate a significant difference in DMP433 content between a batch of tablet cores manufactured with excipients from current suppliers as compared to tablet cores manufactured with low nitrite excipients.

| | | DMP433 content | | | |
|---|---|---|---|---|---|
| | | | 1 Week, | 1 Week, | 1 Week, |
| Sample | Batch | T0 | 60° C. | 70° C. | 80° C. |
| Experimental drug | 012TC | 0.5 ppm | 4.7 ppm | 7.4 ppm | 7.5 ppm |
| product cores with | | | | | |
| excipients from | | | | | |
| current suppliers | | | | | |
| Experimental drug | 011TC | 0.5 ppm | 0.6 ppm | 0.8 ppm | 0.9 ppm |
| product cores with | | | | | |
| low nitrite excipients | | | | | |

DMP433 Content in Commercial Ribociclib Film-Coated Table Manufactured with Standard Quality and Low Nitrite Excipients on Stability

DMP433 levels in three commercial batches of ribociclib 200 mg film-coated tablet manufactured with standard quality crospovidone (Type A) and microcrystalline cellulose were analyzed after storage for 1 week at stress conditions (60° C., 70° C. and 80° C.). Results are provided in the table below.

| | DMP433 content | | | |
|---|---|---|---|---|
| Batch | T0 | 1 Week, 60° C. | 1 Week, 70° C. | 1 Week, 80° C. |
| NJ9758 | 0.2 ppm | 4.3 ppm | 7.2 ppm | 5.6 ppm |
| NK1288 | 0.5 ppm | 4.5 ppm | 7.7 ppm | 6.2 ppm |
| NX1289 | 0.5 ppm | 4.5 ppm | 5.5 ppm | 5.3 ppm |

DMP433 levels in commercial batches of ribociclib 200 mg film-coated tablet manufactured with either standard quality or low nitrite crospovidone (Type A) and microcrystalline cellulose were analyzed after storage at 2-8° C., 40° C./75% RH and 50° C./75% RH. Results up to 2 months are provided in the table below and show that the DMP433 content remained flat at refrigerated conditions (2-8° C.) for all three low nitrite batches, but showed an increase at stress conditions (40° C./75% RH and 50° C./75% RH).

Content of DMP433 (in ppm)

| | | | 2-8° C. | | | | 40° C./75% RH | | | | 50° C./75% RH | | | |
|---|---|---|---|---|---|---|---|---|---|---|---|---|---|---|
| Sample | Batch [symbol] | T0 | T 2 W | T 1 M | T 2 M | T 3 M | T 2 W | T 1 M | T 2 M | T 3 M | T 2 W | T 1 M | T 2 M | T 3 M |
| Commercial² | [symbol] | 0.5 | 0.4 | 0.4 | 0.4 | 0.3 | 2.8 | 4.3 | 6. [symbol] | 7.3 | 7.2 | 8.5 | 8.5 | [symbol] .4 |
| | [symbol] | 0. [symbol] | 0.6 | 0. [symbol] | 0. [symbol] | 0. [symbol] | 2.6 | 3.7 | 6.8 | [symbol] | [symbol] .4 | 7.9 | 8.5 | 8.5 |
| Low | [symbol] | 0.3 | 0.3 | 0.2 | 0.3 | X | 0.5 | 0.5 | 0. [symbol] | X | [symbol] 0 | 1.2 | 1.7 | X |
| nitri [symbol] ³ | [symbol] | 0.3 | 0.2 | 0.2 | 0.2 | X | 0.3 | 0.3 | 0. [symbol] | X | 0. [symbol] | 0. [symbol] | 1. [symbol] | X |
| | [symbol] | 0.3 | 0.2 | 0.2 | 0.3 | X | 0.3 | 0.4 | 0. [symbol] | X | 0. [symbol] | 0.7 | [symbol] | X |

W: Week
M: Month
X: Planned testing [symbol]
Samples stored in welded [symbol] bag
²Crosspovidone Type A and Microcrystalline cellulose batches used in the current commercial [symbol] were not tested for nitrites.
³Crosspovidone Type A batch [symbol] with nitrites <0.1 ppm and Microcrystalline cellulose batch [symbol] with nitrites <0.1 ppm were used in the batches [symbol] [symbol]
indicates data missing or illegible when filed

### Example 5—Storage Conditions, Drying, and Addition of a Desiccant

Verification batches of LN (low nitrite) drug products (DP) were sampled and set on storage conditions of 2-8 C, 40 C/75% RH, or 50 C/75% RH for 2 weeks, 4 weeks, 8 weeks and 12 weeks. Batches were set on stability where one part was the regular product (“NN4375 regular”, “NP0866 regular” and “NP0867 regular”), and one part was dried for 24 hours in an exicator at room temperature to provide dry products. The regular products were each placed and stored in a welded Alu bag. The dry products were each placed and stored in an Alu bag (“NN4375 dry”, “NP0866 dry” and “NP0867 dry”) or in an Alu bag with a desiccant capsule (“NN4375 dry+desiccant”, “NP0866 dry+desiccant” and “NP0867 dry+desiccant”). The products were stored for 2 weeks and 1 month.

The presence and amount of DMP433 (ppm) and the water activity (aw) were measured, and the results are shown in the Table below.

| | Time and storage condition: | | | | | | |
|---|---|---|---|---|---|---|---|
| | | | | T 2 w | T 1 m | T 2 w | T 1 m |
| | | T2 w | T1 m | (40° C./ | (40° C./ | (50° C./ | (50° C./ |
| | T0 | (2-8° C.) | (2-8° C.) | 75% RH) | 75% RH) | 75% RH) | 75% RH) |
| Sample: | DMP433 content/aw | | | | | | |
| NN4375 | 0.3 ppm/ | 0.3 ppm/ | 0.2 ppm/ | 0.5 ppm/ | 0.5 ppm/ | 1.0 ppm/ | 1.2 ppm/ |
| regular | 0.1277 | 0.1189 | 0.1129 | 0.1206 | 0.1178 | 0.1383 | 0.1245 |
| NN4375 dry | 0.3 ppm/ | 0.3 ppm/ | 0.2 ppm/ | 0.5 ppm/ | 0.5 ppm/ | 1.0 ppm/ | 1.1 ppm/ |
| | 0.1189 | 0.1169 | 0.1166 | 0.1179 | 0.1255 | 0.1280 | 0.1242 |
| NN4375 dry + | 0.3 ppm/ | 0.3 ppm/ | 0.3 ppm/ | 0.4 ppm/ | 0.4 ppm/ | 0.7 ppm/ | 0.7 ppm/ |
| desiccant | 0.1189 | 0.0805 | 0.0690 | 0.0776 | 0.0856 | 0.0886 | 0.0811 |
| NP0866 | 0.3 ppm/ | 0.2 ppm/ | n/a | 0.3 ppm/ | n/a | 0.3 ppm/ | n/a |
| regular | 0.1144 | 0.1030 | | 0.1124 | | 0.1181 | |
| NP0866 dry | 0.3 ppm/ | 0.2 ppm/ | n/a | 0.3 ppm/ | n/a | 0.4 ppm/ | n/a |
| | 0.0911 | 0.1044 | | 0.1071 | | 0.1069 | |
| NP0866 dry + | 0.3 ppm/ | 0.2 ppm/ | n/a | 0.2 ppm/ | n/a | 0.3 ppm/ | n/a |
| desiccant | 0.0911 | 0.0701 | | 0.0691 | | 0.0623 | |
| NP0867 | 0.3 ppm/ | 0.2 ppm/ | n/a | 0.3 ppm/ | n/a | 0.5 ppm/ | n/a |
| regular | 0.0826 | 0.1003 | | 0.0920 | | 0.944 | |
| NP0867 dry | 0.3 ppm/ | 0.2 ppm/ | n/a | 0.3 ppm/ | n/a | 0.5 ppm/ | n/a |
| | 0.0821 | 0.0904 | | 0.0918 | | 0.0921 | |
| NP0867 dry + | 0.3 ppm/ | 0.2 ppm/ | n/a | 0.3 ppm/ | n/a | 0.4 ppm/ | n/a |
| desicant | 0.0821 | 0.0600 | | 0.0523 | | 0.0499 | |

### Storage Temperature

As demonstrated by the data above, a higher amount of DMP433 was observed for each of the tested samples when the sample was stored at 40 C or 50 C compared to the sample stored at 2-8 C. Thus, it may be advantageous to store compositions comprising or consisting of ribociclib at a temperature of 2 to 8 C.

### Desiccant

As demonstrated by the data above, a higher amount of DMP433 was observed in the regular moisture LN products compared to the dried products stored with desiccant at temperatures of 40 and 50° C., indicating that the higher moisture content in the film-coated tablet (FCT) product could have an impact on the faster increase of the DMP433.

### LN Compositions

In comparison with the increase of the DMP433 observed on the standard compositions (see Example 7 below, based on NG4411, NG4412, NG4413 and NH4538), an approximately 10-fold decrease was observed at both 40° C./75% RH and 50° C./75% RH on the LN (low nitrite) products.

### Example 6—Storage Conditions

Two LN (low nitrite) Kisqali batches (“NN4375/32546379”, and “NN4375/32547110”) were prepared. The samples were set on stability conditions of 2-8 C, 25° C./60% RH, 30° C./75% RH and 40° C./75% RH for the long term (LT) conditions (25° C./60% RH) on 1, 2, 3, 5, 6, 9, 12, 18, 24 and 36 months and for the accelerated conditions at 1, 2, 3, 5 and 6 months, with additional timepoints to be added up to 12 months.

The compositions were tested for the presence and amount of DMP433 (ppm) and the water activity (aw). Results are shown in the Table below.

| | Time and storage condition: | | | | |
|---|---|---|---|---|---|
| | | | T 1 m | T 1 m | T 1 m |
| | | T 1 m | (25° C./ | (30° C./ | (40° C./ |
| Sample | T0 | (2-8° C.) | 60% RH) | 75% RH) | 75% RH) |
| (DP/FP): | DMP433 content (ppm)/aw: | | | | |
| NN4375/ | 0.3 ppm | 0.3 ppm | / | 0.4 ppm | 0.6 ppm |
| 32546379 | | | | | |
| NN4375/ | 0.3 ppm | 0.3 ppm | 0.3 ppm | / | 0.6 ppm |
| 32547110 | | | | | |

As demonstrated by the data above, a higher amount of DMP433 was observed for each of the tested samples when the sample was stored at 30 C or 40 C compared to the sample stored at 2-8 C. Thus, it may be advantageous to store compositions comprising or consisting of ribociclib at a temperature of 2 to 8 C.

### Example 7—Storage Conditions

Four samples of the drug product DP LEE011 (NG4411, NG4412, NG4413, NH4538) were prepared. The samples were stored under storage conditions of 2-8° C., 30° C./75% RH, 40° C./75% RH and 50° C./75% RH. The samples were stored in Aclar blisters.

The samples were tested for the presence and amount of DMP433 (ppm), and the results are shown in the Table below. The kinetics of DMP433 on the FP (Aclar blisters) was evaluated.

As demonstrated by the data below, there is a fast (up to 16-fold) increase of DMP433 observed after one month storage at 40° C./75% RH with an additional, but smaller, increase at 1.5 months and with a significant increase after 3 months. This could suggest a continued consumption of nitrites. A significant increase is also observed for compositions stored at the 30° C./75% RH condition (even though lower than at 40° C./75% RH), namely a 6-fold increase at 1 month and up to a 10-fold increase at 3 months, indicating a fast conversion even at these lower temperatures.

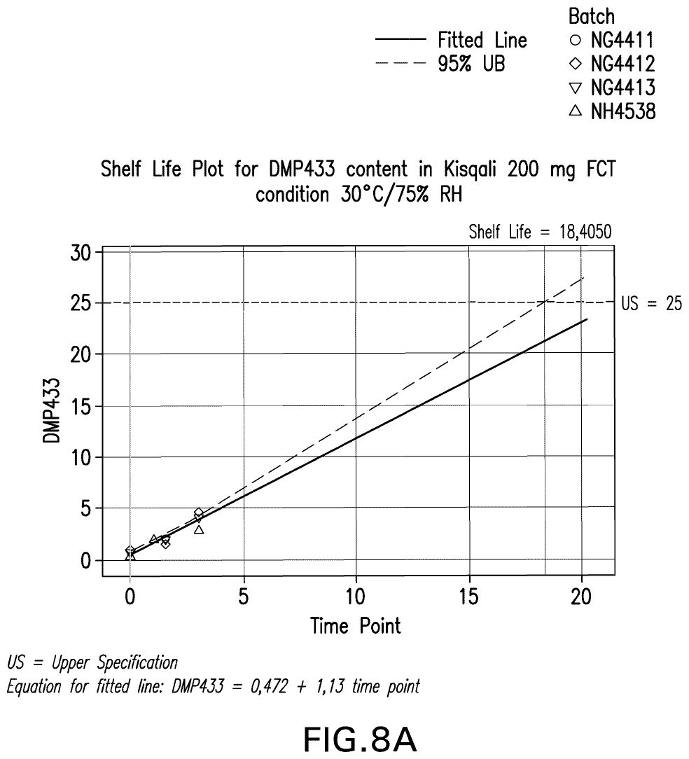

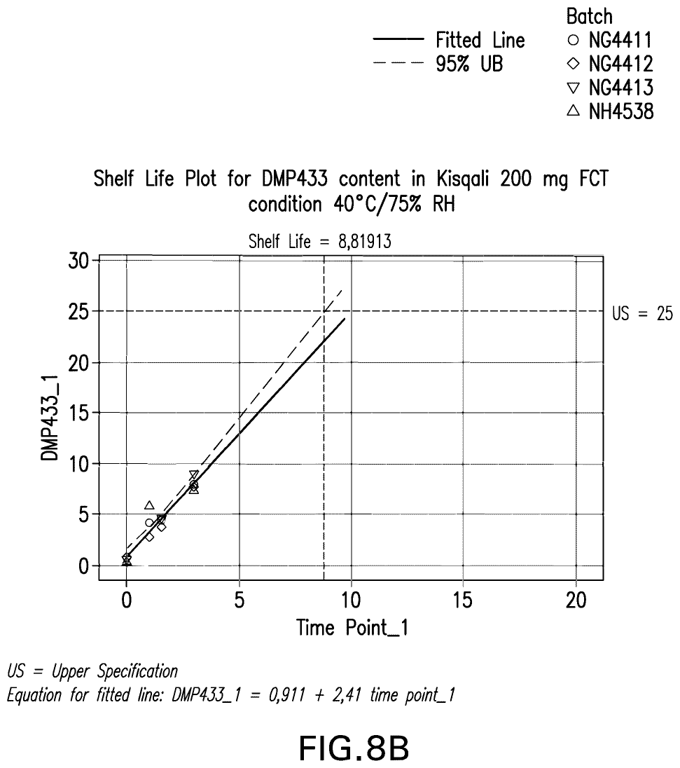

From the shelf-life plots (shown in FIGS. 8A and 8B) for conditions 30° C./75% RH and 40° C./75% RH, differences in slope are observed, which indicates a significant difference in the rate of conversion of DMP at elevated temperatures.

Batch NH4538 was packed more than two months after manufacture. Thus, the DMP433 increase observed after storage conditions of 2-8° C. is most probably due to the time prior to packaging and setting on refrigerated conditions. In any case, a higher amount of DMP433 was observed when the sample was stored at 30° C., 40° C. or 50° C. compared to the sample stored at 2-8° C. Thus, it may be advantageous to store compositions comprising or consisting of ribociclib at a temperature of 2 to 8° C.

| | | | | T 1 m | T 1.5 m | T 3 m | T 1 m | T 1.5 m | T 3 m | T 1 m | T 3 m |
|---|---|---|---|---|---|---|---|---|---|---|---|
| | | T 1 m | T 3 m | (30° C./ | (30° C./ | (30° C./ | (40° C./ | (40° C./ | (40° C./ | (50° C./ | (50° C./ |
| Sample | T0 | (2-8° C.) | (2-8° C.) | 75% RH) | 75% RH) | 75% RH) | 75% RH) | 75% RH) | 75% RH) | 75% RH) | 75% RH) |
| NG4411 | 0.47 | / | / | / | 2.0 | 4.5 | 4.18 | 4.5 | 7.9 | / | / |
| | ppm | | | | ppm | ppm | ppm | ppm | ppm | | |
| NG4412 | 0.81 | / | / | / | 1.5 | 4.5 | 2.78 | 3.7 | 7.7 | / | / |
| | ppm | | | | ppm | ppm | ppm | ppm | ppm | | |
| NG4413 | 0.53 | / | / | / | 1.9 | 4.0 | / | 4.6 | 9.0 | / | / |
| | ppm | | | | ppm | ppm | | ppm | ppm | | |
| NH4538 | 0.35 | 0.8 | 0.7 | 2.0 | / | 2.8 | 5.8 | / | 7.4 | 9.3 | 7.9 |
| | ppm | ppm | ppm | ppm | | ppm | ppm | | ppm | ppm | ppm |

### Example 8—Magnesium Stearate

Binary mixtures with magnesium stearate were prepared and set on stability conditions 40° C./75% RH and 50° C./75% RH for 2 and 4 weeks. Binary mixtures were prepared in the same portion as present in the formulation. The samples were packed in alu bags. Drug substance DS06 (batch no. B699003AA) was used for the preparation of binary mixtures and the blends had DMP433 content 0.655 ppm (corrected value, due to deviation) at release. The compositions were tested for the presence and amount of DMP433 (ppm), and the results are shown in the Table below.

| | | | T 2 w | T 1 m | T 2 w | T 1 m |
|---|---|---|---|---|---|---|
| | Nitrite | | (40° C./ | (40° C./ | (50° C./ | (50° C./ |
| Sample | content | T0 | 75% RH) | 75% RH) | 75% RH)) | 75% RH) |
| DS (B699003AA) | DS 0.1 ppm | 0.7 ppm | 0.9 ppm | Not | 0.8 ppm | 1.1 ppm |
| (compact) | | | | performed | | |
| DS + MgST (Faci) | MgST 3.5 ppm | 0.5 ppm | 0.6 ppm | 0.7 ppm | 0.6 ppm | 0.8 ppm |
| (blend) | | | | | | |
| DS + MgST (Faci) | MgST 3.5 ppm | 0.6 ppm | 0.7 ppm | Not | 0.8 ppm | 0.9 ppm |
| (compact) | | | | performed | | |
| DS + MgST | MgST 13.4 | 0.7 ppm | 1.1 ppm | / | 1.3 ppm | / |
| (Mallinckrodt) | ppm/5.0 ppm | | | | | |
| (blend) | | | | | | |
| DS + MgST | MgST 14.3 | 0.7 ppm | 1.1 ppm | / | 1.6 ppm | / |
| (P. Greven) | ppm/2.0 ppm | | | | | |
| (blend) | | | | | | |
| Tablet core | / | 0.6 ppm | 0.6 ppm | 0.7 ppm | 0.6 ppm | 0.7 ppm |
| blend LN | | | | | | |
| (blend) | | | | | | |
| Tablet core | / | 0.6 ppm | 0.6 ppm | 0.6 ppm | 0.7 ppm | 0.8 ppm |
| blend LN | | | | | | |
| (compact) | | | | | | |
| FCT blend LN | / | 0.6 ppm | 0.5 ppm | 0.7 ppm | 0.6 ppm | 0.8 ppm |
| (blend) | | | | | | |

### Compacts—Accelerated Predictive Stability (APS) Study, TRD

An increase of DMP433 for the binary mixtures with the magnesium stearate from sources Mallinckrodt and Peter Greven were observed, indicating that the magnesium stearate could have an impact on DMP433 content. However, no significant increase on DMP433 content in the samples with the magnesium stearate from the supplier Faci was observed on the binary blends and compacts at accelerated and stress conditions.

### Example 9—Storage Conditions, Drying, and Addition of a Desiccant

Two batches of product Kisqali were sampled and stored under different storage conditions of 2-8° C., 40° C./75% RH and 50° C./75% RH. Batches were set on stability, where one part was dried and the other part was a regular product. The regular product was stored in a welded Alu bag. The dried product was stored in an Alu bag with a desiccant capsule. The products were tested for the presence and amount of DMP433 (ppm), and the results are shown in the Tables below.

| | | T 2 w | T 1 m | T 2 m |
|---|---|---|---|---|
| Sample | T0 | (2-8° C.) | (2-8° C.) | (2-8° C.) |
| NK1288 regular (aw 0.15) | 0.5 ppm | 0.6 ppm | 0.4 ppm | 0.4 ppm |
| NK1288 dry + | / | 0.7 ppm | 0.4 ppm | 0.4 ppm |
| desiccant (aw 0.09) | | | | |
| NK6069 regular (aw 0.13) | 0.6 ppm | 0.5 ppm | 0.6 ppm | 0.6 ppm |
| NK6069 dry (aw 0.10) | / | 0.5 ppm | 0.6 ppm | 0.6 ppm |

From the results under refrigerator condition of 2-8° C., there is no significant increase of DMP433 on the regular and dry products.

| | | T 2 w | T 1 m | T 2 m |
|---|---|---|---|---|
| | | (40° C./ | (40° C./ | (40° C./ |
| Sample | T0 | 75% RH) | 75% RH) | 75% RH) |
| NK1288 regular (aw 0.15) | 0.5 ppm | 2.6 ppm | 4.3 ppm | 6.3 ppm |
| NK1288 dry + | / | 2.0 ppm | 3.2 ppm | 4.4 ppm |
| desiccant (aw 0.09) | | | | |
| NK6069 regular (aw 0.13) | 0.6 ppm | 2.8 ppm | 3.7 ppm | 5.8 ppm |
| NK6069 dry (aw 0.10) | / | 2.3 ppm | 2.8 ppm | 4.0 ppm |

| | | T 2 w | T 1 m | T 2 m |
|---|---|---|---|---|
| | | (50° C./ | (50° C./ | (50° C./ |
| Sample | T0 | 75% RH) | 75% RH) | 75% RH) |
| NK1288 regular (aw 0.15) | 0.5 ppm | 6.4 ppm | 8.5 ppm | 8.5 ppm |
| NK1288 dry + | / | 4.4 ppm | 5.8 ppm | 5.4 ppm |
| desiccant (aw 0.09) | | | | |
| NK6069 regular (aw 0.13) | 0.6 ppm | 7.2 ppm | 7.9 ppm | 8.5 ppm |
| NK6069 dry (aw 0.10) | / | 4.8 ppm | 5.4 ppm | 5.3 ppm |

A higher content of DMP433 was observed in the regular product compared to the dried product at temperatures of 40 and 50° C., indicating that a higher moisture content in the product could have an impact on the faster increase of the DMP.

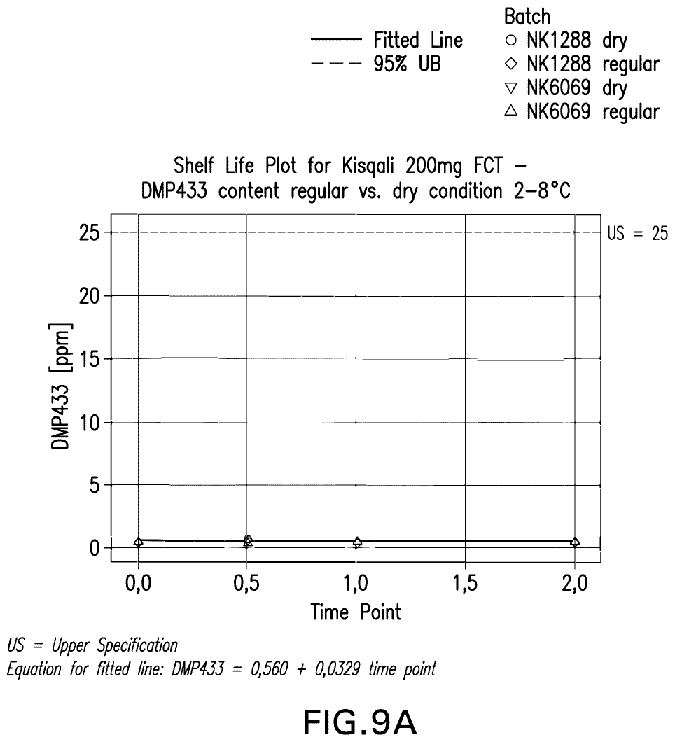

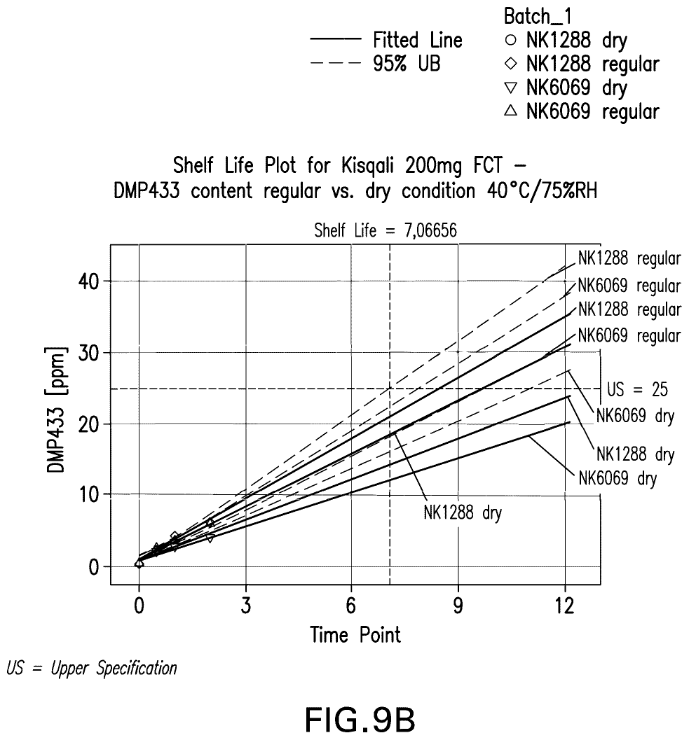

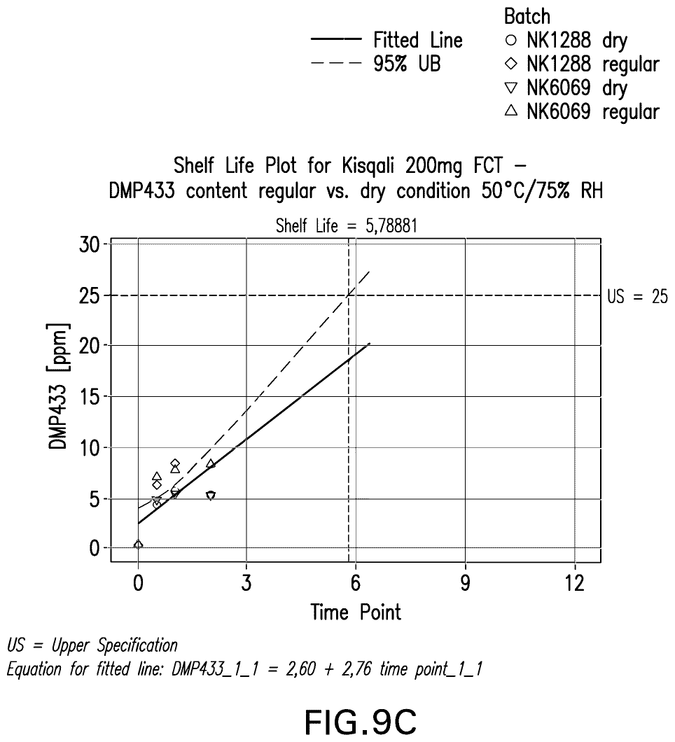

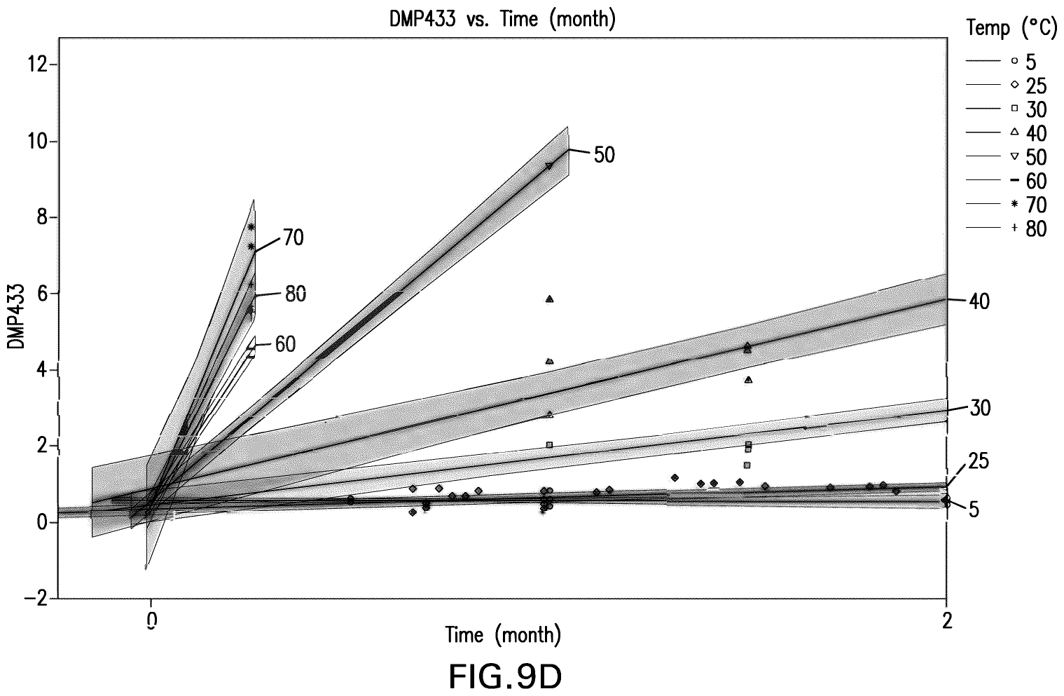

From the shelf life plots (shown in FIGS. 9A-9C) for conditions 40° C./75% RH and 50° C./75% RH we can observe a difference in the slope, indicating a significant difference in the rate of conversion of DMP. At condition 50° C./75% RH, we can observe that there was no increase of DMP433 at the 2m time point, indicating a possible plateauing of the DMP433.

### Statistical Prediction

Currently available TechOps generated data on standard Kisqali product (DP LEE011) up to 2 months was used for a statistical evaluation of the stability prediction and the DMP433 content. From FIG. 9D, it is observed that the slopes for temperatures 60, 70 and 80° C. are very steep and these temperatures would have limited possibility of statistical stability prediction for conditions 25° C. and 2-8° C. It was also observed that, for up to 2 months, no significant differences were observed between temperatures 25° C. and 2-8° C.

### Example 10—Nitrite Testing in DP Excipients

The presence and amount of nitrites was evaluated in a number of excipients.

| | | | Nitrite |
|---|---|---|---|
| | | Batch | content |
| Material name | Supplier | number | (ppm) [symbol] |
| MCC PH102 | JRS* | 32411278 | 0.2 |
| | | 32481439A | 0.1 |
| | | 32481440 | 0.1 |
| | | 32496859 | 0.2 |
| | IFF | 32426446 | 0.6 |
| | Mingtai | 32456006 | 0.8 |
| | | 32475012 | 0.7 |
| | | 32478477 | 0.8 |
| | [symbol] ** | 32529635 | 0.2 |
| L-HPC LH-21 | Shin-Etsu * | 32488528 closed | 0.1 |
| | | 32488528 open | <0.1 |
| | | 32500870 b | 0.1 |
| | | 32500870 open | <0.1 |
| | | 32500869 | <0.1 |
| POLYPLASDONE XL | Ashland* | 32493892 | 21.5 |
| | | 32504922 | 8.6 |
| | JRS | 32421649 | <0.1 |
| | | 32454006 | <0.1 |
| | Ashland [symbol] | 32530141 | <0.1 |
| | | 32530142 | <0.1 |
| | | 32530143 | <0.1 |
| | | 32536179 | <0.1 |
| | | 32536180 | <0.1 |
| | | 32536181 | <0.1 |
| AEROSIL 200 | Evonik* | 32435020 | 0.1 |
| | | 32461580 | <0.1 |
| | | 32507190 | <0.1 |
| | | 32461580 | <0.1 |
| Magnesium [symbol] | FAC [symbol] | 32470497 open | [symbol] |
| | | 32470497 closed | [symbol] |
| | | 32470498 | [symbol] |
| | | 32494780 | [symbol] |
| | [symbol] | 32172222 | [symbol] |
| | [symbol] | 32138648 | [symbol] |
| [symbol] White | [symbol] | 32511374 | <0.1 |
| | | 32515369 | <0.1 |
| [symbol] Red | | 32405204 | <0.1 |
| [symbol] Black | | 32405203 | [symbol] |
| | | 32499072 | [symbol] |

*Indicates the current suppliers for Kisqali.
**Indicates newly introduced suppliers. [symbol]
indicates data missing or illegible when filed

### Example 12—Form E of Ribociclib Succinate

### Production of the Seed Material for Form E

Modification E is extremely difficult to produce in absence of seed material. In order to produce Mod. E the following experiments were performed.

To 3.5 mL of a solution at a concentration ranging from 0.35 to 0.50 mg/mL of ribociclib succinate in dry isopropanol at 60° C., 3 beads of activated molecular sieves 3 Å were added. The resulting mixture was kept for 2 weeks under gentle shaking (e.g., via a vibrating shaker with low frequency. About 10 mg of ribociclib succinate Form A (as described in WO 2020/152629) were added to the reaction mixture and the solution was maintained under shaking for 2 additional days at 60° C. The molecular sieve beads were removed and the solution was gently evaporated until dryness. The resulting powder which consisted of Form E and a trace of molecular sieve, as confirmed by XRPD analysis, was used for further experiments, as described below.

### Production of Form E Using Seed Material

Free base of ribociclib was dissolved in 2-propanol at 72-78° C. (The free base typically dissolves above 62° C.). A clear or slightly hazy solution was obtained after stirring for up to 30 minutes.

The solution of free base of ribociclib in 2-propanol was transferred via pre-heated (75° C.) transfer lines over a pre-heated plate filter, an active carbon filter cartridge and a particle filter into the crystallization vessel (75° C. jacket temperature). The transfer line was rinsed with warm 2-propanol.

The solution of succinic acid in 2-propanol was transferred over a period of approximately 60 min. over a particle filter to the ribociclib free base solution in the crystallization vessel at 73-77° C. Immediately after the addition was complete, a suspension of seed crystals of pure Form E in 2-propanol (minimum of 1 mass/% seed related to expected ribociclib succinate yield) was added. Stirring was continued at medium to high stirrer speed and after approximately 15 minutes, turbidity should be observed. The vessel that contained the succinic acid was rinsed with 2-propanol and the suspension was cooled slowly to 10° C. internal temperature in approximately 20h.

The product was isolated by filtration over a Nutsche filter and the wet cake was rinsed with 2-propanol. The wet filter cake was then dried at 60° C. jacket temperature and <20 mbar.

### Xrpd Analysis of Form E

Bruker D8 Advance; LynxEye detector; Cu-Kα radiation; standard measurement conditions: Bragg-Brentano reflection geometry, 40 kV and 40 mA tube power, 0.02° 20 step size, 37-s step time, 2.5-50° 2θ scanning range. The powder samples were measured in 0.1-mm-deep, silicon single-crystal sample holders. No special treatment was used in preparing the samples other than the application of slight pressure to get a flat surface. An ambient air atmosphere was used for all measurements. All samples were rotated during the measurement.

Form E of ribociclib succinate was characterized by XRPD in reflection mode (see FIG. 10 (corresponding to FIG. 2 of WO 2020/152629)). The Table below shows the XRPD peaks for anhydrous Form E of ribociclib succinate, measured in reflection mode with a Copper K alpha source (corresponding to Table 4 of WO 2020/152629).

### Xrpd Peak Table for Form E of Ribociclib Succinate

| Angle (°2Θ) | Intensity (qualitative) |
|---|---|
| 7.9 | Low |
| 8.8 | Medium |
| 11.0 | Medium |
| 12.4 | Low |
| 13.0 | High |
| 13.7 | Medium |
| 15.7 | Medium |
| 17.4 | Low |
| 18.7 | Medium |
| 20.0 | High |
| 21.1 | Medium |
| 23.0 | High |
| 24.9 | Medium |

(High intensity > 50%; 50% > Medium > 20%; Low < 20%)
The value of each of the 2Θ values is accurate within ±0.2.

### Differential Scanning Calorimetry (DSC)

DSC studies were conducted at the following settings:

| | |
|---|---|
| Instrument | Perkin Elmer Diamond, Mettler DSC1or |
| | DSC822e, or TA instrument DSC Q2000 |
| Temperature range | 30-300° C. |
| Scan rate | If not specified 20K/min |
| Nitrogen flow | Instrument specific |

DSC curves of ribociclib succinate Form E are strongly affected by the heating rate in relation to decomposition. Since the thermal event is related to melting/decomposition, the related enthalpy value is reported for information only, as shown in the Table below (corresponding to Table 9 of WO 2020/152629).

| Heating rate | Endothermic | Enthalpy (J/g) |
|---|---|---|
| (in ° C./min) | onset (in ° C.) | and comments |
| 10 (with pinhole) | 210.0 | 194.8 |

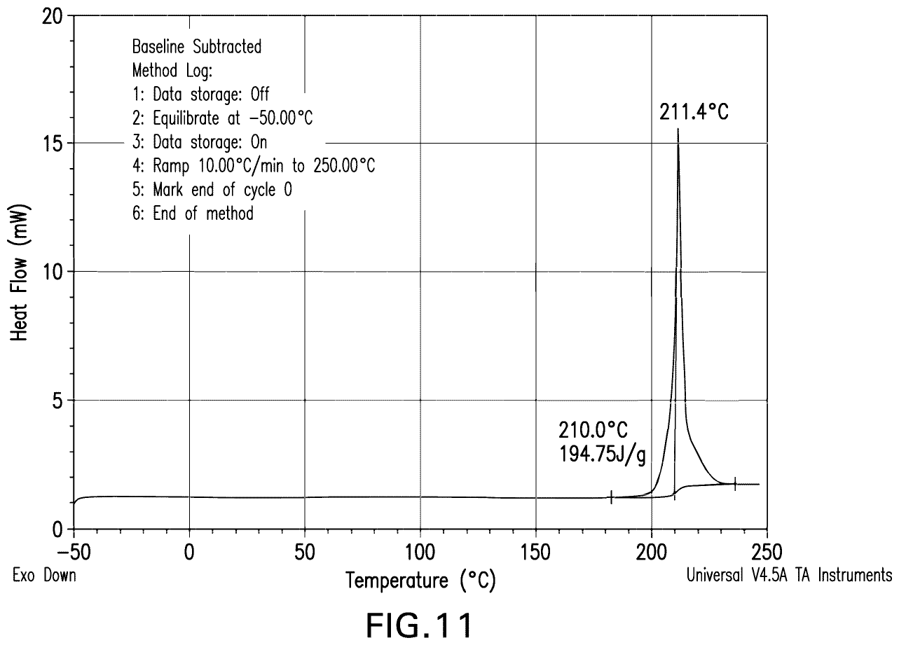

FIG. 11 (corresponding to FIG. 7 of WO 2020/152629) shows the DSC plot of ribociclib succinate Form E at a heating rate of 10 degrees Celsius per minute.

### Thermogravimetry of Form E

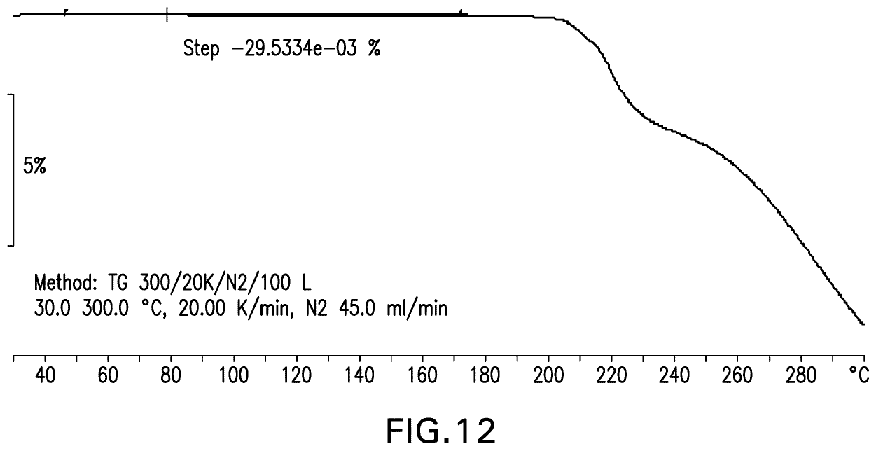

In good agreement with the DSC study, ribociclib succinate Form E undergoes decomposition at about 200° C. From 30 to 180° C., the loss on drying value is less than 0.05%. FIG. 12 (corresponding to FIG. 10 of WO 2020/152629) shows the thermogravimetric curve of Form E at heating rate at 10 degrees Celsius per minute.

### Example 13—Solvent Distillation and Dissolving Ribociclib

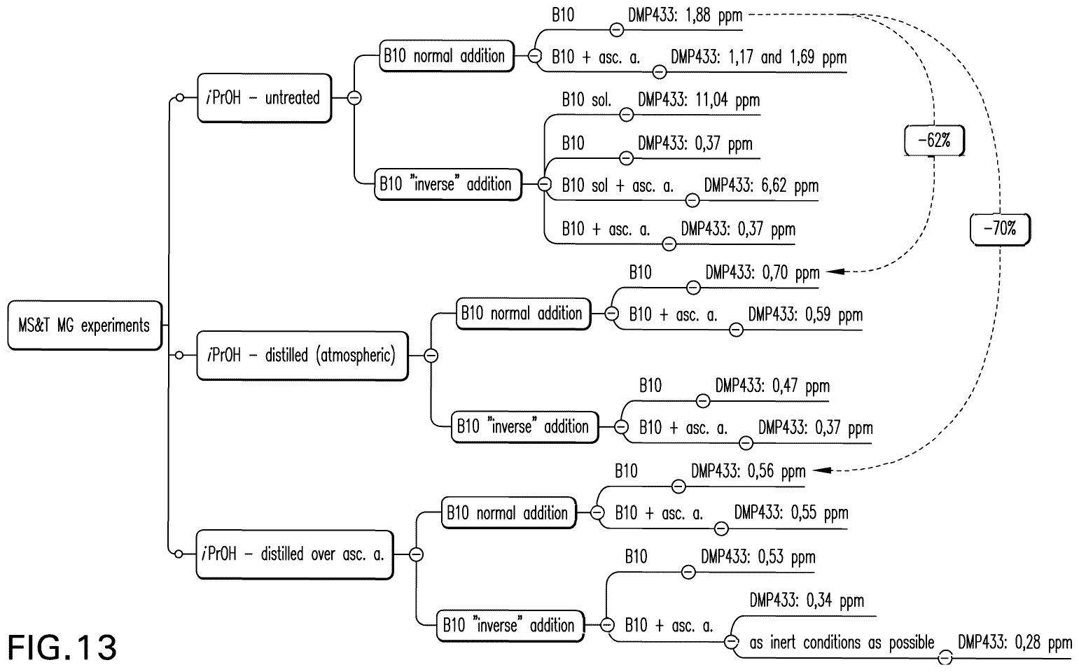

The method of preparing a pharmaceutically acceptable salt of ribociclib may comprise the use of one or more solvents, such as iPrOH in the salt formation step (the chemical step in the scheme above in Example 1, starting from compound B10 and generating B12). There are multiple approaches to using the solvent. Some of these approaches were carried out, and the DMP433 content measured, as shown in FIG. 13. These approaches include:

### Option 1

Step 1: Obtain/PrOH and use in its untreated form
Step 2: Mixing B10 and/PrOH, by (i) adding B10 first to the mixing vessel, and subsequently adding the iPrOH, or (ii) adding iPrOH first to the mixing vessel, and subsequently adding the B10.

### Option 2:

Step 1: Obtain/PrOH and distill it
Step 2: Mixing B10 and the distilled/PrOH, by (i) adding B10 first to the mixing vessel, and subsequently adding the distilled iPrOH, or (ii) adding the distilled/PrOH first to the mixing vessel, and subsequently adding the B10.

### Option 3:

Step 1: Obtain iPrOH and distill it over abscorbic acid
Step 2: Mixing B10 and the distilled iPrOH, by (i) adding B10 first to the mixing vessel, and subsequently adding the distilled/PrOH, or (ii) adding the distilled iPrOH first to the mixing vessel, and subsequently adding the B10.

Each of options 1-3 may further comprise mixing the solution of B10 obtained from Step 2 with a second solution of acid, such as succinic acid. Thus, each of options 1-3 may further comprise:
Step 3: Mixing the solution of B10 with a solution of acid, by (i) adding the B10 solution first to the mixing vessel, and subsequently adding the acid, or (ii) adding the acid first to the mixing vessel, and subsequently adding the B10 solution.

As shown in FIG. 13, the experiments using the distilled iPrOH (particularly the iPrOH distilled over ascorbic acid) resulted in lower DMP433 than the experiment using untreated iPrOH. The iPrOH recovery may be carried out at 30-40° C. at 200 mbar.

As shown in the results in FIG. 13, if B10 added last then less DMP433 formation is observed. The best result (0.34 ppm) was obtained with distilled isopropanol over ascorbic acid, and then B10 added last.

### Example 14—Validation Study of HPLC-HRMS Method for Determination of N-Nitroso-Ribociclib in Ribociclib Succinate Drug Substance and Ribociclib Film-Coated Tablets (FCT: KISQALI®)

A high-performance liquid chromatography-high resolution mass spectrometry (HPLC-HRAM) analytical method was studied, in order to validate and confirm its suitability for the determination of the impurity N-nitroso-ribociclib (NVP-DMP433) in ribociclib succinate drug substance and ribociclib film-coated tablets (FCT; KISQALI®).

The method was validated in the range 0.0625 ppm to 12.5 ppm N-nitroso-ribociclib (NRR) with regard to ribociclib free base. For the purpose of this validation only, the 100% target limit was set at 3.75 ppm (1500 ng NRR/400 mg ribociclib free base).

### 1. Operating Conditions and Laboratory Equipment Chromatographic Conditions

| System 1 | QC_DIP_UPLC_HRMS1 (Orbitrap Exploris 120) | | |
|---|---|---|---|
| System 2 | QC_DIP_UPLC_HRMS2 (Orbitrap Exploris 120) | | |
| Column | C18, 1.7 μm, 100 × 2.1 mm (Acquity | | |
| | UPLC BEH or other suitable) | | |
| Column | 40° C. | | |
| Temperature | | | |
| AS temperature | 22.5° C. | | |
| Volume of | 10 μL | | |
| injection | | | |
| Detection | High Resolution Mass Spectrometry Detector | | |
| Flow rate | 0.35 mL/min | | |
| | Time | Mobile | Mobile |
| | [min] | phase A | phase B |
| Gradient | 0 | 60 | 40 |
| | 2.5 | 60 | 40 |
| | 9.5 | 5 | 95 |
| | 10 | 5 | 95 |
| | 10.5 | 60 | 40 |
| | 14 | 60 | 40 |

| | |
|---|---|
| Software | Software for calculation of statistics |
| Chromeleon 7.2.10 | Chromeleon 7.2.10; Data Analysis |
| | statistic tool of Excel 2016 |

### MS Operating Conditions

MS settings

| | Parameter | Values | |
|---|---|---|---|
| MS Ion Source | Ion source type | HESI | |
| Settings for | Polarity | Positive | |
| Orbitrap | Sheath Gas Flow Rate | 45 arbitrary units | |
| Exploris 120 | Aux Gas Flow Rate | 10 arbitrary units | |
| | Sweep Gas Flow Rate | 0 | units |
| | Spray Voltage | 3.5 | kV |
| | Ion transfer Tube Temp. | 325° | C. |
| | Vaporizer Temp. | 350° | C. |
| | N-Nitroso-Ribociclib (NNR) | | |
| Scan Settings | Scan Type | tMS² | |
| for Orbitrap | HCD Collision Energy (%) | 20 (Normalized) | |
| Exploris 120 | Scan Start-End (min) | 4-6 | |
| | Precursor ion (m/z) | 464.2517 [MH]⁺ | |
| | Product ion (m/z) | 434.2537 | |
| | | (Quant.) ± 15 ppm | |
| | | 392.2210 | |
| | | (Conf.) ± 15 ppm | |
| | Isolation Window | 2 | m/z |

| | |
|---|---|
| Resolution | 60000 |
| Maximum IT | Auto |
| RF lens (%) | 70 |
| Expected LC Peak width (s) | 20 |

### Materials

Reference standards:
   DMP433-NX (N-nitroso-ribociclib), Novartis, Material no: RS03139, Batch 20/2
Samples:
   KISQALI FCT 200 MG SI41 GE 01, Material No: 42033545, Batch no. ND3493
   VALAMOR FCT 200 MG, Material No: 753868, Batch KAD52
   Ribociclib succinate, Material No: 834325, Batch no. 834325A001
   Ribociclib succinate, Material No: 834325, Batch no. 834325A002.
   Ribociclib succinate, Material No: 834325, Batch no. 834325A003

### 2. Solution Preparation According to Analytical Procedure

### 2.1. Solutions and Mobile Phase

Solvent: Water: Methanol=50:50 (V/V)
Mobile phase A: 0.1% (V/V) formic acid in water (e.g. add 1000 μL of formic acid to 1000 mL of water and mix well)
Mobile phase B: Methanol
Blank solution: Solvent
   2.2. NNR Stock Solution (˜40 μg/mL)

Accurately weight about 4 mg of NNR reference standard into a 100 mL amber volumetric flask, dissolve in acetonitrile using vortex and fill to the final volume with acetonitrile.
C_{NNR}=40 μg/mL
   2.3. NNR Reference Solution 1 (NNR1; 1.0 μg/mL)

Dilute exact volume (about 1250 μL) of NNR stock solution, taking into account exact mass and purity of NNR reference standard, into 50 mL with solvent (50% MeOH) to obtain 1.0 μg/LI solution:

$$V = \frac{4 \times 100}{m \times P} \times 1250$$

V . . . Volume to be diluted (μL)
m . . . Mass of NNR reference standard (mg)
P . . . Purity of NNR reference standard (%)
   2.4. NNR Reference Solution 2 (NNR2; 100 ng/mL)

Dilute 5.0 mL of NNR reference solution 1 into 50 mL amber volumetric flask and fill to the mark with solvent (50% MeOH).

$${C}_{NNR} \approx 100 ng / mL$$

2.5. NNR Reference Solution 3 (NNR3; 5 ng/Ml)

Dilute 1000 μL of NNR reference solution 2 into 20 mL amber volumetric flask and fill to the mark with solvent (50% MeOH).

$${C}_{NNR} \approx 5 ng / mL$$

2.6. Calibration Standard Solution (0.5-100 ng/ml)

In a HPLC amber vial mix the volume of NNR2 or NNR3 solution and the volume of solvent, as prescribed in the Table below, and vortex for 20 seconds.

Preparation of calibration standards

| Standard | Volume | Volume | | Volume of | | Conc. | |
|---|---|---|---|---|---|---|---|
| Solution | of NNR2 | of NNR3 | | solvent | | NNR | |
| L1 | — | 150 | μL | 1350 | μL | 0.5 | ng/mL |

| | | | | | | |
|---|---|---|---|---|---|---|
| L2 | — | 1500 | μL | — | 5.0 | ng/mL |

| | | | | | | | |
|---|---|---|---|---|---|---|---|
| L3 | 150 | μL | — | 1350 | μL | 10 | ng/mL |
| L4 | 450 | μL | — | 1050 | μL | 30 | mg/mL |
| L5 | 900 | μL | — | 600 | μL | 60 | ng/mL |

| | | | | | | |
|---|---|---|---|---|---|---|
| L6 | 1500 | μL | — | — | 100 | ng/mL |

2.7. Sample solution-ribociclib succinate drug substance
1. Accurately weigh about 102 mg of ribociclib succinate drug substance (corresponding to 80 mg of ribociclib free base) into a 10 mL amber volumetric flask.
2. Dissolve the substance using vortex and fill to volume with solvent (50% MeOH).
3. Filter through 0.2 μm PVDF (polyvinylidene difluoride) or PTFE (polytetrafluoroethylene) filter into a HPLC amber vial, discard the first 0.5 ml.

$${C}_{Ribociclib} \approx 8 mg / mL$$

2.8. Sample solution-ribociclib FCT (KISQALI®)
1. Grind and homogenize 10 tablets (IKA mill can be used).
2. Into a 10 mL amber volumetric flask accurately about 180 mg of tablet powder (corresponding to 80 mg of ribociclib free base).
3. Add solvent (50% MeOH) up to ⅔ of flask volume. Vortex the sample for 1 minute and shake on a mechanical shaker for at least 15 min. Fill to volume with solvent and use vortex for 1 minute.
4. Filter through 0.2 μm PVDF or PTFE filter into a HPLC amber vial, discard the first 0.5 mL.

$${C}_{Ribociclib} \approx 8 mg / mL$$

### 3. Solutions for Accuracy

### 3.1 Accuracy for Ribociclib Succinate Drug Substance

1. Into a 10 mL amber volumetric flask accurately weigh about 102 mg of ribociclib succinate drug substance (corresponding to 80 mg of ribociclib free base).
2. Add volume of NNR reference solution 2 or 3 (see Table below) and fill to volume with solvent (50% MeOH). Dissolve the substance using vortex.
3. Filter through 0.2 μm PVDF or PTFE filter into a HPLC amber vial, discard the first 0.5 mL.

Preparation of samples for accuracy

| | Volume | Volume | Conc. | |
|---|---|---|---|---|
| Solution | of NNR2 | NNR3 | NNR added | |
| S0-1, 2, 3 | — | — | 0 ng/mL | |
| S1-1, 2, 3 | — | 1.0 mL | 0.5 ng/mL | (0.0625 ppm) |
| S2-1, 2, 3 | 3.0 mL | — | 30 ng/mL | (3.75 ppm) |
| S3-1, 2, 3 | 10.0 mL | — | 100 mg/mL | (12.5 ppm) |

### 3.2 Accuracy for Ribociclib FCT

Composite sample: Weigh individual components, as prescribed in the Table below, transfer them into a mortar and mix well.

Preparation of composite sample

| Component | Weight (mg) |
|---|---|
| Ribociclib succinate | 2544 |
| Microcrystalline cellulose Vivapur 102 | 674 |
| Hydroxypropyl cellulose (L-HPC) | 481 |
| Crospovidone (Type A) | 420 |
| Aerosil | 32 |
| Magnesium Stearate | 148 |
| OPADRY AMB WHITE (PVA) OY-B-28920 | 168 |
| OPADRY AMB BLACK (PVA) 80W277000 | 3 |
| OPADRY AMB RED (PVA) 80W250002 | 2 |
| Total | 4472 |

1. Into a 10 mL amber volumetric flask accurately weigh about 180 mg of composite sample (corresponding to 80 mg of ribociclib free base).
2. Add volume of NNR reference solution 2 or 3 (see Table below) and solvent (50% MeOH) up to ⅔ of flask volume. Vortext the sample for 1 minute and mix on shaker for at least 15 min. Fill to volume with solvent and vortex for 1 minute.
3. Filter through 0.2 μm PVDF or PTFE filter into a HPLC amber vial, discard the first 0.5 mL.

Preparation of samples for accuracy

| | Volume | Volume | Conc. | |
|---|---|---|---|---|
| Solution | of NNR2 | NNR3 | NNR added | |
| S0-1, 2, 3 | — | — | 0 ng/mL | |
| S1-1, 2, 3 | — | 1.0 mL | 0.5 ng/mL | (0.0625 ppm) |
| S2-1, 2, 3 | 3.0 mL | — | 30 ng/mL | (3.75 ppm) |
| S3-1, 2, 3 | 10.0 mL | — | 100 mg/mL | (12.5 ppm) |

### 4. Solutions for Robustness: Sample Preparation—Solvent Volume

1. Grind and homogenize 10 tablets (IKA mill can be used).
2. Accurately weigh about 180 mg of tablet powder (corresponding to 80 mg of ribociclib free base) into a 10 mL, 25 mL or 50 mL amber volumetric flask.
3. Add solvent (50% MeOH) up to ⅔ of flask volume. Vortex the sample for 1 minute and shake on a mechanical shaker for at least 15 min. Fill to volume with solvent and use vortex for 1 minute.
4. Filter through 0.2 μm PVDF or PTFE filter into a HPLC amber vial, discard the first 0.5 mL.

$${C}_{Ribociclib} \approx 8 mg / mL$$

### 5. Precision of the Method

### 5.1. Repeatability—Intraday Precision

Six replicates of the “Sample solution” were prepared according to the procedure described above and tested on the same day. The results are expressed as ppm of NNR with regard to ribociclib free base (μg NNR/g ribociclib free base)

Intraday precision ribociclib succinate
drug substance sample solution

| Replicate | NNR (ppm) |
|---|---|
| R1 | 0.243 |
| R2 | 0.239 |
| R3 | 0.235 |
| R4 | 0.238 |
| R5 | 0.236 |
| R6 | 0.238 |
| Average (ppm) | 0.238 |
| SD (standard deviation) (ppm) | 0.003 |
| RSD (relative standard deviation) (%) | 1.12 |
| CI (confidence interval) at 95% | 0.236-0.241 |
| probability | |

Intraday precision ribociclib FCT sample solution

| Replicate | NNR (ppm) |
|---|---|
| R1 | 0.930 |
| R2 | 0.917 |
| R3 | 0.916 |
| R4 | 0.918 |
| R5 | 0.924 |
| R6 | 0.933 |
| Average (ppm) | 0.923 |
| SD (standard deviation) (ppm) | 0.007 |
| RSD (relative standard deviation) (%) | 0.78 |
| CI (confidence interval) at 95% | 0.915-0.930 |
| probability | |

Criteria for precision of the method (intraday precision-repeatability): RSD (n≥6): ≤20% Conclusion: With regard to the criteria of repeatability, the method is precise for ribociclib succinate drug substance and Kisqali® FCT.

5.2. Ruggedness—Intermediate precision

Two series of six replicates of the “Sample solutions” were prepared according to the procedure described above and tested by two different analysts, on two different days, using different LC/MS systems and different columns. The results are expressed as ppm of NNR with regard to ribociclib free base (μg NNR/g ribociclib free base)

Intermediate precision ribociclib succinate drug substance

NNR (ppm)

| Replicate | Intraday | Ruggedness |
|---|---|---|
| R1 | 0.243 | 0.287 |
| R2 | 0.239 | 0.243 |
| R3 | 0.235 | 0.244 |
| R4 | 0.238 | 0.176 |
| R5 | 0.236 | 0.262 |
| R6 | 0.238 | 0.235 |
| Average (ppm) | 0.238 | 0.241 |
| SD (ppm) (n = 6) | 0.003 | 0.037 |
| RSD (%) (n = 6) | 1.2 | 15.3 |

| | |
|---|---|
| Relative difference (%) | 1.2 |
| Total average (ppm) | 0.240 |
| SD (ppm) (n = 12) | 0.025 |
| RSD (%) (n = 12) | 10.4 |
| CI at 95% probability | 0.224-0.256 |

Intermediate precision of the method for ribociclib FCT

NNR (ppm)

| Replicate | Intraday | Ruggedness |
|---|---|---|
| R1 | 0.930 | 0.879 |
| R2 | 0.917 | 0.878 |
| R3 | 0.916 | 0.872 |
| R4 | 0.918 | 0.895 |
| R5 | 0.924 | 0.892 |
| R6 | 0.933 | 0.882 |
| Average (ppm) | 0.923 | 0.883 |
| SD (ppm) (n = 6) | 0.007 | 0.009 |
| RSD (%) (n = 6) | 0.8 | 1.0 |

| | |
|---|---|
| Relative difference (%) | 4.4 |
| Total average (ppm) | 0.903 |
| SD (ppm) (n = 12) | 0.022 |
| RSD (%) (n = 12) | 2.5 |
| CI at 95% probability | 0.889-0.917 |

Criteria for precision of the method (intermediate precision): RSD (n≥12): ≤25% Conclusion: With regard to the criteria for intermediate precision, the method is rugged.

### 6. Robustness of the Method

### 6.1. Standard Solution Stability

Three replicates of calibration standard solutions L1 (c=0.5 ng/ml) and L6 (c=100 ng/ml) were prepared according to the procedure described above. Solutions were stored in the dark at room temperature (autosampler (AS) vials). After a period of time, the solutions were reanalyzed. The stability of standard solutions is evaluated by determining the relative difference between the responses of the “Sample solution” were prepared according to the procedure described above and tested on the same day. The results are expressed as concentration in ng/ml, determined from calibration curve in each sequence (time point).

Stability of L1 and L6 calibration standard solution
Stability of standard solution, AS at room temperature

| | L1 | | | L6 | | |
|---|---|---|---|---|---|---|
| | 0 h | 24 h | 48 h | 0 h | 24 h | 48 h |
| R₁ | 0.500* | 0.494 | 0.517 | 100.0* | 97.3 | 97.6 |
| R₂ | 0.554 | 0.506 | 0.542 | 105.5 | 99.9 | 99.7 |
| R₃ | 0.573 | 0.531 | 0.540 | 104.2 | 99.3 | 99.7 |
| Average (ng/mL) | 0.542 | 0.510 | 0.540 | 104.2 | 99.3 | 99.7 |
| Relative | — | −5.9 | −0.4 | — | −4.7 | −4.4 |
| Difference (%) | | | | | | |

*Replicates R₁ at time point 0 h were part of the calibration curve.

Criteria for standard solution stability: Relative difference of average response (n≥3) compared to t=0 h. Relative difference of mean values: ≤40%. S/N (signal to noise ratio) for LOQ level (L1)≥10 Conclusion: With regard to the criteria for robustness—standard solutions stability, it is confirmed that the calibration standard solutions are stable for at least 48 hours, if stored at room temperature, in dark.

### 6.2. Sample Solution Stability

Three replicates of Sample solution were prepared according to AP. The replicates were analysed at t=0 h and after app. 24 hours and 48 hours of storage in vials AS at room temperature. The results are expressed as ppm of NNR wrt ribociclib free base. (μg NNR/g ribociclib free base).

Stability of sample solutions, AS at room temperature

| | Ribociclib succinate drug substance | | | Ribociclib FCT | | |
|---|---|---|---|---|---|---|
| | 0 h | 24 h | 48 h | 0 h | 24 h | 48 h |
| R₁ | 0.243 | 0.227 | 0.229 | 0.930 | 0.907 | 0.951 |
| R₂ | 0..239 | 0.225 | 0.223 | 0.917 | 0.891 | 0.941 |
| R₃ | 0.235 | 0.222 | 0.225 | 0.916 | 0.888 | 0.943 |
| Average (ppm) | 0.239 | 0.225 | 0.226 | 0.921 | 0.895 | 0.945 |
| Relative | — | −6.0 | −5.6 | — | −2.8 | 2.6 |
| Difference (%) | | | | | | |

In addition, FCT samples stored in flasks on the bench were filtered at defined time point and analysed. For 48 hours stability, composite samples (from Accuracy) were used.

Stability of sample solutions, flasks at room temperature

| | |
|---|---|
| Ribociclib FCT | Composite samples |

| | 0 h | 48 h | 0 h | 48 h |
|---|---|---|---|---|
| R₁ | 0.930 | 0.901 | 0.252 | 0.282 |
| R₂ | 0.917 | 0.895 | 0.253 | 0.281 |
| R₃ | 0.916 | 0.913 | 0.251 | 0.276 |
| Average (ppm) | 0.921 | 0.903 | 0.252 | 0.280 |
| Relative Difference | — | −2.0 | — | 11.00 |
| (%) | | | | |

Criteria for sample solution stability: No additional response(s) above the reporting limit. Relative difference of average response (n≥3) compared to t=0 h≤40%.

Conclusion: With regard to the criteria for robustness-sample solution stability, sample solutions are stable for at least 48 hours, if stored in the AS at room temperature, or in the flasks on bench.

### 6.3. Sample Preparation—Solvent Volume

Sample solutions were prepare according to the procedure described above using 10 mL, 25 mL and 50 mL flasks, three replicates for each volume. Results for 25 mL and 50 mL were compared to 10 mL volume that is prescribed in the AP. The results are expressed as ppm of NNR with regard to ribociclib free base (μg NNR/g ribociclib free base)

Effect of extraction solvent volume

| | NNR (ppm) | | |
|---|---|---|---|
| Replicate | V = 10 mL | V = 25 mL | V = 50 mL |
| R1 | 10.25 | 11.25 | 11.96 |
| R2 | 10.02 | 11.09 | 11.93 |
| R3 | 10.21 | 11.20 | 12.09 |
| Average (ppm) | 10.16 | 11.18 | 12.00 |
| SD (ppm) (n = 3) | 0.121 | 0.083 | 0.086 |
| RSD (%) (n = 3) | 1.19 | 0.74 | 0.72 |
| Relative difference | — | 10.0 | 18.1 |
| (%) | | | |

Criteria for sample preparation-solvent volume: Relative difference of mean values (n≥3) compared to 10 mL≤30%.

Conclusion: With regard to the criteria for robustness—effect of extraction solvent volume, higher extraction volumes can be used.

### 7. Accuracy of the Method

For ribociclib succinate drug substance, the same drug substance batch (834325A001) was tested unspiked and spiked at three levels in three independent replicates.

For ribociclib FCT, one composite sample preparation and three replicates of spiked composite sample solutions (prepared as described above) were prepared and tested on the same day.

The samples (drug substance or composite sample) were spiked at 0.5 ng/ml (0.0625 ppm), 30 ng/mlL (3.75 ppm) and 100 ng/ml (12.5 ppm) levels. Amount of NNR present in the unspiked sample was subtracted and recoveries were calculated as a ratio between amounts found and amounts added.

Results are expressed in the Table below as recoveries, in percentage, between amounts of NNR found and amounts of NNR added. Amounts are expressed as ppm of NNR wrt ribociclib free base.

### Accuracy of the Method for Ribociclib Succinate Drug Substance

| | | Amount | Amount | Amount | | Average | SD of | RSD |
|---|---|---|---|---|---|---|---|---|
| | | found | corrected | added | Recovery | Recovery | Recovery | (n = 3) |
| Level | Replicate | (ppm) | (ppm) | (ppm) | (%) | (n = 3) (%) | (n = 3) (%) | (%) |
| S₀ | S₀-1 | 0.3328 | (−0.3277) | — | — | — | | — |
| | S₀-2 | 0.3246 | | — | — | | | |
| | S₀-3 | 0.3256 | | — | — | | | |
| S₁ | S₁-1 | 0.3754 | 0.0477 | 0.0625 | 76.4 | 79.9 | 3.4 | 4.2 |
| | S₁-2 | 0.3778 | 0.0501 | 0.0625 | 80.2 | | | |
| | S₁-3 | 0.3796 | 0.0519 | 0.0625 | 83.1 | | | |
| S₂ | S₂-1 | 3.5823 | 3.2546 | 3.7500 | 86.8 | 90.8 | 4.0 | 4.4 |
| | S₂-2 | 3.7302 | 3.4025 | 3.7500 | 90.7 | | | |
| | S₂-3 | 3.8822 | 3.5545 | 3.7500 | 94.8 | | | |
| S₃ | S₃-1 | 11.0295 | 10.7018 | 12.500 | 85.6 | 89.5 | 3.5 | 3.9 |
| | S₃-2 | 11.6248 | 11.2971 | 12.500 | 90.4 | | | |
| | S₃-3 | 11.8859 | 11.5582 | 12.500 | 92.5 | | | |

| | |
|---|---|
| Average recovery (n = 9) (%) | 86.7 |
| SD (n = 9) (%) | 6.0 |
| RSD (n = 9) (%) | 7.0 |

### Accuracy of the Method for Ribociclib FCT

| | | Amount | Amount | Amount | | Average | SD of | RSD |
|---|---|---|---|---|---|---|---|---|
| | | found | corrected | added | Recovery | Recovery | Recovery | (n = 3) |
| Level | Replicate | (ppm) | (ppm) | (ppm) | (%) | (n = 3) (%) | (n = 3) (%) | (%) |
| S₀ | S₀-1 | 0.2519 | (−0.2519) | — | — | — | — | — |
| | S₀-2 | 0.2532 | | — | — | | | |
| | S₀-3 | 0.2505 | | — | — | | | |
| S₁ | S₁-1 | 0.3030 | 0.0511 | 0.0625 | 81.8 | 85.5 | 3.2 | 3.8 |
| | S₁-2 | 0.3067 | 0.0548 | 0.0625 | 87.7 | | | |
| | S₁-3 | 0.3062 | 0.0543 | 0.0625 | 86.9 | | | |
| S₂ | S₂-1 | 3.4785 | 3.2266 | 3.7500 | 86.0 | 90.4 | 4.3 | 4.7 |
| | S₂-2 | 3.6469 | 3.3950 | 3.7500 | 90.5 | | | |
| | S₂-3 | 3.7989 | 3.5470 | 3.7500 | 94.6 | | | |
| S₃ | S₃-1 | 10.7688 | 10.5169 | 12.500 | 84.1 | 86.7 | 3.2 | 3.6 |
| | S₃-2 | 10.9712 | 10.7193 | 12.500 | 85.8 | | | |
| | S₃-3 | 11.5312 | 11.2793 | 12.500 | 90.2 | | | |

| | |
|---|---|
| Average recovery (n = 9) (%) | 87.5 |
| SD (n = 9) (%) | 3.8 |
| RSD (n = 9) (%) | 4.4 |

Criteria for accuracy of the method: For ribociclib succinate drug substance: Average recovery: 60-140%; RSD (n≥3): ≤20% (each level). For ribociclib FCT: Average recovery: 70-150%; RSD (n≥3): ≤20% (each level).

Conclusion: With regard to the criteria for accuracy, the method is accurate for determination of NNR in ribociclib succinate drug substance and ribociclib FCT from 0.5 ng/ml to 100 ng/ml (0.0625 ppm to 12.5 ppm wrt ribociclib free base).

### 8. Linearity of the Method

Calibration standard solutions of NNR in the concentration range from 0.5 ng/ml to 100 ng/ml (0.065 ppm-12.5 ppm wrt to the concentration of ribociclib free base) are part of the analytical procedure as described above and were prepared for each sequence.

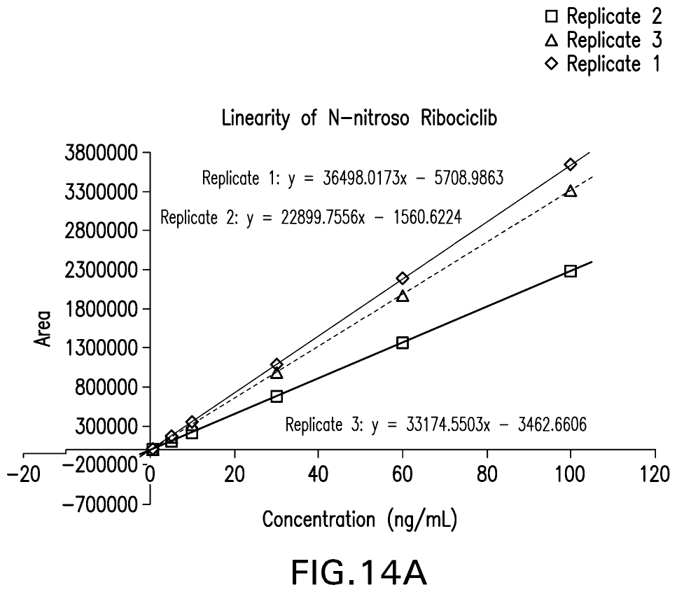

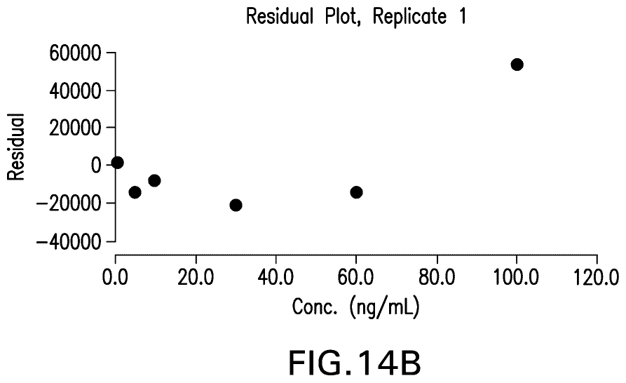

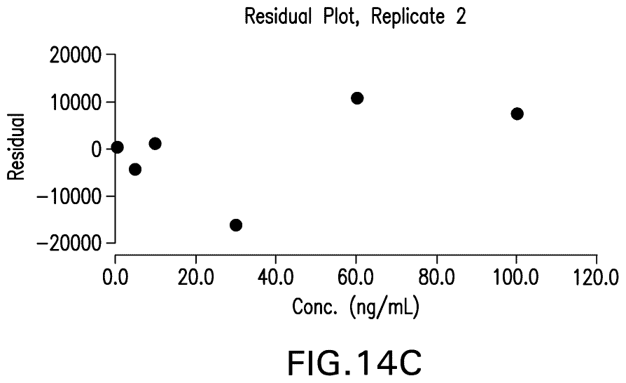

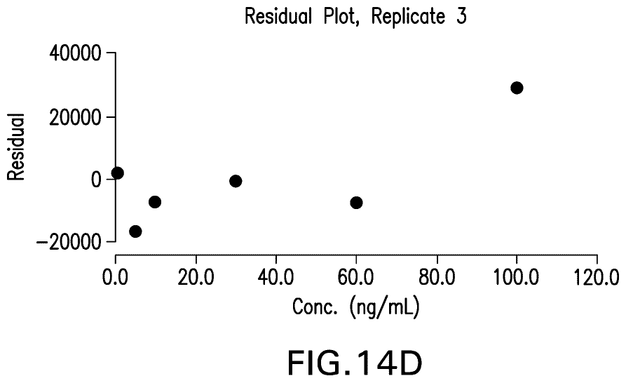

Three replicates of calibration solutions that were prepared for different sequences are presented in the Table below and in FIGS. 14A-14D. In each sequence, calibration solution L1 was injected six times as part of SST test (Repeatability of injections) and the average response was taken for the calculation of calibration curve. Linear regression was weighed with 1/amount.

### Linearity of the Method

| | NNR Conc. | Peak Area | | |
|---|---|---|---|---|
| Solution | (ng/mL) | R 1 | R 2 | R 3 |
| L1 | 0.5 | 14481 | 10519 | 15266 |
| L1 | 0.5 | 14449 | 10411 | 15278 |
| L1 | 0.5 | 14453 | 10249 | 15109 |
| L1 | 0.5 | 14588 | 10195 | 14734 |
| L1 | 0.5 | 14489 | 10416 | 14884 |
| L1 | 0.5 | 14444 | 10500 | 14945 |
| L2 | 5 | 163027 | 108750 | 145987 |
| L3 | 10 | 351547 | 228731 | 321354 |
| L4 | 30 | 1069200 | 669455 | 991317 |
| L5 | 60 | 2170276 | 1383270 | 1979666 |
| L6 | 100 | 3697555 | 2295948 | 3343233 |

| | | | |
|---|---|---|---|
| R² | 0.9996 | 0.9998 | 0.9996 |
| R | 0.9998 | 0.9999 | 0.9998 |
| y-intercept | −5708.9863 | −1560.6224 | −3462.6606 |
| Slope | 36498.0173 | 22899.7556 | 33174.5503 |
| *y-at x = 100% declared content | 131159 | 84313 | 120942 |
| (y = k*x + n) | | | |
| y-intercept (%)* | −4.4 | −1.9 | −2.9 |
| Residual standard deviation (%) | 16.33 | 10.91 | 9.4 |

*w.r.t. the theoretical peak response at the target reference concentration 30 ng/ml (represents 3.75 ppm)

Criteria for Linearity of the method: At visual inspections, the residuals/responses are randomly distributed. R≥0.98 Intercept with y-axis: ≤25%. Residual standard deviation: ≤20%.

Conclusion: The method is linear within the concentration range from 0.5 ng/ml to 100 ng/ml (0.0625 ppm to 12.5 ppm wrt ribociclib free base).

### 9. Determination of Quantitation Limit

The following SST criteria are part of general analytical procedure:
S/N≥10 for NNR peak in L1 solution (conc. 0.5 ng/ml)
RSD≤20% for peak responses for 6 replicate injections of L1

S/N (signal to noise ration) cannot be determined because solvent (i.e. blank) has no noise (noise=0). Therefore, S/N approaches towards infinity.

For LOQ confirmation, 6 samples of ribociclib succinate drug substance spiked at LOQ level (S1) and 6 samples of Composite sample spiked at S1 were prepared and analysed. Amount of NNR present in the unspiked sample was subtracted (see above) and RSD of corrected amounts calculated.

### Confirmation of LOQ for Ribociclib Succinate Drug Substance

NNR (ppm)

| Replicate | Amount found | Amount corrected |
|---|---|---|
| S1-1 | 0.3754 | 0.0477 |
| S1-2 | 0.3778 | 0.0501 |
| S1-3 | 0.3796 | 0.0519 |
| S1-4 | 0.3774 | 0.0497 |
| S1-5 | 0.3781 | 0.0504 |
| S1-6 | 0.3824 | 0.0547 |

| | |
|---|---|
| Average (ppm) | 0.0508 |
| SD (ppm) (n = 6) | 0.0024 |
| RSD (%) (n = 6) | 4.6 |

### Confirmation of LOQ for Ribociclib FCT

NNR (ppm)

| Replicate | Amount found | Amount corrected |
|---|---|---|
| S1-1 | 0.3030 | 0.0511 |
| S1-2 | 0.3067 | 0.0548 |
| S1-3 | 0.3062 | 0.0543 |
| S1-4 | 0.3053 | 0.0534 |
| S1-5 | 0.3077 | 0.0558 |
| S1-6 | 0.3106 | 0.0587 |

| | |
|---|---|
| Average (ppm) | 0.0547 |
| SD (ppm) (n = 6) | 0.0025 |
| RSD (%) (n = 6) | 4.6 |

Criteria for determination of quantitation limit: QL≤reporting limit. Signal-to-noise ratio≥10:1 or RSD (n≥6)≤20%.

Conclusion: With regard to the criteria for Determination of quantitation limit, the LOQ for NNR is 0.5 ng/ml (0.0625 ppm to 12.5 ppm wrt ribociclib free base).

### 10. Selectivity/Specificity of the Method

Chromatograms of the following solutions were recorded, respectively:
Solvent (Blank)
Mobile phase A
Mobile phase B
Calibration standard L1 (LOQ solution)
Sample solution

Criteria for the selectivity/specificity of the method: Chromatographic peaks should be sufficiently resolved from each other, there is no interference with the analysed compound. For MS methods: no ion suppression indicated from recovery experiments (accuracy)

Conclusion: The method is selective.

### 11. Evaluation, Assessment and Calculation of the Amount of DMP433 in Ribociclib Film-Coated Tablets (FCT; KISQALI®)

A calibration curve for DMP433 is created, using calibration standard solutions L1-L6, and expressing the relationship between responses (peak areas) and standard concentrations (ng/ml). For L1, the average of 6 injections is used. Linear regression is weighed with 1/amount.

The amount of DMP433 with regard to the ribociclib free base is calculated as follows:

$$DMP 433 ( ppm ) = \frac{[ \frac{A v z - n}{k} ] XV \times 1000}{m \times 44.72}$$

wherein:
Avz is the area of the DMP433 peak in the sample solution
N is the y-intercept of the calibration curve
K is the slope of calibration curve
M is the weight of the tablet powder (mg)
V is the volume of the sample solution (mL)
44.72 is the percentage (%) of Ribociclib free base in FCT (200 mg/447.2 mg)

### Example 15-DMP433 Transgenic Gene Mutation Assay in Muta™ Mice

The toxicity of nitrosamine compounds (such as DMP433) can be assessed and quantified. One suitable way to assess the toxicity of the nitrosamine compound in vivo is using a transgenic rodent mutation assay, such as the gene mutation assay in Muta™ Mice described hereinbelow.

### General Study Information

A study is on-going, with the objective of evaluating the potential of DMP433 to induce gene mutations in the lacZ transgene within the bone marrow, liver, kidney, and duodenum from treated Muta™ Mice. The bone marrow and liver will be examined as these tissues are expected to be highly exposed to DMP433 upon oral gavage dosing and/or representative of organs with fast and slow dividing cells respectively. The duodenum will be assessed as a site of contact tissue following oral gavage dosing. The kidney will also be examined as a standard target organ for tumours for nitrosamines.

Transgenic rodent mutation assays, such as Muta™ Mouse, are recommended by various regulatory authorities as an appropriate test to determine the genotoxic potential of a compound in vivo. Muta™ Mouse is the most commonly reported transgenic system in use and forms the majority of entries in the Transgenic Rodent Assays Information Database.

The Muta™ Mouse (lacZ/galE) assay was developed by Gossen et al. (see Gossen J, de Leeuw W J, Tan C H, Zwarthoff E C, Berends F, Lohman P H, Knook D L, Vijg J (1989). Efficient Rescue of Integrated Shuttle Vectors from Transgenic Mice: A Model for Studying Mutations in Vivo. Proc Natl Acad Sci USA, 86, 7971-7975) and further refined by the development of a positive selection system developed by Ingeny BV in The Netherlands (see Gossen J and Vijg J (1993). A selective system for LacZ-phage using a galactosesensitive E. coli host. Biotechniques, 14, 326-330).

The assay is an in vivo genotoxicity assay that is capable of detecting the induction of point mutations and small deletions (as opposed to gross chromosomal damage/loss) and has been widely demonstrated to detect mutation in a range of tissues using known mutagens/carcinogens.

For consistency, in this example, the following are considered equivalent:

$$Day 1 - 7 ( dosing ) = Week 1$$

$$Day 8 - 14 ( dosing ) = Week 2$$

$$Day 15 - 21 ( dosing ) = Week 3$$

$$Day 22 - 28 ( dosing ) = Week 4$$

$$Day 29 - 31 ( no dosing ) = Expression period$$

$$Day 31 ( necropsy ) = End of in - life .$$

### Materials

The test item is DMP433, batch number 23/3, with a molecular weight of 463.55 g/mol, a purity of 99.1%, a correction factor 1.01 and storage conditions of −20° C. (nominal) protected from light in very tight packaging. DMP433 is classed as a nitrosamine impurity.

The vehicle control is 2.2 eq HCl 1.ON (˜40%)+28% as powder sulfobutylether-β-cyclodextrin (pH 2.0+0.5), with storage conditions in dispensary of 2 to 8° C. protected from light.

The test item formulation will be prepared daily, unless stability data supports less frequent preparation. The stability data will be generated under Labcorp Study Number 8528626. DMP433 formulations in vehicle at concentrations of 0.5 and 50 mg/mL have been confirmed as stable for up to 8 days at 2 to 8° C. and 15 to 25° C., both protected from light. The storage conditions in central dispensary will be 15 to 25° C. protected from light.

The formulation will be analysed (HPLC/UV analysis, Lapcorp method 8528626-01F, version in force on 22nd February 2024) to confirm that it achieves concentration and homogeneity. Samples (as shown in the following tables, where n/a means not applicable) will be taken from the main experiment during week 1 and week 4 and analysed accordingly.

Sampling (males)

| | Location | | | |
|---|---|---|---|---|
| Group | Top | Middle | Bottom | Total samples |
| Vehicle 0 mg/mL | n/a | 1 × 1.0 mL | n/a | 1 × 1.0 mL |
| Low 2.5 mg/mL | 2 × 1.0 mL | 2 × 1.0 mL | 2 × 1.0 mL | 6 × 1.0 mL |
| Intermediate 5 | 2 × 1.0 mL | 2 × 1.0 mL | 2 × 1.0 mL | 6 × 1.0 mL |
| mg/mL | | | | |
| High 10 mg/mL | 2 × 1.0 mL | 2 × 1.0 mL | 2 × 1.0 mL | 6 × 1.0 mL |

Sampling (females)

| | Location | | | |
|---|---|---|---|---|
| Group | Top | Middle | Bottom | Total samples |
| Vehicle 0 mg/mL | n/a | 1 × 1.0 mL | n/a | 1 × 1.0 mL |
| High 10 mg/mL | 2 × 1.0 mL | 2 × 1.0 mL | 2 × 1.0 mL | 6 × 1.0 mL |

The acceptance criteria will be that the solution achieves a mean % of target of 90-110%, with a % RSD of ≤5%. The solution will be contained in uniquely labelled glass vials, and stored at 15 to 25° C., protected from light. The entire sample will be used for analysis.

### Test System

The animals used will be transgenic mouse of the strain Muta™ Mouse CD2-lacZ80/HazfBR (supplied by Charles River, UK). The animals will have an age range of approximately 8 to 12 weeks on the first day of dosing, and a weight range of approximately 18 to 35 g.

The mice will be housed inside wire topped solid bottom cages with suitable wood bedding (Aspect) and wooden Aspen chew blocks and nesting material. Mice will be housed in groups of up to three per cage, although males may be singly housed. The environment will have a target temperature range of 19 to 25° C. and 40 to 70% relative humidity. A minimum of 15 air changes/hour will be provided. A photo-period of 12 hours nominal will be provided. Diet will be provided with ad libitum access to 5LF2 EU rodent diet (and a moist diet may be provided if required), and water will be provided with ad libitum access.

There will be total randomisation of the allocation to the treatment group. The genotoxicity animals (subgroup 1) will be groups of seven animals of the same sex, randomly allocated on arrival. The satellite animals (subgroups 2, 3 and 4) will be groups of up to eighteen animals of the same sex, randomly allocated on arrival.

The host bacteria for transfection will be as follows:
Strain: E. coli C lac-galE Kan^{r} (galE⁻ Amp^{r})
Source: J Gossen and J Vijg, Ingeny BV, Leiden, The Netherlands
Maintenance of frozen stocks: Cultures are checked for strain characteristics (resistance to kanamycin and ampicillin) and stored in vials with 50% (v/v) glycerol, deep frozen in the gaseous phase of liquid nitrogen.
Overnight culture conditions: Frozen stocks will be used to inoculate 10 mL of Luria broth (LB) containing maltose (0.2% w/v), Kanamycin (50 μg/mL) and Ampicillin (50 μg/mL). Placed in a shaking incubator set to 37° C. overnight (approximately 16 hours).
Cultures for transfection: 1 mL of overnight culture will be inoculated into 98 mL LB medium containing 1 mL of 20% (w/v) maltose, and placed in a shaking incubator set to 37° C. for approximately 4-5 hours. Cells will be pelleted (centrifugation at approximately 2020×g, 10 minutes) and resuspended in approximately 80 mL of LB medium containing 10 mM magnesium sulphate. Working cell suspensions will be stored refrigerated (2-8° C.) or on ice until used for phage adsorption.

### Available In Vivo Toxicity Data on DMP433

In a previously conducted range-finding study (Labcorp Study Number 8526660) DMP433 was dosed orally in groups of 6 males and 6 females at 500, 100 or 50 mg/kg/day daily for up to 14 days. Observations are as follows:
Range-Finder 1 was conducted at 500 mg/kg/day, and was terminated on day 1 due to animal mortality/morbidity and convulsions.
Range-Finder 2 was conducted at 100 mg/kg/day. In the Subgroup 2 animals, clinical signs were noted as squinting, partially closed eyes, fast respiration, mild hunched posture and piloerection, seen spuriously during the dosing period. Where clinical signs were seen, animals had recovered prior to dosing the following day. A single Day 1 (Subgroup 1) female satellite animal was removed from the study due to the severity of the clinical observations. Bodyweights were variable, but all animals gained bodyweight during the 14-day dosing period.
Range-Finder 3 was administered at 50 mg/kg/day. In the Subgroup 2 animals, clinical signs were limited to squinting, mild decreased activity and piloerection, again seen spuriously during the dosing period. Where clinical signs were seen, animals had recovered prior to dosing the following day. Bodyweights were variable, but all animals except one (male M5209) had gained bodyweight during the 14-day dosing period.
The bioanalytical data generated under this range-finding study demonstrated a sex difference of approximately 2-fold at 100 mg/kg/day on Day 14. Therefore, dosing in males and females will be conducted.

### Study Design

Based on the toxicity and bioanalytical data generated under Labcorp Study Number 8526660, the maximum tolerated dose level (MTD) of 100 mg/kg/day* will be used as the high dose level. Two further doses of 50 and 25 mg/kg/day, will also be tested in males (OECD, 2022). As there is a crossover in exposure between males and females, vehicle controls and the MTD of 100 mg/kg/day only will be evaluated in females.
group 4 male animals given 80 mg/kg/day from Day 4 of dosing onwards due to mortality/morbidity.

The dose levels are set out in the following tables:

| | | | Genotoxicity Animal | Satellite Animal ID Tissue |
|---|---|---|---|---|
| | | Dose Level | ID (Subgroup 1) | Bioanalysis (Subgroup 2) |
| Group | Group Description | (mg/kg/day)* | Male | Male |
| 1 | Vehicle control | 0 | M0001-M0007 | — |
| 2 | DMP433 | 25 | M0101-M0107 | M0108-M0110 |
| | Low | | | |
| 3 | DMP433 | 50 | M0201-M0207 | M0208-M0210 |
| | Intermediate | | | |
| 4 | DMP433 | 100/80^{†} | M0301, M0304, M0307, | M0308-M0310 |
| | High | | M0317-M0319⁺ | |

| | | | Satellite Animal ID | Satellite Animal ID Day |
|---|---|---|---|---|
| | | Dose Level | Day 1 (Subgroup 3) | 28 (Subgroup 4) |
| Group | Group Description | (mg/kg/day)* | Male | Male |
| 1 | Vehicle control | 0 | — | — |
| 2 | DMP433 | 25 | M0111-M0116 | M0117-M0122 |
| | Low | | | |
| 3 | DMP433 | 50 | M0211-M0216 | M0217-M0222 |
| | Intermediate | | | |
| 4 | DMP433 | 100/80^{†} | M0311-M0316 | M0320-M0322 |
| | High | | | |

^{†}Due to mortality of animals M0302, M0303, M0305 and M0306, remaining animals given 80 mg/kg/day from Day 4 of dosing onwards.
⁺Animals M0317-M0319 to be transferred from Subgroup 4 to Subgroup 1 from Day 7 onwards.

| | | | | Satellite Animal ID |
|---|---|---|---|---|
| | | | Genotoxicity Animal ID | Tissue Bioanalysis |
| | | Dose Level | (Subgroup 1) | (Subgroup 2) |
| Group | Group Description | (mg/kg/day)* | Female | Female |
| 5 | Vehicle control | 0 | M0401-M0407 | — |
| 6 | DMP433 | 100 | M0501-M0507 | M0508-M0510 |
| | High | | | |

| | | | Satellite Animal ID Day 1 | Satellite Animal ID Day |
|---|---|---|---|---|
| | | Dose Level | (Subgroup 3) | 28 (Subgroup 4) |
| Group | Group Description | (mg/kg/day)* | Female | Female |
| 5 | Vehicle control | 0 | — | — |
| 6 | DMP433 | 100 | M0511-M0516 | M0516-M0522 |
| | High | | | |

### In Life Procedures

Route: oral by gavage (unfasted animals)
Frequency of dosing:
   Genotoxity animals (subgroup 1): daily administrations, for 28 consecutive days.
   Satellite animals (subgroups 2 and 4): daily administrations, for 28 consecutive days.
   Satellites animals (subgroup 3): single administration
Sampling:
   Genotoxicity animals (subgroup 1): animals sampled on day 31.
   Satellite animals (subgroups 2, 3 and 4): as per section 6.1 below.
   Dose volume: 10 mL/kg
All animals will be assessed on arrival for ill health, and assessed prior to start of dosing. The acclimatisation period will be at least seven days.
Experimental observations: all animals will receive a health monitoring twice daily (at the beginning and end (nominal) of the working day). Any animal which shows marked signs of ill health will be isolated and may be killed and necropsied.
Clinical observations: genotoxicity animals (subgroup 1) and satellite animals (subgroups 2, 3 and 4) will be observed once weekly. The animals will be removed from the cage and a physical examination performed, and an individual animal record maintained.
Post dosing observations: all animals will be observed with the following frequency:
   Genotoxicity animals (subgroup 1): day 1 to day 28 prior to dose, immediate, and 1, 2 and 4 hours post-dose. Day 29 to day 31 once daily.
   Satellite animals (subgroups 2, and 4): day 1 to day 27 prior to dose, immediate, and 1, 2 and 4 hours post dose. Day 28 prior to dose.
   Satellite animals (subgroup 3): not required.
The body weights and food consumptions will be observed.

### Bioanalysis and Toxicokinetic Evaluation

6.1 Bioanalysis Blood Sampling Procedures (Males)

| | | | | | |
|---|---|---|---|---|---|
| | | Nominal | | Sample Time | |
| | Analysis | Sample | Groups: No. | (Hours Post | No. of |
| Sample Site | (anticoagulant) | Volume (mL) | Animals/Group | Last Dose) | Samples |

Occasion: Day 1 Satellite Animals (Subgroup 3) - In-Life Bleed¹

| Jugular Vein | Bioanalysis | 0.25 | mL* | Groups 2 to 4 | 0.5, 3 | 18 |
|---|---|---|---|---|---|---|
| | (K₂EDTA) | | | (M0111-M0113) | | |
| | | | | (M0211-M0213) | | |
| | | | | (M0311-M0313) | | |
| Jugular Vein | Bioanalysis | 0.25 | mL* | Groups 2 to 4 | 1 | 9 |

| | |
|---|---|
| (K₂EDTA) | (M0114-M0116) |
| | (M0214-M0216) |
| | (M0314-M0316) |

Occasion: Day 1 Satellite Animals (Subgroup 3) - Terminal Bleed¹

| Cardiac Puncture | Bioanalysis | 0.25 | mL | Groups 2 to 4 | 7 | 9 |
|---|---|---|---|---|---|---|
| | (K₂EDTA) | | | (M0111-M0113) | | |
| | | | | (M0211-M0213) | | |
| | | | | (M0311-M0313) | | |
| Cardiac Puncture | Bioanalysis | 0.25 | mL | Groups 2 to 4 | 24 | 9 |

| | |
|---|---|
| (K₂EDTA) | (M0114-M0116) |
| | (M0214-M0216) |
| | (M0314-M0316) |

Occasion: Day 28 Satellite Animals (Subgroup 4) - In-Life Bleed¹

| Jugular Vein | Bioanalysis | 0.25 | mL* | Groups 2 to 3 | 0.5, 3 | 12 |
|---|---|---|---|---|---|---|
| | (K₂EDTA) | | | (M0117-M0119) | | |
| | | | | (M0217-M0219) | | |
| Jugular Vein | Bioanalysis | 0.25 | mL* | Groups 2 to 3 | 1 | 6 |
| | (K₂EDTA) | | | (M0120-M0122) | | |
| | | | | (M0220-M0222) | | |
| Jugular Vein | Bioanalysis | 0.25 | mL* | Group 4 | 0.5, 1 | 6 |

| | |
|---|---|
| (K₂EDTA) | (M0320-M0322) |

Occasion: Day 28 Satellite Animals (Subgroup 4) - Terminal Bleed¹

| Cardiac Puncture | Bioanalysis | 0.25 | mL* | Groups 2 to 3 | 7 | 6 |
|---|---|---|---|---|---|---|
| | (K₂EDTA) | | | (M0117-M0119) | | |
| | | | | (M0217-M0219) | | |
| Cardiac Puncture | Bioanalysis | 0.25 | mL* | Groups 2 to 3 | 24 | 6 |
| | (K₂EDTA) | | | (M0120-M0122) | | |
| | | | | (M0220-M0222) | | |
| Cardiac Puncture | Bioanalysis | 0.25 | mL | Group 4 | 3 | 6 |

| | |
|---|---|
| (K₂EDTA) | (M0320-M0322) |

¹Decedents: No blood samples will be collected from decedents.
Terminal samples - Blood samples will be taken immediately prior to exsanguination.
*If the animal does not reach the required bodyweight to collect the volume stated, the maximum volume will be calculated and collected as appropriate.

6.2 Bioanalysis Blood Sampling Procedures (Females)

| | | | | | |
|---|---|---|---|---|---|
| | | Nominal | | Sample | |
| | | Sample | | Time | |
| | Analysis | Volume | Groups: No. | (Hours Post | No. of |
| Sample Site | (anticoagulant) | (mL) | Animals/Group | Last Dose) | Samples |

Occasion: Day 1 Satellite Animals (Subgroup 3) - In-Life Bleed¹

| Jugular Vein | Bioanalysis | 0.25 | mL* | Group 6 | 0.5, 3 | 6 |
|---|---|---|---|---|---|---|
| | (K₂EDTA) | | | (M0511-M0513) | | |
| Jugular Vein | Bioanalysis | 0.25 | mL* | Group 6 | 1 | 3 |

| | |
|---|---|
| (K₂EDTA) | (M0514-M0516) |

Occasion: Day 1 Satellite Animals (Subgroup 3) - Terminal Bleed¹

| Cardiac Puncture | Bioanalysis | 0.25 | mL | Group 6 | 7 | 3 |
|---|---|---|---|---|---|---|
| | (K₂EDTA) | | | (M0511-M0513) | | |
| Cardiac Puncture | Bioanalysis | 0.25 | mL | Group 6 | 24 | 3 |

| | |
|---|---|
| (K₂EDTA) | (M0514-M0516) |

Occasion: Day 28 Satellite Animals (Subgroup 4) - In-Life Bleed¹

| Jugular Vein | Bioanalysis | 0.25 | mL* | Group 6 | 0.5, 3 | 6 |
|---|---|---|---|---|---|---|
| | (K₂EDTA) | | | (M0517-M0519) | | |
| Jugular Vein | Bioanalysis | 0.25 | mL* | Group 6 | 1 | 3 |

| | |
|---|---|
| (K₂EDTA) | (M0520- |
| | M0522) |

Occasion: Day 28 Satellite Animals (Subgroup 4) - Terminal Bleed¹

| Cardiac Puncture | Bioanalysis | 0.25 | mL* | Group 6 | 7 | 3 |
|---|---|---|---|---|---|---|
| | (K₂EDTA) | | | (M0517-M0519) | | |
| Cardiac Puncture | Bioanalysis | 0.25 | mL* | Group 6 | 24 | 3 |

| | |
|---|---|
| (K₂EDTA) | (M0520-M0522) |

¹Decedents: No blood samples will be collected from decedents.
Terminal samples - Blood samples will be taken immediately prior to exsanguination.
*If the animal does not reach the required bodyweight to collect the volume stated, the maximum volume will be calculated and collected as appropriate.

The blood samples will be processed. Determination of DMP433 in mouse plasma containing K₂EDTA as an anticoagulant will be conducted by Labcorp Bioanalytical in accordance with Labcorp Policies, and all procedures will be conducted following method DMP4MPP validated under Labcorp study number 8527178.

Toxicokinetic parameters will be estimated using Phoenix® WinNonlin® version 8.1 or higher (Certara USA, Inc., Princeton, New Jersey). A non-compartmental approach consistent with the oral route of administration will be used for parameter estimation. The composite plasma concentration-time data will be used for toxicokinetic calculations.

For determination of mean DMP433 concentrations, concentration values that are below the limit of quantitation will be treated as zero. For determination of toxicokinetic parameters, mean concentrations of zero will be treated as such in the toxicokinetic analysis. Embedded mean values equal to zero may be excluded from toxicokinetic analysis.

All parameters will be generated from composite DMP433 concentrations in plasma from test item-treated groups on Days 1 and 28 of the Main experiment. Parameters will be estimated using nominal dose levels, unless out of specification dose formulation analysis results are obtained, in which case actual dose levels may be used. Parameters will be estimated using nominal sampling times. If predose samples are not collected, predose concentrations will be assumed to be 0 on Day 1 of each phase and will be set equal to the 24-hour concentration on Day 28.

Bioanalytical data will be used as received for the toxicokinetic analysis. Toxicokinetic parameters will be calculated. Descriptive statistics (e.g. mean, standard deviation, coefficient of variation) and toxicokinetic parameters will be reported.

Toxicokinetic parameters to be reported, as data permit, may include but will not be limited to the following:

| | |
|---|---|
| Cmax | Maximum observed concentration. |
| Cmax/D | Dose normalized maximum concentration, |
| | calculated as Cmax/dose. |
| Tmax | Time of maximum observed concentration. |
| AUC0-24 | Area under the curve from time 0 to hour 24, |
| | calculated using the linear trapezoidal rule. |
| AUC0-24/D | Dose normalized AUC0-24, calculated as AUC0-24/dose. |
| AR | Accumulation ratio, calculated as: (Day 28 |
| | Cmax or AUC0-24)/(Day 1 Cmax or AUC0-4). |

Additional parameters and comparisons (e.g., sex ratios, dose proportionality ratios, etc.) may be reported. Additional parameters and comparisons using the non-GLP tissue bioanalysis data (e.g., plasma/tissue exposure ratios) may be reported.

### Terminal Procedures

Animal euthanasia and necropsy

| | |
|---|---|
| Moribund Animals | Exsanguination under isoflurane anaesthesia. An overdose of |
| and Decedents | sodium pentobarbitone, given via intraperitoneal injection and |
| | subsequently ensured by cervical dislocation may be used in |
| | extremis. |
| | Necropsy: macro-observations to determine possible cause of |
| | morbidity/death. |
| Genotoxicity | Cervical dislocation, under isofluorane anaesthesia, subsequently |
| Animals | ensured by exsanguination. |
| (Subgroup 1) | Macroscopic observations of liver, duodenum, kidney and femurs |
| | required. |
| | Animals will be necropsied in group order. |
| Satellite Animals | Animals will not be allowed to recover from the isofluorane |
| (Subgroup 2) | anaesthesia. Death will be by exsanguination. |
| | Macroscopic observations of liver, duodenum, kidney and femurs |
| | required. |
| | Animals will be necropsied in group order. |
| Satellite Animals | Euthanised. |
| (Subgroups 3 and 4) | Animals will not be allowed to recover from the isofluorane |
| | anaesthesia. Death will be by exsanguination. |

Tissue samples

| | Group | Tissue for analysis |
|---|---|---|
| Genotoxicity | 1 | BM, KI, LI, DU |
| Animals | 2 | BM, KI, LI, DU |
| (Subgroup 1) | 3 | BM, KI, LI, DU |
| | 4 | BM, KI, LI, DU |
| | 5 | BM, KI, LI, DU |
| | 6 | BM, KI, LI, DU |
| | | The LI organ weights will be recorded prior to storage. |
| | Tissues will be taken as per the following table at Cmax | |
| | (approximately 30 minutes post-dose) on Day 28. Where possible, | |
| | tissue samples of approx. 100-200 mg will be collected from all | |
| | animals at necropsy. Tissue sample weight will be recorded and the | |
| | samples stored at prior to analysis. | |
| | Group | Tissue for analysis |
| Satellite | 2 | BM, KI, LI, DU |
| Animals | 3 | BM, KI, LI, DU |
| (Subgroup 2) | 4 | BM, KI, LI, DU |
| | 6 | BM, KI, LI, DU |
| Satellite Animals | No tissues to be retained. | |
| (Subgroups 3 and 4), | | |
| moribund animals, | | |
| decedents | | |

In the table above, DU is duodenum, BM is femur and marrow (both femurs), KI is kidney and LI is liver.

The tissue samples will be bioanalysed. Determination of DMP433 in mouse liver, kidney, duodenum and bone marrow will be conducted by Labcorp Discovery Analytical-Metabolism, conducted in accordance with Labcorp Policies. All procedures associated with this analysis will be conducted following methods DMP4MPP.

### Genetic Toxicology Methods

DNA isolation and purification kits such as Recoverease™ (Agilent Technologies UK Ltd., Stockport, UK) and Masterpure™ (Cambio, Cambridge, UK) may be used for DNA extraction.

Transpack kits, providing necessary buffers and reagents for DNA packaging into bacteriophage “heads” and subsequent transfection into the host bacteria, will be supplied by Agilent Technologies UK Ltd., Stockport, UK.

Tissue specific methods will be used to homogenise and lyse tissues as required prior to DNA extraction using commercially available DNA extractions kits or phenol: chloroform methods. All tissue processing methods and DNA extraction techniques will be performed according to Labcorp SOP.

Approximately 5 μL of DNA solution (ideally equivalent to 7.5 μg of DNA) will be mixed with an appropriate packaging extract according to the specific manufacturer's instructions. At the end of the packaging reaction, 10 μL packaged DNA will be diluted with 190 μL SM buffer and used immediately for transfection. For the transfection of host bacteria:
Titre plates—10 μL of diluted packaged DNA will be adsorbed to each of three 500 μL aliquots of working bacterial suspension for 20-30 minutes at room temperature. After adsorption, the phage/bacteria will be suspended in 12 mL 1:3 LB:NaCl, 0.75% w/v agar (top agar) containing 10 mM magnesium sulphate and plated onto 14 cm diameter petri dishes containing 12 mL 1:3 LB:NaCl, 1.5% w/v agar (bottom agar). Once gelled, plates will be inverted and placed in incubator set to 37° C. overnight.
Positive selection plates: Following removal of 3×10 μL of aliquots for the titre plating, the remaining diluted packaged DNA will be split into 3 approximately equal aliquots and adsorbed to each of three 500 μL aliquots of working bacterial suspension for 20-30 minutes at room temperature. After adsorption, the phage/bacteria will be suspended in top agar (as specified above) containing 10 mM magnesium sulphate and 0.3% (w/v) phenylgalactose (P-gal) and plated as described for the titre plates. Once gelled, plates will be inverted and placed in incubator set to 37° C. overnight.

Following overnight incubation, plates may be scored immediately or stored at 2-8° C. and scored as soon as possible. The number of clear plaques on each plate will be counted manually and recorded in the raw data. This will be used to determine the number of plaque forming units (pfu) per reaction.

The plaque scoring criteria will be as follows:
1. Only clearly defined individual plaques are scored
2. Lysed plaques will not be scored
3. Plaques with unusual morphology/size will not be scored.

Positive control DNA will be extracted from frozen tissue samples taken from animals treated with a known mutagen such as ethylnitrosourea.

### Data Evaluation

At least 200,000 plaque forming units (pfu) per tissue per animal, generated from at least 3 independent packaging reactions, will be analysed. A total of at least 1 million pfu per tissue per treatment group; i.e. valid packaging data from at least 5 animals dosed per group.

The mutant frequency (MF) will be calculated per tissue per animal. The mean MF per tissue per treatment group will be calculated. MF is expressed as the number of mutants per 10⁻⁶ pfu.

The mean MF per tissue for treated groups (Groups 2-4 (males) or Group 6 (females)) will be compared to the mean MF for that tissue in Group 1 (males) or Group 5 (females); (vehicle control).

The vehicle control (Group 1 (males) or Group 5 (females)) and the treated groups (Groups 2-4 (males) or Group 6 (females)) will be analysed using one-way analysis of variance (ANOVA) and Dunnett's test, performed with a one-sided risk for increased response in the treated groups. A linear contrast will be used to test for dose response, onesided looking for an increasing response with increasing dose (males only). No linear contrast will be performed in females.

The positive control data* will also be compared to Group 1 (males) or Group 5 (females) using a two-sample t-test, performed with a one-sided risk for increased response in the positive control. No linear contrast will be performed. Levene's test for equality of variances between the groups will be performed and where this shows evidence of heterogeneity (P<0.01), the data will be rank transformed prior to analysis.
Where concurrent positive control animals are not used, data will be generated using DNA from banked samples taken from animals previously dosed with ENU. The data generated will be from unique individual animals and no data will be generated from ‘pooled’ responses.

For valid data, the test item will be considered positive, i.e. capable of inducing mutation, if:
1. A least one of the test doses exhibits a statistically significant increase in the mutant frequency, in any tissue, compared with the concurrent vehicle control, and
2. The increase exceeds the laboratory's historical vehicle control 95% reference range (where applicable) for that tissue, and
3. When evaluated for trend, the results are dose-related.

The test item will be considered negative in this assay if none of the above criteria are met and tissue exposure is confirmed.

### Results

The study was performed according to the study described hereinabove and the test item was positive.

### Example 16—Evaluation of Nitrite Content

Principle: Limit test with gradient HPLC chromatography with FLR (fluorescence) detector.
Standards: Sodium nitrite (NaNO₂) reference standard or certified chemical, e.g. Sodium nitrite Supelco 1.06549
Reagents: 1. Additionally purified water; e.g. Milli Q
   2. Potassium dihydrogen phosphate, KH₂PO₄, e.g. Merck, Cat. No. 1.04873.5000
   3. 2,3-diaminonaphthalene (DAN); e.g. Sigma Aldrich, Cat. No. 88461-100 MG-F
   4. 37% hydrochloric acid, concentrated HCl; e.g. VWR, Cat. No. 6081.2500
   5. Methanol, CH₃OH; e.g. Supelco, Cat. No. 1.06007
   6. Sodium hydroxide, NaOH; e.g. Supelco, Cat No. 1.06498

### Preparation of Solutions:

10 mM KH₂PO₄ Weigh accurately about 1.361 g of KH₂PO₄ into a 1 L volumetric flask, dissolve it in approximately 950 mL of additionally purified water and adjust the pH to 8.0 (with 2.7 M NaOH). Make up to the mark with additionally purified water.
0.62 M HCl Add a few millilitres (e.g. 20 mL) of additionally purified water to a 200 mL volumetric flask, add 10.280 mL of concentrated HCl, mix and make up to the mark with additionally purified water.
2.7 M NaOH Weigh accurately about 1.092 g of NaOH pellets into a 10 mL volumetric flask and dissolve it in additionally purified water. Cool to room temperature and make up to the mark with the same solvent.
Reagent DAN Weigh accurately about 5 mg of 2,3-diaminophthalene into a 100 mL volumetric flask and dissolve it with the solvent (methanol/water 1:1 (v/v)). Dilute up to the mark and ensure visual full dissolution. Use an amberglass flask.
Mobile phase A Mix 950 mL of 10 mM KH₂PO₄ solution (pH=8) and add 50 mL methanol.
Mobile phase B Methanol.
Solvent Methanol/water 1:1 (v/v), e.g. mix 2000 mL of additionally purified water and 2000 mL methanol.

### Equipment

Instrument: HPLC with FLR detector; e.g. Waters Arc or equivalent
Column: C18 50×4.6 mm, 2.5 μm, e.g. XBridge C18 2.5 μm, 4.650 mm or equivalent

### Chromatographic Conditions

Column temperature: 50° C.
Flow rate: 2 mL/min
Wavelength: FLD 375/415 nm
Injection volume: 5 μL
Recording time: 5 min
Sampler temperature: Controlled room temperature e.g. 20° C.

| | t [min] | % Mobile Phase A | & Mobile Phase B |
|---|---|---|---|
| Gradient: | 0 | 80 | 20 |
| System suitability test | 3.5 | 40 | 60 |
| (SST) | 3.6 | 80 | 20 |
| | 5 | 80 | 20 |

Blank solution 1 and Area of the chromatographic peak at the retention time of NAT in blank
comparison solution (ST) solution 1 has to be lower than one third of area of chromatographic peak NAT in comparison solution.
Comparison solution (ST) S/NNAT≥10
   S_{rel(Area,n≥5,NAT)}≤20%
   R_{DANNAT}>3
   Standard agreement ST1/ST2 (%):

$$70 \% \leq \frac{{Area}_{ST 2} \times {m}_{ST 1} \times 100}{{Area}_{ST 1} \times {m}_{ST 2}} \leq 130 \%$$

### Procedure

Notes It is recommended to wear additional protective equipment (face mask and gloves) during weighing.
   It is recommended to wash the column with 50% methanol after the analysis (example of preparation: mix 500 mL methanol and 500 m1 of additionally purified water).
   It is recommended to rinse the injector with solvent after each injection.
   All glassware and tools should be rinsed with additionally purified water before use. It is not recommended to leave the solutions open for a longer time before measurement.
Blank solution 1 Transfer 1 mL of solvent into a HPLC vial and perform derivatization as described below.
Blank solution 2 Transfer 10 mL of solvent into a single use glass vial and mix it on a magnetic stirrer for 30 min. Transfer the solution into a 5 mL centrifuge tube and centrifuge for 10 min at 12000 rpm. Transfer 1 mL of this solution into a HPLC vial and perform the derivatization as described below.
Nitrite stock solution Accurately weight around 18.75 mg of NaNO₂ into a 500 mL volumetric
(2 parallels) flask. Fill up to the mark with solvent and mix.
   (Cₙᵢₜᵣᵢₜₑ≈0.0005 mg/mL)
Comparison solution Transfer 10 mL of nitrite stock solution into a 500 mL volumetric flask.
(2 parallels) Fill up to the mark with solvent and mix.
   Transfer 1 mL solution into a HPLC vial and perform the derivatization as described below.
   (Cₙᵢₜᵣᵢₜₑ≈100 ppb)
Sample solution Accurately weigh about 1000 mg of the sample into a glass vial for single use and add 10 mL of solvent. Mix on a magnetic stirrer for 30 min.
   Transfer the solution into a 5 mL centrifuge tube and centrifuge for 10 min at 12000 rpm. Transfer 1 mL of supernatant into a HPLC vial and perform the derivatization as described below.

$$( {c}_{sample} \approx 100 mg / mL )$$

Derivatization Transfer 1 mL of sample, standard or blank solution into the HPLC vial and add 100 μL 0.062 M HCl. Mix.
   Add 100 μL of the reagent DAN and shake the vials for 10 min for the reaction to occur. After 10 minutes stop the reaction with 23 μL 2.7 M NaOH, close the vial and mix.
Stability of solutions Sample solution, comparison solution, blank solution 1, blank solution 2 are stable for at least 22h in autosampler at 20° C. in the dark.
Sequence e.g. Solvent (1x), Blank solution 1 (1x), Blank solution 2 (1x), Comparison solution ST1 (5x), Comparison solution ST2 (1x), Sample solutions, Solvent (1x), Comparison solution ST1 (1x)
Note Before injections, stabilize the system at an initial conditions for at least 30 minutes, then record the solvent chromatogram and ensure the stable baseline.
Evaluation/Assessment Before comparison, subtract the area of the NAT peak of blank solution 2 from the area of the sample NAT peak and the area of the NAT peak from blank solution 1 from the area of the standard NAT peak. The NAT chromatographic peak in the sample solution chromatogram is compared with the NAT chromatographic peak in the comparison solution ST. The result complies if the area of the chromatographic peak of NAT in the sample is equal to or smaller than the chromatographic peak of NAT in the comparison solution ST. The result is reported as:
   Nitrites≤100 ppb
If the area of chromatographic peak of the NAT in the sample is larger than the chromatographic peak of NAT in the comparison solution ST, the result does not comply and is reported as:

$$Nitrites > 100 ppb$$

Only NAT peak should be evaluated.
Chromatograms Solvent peaks can elute at times around 0.3 and around 3.8 min.

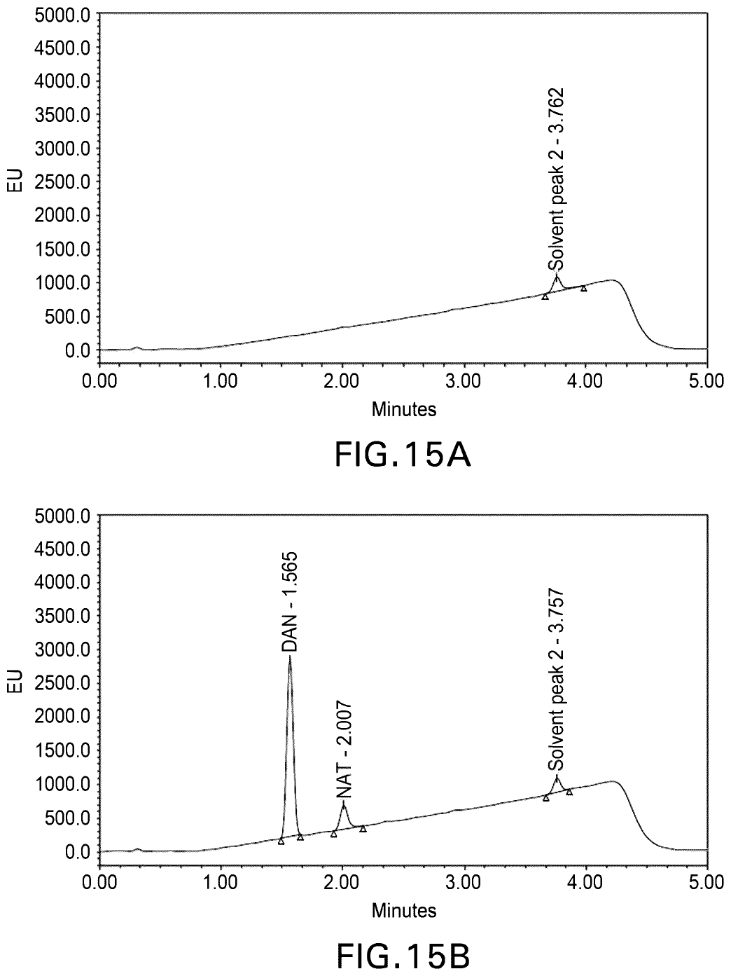

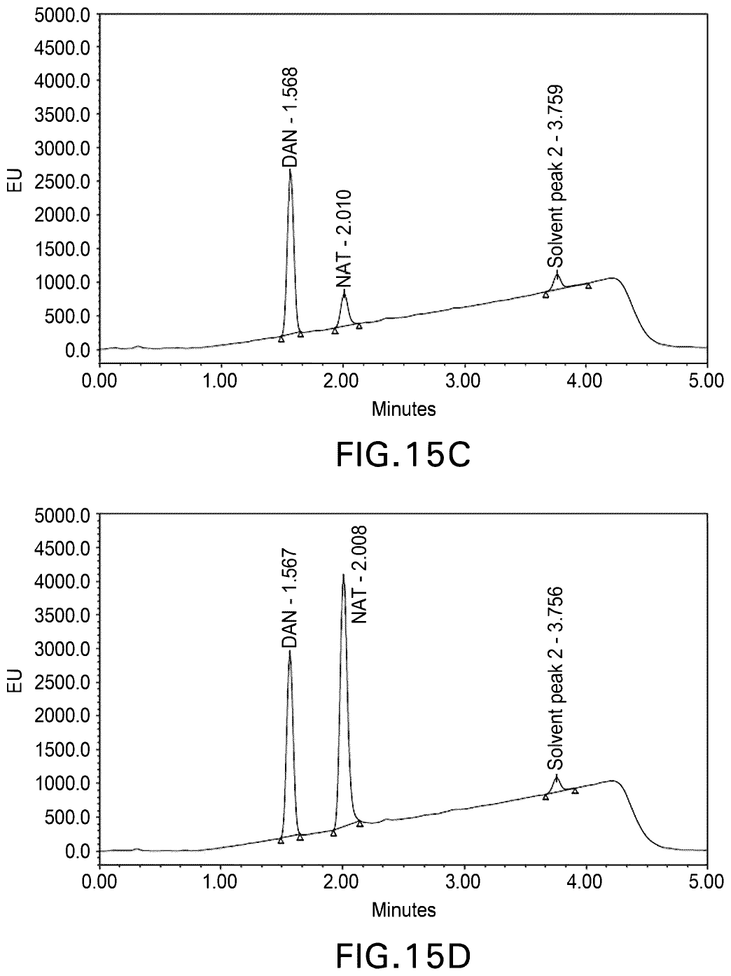

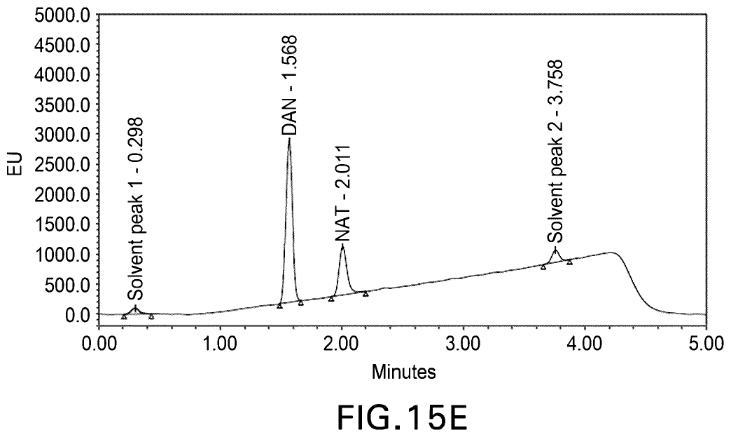

An example of the above-described detection method is provided by FIGS. 15-1 to 15-5.

### Example 17—Accelerated Predictive Stability Modelling for DMP433

Long-term stability behaviour for target storage conditions was predicted based on kinetic modelling of experimental DMP433 impurity data using an accelerated predictive stability study. The evaluation was focused on the DMP433 impurity stability profiles for drug substance and drug product batches (NN4375, NP0866 and NP0867, manufactured with low nitrite microcrystalline cellulose and crospovidone Type A). The drug product is the Kisqali 200 mg film-coated tablet.

### Kinetic Modelling and Statistical Intervals Approach

The Arrhenius equation provides a scientific underpinning for shelf-life estimation of drug substances and products based on quantification of shelf-life limiting reaction rates.

The basic Arrhenius equation can be defined as:

$$k = A \cdot {e}^{( \frac{- {E}_{a}}{R T} )}$$

where:
k is the rate of the chemical reaction, e.g. the degradation rate of the drug substance.
A is the pre-exponential factor or the frequency factor, which delineates the maximum possible rate of reaction,
Eₐ is the activation energy, which is the energy threshold that needs to be overcome for the reaction to proceed,
R is the universal gas constant, and
T is the absolute temperature in Kelvin

Use of the Arrhenius equation is well-established in studying solution-phase reactions. Recent studies have demonstrated that the Arrhenius equation is also reliably applicable to solid-state pharmaceutical materials. Besides the various humidity-corrected equations, a number of studies have shown that if relative humidity (RH) is kept constant, the basic Arrhenius equation is also applicable. This approach is used in the evaluation herein, given that the product is stored inside protective packaging, and to avoid non-representative behaviour to open product exposed to moisture.

One practically useful approach to validate that such assumptions are met and that the particular reaction is predictable using the Arrhenius theorem is the Arrhenius plot, which additionally serves as a simplified approach to estimate the reaction-specific Arrhenius parameters. Arrhenius plots (log(k) versus 1/T) graphically elucidate the relationship drawn in the Arrhenius equation. Owing to the negative correlation between log (k) and 1/T, this plot produces a straight line with a negative slope (−Ea/R). It stresses upon the fundamental stipulation that the rate of chemical reaction decreases as the temperature diminishes. Note that log in this case is the natural log.

The kinetic model was employed to model experimental data herein as follows:

The NVP-DMP433 level (y) over time (t) at fixed T temperature can be modeled by the first-order reaction model:

$$y ( t ) = y ( t , T ) = ( {y}_{0} - {y}_{∞} ) {e}^{- k ( T ) t} + {y}_{∞}$$

with rate k being the temperature-dependent formation rate. The temperature dependence of the rate k is modeled using the Arrhenius formula described above. Another parametrization in use is expressing constants with respect to some reference temperature T_{ref}[K], namely:

$$k ( T ) = k ( {t}_{ref} ) exp ( - \frac{{E}_{a}}{R} [ \frac{1}{T} - \frac{1}{{T}_{ref}} ] ) = k ( {t}_{ref} ) exp ( - \frac{{E}_{a}}{{RT}_{ref}} [ \frac{T - {T}_{ref}}{T} ] )$$

where the parameters are k(T_{ref}) i.e. k_{ref} and Eₐ are considered as parameters and were computationally estimated using model fitting against experimental data. Furthermore, one-sided 95% upper confidence intervals were calculated as:

$$\hat{y} ( t , T ) + {t}_{y ; v} \cdot \frac{s}{\sqrt{{n}_{eff}}}$$

where s is the residual standard deviation, t_{y,v} denotes the y-quantile of the (central) t-distribution with v degrees of freedom and n_{eff}=n_{eff}(t; T) stands for the effective sample size at t and T. This is computed as:

$${n}_{eff} = \frac{{s}^{2}}{{se}^{2} ( t , T )}$$

where se(t; T) denotes the standard error of prediction at time t and temperature T. Since the model is nonlinear, there are no explicit formulas to compute the standard error, and residual bootstrapping is used to estimate standard error and in turn the effective sample size and confidence intervals.

### Experimental Data for the Drug Product

The data used for model fitting and shelf-life predictions for the drug product (DP) are based on film coated samples placed in closed vial samples and subjected to 30°, 40° and 50° C. stress temperatures for 28 days in an accelerated predictive stability study.

The data as used in the model is given in the table below.

| | | DMP433 | Time | Temperature |
|---|---|---|---|---|
| Sample | Batch | [ppm] | [day] | [° C.] |
| NN4375 5° C. 0 days - Replicate 1 | NN4375 | 0.259 | 0 | Initial |
| NN4375 5° C. 0 days - Replicate 2 | NN4375 | 0.249 | 0 | Initial |
| NN4375 30° C. CLOSED 7 days | NN4375 | 0.264 | 7 | 30 |
| NN4375 40° C. CLOSED 7 days | NN4375 | 0.322 | 7 | 40 |
| NN4375 50° C. CLOSED 7 days | NN4375 | 0.520 | 7 | 50 |
| NN4375 30° C. CLOSED 14 days | NN4375 | 0.280 | 14 | 30 |
| NN4375 40° C. CLOSED 14 days | NN4375 | 0.380 | 14 | 40 |
| NN4375 50° C. CLOSED 14 days | NN4375 | 0.745 | 14 | 50 |
| NN4375 30° C. CLOSED 21 days | NN4375 | 0.300 | 21 | 30 |
| NN4375 40° C. CLOSED 21 days | NN4375 | 0.443 | 21 | 40 |
| NN4375 50° C. CLOSED 21 days | NN4375 | 0.857 | 21 | 50 |
| NN4375 30° C. CLOSED 28 days | NN4375 | 0.359 | 28 | 30 |
| NN4375 40° C. CLOSED 28 days | NN4375 | 0.542 | 28 | 40 |
| NN4375 50° C. CLOSED 28 days | NN4375 | 1.066 | 28 | 50 |
| NP0866 5° C. 0 days - Replicate 1 | NP0866 | 0.212 | 0 | Initial |
| NP0866 5° C. 0 days - Replicate 2 | NP0866 | 0.210 | 0 | Initial |
| NP0866 30° C. CLOSED 7 days | NP0866 | 0.223 | 7 | 30 |
| NP0866 40° C. CLOSED 7 days | NP0866 | 0.246 | 7 | 40 |
| NP0866 50° C. CLOSED 7 days | NP0866 | 0.336 | 7 | 50 |
| NP0866 30° C. CLOSED 14 days | NP0866 | 0.236 | 14 | 30 |
| NP0866 40° C. CLOSED 14 days | NP0866 | 0.268 | 14 | 40 |
| NP0886 50° C. CLOSED 14 days | NP0866 | 0.402 | 14 | 50 |
| NP0866 30° C. CLOSED 21 days | NP0866 | 0.239 | 21 | 30 |
| NP0866 40° C. CLOSED 21 days | NP0866 | 0.298 | 21 | 40 |
| NP0866 50° C. CLOSED 21 days | NP0866 | 0.478 | 21 | 50 |
| NP0866 30° C. CLOSED 28 days | NP0866 | 0.260 | 28 | 30 |
| NP0866 40° C. CLOSED 28 days | NP0866 | 0.319 | 28 | 40 |
| NP0866 50° C. CLOSED 28 days | NP0866 | 0.538 | 28 | 50 |
| NP0867 5° C. 0 days - Replicate 1 | NP0867 | 0.279 | 0 | Initial |
| NP0867 5° C. 0 days - Replicate 2 | NP0867 | 0.279 | 0 | Initial |
| NP0867 30° C. CLOSED 7 days | NP0867 | 0.316 | 7 | 30 |
| NP0867 40° C. CLOSED 7 days | NP0867 | 0.352 | 7 | 40 |
| NP0867 50° C. CLOSED 7 days | NP0867 | 0.492 | 7 | 50 |
| NP0867 30° C. CLOSED 14 days | NP0867 | 0.319 | 14 | 30 |
| NP0867 40° C. CLOSED 14 days | NP0867 | 0.389 | 14 | 40 |
| NP0867 50° C. CLOSED 14 days | NP0867 | 0.624 | 14 | 50 |
| NP0867 30° C. CLOSED 21 days | NP0867 | 0.345 | 21 | 30 |
| NP0867 40° C. CLOSED 21 days | NP0867 | 0.420 | 21 | 40 |
| NP0867 50° C. CLOSED 21 days | NP0867 | 0.754 | 21 | 50 |
| NP0867 30° C. CLOSED 26 days | NP0867 | 0.287 | 28 | 30 |
| NP0867 40° C. CLOSED 28 days | NP0867 | 0.396 | 28 | 40 |
| NP0867 50° C. CLOSED 28 days | NP0867 | 0.685 | 28 | 50 |

### Experimental Data for the Drug Substance

The data used for model fitting and impurity level prediction for drug substance storage are based on open samples subjected to stress temperatures (30°, 40°, 50° and 60° C.) in the form of an accelerated predictive stability study. All data is based on 75% relative humidity and varying temperatures. This allows the use of the basic Arrhenius equation without a humidity term, given the constant humidity across all samples in scope.

The data as used in the model is given in the table below.

| | | DMP433 | Time | Temperature |
|---|---|---|---|---|
| Sample | Batch | [ppm] | [day] | [° C.] |
| B711440AA T0 −1 | B711440AA | 0.322 | 0 | Initial |
| B711440AA T0 −2 | B711440AA | 0.321 | 0 | Initial |
| B711440AA T0 −3 | B711440AA | 0.326 | 0 | Initial |
| B711440AA 60° C. 75% RH 3 days | B711440AA | 0.430 | 3 | 60 |
| B711440AA 30° C. 75% RH 7 days | B711440AA | 0.342 | 7 | 30 |
| B711440AA 40° C. 75% RH 7 days | B711440AA | 0.364 | 7 | 40 |
| B711440AA 50° C. 75% RH 7 days | B711440AA | 0.436 | 7 | 50 |
| B711440AA 60° C. 75% RH 7 days | B711440AA | 0.555 | 7 | 60 |
| B711440AA 30° C. 75% RH 14 days | B711440AA | 0.367 | 14 | 30 |
| B711440AA 40° C. 75% RH 14 days | B711440AA | 0.441 | 14 | 40 |
| B711440AA 50° C. 75% RH 14 days | B711440AA | 0.649 | 14 | 50 |
| B711440AA 60° C. 75% RH 14 days | B711440AA | 0.513 | 14 | 60 |
| B711440AA 30° C. 75% RH 21 days | B711440AA | 0.383 | 21 | 30 |
| B711440AA 40° C. 75% RH 21 days 1 | B711440AA | 0.417 | 21 | 40 |
| B711440AA 40° C. 75% RH 21 days 2 | B711440AA | 0.426 | 21 | 40 |
| B711440AA 50° C. 75% RH 21 days | B711440AA | 0.471 | 21 | 50 |
| 833268A144 T0 −1 | 833268A144 | 0.191 | 0 | Initial |
| 833268A144 T0 −2 | 833268A144 | 0.193 | 0 | Initial |
| 833268A144 T0 −3 | 833268A144 | 0.193 | 0 | Initial |
| 833268A144 60° C. 75% RH 3 days | 833268A144 | 0.241 | 3 | 60 |
| 833268A1444 30° C. 75% RH 7 days | 833268A144 | 0.191 | 7 | 30 |
| 833268A144 40° C. 75% RH 7 days | 833268A144 | 0.205 | 7 | 40 |
| 833268A144 50° C. 75% RH 7 days | 833268A144 | 0.241 | 7 | 50 |
| 833268A144 60° C. 75% RH 7 days | 833268A144 | 0.297 | 7 | 60 |
| 833268A144 30° C. 75% RH 14 days | 833268A144 | 0.215 | 14 | 30 |
| 833268A144 40° C. 75% RH 14 days | 833268A144 | 0.255 | 14 | 40 |
| 833268A144 50° C. 75% RH 14 days | 833268A144 | 0.351 | 14 | 50 |
| 833268A144 60° C. 75% RH 14 days | 833268A144 | 0.294 | 14 | 60 |
| 833268A144 30° C. 75% RH 21 days | 833268A144 | 0.214 | 21 | 30 |
| 833268A144 40° C. 75% RH 21 days 1 | 833268A144 | 0.235 | 21 | 48 |
| 833268A144 40° C. 75% RH 21 days 2 | 833268A144 | 0.234 | 21 | 40 |
| 833268A144 50° C. 75% RH 21 days | 833268A144 | 0.255 | 21 | 50 |

Calculations and plots were implemented using the statistical programming language R, version 4.1.

### Drug Product and Drug Substance Results and Predictions

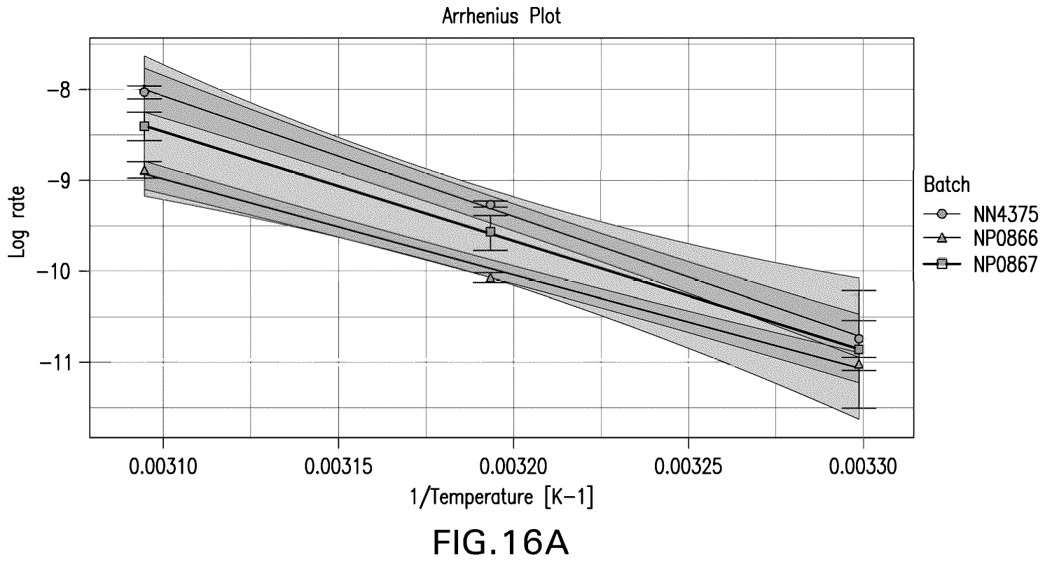

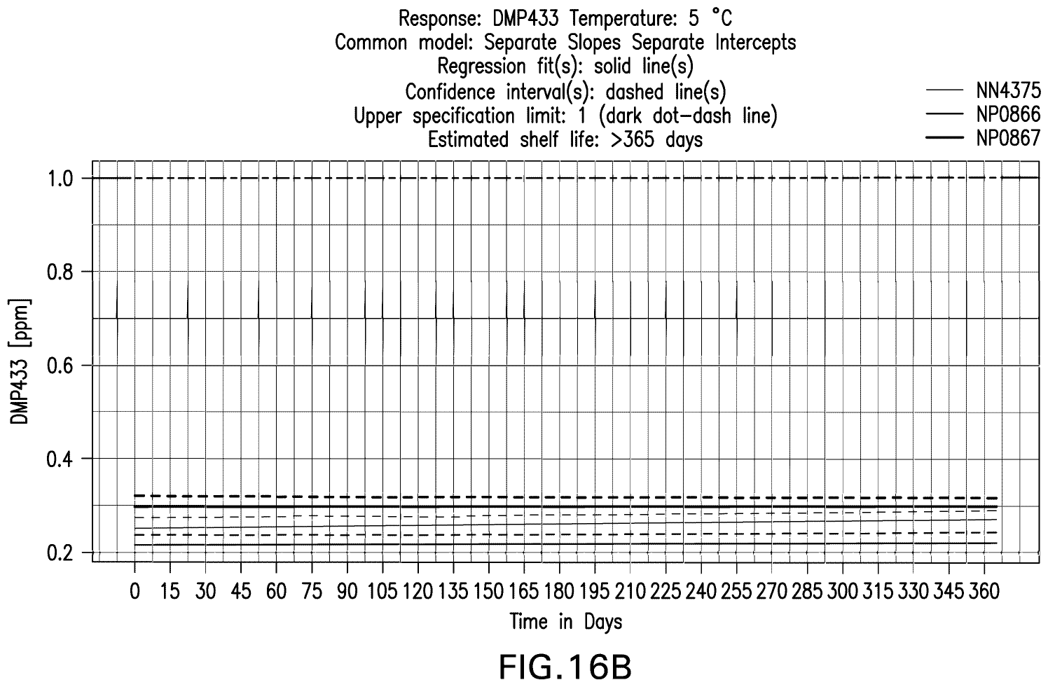

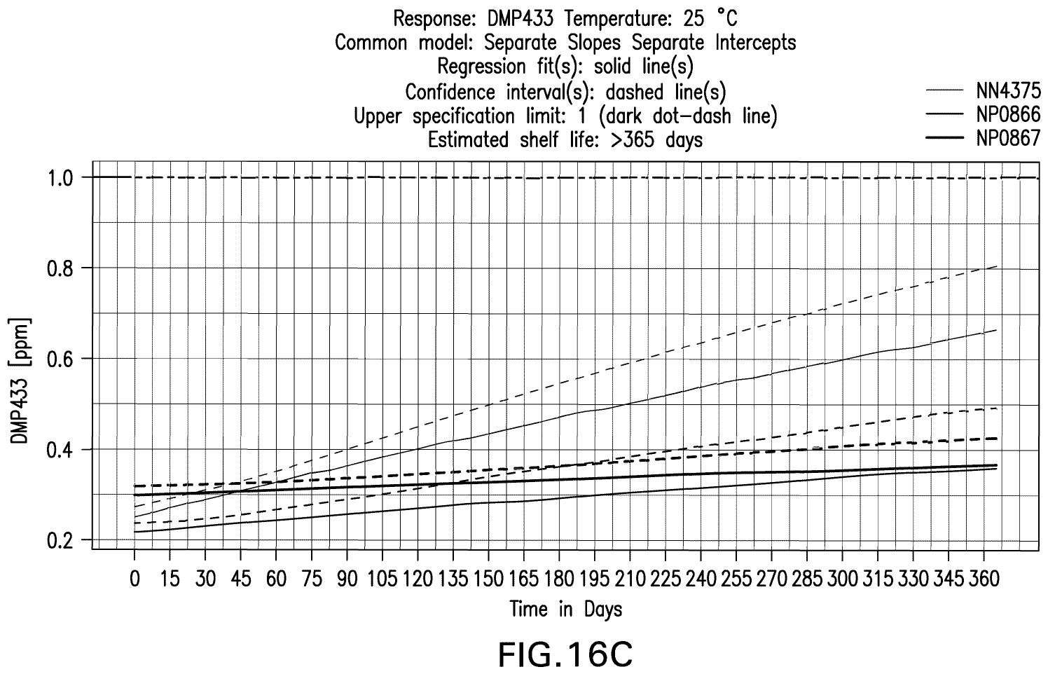

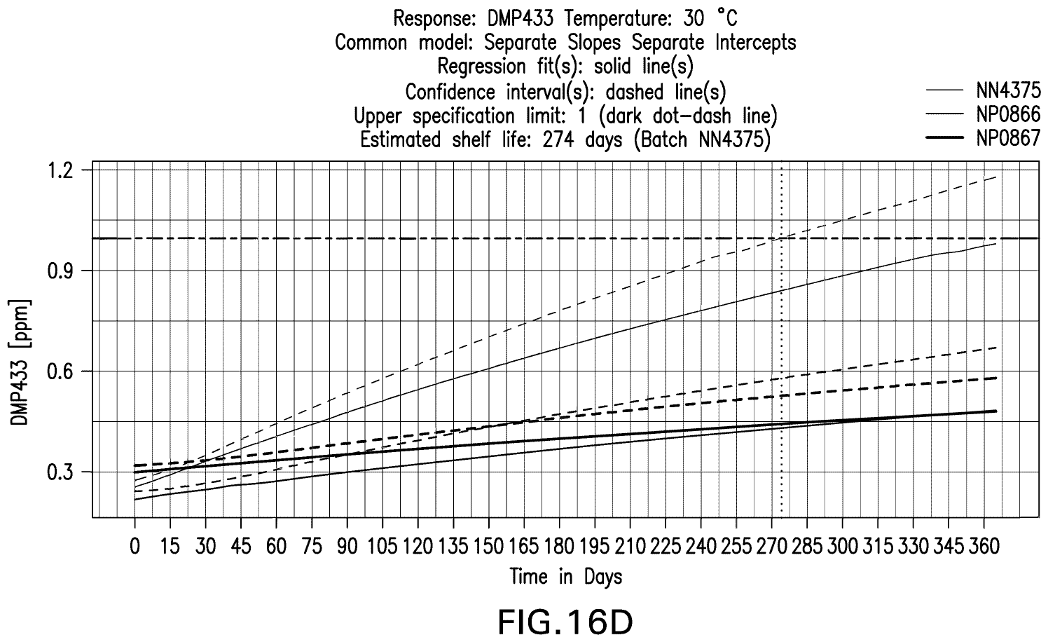

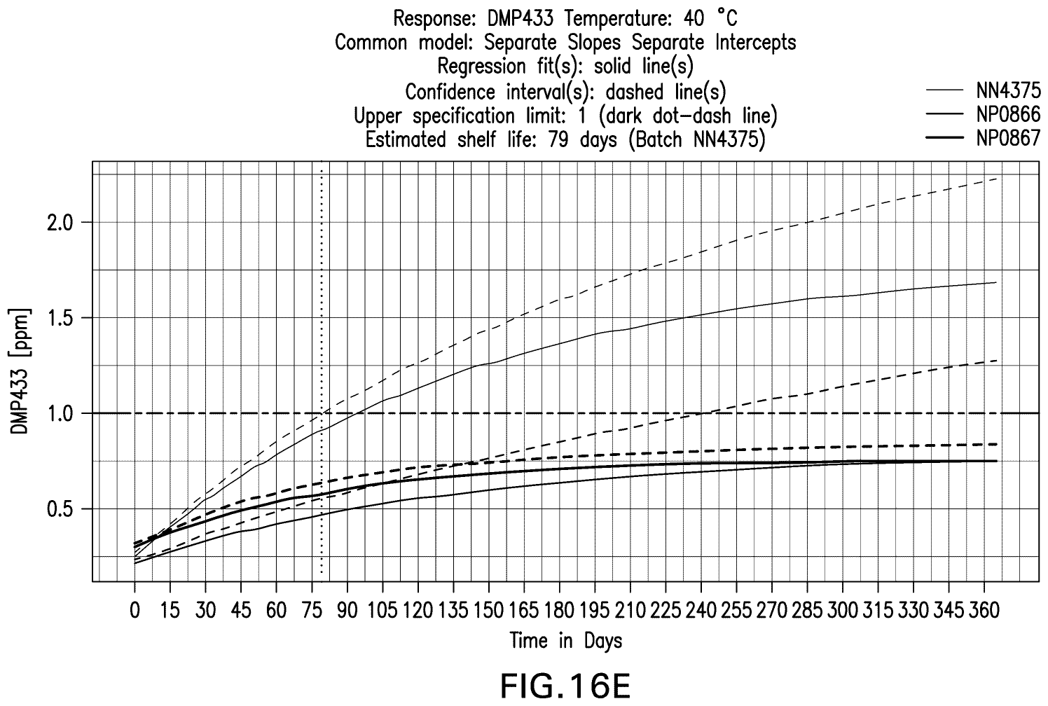

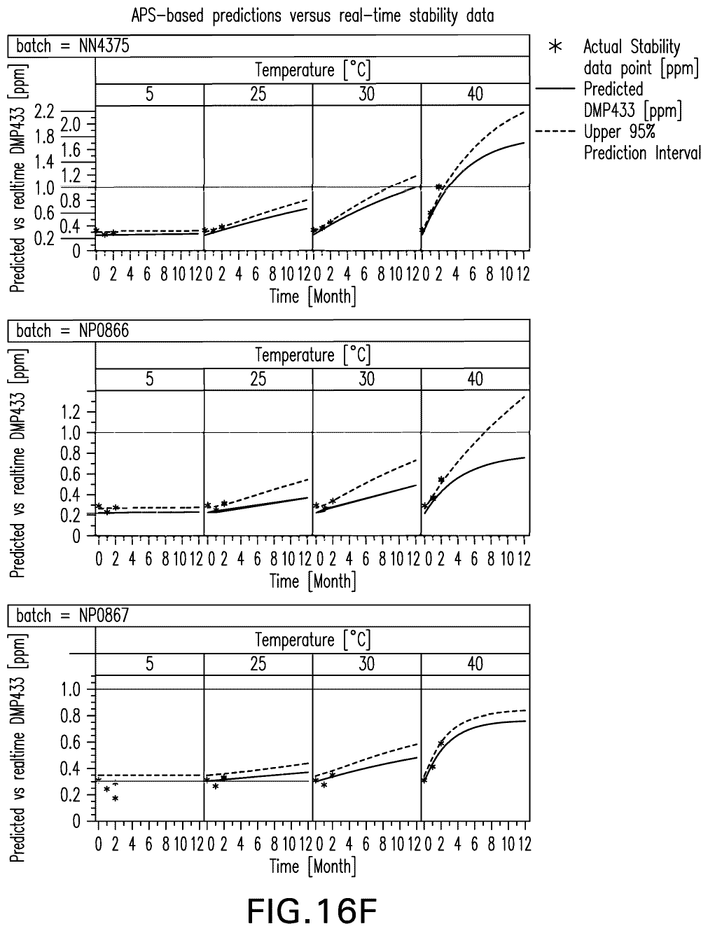

For the drug product, the Arrhenius plot is shown in FIG. 16A. As a basis for the validity of the kinetic model to predict the DMP433 impurity, the formation rate should have a temperature dependence that matches the Arrhenius equation, namely a linear relationship between log rate and the reciprocal of temperature (in Kelvin). This is clearly demonstrated in the plot of FIG. 16A, showing a linear relationship between the experimentally derived reaction rates against their respective study temperatures.

The shelf-life predictions for the drug product are shown in FIGS. 16B-E.

The predicted stability profiles (fit and the corresponding 95% prediction intervals) are compared against individual real-time data in FIG. 16-F. It can be concluded from the plot of FIG. 16-F that real-time stability data trends are in reasonable agreement with the predicted stability profiles, specifically for the stability temperatures in scope of the proposed product storage (2-8° C. and 25° C.). This further demonstrates the reliability of the predictions. The stability data is provided in the Table below.

| | | | | | Real-time, | |
|---|---|---|---|---|---|---|
| | Time | Temperature | Real-time | Real-time | rounded | |
| Batch | [Month] | [° C.] | [ng] | [ppm] | [ppm] | FP Batch |
| [symbol] | 0 | [symbol] | [symbol] | [symbol] | 0.3 | |
| [symbol] | 1 | [symbol] | [symbol] | [symbol] | 0.3 | [symbol] |
| [symbol] | 1 | [symbol] | [symbol] | [symbol] | 0.3 | [symbol] |
| [symbol] | 2 | [symbol] | [symbol] | [symbol] | 0.3 | [symbol] |
| [symbol] | 2 | [symbol] | [symbol] | [symbol] | 0.3 | [symbol] |
| [symbol] | 0 | 25 | [symbol] | [symbol] | 0.3 | |
| [symbol] | 1 | 25 | [symbol] | 0.32 | 0.3 | [symbol] |
| [symbol] | 2 | 25 | 74 | 0.37 | 0.4 | [symbol] |
| [symbol] | 2 | 25 | 77 | [symbol] | 0.4 | [symbol] |
| [symbol] | 0 | 30 | [symbol] | [symbol] | 0.3 | |
| [symbol] | 1 | 30 | 71 | [symbol] | 0.4 | [symbol] |
| [symbol] | 2 | 30 | 89 | 0.445 | 0.4 | [symbol] |
| [symbol] | 0 | 40 | [symbol] | 0.325 | 0.3 | |
| [symbol] | 1 | 40 | 120 | 0.6 | 0.6 | [symbol] |
| [symbol] | 1 | 40 | 110 | [symbol] | 0.6 | [symbol] |
| [symbol] | 2 | 40 | 201 | [symbol] | 1.0 | [symbol] |
| [symbol] | 2 | 40 | [symbol] | [symbol] | 1.0 | [symbol] |
| [symbol] | 0 | [symbol] | [symbol] | [symbol] | 0.3 | |
| [symbol] | 1 | [symbol] | [symbol] | 0.23 | 0.2 | [symbol] |
| [symbol] | 1 | [symbol] | [symbol] | 0.225 | 0.2 | [symbol] |
| [symbol] | 2 | [symbol] | [symbol] | 0.27 | 0.3 | [symbol] |
| [symbol] | 2 | [symbol] | [symbol] | 0.27 | 0.3 | [symbol] |
| [symbol] | 0 | 25 | [symbol] | 0.29 | 0.3 | |
| [symbol] | 1 | 25 | [symbol] | 0.245 | 0.2 | [symbol] |
| [symbol] | 1 | 25 | [symbol] | 0.24 | 0.2 | [symbol] |
| [symbol] | 2 | 25 | 60 | 0.3 | 0.3 | [symbol] |
| [symbol] | 2 | 25 | 62 | 0.31 | 0.3 | [symbol] |
| [symbol] | 0 | 30 | [symbol] | 0.29 | 0.3 | |
| [symbol] | 1 | 30 | [symbol] | [symbol] | 0.3 | [symbol] |
| [symbol] | 2 | 30 | [symbol] | 0.33 | 0.3 | [symbol] |
| [symbol] | 0 | 40 | [symbol] | 0.29 | 0.3 | |
| [symbol] | 1 | 40 | 71 | [symbol] | 0.4 | [symbol] |
| [symbol] | 1 | 40 | 74 | [symbol] | 0.4 | [symbol] |
| [symbol] | 2 | 40 | 109 | [symbol] | 0.5 | [symbol] |
| [symbol] | 2 | 40 | 107 | [symbol] | 0.3 | [symbol] |
| [symbol] | 0 | 5 | [symbol] | 0.31 | 0.3 | |
| [symbol] | 1 | 5 | [symbol] | 0.24 | 0.2 | [symbol] |
| [symbol] | 1 | 5 | [symbol] | 0.24 | 0.2 | [symbol] |
| [symbol] | 2 | 5 | [symbol] | 0.17 | 0.2 | [symbol] |
| [symbol] | 2 | 5 | [symbol] | 0.29 | 0.3 | [symbol] |
| [symbol] | 0 | 25 | [symbol] | [symbol] | 0.3 | |
| [symbol] | 1 | 25 | 53 | [symbol] | 0.3 | [symbol] |
| [symbol] | 1 | 25 | 53 | [symbol] | 0.3 | [symbol] |
| [symbol] | 2 | 25 | [symbol] | 0.33 | 0.3 | [symbol] |
| [symbol] | 2 | 25 | 64 | 0.32 | 0.3 | [symbol] |
| [symbol] | 0 | 30 | [symbol] | 0.31 | 0.3 | |
| [symbol] | 1 | 30 | [symbol] | 0.275 | 0.3 | [symbol] |
| [symbol] | 2 | 30 | 70 | 0.35 | 0.4 | [symbol] |
| [symbol] | 0 | 40 | [symbol] | 0.31 | 0.3 | |
| [symbol] | 1 | 40 | [symbol] | 0.41 | 0.4 | [symbol] |
| [symbol] | 1 | 40 | [symbol] | 0.415 | 0.4 | [symbol] |
| [symbol] | 2 | 40 | 118 | [symbol] | [symbol] | [symbol] |
| [symbol] | 2 | 40 | 118 | [symbol] | [symbol] | [symbol] |

[symbol]

indicates data missing or illegible when filed

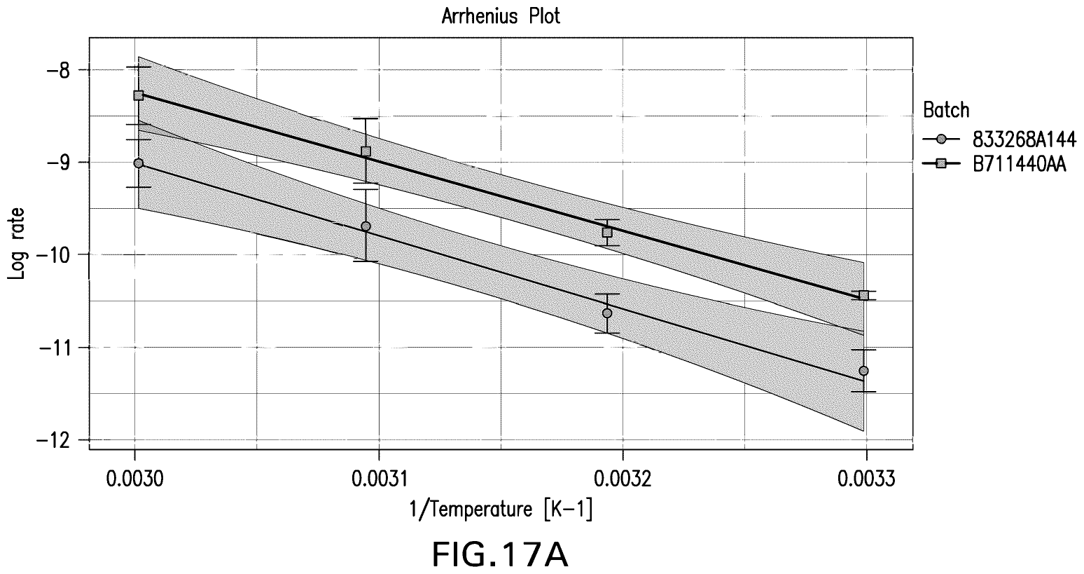

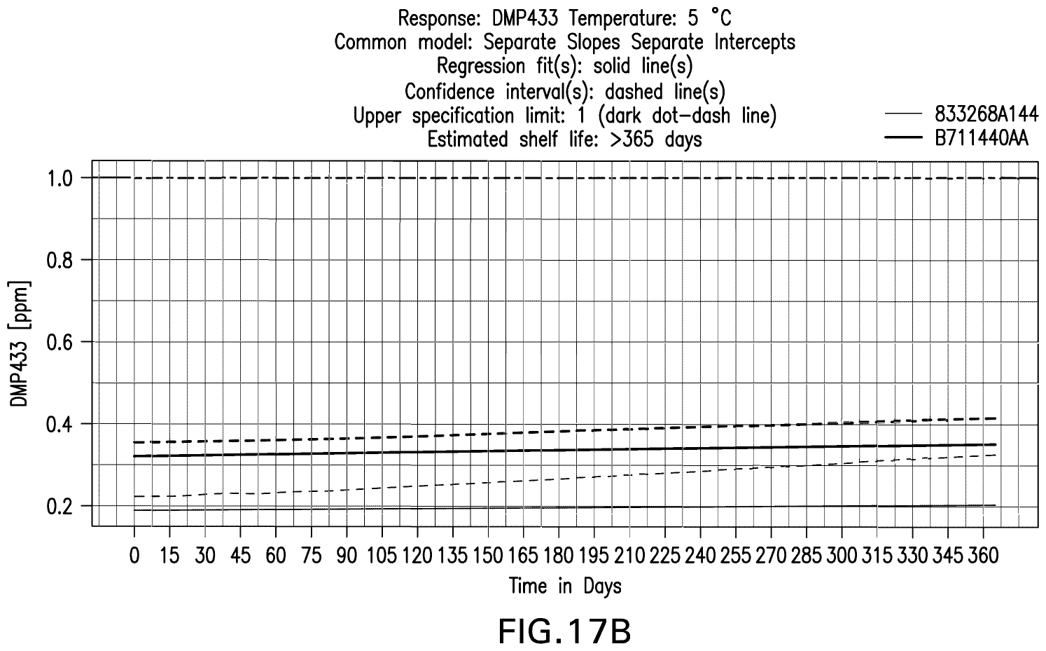

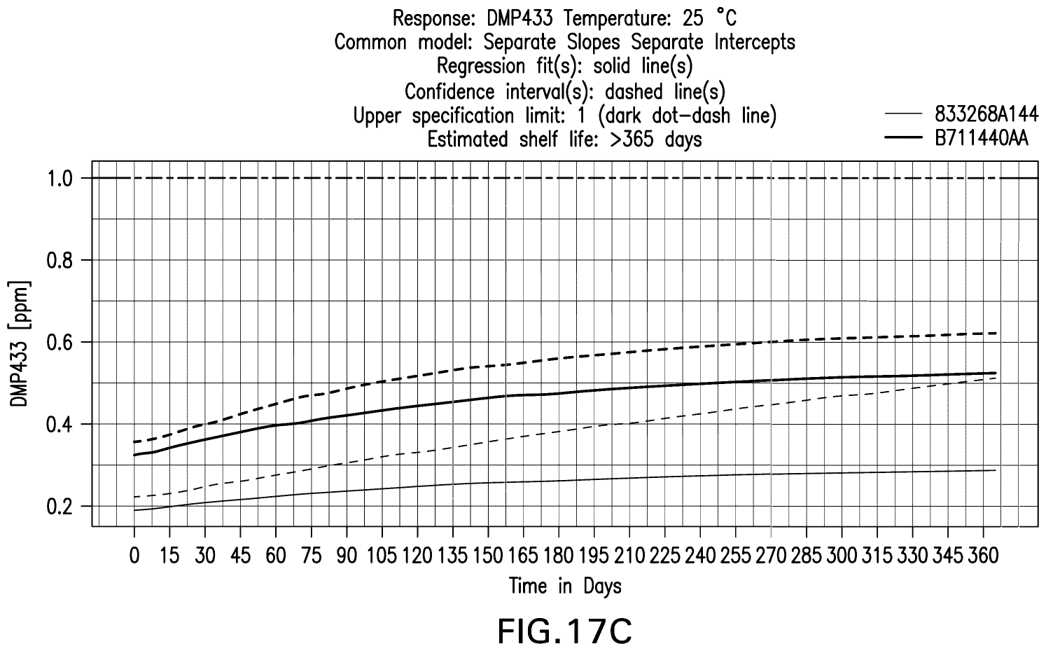

For the drug substance, the Arrhenius plot is shown in FIG. 17A, and the shelf-life predictions are shown in FIGS. 17B-C.

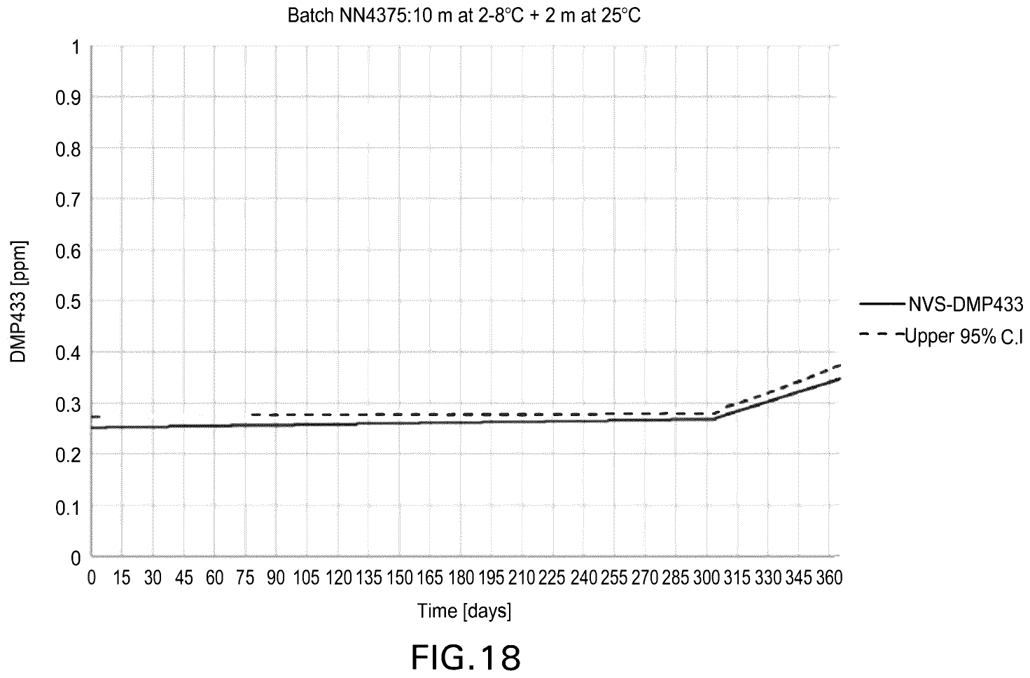

A stability profile is shown in FIG. 18.

### CONCLUSIONS

Based on predictive modelling of accelerated predictive stability data on closed samples from the three representative batches manufactured with low nitrite microcrystalline cellulose and crospovidone type A, stability profiles were elucidated for various stability conditions as shown in FIG. 16-17, which demonstrates an effectively flat DMP433 stability profile at 2-8° C. (denoted as 5° C. in the figures), as well as slow formation at 25° C., both providing levels compliant with an upper limit of 1.0 ppm for at least 1 year. Furthermore, the profiles are shown to reach the 1.0 ppm upper limit after more than 6 months at 30° C., and between 2-3 months at 40° C. (batch NN4375).

The graph presented in FIG. 18 shows the scenario whereby drug product batch NN4375 is stored for 10 months at 2-8° C. followed by period of use storage for 2 months at 25° C. It can be observed that the levels of DMP433 are projected to be flat after 10 months of refrigerated storage and show a minimal increase after two months at 25° C. The overall change across stability can be calculated as the difference between maximum point from the 95% upper confidence interval series, and the minimum point from the predicted fit series, which is 0.122 ppm in this stability scenario. The method precision of 10.4% RSD can be translated as standard deviation=0.104 at the 1 ppm level, which results in an analytical buffer of 0.171 ppm (using 95% coverage one-sided). Therefore, a DP upper release limit of 0.7 ppm is appropriate and accounts for the overall change across stability in the scenario visualized herein. Consequently, the DS upper release limit can be conservatively set at 0.4 ppm to allow for a sufficient margin that ensures meeting the DP upper release limit of 0.7 ppm, even after 12 month of DS storage at 2-8° C.

The predictive stability modelling therefore concludes that the drug product release is expected, with 95% confidence, to be well within the limit of 1.0 ppm after a total of 12 months of storage when a limit of 0.7 ppm (corresponding to 140 ng/tablet) is applied.

It should be noted that the scenario visualized above is based on an effective zero increase at 5° C. and near zero-order kinetics at low temperatures, which is clearly evident by inspecting the stability prediction plots. The term (time) in the x-axis for 25° C. phase in FIG. 18 is representing (t-10 months) of the kinetic model.

### Example 18—Evaluation of an Additional Drying Step

The effect of implementing an additional drying step in order to reduce the formation of DMP433 during the subsequent storage of the ribociclib drug product was investigated. A single technical batch was prepared wherein two different types of drying were evaluated.

### Starting Materials

The product composition of the ribociclib drug product is provided in the table below.

| | | | |
|---|---|---|---|
| | | Composition per unit, | Composition per unit, |
| Ingredients | Function | mg | % |

Inner-phase

| | | | |
|---|---|---|---|
| Ribociclib succinate | Active ingredient | 254.40^{(1), (2)} | 56.9 |
| (LEE011) | | | |
| Microcrystalline | Filler | 67.44 | 15.1 |
| cellulose | | | |
| Hydroxypropyl Cellulose | Binder | 48.12 | 10.8 |
| (L-HPC) | | | |
| Crospovidone (Type A) | Disintegrant | 29.20 | 6.5 |
| Aerosil | Glidant | 2.12 | 0.5 |
| Magnesium stearate | Lubricant | 6.36 | 1.4 |

Outer-phase

| Crospovidone (Type A) | Disintegrant | 12.84 | 2.9 |
|---|---|---|---|
| Aerosil | Glidant | 1.06 | 0.2 |
| Magnesium stearate | Lubricant | 8.46 | 1.9 |
| Total | | 430.00 | |
| Coating premix white | Coating⁽³⁾ | 16.80 | |
| Coating premix black | Coating⁽³⁾ | 0.25 | 3.8 |
| Coating premix red | Coating⁽³⁾ | 0.15 | |
| Purified water⁽⁴⁾ | | | |

| | | |
|---|---|---|
| Total | 447.20 | 100.0 |

⁽¹⁾The salt factor is 1.272; 254.40 mg of LEE011 salt correspond to 200.00 mg of LEE011 free base.
⁽²⁾A compensation of DS is performed if the corrected DS content is <99.5%.
⁽³⁾The coating premix is commercially available.
⁽⁴⁾Removed during processing. Suspension solid content 20% w/w.

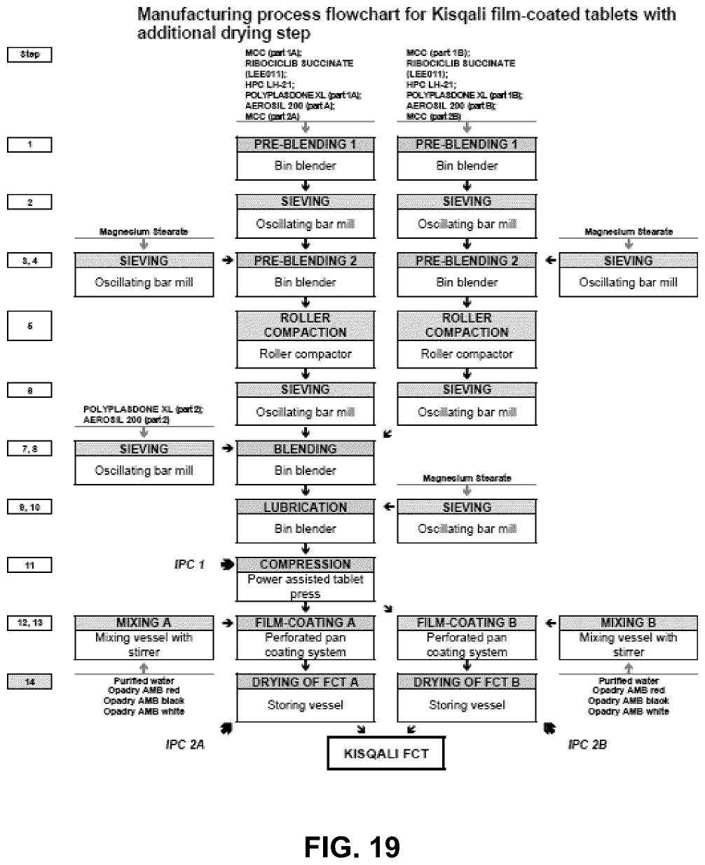

The manufacturing process for the ribociclib drug product consists of blend preparation, roller compaction, compression, and film-coating. This process is shown schematically in FIG. 19 as steps 1-13. As shown in the figure, an additional step occurring after the film-coating of the tablets, i.e. a step of drying the film coated tablets, was evaluated. Two different drying methods were studied (step 14 of FIG. 19).

In process controls for the ribociclib drug product are presented in the table below.

| Process step | IPC test (test principle) | Target value/range |
|---|---|---|
| 11.Compression | Appearance | White to pale yellow coloured, |
| | | no score; round tablet, curved |
| | | with bevelled edges; 11.0 mm ± |
| | | 0.2 mm diameter; debossed |
| | | with “RIC” on one side and |
| | | “NVR” on the other side |
| | Mean Mass | 417.1-442.9 mg [430.0 mg ± 3%] |
| | Mean Thickness | 4.5-4.9 mm |
| | Mean Hardness | 80-100N |
| | Friability | ≤1.0% |
| 14.Drying of Film Coated | Appearance | Light greyish violet, no score; |
| Tablets | | round tablet, curved with |
| | | bevelled edges; 11.1 mm ± 0.2 |
| | | mm diameter; debossed with |
| | | “RIC” on one side and “NVR” on |
| | | the other side |
| | Mean Mass | 433.8-460.6 mg [447.2 mg ± 3%] |
| | Mean Thickness | 4.6-5.0 mm |

Summary of Information about the Technical Batch that was Used in the Study

Technical batch NV1900 was produced in accordance with a validated technological procedure for the production of the ribociclib drug product up until step 13. The contents of nitrites and DMP433 within the starting materials that were used for the production of NV1900 are provided in the table below.

| | | | | Nitrite |
|---|---|---|---|---|
| Material | | | N-nitroso- | content |
| number | Material | Batch number | ribociclib (ppm) | (ppm) |
| 40007341 | RIBOCICLIB SUCCINATE N | B728969 | 0.21 | — |
| | NOVA PW UD 04 GE | (833268A280) | 0.18 | — |
| | | | B728970 | |
| | | | (833268A282) | |
| 45012155 | MICROCR. CELLULOSE | 32559798 | — | 0.02 |
| | PH102 VLN | | | |
| 45012156 | POLYPLASDONE XL LN | 32561140 | — | 0.05 |

Coating was performed in two sub-batches as for regular production, however, for sub-batch A, the drying of the film coating was prolonged from 12 min to 60 min. Sub-batch B was coated without any changes.

Film-coated tablets from both sub-batches underwent 100% automatic visual control under reduced humidity (RH NMT 15%), with film-coated tablets being collected in 320L drying vessels. Each sub-batch was collected in two drying vessels.

Film-coated tablets of sub-batch A were then subjected to an additional drying step using a Huettlin HKC-400 device for 48 hours. Film-coated tablets of sub-batch B were subjected to an alternative additional drying step using drying vessels and a PIP drying system for 72 hours. The PIP drying system is a drying system which can be used to flow air over the tablets at a specified humidity, air flow rate, and temperature. Any equivalent drying system may be used. The water activity of the film-coated tablets was measured every 4 and every 8 hours, respectively.

Results show that both methods of drying are efficient in reducing the water activity of the film coated tablets. Drying using the Huettlin method reduced the water activity from 0.126 to 0.066 in 48 hours. Drying using the PIP drying system reduced the water activity from 0.143 to 0.053 in 72 hours.

### Additional Drying Step

The impact of the additional step of drying the ribociclib film-coated tablets on the formation of DMP433 in the final drug product was evaluated.

### Drying of Film-Coated Tablets Using the Huettlin Method

Sub-batch A (˜260 kg FCT) was coated in accordance with a validated technological procedure but with a prolonged drying of the film coating within the coater GCS-500, i.e. the step was 60 minutes long, with inlet air temperature 45° C. and inlet air humidity 3.5 g/kg (the validated procedure uses drying for 12 min, 55° C., 2.5 g/kg). Film-coated tablets of sub-batch A were 100% automatically visually sorted at RH NMT 15% and stored in 320L vessels (˜130 kg FCT per vessel). Water activity was measured after coating and during Spine visual sorting. The results of these measurements are presented in the table below. All water activity measurements were performed on 8 film-coated tablets (on halves)

| Sample | Water activity |
|---|---|
| A - after coating (after last step “cooling”) | 0.124 |
| A - during Spine sorting | 0.1256 |

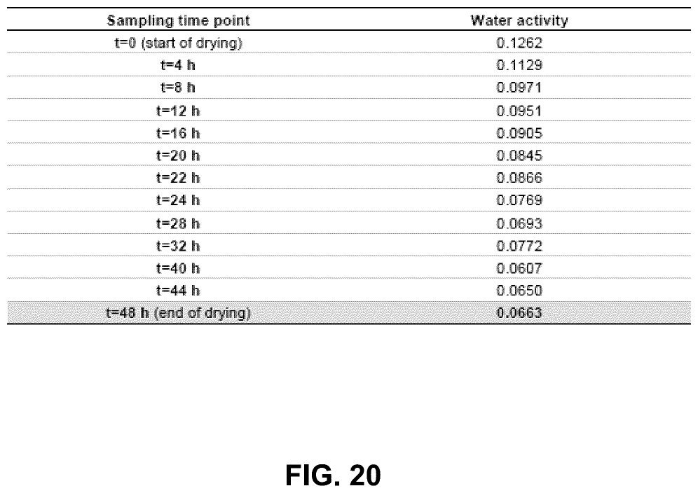

Film-coated tablets of sub-batch A were then loaded into the Huettlin HKC-400 device and subjected to an additional drying step. Specifically, the film-coated tablets of sub-batch A were flushed with dry air for 48 hours (inlet air humidity 0.1-0.5 g/kg; inlet air temperature 30° C., airflow 3000 m³/h). Water activity was measured every 4 hours. The results are presented in the table below and in FIG. 20.

| Sampling time point | Water activity |
|---|---|
| t = 0 (start of drying) | 0.1262 |
| t = 4 h | 0.1129 |
| t = 8 h | 0.0971 |
| t = 12 h | 0.0951 |
| t = 16 h | 0.0905 |
| t = 20 h | 0.0845 |
| t = 22 h | 0.0866 |
| t = 24 h | 0.0769 |
| t = 28 h | 0.0693 |
| t = 32 h | 0.0772 |
| t = 40 h | 0.0607 |
| t = 44 h | 0.0650 |
| t = 48 h (end of drying) | 0.0663 |

It was thereby demonstrated that the Huettlin drying method successfully reduced the water activity from 0.126 to 0.066. Film-coated tablets were also analysed for in-process controls (IPCs) after the additional drying step. All IPCs were found to meet the acceptance criteria. Therefore, sampling of the film-coated tablets after the additional drying step (step 14) or after the coating step (step 13) showed that there was no impact on product quality. The hardness of the film-coated tablets was also measured. Film-coated tablets after the additional drying step have an average hardness of 115 N, which indicates that the additional drying step does not affect the mechanical properties of the tablets (the average hardness of the tablet cores was 91 N).

### Drying of Film-Coated Tablets in Drying Vessels Using the PIP Drying System

Sub-batch B (˜260 kg FCT) was coated in accordance with the validated procedure using coater GCS-500-2, and an inlet air humidity of 2.5 g/kg for 12 min at 55° C. Film-coated tablets of sub-batch B were 100% automatically visually sorted at RH NMT 15% and stored in two 320L drying vessels (˜130 kg FCT per drying vessel). Water activity was measured after coating and during Spine visual sorting. The results of these measurements are presented in the table below.

| Sample | Water activity |
|---|---|
| B - after coating (after last step “cooling”) | 0.130 |
| B - during Spine sorting | 0.1431 |

As the bulk density of the ribociclib film-coated tablets is about 0.77 g/mL, each drying vessel was about 53% filled. Vessels were connected to the PIP drying system, and the film-coated tablets were flushed with dry air for 72 hours. The inlet temperature was set to 30° C., the inlet air humidity was 0.01 g/kg, and the airflow for each drying vessel was 28-30 m³/h.

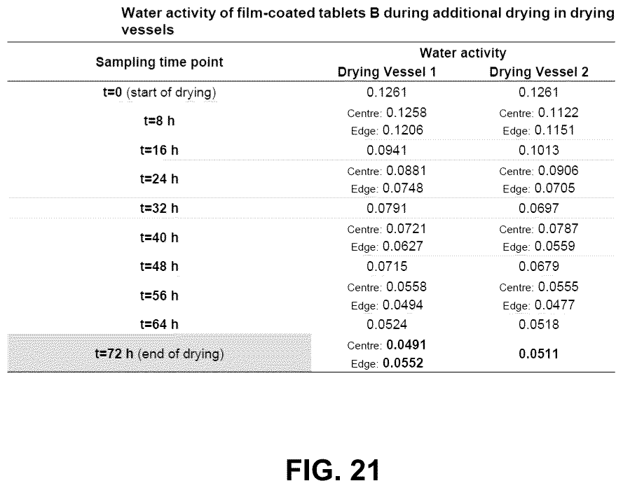

All water activity measurements were performed from each drying vessel every 8 hours (see table below and FIG. 21). At certain time-points samples were taken from the centre of the vessels and from the edge, since the drying vessels have air coming into the tablet bed from the centre. Results thus also give an insight on any potential difference of aw values within different areas of the vessels.

| | Water activity | Water activity |
|---|---|---|
| Sampling time point | (Drying vessel 1) | (Drying vessel 2) |
| t = 0 (start of drying) | 0.1261 | 0.1261 |
| t = 8 h | Centre: 0.1258 | Centre: 0.1122 |
| | Edge: 0.1206 | Edge: 0.1151 |
| t = 16 h | 0.0941 | 0.1013 |
| t = 24 h | Centre: 0.0881 | Centre: 0.0906 |
| | Edge: 0.0748 | Edge: 0.0705 |
| t = 32 h | 0.0791 | 0.0697 |
| t = 40 h | Centre: 0.0721 | Centre: 0.0787 |
| | Edge: 0.0627 | Edge: 0.0559 |
| t = 48 h | 0.0715 | 0.0679 |
| t = 56 h | Centre: 0.0558 | Centre: 0.0555 |
| | Edge: 0.0494 | Edge: 0.0477 |
| t = 64 h | 0.0524 | 0.0518 |
| t = 72 h (end of drying) | Centre: 0.0491 | 0.0511 |
| | Edge: 0.0552 | |

It was thereby demonstrated that the use of the drying vessels in the PIP drying system successfully reduced the water activity of the tablets from 0.126 to 0.055. Whilst there was a small difference between the water activity values of the centre and edge samples, this is not significant: an average sample taken from between the centre and the edge of the vessel will be representative of the water activity that is achieved in the drying vessel.

The film-coated tablets of sub-batch B were also analysed for in-process controls (IPCs) after the additional drying step. All IPCs were found to meet the acceptance criteria. Therefore, sampling of the film-coated tablets after the additional drying step (step 14) or after the coating step (step 13) showed that there was no impact on product quality. The hardness of the film-coated tablets was also measured. Film-coated tablets after the additional drying step have an average hardness of 108 N, which indicates that the additional drying step does not affect the mechanical properties of the tablets (the average hardness of the tablet cores was 91 N).

Measurement of DMP433 within Dried Film-Coated Tablets

Dried film-coated tablets after 48 h of drying using the Huettlin method or after 44 h of drying in drying vessels on the PIP drying system (composite sample) were analysed for their content of DMP433. The results of these measurements are presented in the table below.

| Sample | DMP433, ppm |
|---|---|
| FCT A before Spine | 0.207 |
| FCT B before Spine | 0.195 |
| Dried FCT A (48 h in Huettlin) | 0.230 |
| Dried FCT B (44 h in drying vessels) | 0.196 |

The results show that the additional drying step has a negligible impact on DMP433 content, since all reported results are approx. 0.2 ppm.

Film-coated tablets from each of the two sub-batches (sub-batch A dried for 48 hours using the Huettlin method; sub-batch B dried for 44 hours using drying vessels on the PIP drying system) were subjected to stability testing under stress conditions (70° C. for 1 week). The content of DMP433 was analysed. The results are shown in the table below. The experiment shows that despite equivalent amounts of DMP433 in the samples at T=0, higher values of DMP433 were obtained on the sample that was dried using the Huettlin method. Without being bound to this theory, the inventors believe that the difference may be due to the smaller inlet airflow (28 to 30 m³/h as compared to 3000 m³/h) that was used for drying sub-batch B as compared to sub-batch A. This results in a reduced exposure of the FCTs to NOx species within the airflow during the additional drying step.

| Drying approach | Sample | T = 0 | T = 1 week (70° C.) |
|---|---|---|---|
| Huettlin sub-batch A | NV1900 after coating* | 0.2 ppm | / |
| | NV1900 after drying 48 hr | 0.2 ppm | 0.8 ppm |
| Drying vessel sub-batch B | NV1900 after coating | 0.2 ppm | / |
| | NV1900 after drying 44 hr | 0.2 ppm | 0.4 ppm |

*Prolonged drying in pan coater (60 min) was performed on this sample, prior to drying in the Huettlin.

### Water Activity of Dried FCTs Exposed to RH NMT 15%

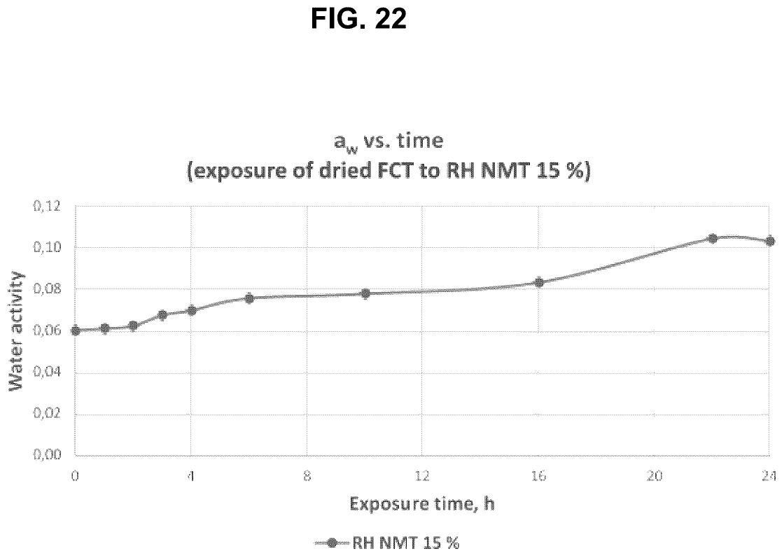

To assess the water uptake of dried ribociclib film-coated tablets, dried sub-batch A (from the Huettlin method) was exposed to environmental conditions with a lower air humidity (RH NMT 15%; actual relative humidity 5.3-8.9%). Water activity was measured for 24 hours. The results are presented in the table below and in FIG. 22. As seen from these results, water activity starts to increase after 4 hours of exposure time.

| Sampling time point | Water activity |
|---|---|
| t = 0 | 0.0604 |
| (first exposure to ambient air with RH NMT 15%) | |
| t = 1 h | 0.0611 |
| t = 2 h | 0.0624 |
| t = 3 h | 0.0675 |
| t = 4 h | 0.0698 |
| t = 6 h | 0.0757 |
| t = 10 h | 0.0778 |
| t = 16 h | 0.0883 |
| t = 22 h | 0.1044 |
| t = 24 h | 0.1033 |
| FCT A after coating, before additional drying | 0.124 |

Therefore, when operating with “dried FCTs”, i.e., when storing or packaging ribociclib FCTs, conditions of low humidity should be used, e.g. RH NMT 15%.

### Example 19—Evaluation of Packaging Materials

Batches of ribociclib film coated tablets (200 mg) were prepared in accordance with the process used in Example 18 for sub-batch B, i.e. wherein the film coated tablets were subjected to an additional step of drying using drying vessels in the PIP drying system. The tablets were packaged in either Aclar blisters or Alu-Alu blisters and the water activity was measured at various time points, as shown in the table below.

| | | 2-8° C. | | | 25° C./60% RH | | | 30° C./75% RH | | | 40° C./75% RH | |
|---|---|---|---|---|---|---|---|---|---|---|---|---|
| | T0 | 4 m | 6 m | 11 m | 4 m | 6 m | 11 m | 4 m | 6 m | 11 m | 4 m | 6 m |
| Aclar | 0.04 | 0.06 | 0.05 | na | 0.07 | 0.07 | na | 0.12 | 0.10 | na | 0.16 | 0.19 |
| blister | | | | | | | | | | | | |
| Alu-Alu | 0.05 | 0.06 | 0.03 | na | 0.04 | 0.03 | na | 0.05 | 0.04 | na | 0.04 | 0.04 |
| blister | | | | | | | | | | | | |

Samples from the same batches of were also subjected to stability testing under stress conditions (40° C., 75% RH). Representative data is provided in the table below.

| | Time point | | | | | | |
|---|---|---|---|---|---|---|---|
| | | 1 | 2 | 3 | 4 | 5 | 6 |
| | Initial | month | months | months | months | months | months |
| Aclar | 0.29 | 0.30 | 0.46 | 0.42 | 0.54 | 0.55 | 0.45 |
| blister | | | | | | | |
| Alu-Alu | 0.28 | 0.25 | 0.35 | 0.36 | 0.35 | 0.29 | 0.31 |
| blister | | | | | | | |

These results demonstrate that while the water activity in the ribociclib FCTs that are stored in Aclar blisters increases during storage, it remains stable in the ribociclib FCTs that are stored in Alu-Alu blisters. Furthermore, while the amount of DMP433 in the ribociclib FCTs that are stored in Aclar blisters increases during storage under stress conditions, it remains stable in the ribociclib FCTs that are stored in Alu-Alu blisters. Without being bound by this theory, the inventors believe that this difference arises from the reduced gas permeability of the Alu-Alu blisters. This reduces the exposure of the FCTs to air at ambient humidity resulting in increased water activity and/or to air containing NOx species.

Equivalent samples stored in Alu-Alu blisters were tested under different temperature and humidity conditions for up to 2 months and the amount of DMP433 in the samples was measured. Based on these results, stability profiles were predicted for 25° C., 60% and 30° C., 75% RH. These stability profiles support a shelf life of at least 18 months at 25° C., 60% and of at least 18 months at 30° C., 75% RH. According to the profiles, the amount of DMP433 in the ribociclib FCTs that are dried in drying vessels and stored in Alu-Alu blisters would not exceed 0.7 ppm, relative to the total amount of ribociclib in free or salt form, during either of these types of storage.

### Clauses—List A

The invention provides the following numbered clauses:

1. A composition comprising or consisting of ribociclib or a pharmaceutically acceptable salt of ribociclib, wherein said composition is substantially free of N-nitroso-ribociclib and salts thereof.

2. A composition comprising or consisting of ribociclib or a pharmaceutically acceptable salt thereof, wherein the total amount of N-nitroso-ribociclib and salts thereof in the composition is no more than 1 ppm, relative to the total amount of ribociclib in free or salt form.

3. A composition comprising or consisting of ribociclib or a pharmaceutically acceptable salt thereof, wherein the total amount of N-nitroso-ribociclib and salts thereof in the composition is no more than the maximum amount of nitrosamines that is permitted in such a composition, by a regulatory authority, at the time when the composition is prepared and/or administered.

4. A composition comprising or consisting of ribociclib or a pharmaceutically acceptable salt thereof which has been tested and found to have a total amount of N-nitroso-ribociclib and salts thereof of no more than 1 ppm, relative to the total amount of ribociclib in free or salt form.

5. A composition comprising or consisting of ribociclib or a pharmaceutically acceptable salt thereof which has been tested and found to have a total amount of N-nitroso-ribociclib and salts thereof that is no more than the maximum amount of nitrosamines that is permitted in such a composition, by a regulatory authority, at the time when the composition is prepared and/or administered.

6. A pharmaceutical product comprising the composition of any one of clauses 1 to 5 and instructions to a patient as to how to administer the composition.

7. A pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib and salts thereof in the composition is no more than 1 ppm, relative to the total amount of ribociclib in free or salt form.

8. A pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib and salts thereof in the composition is no more than the maximum amount of nitrosamines that is permitted in such a composition, by a regulatory authority, at the time when the composition is prepared and/or administered.

9. The pharmaceutical product of any one of clauses 6 to 8, wherein the product further comprises instructions to a patient as to how to administer the composition.

10. The composition or pharmaceutical product of any one of clauses 1 to 9, wherein the total amount of ribociclib or a pharmaceutically acceptable salt thereof in the composition provides the same amount of ribociclib as 200 mg of ribociclib free base.

11. A composition or pharmaceutical product according to any one of clauses 1 to 10, wherein the composition is an oral dosage form, optionally wherein the oral dosage form is a tablet.

12. A composition or pharmaceutical product according to any one of clauses 1 to 10, wherein the composition consists of ribociclib or a pharmaceutically acceptable salt thereof within an orally administrable capsule.

13. A composition or pharmaceutical product according to any one of clauses 1 to 12, wherein the composition is present in a sealed container and is under an atmosphere in which the percentage of oxygen is less than 21% by volume, optionally wherein the atmosphere is nitrogen.

14. A composition or pharmaceutical product according to any one of clauses 1 to 13, wherein the composition is present in a sealed container together with desiccant.

15. A composition or pharmaceutical product according to any one of clauses 1 to 14, wherein the composition further comprises one or more pharmaceutically acceptable excipients.

16. A method of preparing ribociclib or a pharmaceutically acceptable salt of ribociclib with an acid, wherein the method comprises the use of one or more solvents which, immediately prior to their use in the method, are stored under an atmosphere in which the percentage of oxygen is less than 21% by volume, optionally wherein the atmosphere is nitrogen.

17. The method of clause 16, wherein said method comprises the use of said one or more solvents for dissolving ribociclib and/or said acid.

18. The method of clause 17, wherein the method is for preparing a pharmaceutically acceptable salt of ribociclib with an acid, and the method comprises the use of said one or more solvents for dissolving ribociclib and said acid, prior to their combination to form the pharmaceutically acceptable salt.

19. A method of preparing ribociclib or a pharmaceutically acceptable salt of ribociclib with an acid, wherein the method comprises: (a) obtaining one or more solvents, where the one or more solvents are contained in a sealed container under an atmosphere in which the percentage of oxygen is less than 21% by volume, optionally wherein the atmosphere is nitrogen; and (b) using the one or more solvents to dissolve ribociclib and/or said acid.

20. The method of any one of clauses 16 to 19, wherein the total amount of nitrites in the one or more solvents are no more than 5 ppb, preferably no more than 3.5 ppb, and more preferably no more than 1 ppb, relative to the amount of the respective solvent.

21. A method of preparing ribociclib or a pharmaceutically acceptable salt thereof, wherein the method comprises the use of one or more solvents, and wherein the method comprises distilling said one or more solvents to reduce their content of nitrites, prior to their use.

22. A method of preparing ribociclib or a pharmaceutically acceptable salt of ribociclib with an acid, wherein the method comprises the use of one or more solvents for dissolving ribociclib and/or said acid, and wherein the method comprises distilling the one or more solvents to reduce their content of nitrites, prior to said use.

23. A method of preparing ribociclib or a pharmaceutically acceptable salt of ribociclib with an acid, wherein the method comprises: (a) obtaining one or more solvents, optionally wherein the one or more solvents are contained in a sealed container under an atmosphere in which the percentage of oxygen is less than 21% by volume, preferably wherein the atmosphere is nitrogen; (b) distilling said one or more solvents to reduce their content of nitrites; and (c) using the one or more distilled solvents to dissolve ribociclib and/or said acid.

24. The method of any one of clauses 21 to 23, wherein the total amount of nitrites in the one or more solvents, following the distillation, is no more than 5 ppb, preferably no more than 3.5 ppb, and more preferably no more than 1 ppb, relative to the amount of the respective solvent.

25. A method of preparing ribociclib or a pharmaceutically acceptable salt thereof, wherein the method comprises the use of one more solvents, and wherein the method comprises passing said one or more solvents through an ion exchange resin that is capable of adsorbing nitrites.

26. A method of preparing ribociclib or a pharmaceutically acceptable salt of ribociclib with an acid, wherein the method comprises the use of one or more solvents for dissolving ribociclib and/or said acid, and wherein the method comprises passing the one or more solvents through an ion exchange resin which is capable of adsorbing nitrites, prior to said use.

27. The method of clause 25 or 26, wherein the method comprises: (a) distilling the one or more solvents to reduce their content of nitrites; and (b) passing the one or more distilled solvents through said ion exchange resin.

28. A method of preparing ribociclib or a pharmaceutically acceptable salt of ribociclib with an acid, wherein the method comprises a step of passing a solution comprising or consisting of: (a) ribociclib or a pharmaceutically acceptable salt thereof; and (b) a solvent, through an ion exchange resin that is capable of adsorbing nitrites from the solution.

29. The method of clause 28, wherein the ion exchange resin adsorbs no more than 5% by weight of said ribociclib or said pharmaceutically acceptable salt thereof.

30. A method of preparing ribociclib or a pharmaceutically acceptable salt of ribociclib with an acid, wherein the method comprises a step of passing a solution comprising or consisting of: (a) said acid; and (b) a solvent, through an ion exchange resin that is capable of adsorbing nitrites from the solution.

31. The method of any one of clause 28 to 30, wherein the method comprises: (a) obtaining one or more solvents, optionally wherein the one or more solvents is contained in a sealed container under an atmosphere in which the percentage of oxygen is less than 21% by volume, preferably wherein the atmosphere is nitrogen; (b) distilling the one or more solvents to reduce their content of nitrites; and (c) using the one or more distilled solvents to dissolve ribociclib and/or said acid.

32. The method of clause 31, wherein the method further comprises: (d) passing the solution of ribociclib and/or the solution of said acid through an ion exchange resin that is capable of adsorbing nitrites from a solution.

33. The method of any one of clauses 25 to 32, wherein the one or more ion exchange resin that is capable of adsorbing nitrites is selected from the group consisting of an activated charcoal resin, a silica gel resin, a celite resin, or an aluminium oxide resin.

34. The method of any one of clauses 16 to 33, wherein the one or more solvents is or comprises isopropanol, optionally wherein the isopropanol is essentially free of isopropyl nitrite.

35. The method of any one of clauses 21 to 24, 27 or 31, wherein the one or more solvents is or comprises isopropanol and wherein the distillation is over ascorbic acid.

36. A method of preparing ribociclib or a pharmaceutically acceptable salt of ribociclib with an acid, wherein the method comprises the use of one or more filters which do not comprise a nitrocellulose membrane.

37. The method of clause 36, wherein the one or more filters are used to filter a solution of ribociclib and/or a solution of said acid.

38. A method of preparing ribociclib or a pharmaceutically acceptable salt of ribociclib with an acid, wherein the method comprises: (a) obtaining a solution of ribociclib and/or a solution of said acid; and (b) filtering the one or more solutions with a filter that does not comprise a nitrocellulose membrane.

39. The method of any one of clauses 36 to 38, wherein the one or more filters are a metal filter or stainless steel filter, optionally wherein the mesh size of the filter is 0.5 μm.

40. A method of preparing a pharmaceutically acceptable salt of ribociclib with an acid, wherein the method comprises mixing a first solution of ribociclib with a second solution of said acid, and wherein the method comprises one or more steps of reducing the content of nitrites in said first and/or second solutions, prior to their combination.

41. The method of clause 40, wherein the one or more steps involves passing the first and/or second solutions through an ion exchange resin that is capable of adsorbing nitrites.

42. A method of preparing ribociclib, wherein the method comprises the following deprotection step in which the amount of nitrites in the potassium carbonate is no more than 1 ppm.

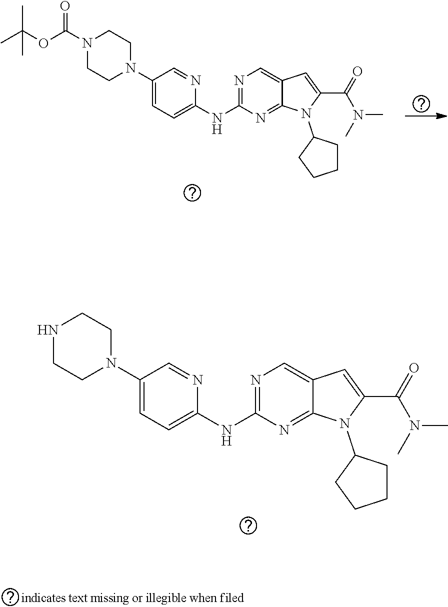 [SMILES: CN(C)C(=O)c1cc2cnc(Nc3ccc(N4CCN(C(=O)OC(C)(C)C)CC4)cn3)nc2n1C1CCCC1 ; CN(C)C(=O)c1cc2cnc(Nc3ccc(N4CCNCC4)cn3)nc2n1C1CCCC1]

43. A method of preparing a pharmaceutical composition comprising mixing ribociclib with one or more pharmaceutically acceptable excipients, wherein the one or more excipients have a content of nitrites of no more than 5 ppm, preferably no more than 1 ppm, and more preferably no more than 0.1 ppm, relative to the amount of the respective excipient.

44. The method of clause 43, wherein the excipient is magnesium stearate.

45. The method of clause 43, wherein the excipient is microcrystalline cellulose, optionally wherein the content of nitrites in the microcrystalline cellulose is no more than 100 ppb.

46. The method of clause 43, wherein the excipient is crospovidone, optionally wherein the content of nitrites in the crospovidone is no more than 100 ppb.

47. The method of clause 43, wherein the excipient is hydroxypropyl cellulose.

48. A method of preparing a pharmaceutical composition comprising ribociclib or a pharmaceutically acceptable salt thereof, and one or more pharmaceutically acceptable excipients, wherein the method comprises: (a) mixing ribociclib or said pharmaceutically acceptable salt thereof with the one or more excipients; and (b) drying the resulting mixture.

49. The method of clause 48, wherein the method further comprises, either before or after the step of drying, a step of compressing the composition into an orally administrable dosage form, optionally a tablet.

50. A method of evaluating a composition comprising or consisting of ribociclib or a pharmaceutically acceptable salt thereof, the method comprising testing the composition for the presence or amount of N-nitroso-ribociclib and salts thereof.

51. A method of validating a process for the production of a composition comprising or consisting of ribociclib or a pharmaceutically acceptable salt thereof, the method comprising testing the composition produced by said process for the presence or amount of N-nitroso-ribociclib and salts thereof.

52. A method of obtaining regulatory approval for a pharmaceutical composition which comprises or consists of ribociclib or a pharmaceutically acceptable salt thereof, wherein the method comprises testing a sample of the composition for the presence or amount of N-nitroso-ribociclib and salts thereof and submitting the results of said testing to a regulatory authority.

53. The method of any one of clauses 50 to 52, wherein a batch of the composition is tested.

54. The method of any one of clauses 50 to 53, wherein the composition comprises: (a) ribociclib or a pharmaceutically acceptable salt thereof; and (b) one or more pharmaceutically acceptable excipients.

55. The method of any one of clauses 50 to 54, wherein the method of testing for the presence or amount of nitrites is the Griess test.

56. A process for preparing a pharmaceutical product comprising ribociclib or a pharmaceutically acceptable salt thereof, and one or more pharmaceutically acceptable excipients, the process comprising:
a. obtaining a batch of ribociclib or a pharmaceutically acceptable salt thereof;
b. determining the total amount of N-nitroso-ribociclib in free or salt form in said batch; and
c. preparing the pharmaceutical product from the batch only if the batch is determined to have a total amount of N-nitroso-ribociclib in free or salt form of no more than 1 ppm, relative to the total amount of ribociclib in free or salt form.

57. A process of distributing a validated batch of a pharmaceutical product comprising ribociclib or a pharmaceutically acceptable salt thereof and one or more pharmaceutically acceptable excipients, the process comprising:
a. producing a batch of the pharmaceutical product;
b. performing stability testing with a sample of the batch;
c. determining the total amount of N-nitroso-ribociclib in free or salt form in said batch after stability testing; and
d. validating the batch for distribution only if the sample of the batch after stability testing is determined to have a total amount of N-nitroso-ribociclib in free or salt form of no more than 1 ppm, relative to the total amount of ribociclib in free or salt form.

58. The process of clause 56 or 57, wherein the step of determining the total amount of N-nitroso-ribociclib in free or salt form is performed using the Griess test.

59. Use of N-nitroso-ribociclib or a salt thereof as a reference standard to detect an impurity in a composition comprising ribociclib or a pharmaceutically acceptable salt thereof.

60. The use of clause 59, wherein the impurity is N-nitroso-ribociclib or a salt thereof.

61. The use of clause 59 or 60, wherein the detection is performed by the Griess test.

62. The use of any one of clauses 59 to 61, wherein the composition comprises ribociclib or a pharmaceutically acceptable salt thereof and one or more pharmaceutically acceptable excipients.

63. A method of storing a composition comprising or consisting of ribociclib, wherein the method comprises storing the composition in the presence of a desiccant, optionally wherein the method comprises testing the composition for the presence or amount of N-nitroso-ribociclib in free or salt form.

64. Use of a desiccant for reducing or preventing the formation of N-nitroso-ribociclib in free or salt form in a composition comprising or consisting of ribociclib or a pharmaceutically acceptable salt thereof, wherein the use comprises storing the composition in the presence of the desiccant.

65. A method of storing a composition comprising or consisting of ribociclib, wherein the method comprises storing the composition under an atmosphere in which the percentage of oxygen is less than 21% by volume, optionally wherein the method comprises testing the composition for the presence or amount of N-nitroso-ribociclib in free or salt form.

66. Use of an atmosphere in which the percentage of oxygen is less than 21% by volume for reducing the total amount of N-nitroso-ribociclib in free or salt form in a composition comprising or consisting of ribociclib, wherein the composition is stored under the atmosphere.

67. The method of clause 65, or the use of clause 66, wherein the atmosphere is nitrogen.

68. The method or use of any one of clauses 65 to 67, wherein the composition is stored in the presence of a desiccant.

69. A method of storing a composition comprising or consisting of ribociclib, wherein the method comprises storing the composition at a temperature of 2 to 8° C., optionally wherein the method comprises testing the composition for the presence or amount of N-nitroso-ribociclib in free or salt form.

70. The method of clause 69, wherein the composition is stored: (a) under an atmosphere in which the percentage of oxygen is less than 21% by volume, optionally wherein the atmosphere is nitrogen; and/or (b) in the presence of a desiccant.

71. The method or use of any one of clauses 63 to 70, wherein the composition comprises ribociclib or a pharmaceutically acceptable salt thereof and one or more pharmaceutically acceptable excipients.

72. The method or use of clause 71, wherein the composition is present in a pharmaceutical product which further comprises instructions to a patient as to how to administer the composition.

73. A composition or pharmaceutical product according to any one of clauses 1 to 15, wherein said composition has a shelf life of at least 1, 2, 3, 4, 5, 6, 7, 8, 9, 10, 11, 12, 13, 14, 15, 16, 17, 18, 24, 30, 36, 42, 48, 54 or 60 months when stored at a temperature of no more than 40° C.

74. The composition or pharmaceutical product according to clause 73, wherein said shelf life is under storage at a temperature of 2 to 8° C.

75. The composition or pharmaceutical product according to clause 73, wherein said shelf life is under storage at a temperature of no more than 25° C., optionally at a temperature of 20 to 25° C.

76. The composition or pharmaceutical product according to any one of clauses 73 to 75, wherein said composition has a shelf life of at least 9 months.

77. The composition or pharmaceutical product according to any one of clauses 73 to 75, wherein the shelf life is the period of time in which the total amount of N-nitroso-ribociclib in free or salt form in the composition remains no more than 1 ppm, relative to the total amount of ribociclib in free or salt form.

78. A method of producing N-nitroso-ribociclib or a salt thereof, wherein the method comprises reacting ribociclib with nitrous acid or a derivative thereof so as to produce the compound.

79. A composition comprising or consisting of ribociclib or a pharmaceutically acceptable salt thereof, wherein said composition is substantially free of nitrites.

80. A composition comprising or consisting of ribociclib or a pharmaceutically acceptable salt thereof, wherein the total amount of nitrites in the composition is no more than 100 ppb.

81. The composition of clause 79 or 80, wherein the composition is a solution of ribociclib or a pharmaceutically acceptable salt thereof in a solvent.

82. The composition according to clause 81, wherein the solvent is isopropyl alcohol.

83. The composition of any one of clauses 1 to 5, 10 to 15, 75 to 77 or 79 to 82, or the pharmaceutical product of any one of clauses 6 to 15, or the method of any one of clauses 43 to 55, or the process of any one of clauses 56 to 58, or the method or use of any one of clauses 59 to 72, for use in the treatment of breast cancer.

84. The composition, pharmaceutical product, method, process, or use of clause 83, wherein the breast cancer is locally advanced or metastatic breast cancer.

85. The composition, pharmaceutical product, method, process, or use of clause 83, wherein the breast cancer is early breast cancer.

86. The composition, pharmaceutical product, method, process, or use of any one of clauses 83 to 85, wherein the breast cancer is hormone receptor (HR)-positive, human epidermal growth factor receptor 2 (HER2)-negative breast cancer.

87. The composition, pharmaceutical product, method, process, or use of clause 84, wherein the breast cancer is hormone receptor (HR)-positive, human epidermal growth factor receptor 2 (HER2)-negative breast cancer.

88. The composition, pharmaceutical product, method, process, or use of clause 85, wherein the breast cancer is hormone receptor (HR)-positive, human epidermal growth factor receptor 2 (HER2)-negative breast cancer.

89. The composition, pharmaceutical product, method, process, or use of clause 83 or 84, for use in the treatment of a woman with hormone receptor (HR)-positive, human epidermal growth factor receptor 2 (HER2)-negative locally advanced or metastatic breast cancer in combination with an aromatase inhibitor or fulvestrant as initial endocrine-based therapy, or in the treatment of a woman with hormone receptor (HR)-positive, human epidermal growth factor receptor 2 (HER2)-negative locally advanced or metastatic breast cancer of women who has received prior endocrine therapy.

90. The composition, pharmaceutical product, method, process, or use of clause 89, wherein the woman is pre- or perimenopausal and wherein the endocrine therapy is combined with a luteinising hormone-releasing hormone (LHRH) agonist.

91. The composition, pharmaceutical product, method, process, or use of clause 89 or 90, wherein the aromatase inhibitor is letrozole, exemestane or anastrozole.

92. The composition, pharmaceutical product, method, process, or use of any one of clauses 83 to 91, wherein the treatment comprises administering a daily amount of ribociclib of 400 mg, or a daily amount of the pharmaceutically acceptable salt of ribociclib which corresponds to 400 mg of ribociclib free base.

93. The composition, pharmaceutical product, method, process, or use of any one of clauses 83 to 92, wherein the patient to be treated receives a daily amount of N-nitroso-ribociclib of no more than 400 ng, or a daily amount of salts of N-nitroso-ribociclib which corresponds to no more than 400 ng of N-nitroso-ribociclib.

94. The composition, pharmaceutical product, method, process, or use of any preceding clause, wherein the pharmaceutically acceptable salt of ribociclib is ribociclib succinate.

95. The composition, pharmaceutical product, method, process, or use of clause 94, wherein the ribociclib succinate is Form E as defined herein.

96. A method of preparing a composition comprising or consisting of ribociclib succinate, wherein the method comprises: (a) preparing a crystalline form of ribociclib succinate; and (b) washing the crystalline form with a solvent.

97. The method of clause 96, wherein the solvent is isopropyl alcohol.

98. The method of clause 96, wherein the solvent is ethanol.

### Clauses—List B

The invention provides the following numbered clauses:

1. A composition comprising or consisting of ribociclib or a pharmaceutically acceptable salt thereof, wherein said composition is substantially free of N-nitroso-ribociclib in free or salt form.

2. A composition comprising or consisting of ribociclib or a pharmaceutically acceptable salt thereof, wherein the total amount of N-nitroso-ribociclib in free or salt form in the composition is no more than 1 ppm, relative to the total amount of ribociclib in free or salt form, preferably wherein the total amount of N-nitroso-ribociclib in free or salt form in the composition is no more than 0.7 ppm, more preferably no more than 0.6 ppm, relative to the total amount of ribociclib in free or salt form.

3. A composition comprising or consisting of ribociclib or a pharmaceutically acceptable salt thereof, wherein the total amount of N-nitroso-ribociclib in free or salt form in the composition is no more than the maximum amount of nitrosamines that is permitted in such a composition, by a regulatory authority; optionally wherein the total amount of N-nitroso-ribociclib in free or salt form in the composition is no more than the maximum amount of N-nitroso-ribociclib that is permitted in such a composition, by a regulatory authority.

4. A composition comprising or consisting of ribociclib or a pharmaceutically acceptable salt thereof which has been tested and found to have a total amount of N-nitroso-ribociclib in free or salt form of no more than 1 ppm, relative to the total amount of ribociclib in free or salt form, preferably wherein the total amount of N-nitroso-ribociclib in free or salt form is no more than 0.7 ppm, more preferably no more than 0.6 ppm, relative to the total amount of ribociclib in free or salt form.

5. A composition comprising or consisting of ribociclib or a pharmaceutically acceptable salt thereof which has been tested and found to have a total amount of N-nitroso-ribociclib in free or salt form that is no more than the maximum amount of nitrosamines that is permitted in such a composition, by a regulatory authority, optionally wherein the total amount of N-nitroso-ribociclib in free or salt form is no more than the maximum amount of N-nitroso-ribociclib that is permitted in such a composition, by a regulatory authority.

6. A pharmaceutical product comprising the composition of any one of clauses 1 to 5 and a document which provides, either directly or via a link to an electronic database, instructions to a patient as to how to administer the composition.

7. A pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib in free or salt form in the composition is no more than 1 ppm, relative to the total amount of ribociclib in free or salt form, preferably wherein the total amount of N-nitroso-ribociclib in free or salt form in the composition is no more than 0.7 ppm, more preferably no more than 0.6 ppm, relative to the total amount of ribociclib in free or salt form.

8. A pharmaceutical product containing: (a) a pharmaceutical composition comprising ribociclib or a pharmaceutically acceptable salt thereof; and (b) a document which certifies, either directly or via a link to an electronic database, that the total amount of N-nitroso-ribociclib in free or salt form in the composition is no more than the maximum amount of nitrosamines that is permitted in such a composition, by a regulatory authority, optionally wherein the total amount of N-nitroso-ribociclib in free or salt form in the composition is no more than the maximum amount of N-nitroso-ribociclib that is permitted in such a composition, by a regulatory authority.

9. The pharmaceutical product of clause 7 or clause 8, wherein the product further comprises a document providing, either directly or via a link to an electronic database, instructions to a patient as to how to administer the composition.

10. The composition or pharmaceutical product of any one of clauses 1 to 9, wherein the total amount of ribociclib or pharmaceutically acceptable salt(s) thereof in the composition provides the same amount of ribociclib as 200 mg of ribociclib free base.

11. A composition or pharmaceutical product according to any one of clauses 1 to 10, wherein the composition is an oral dosage form, optionally wherein the oral dosage form is a tablet.

12. A composition or pharmaceutical product according to any one of clauses 1 to 10, wherein the composition comprises or consists of ribociclib or a pharmaceutically acceptable salt thereof within an orally administrable capsule.

13. A composition or pharmaceutical product according to any one of clauses 1 to 12, wherein the composition is present in a sealed container and is under an atmosphere in which the percentage of oxygen is less than 21% by volume, optionally wherein the atmosphere is nitrogen.

14. A composition or pharmaceutical product according to any one of clauses 1 to 13, wherein the composition is present in a sealed container which also contains a desiccant, or the composition is present in a sealed pharmaceutical package which also contains a desiccant in a separate container.

15. A composition or pharmaceutical product according to any one of clauses 1 to 14, wherein the composition further comprises one or more pharmaceutically acceptable excipients.

16. A method of preparing ribociclib or a pharmaceutically acceptable salt thereof with an acid, wherein the method comprises the use of one or more solvents which, immediately prior to their use in the method, are stored under an atmosphere in which the percentage of oxygen is less than 21% by volume, optionally wherein the atmosphere is nitrogen.

17. The method of clause 16, wherein said method comprises the use of said one or more solvents for dissolving ribociclib and/or said acid.

18. The method of clause 17, wherein the method is for preparing a pharmaceutically acceptable salt of ribociclib with an acid, and the method comprises the use of said one or more solvents for dissolving ribociclib and said acid, prior to their combination to form the pharmaceutically acceptable salt.

19. A method of preparing ribociclib or a pharmaceutically acceptable salt thereof with an acid, wherein the method comprises: (a) obtaining one or more solvents, where the one or more solvents are contained in a sealed container under an atmosphere in which the percentage of oxygen is less than 21% by volume, optionally wherein the atmosphere is nitrogen; and (b) using the one or more solvents to dissolve ribociclib and/or said acid.

20. The method of any one of clauses 16 to 19, wherein the total amount of nitrites in each of the one or more solvents is no more than 5 ppb, preferably no more than 3.5 ppb, and more preferably no more than 1 ppb, relative to the amount of the respective solvent;
optionally wherein, in those embodiments in which the one or more solvents is more than one solvent, the combined total amount of nitrites in the solvents is no more than 5 ppb, preferably no more than 3.5 ppb, and more preferably no more than 1 ppb, relative to the combined amount of the solvents.

21. A method of preparing ribociclib or a pharmaceutically acceptable salt thereof, wherein the method comprises distilling one or more solvents to reduce their content of nitrites.

22. A method of preparing ribociclib or a pharmaceutically acceptable salt thereof with an acid, wherein the method comprises the use of one or more solvents for dissolving ribociclib and/or said acid, and wherein the method comprises distilling the one or more solvents to reduce their content of nitrites, prior to said use.

23. A method of preparing ribociclib or a pharmaceutically acceptable salt thereof with an acid, wherein the method comprises: (a) obtaining one or more solvents, optionally wherein the one or more solvents are contained in a sealed container under an atmosphere in which the percentage of oxygen is less than 21% by volume, preferably wherein the atmosphere is nitrogen; (b) distilling said one or more solvents to reduce their content of nitrites; and (c) using the one or more distilled solvents to dissolve ribociclib and/or said acid.

24. The method of any one of clauses 21 to 23, wherein the total amount of nitrites in each of the one or more solvents, following the distillation, is no more than 5 ppb, preferably no more than 3.5 ppb, and more preferably no more than 1 ppb, relative to the amount of the respective solvent, optionally wherein, in those embodiments in which the one or more solvents is more than one solvent, the combined total amount of nitrites in the solvents is no more than 5 ppb, preferably no more than 3.5 ppb, and more preferably no more than 1 ppb, relative to the combined amount of the solvents.

25. A method of preparing ribociclib or a pharmaceutically acceptable salt thereof, wherein the method comprises passing one or more solvents through an ion exchange resin that is capable of adsorbing nitrites.

26. A method of preparing ribociclib or a pharmaceutically acceptable salt thereof with an acid, wherein the method comprises the use of one or more solvents for dissolving ribociclib and/or said acid, and wherein the method comprises passing the one or more solvents through an ion exchange resin which is capable of adsorbing nitrites, prior to said use.

27. The method of clause 25 or 26, wherein the method comprises: (a) distilling the one or more solvents to reduce their content of nitrites; and (b) passing the one or more distilled solvents through said ion exchange resin.

28. A method of preparing ribociclib or a pharmaceutically acceptable salt thereof with an acid, wherein the method comprises a step of passing a solution comprising or consisting of: (a) ribociclib or a pharmaceutically acceptable salt thereof; and (b) a solvent, through an ion exchange resin that is capable of adsorbing nitrites from the solution.

29. The method of clause 28, wherein the ion exchange resin adsorbs no more than 5% by weight of the total amount of ribociclib in free or salt form.

30. A method of preparing ribociclib or a pharmaceutically acceptable salt thereof with an acid, wherein the method comprises a step of passing a solution comprising or consisting of: (a) said acid; and (b) a solvent, through an ion exchange resin that is capable of adsorbing nitrites from the solution.

31. The method of any one of clauses 28 to 30, wherein the method comprises: (a) obtaining one or more solvents, optionally wherein the one or more solvents are contained in a sealed container under an atmosphere in which the percentage of oxygen is less than 21% by volume, preferably wherein the atmosphere is nitrogen; (b) distilling the one or more solvents to reduce their content of nitrites; and (c) using the one or more distilled solvents to dissolve ribociclib and/or said acid.

32. The method of clause 31, wherein the method further comprises: (d) passing the solution of ribociclib and/or the solution of said acid through an ion exchange resin that is capable of adsorbing nitrites from a solution.

33. The method of any one of clause 25 to 32, wherein the ion exchange resin that is capable of adsorbing nitrites is selected from the group consisting of an activated charcoal resin, a silica gel resin, a celite resin, or an aluminium oxide resin.

34. The method of any one of clauses 16 to 33, wherein the one or more solvents is or comprises isopropanol, optionally wherein the isopropanol is or becomes substantially free of isopropyl nitrite.

35. The method of any one of clauses 21 to 24, 27, 31, or 32, wherein the one or more solvents is or comprises isopropanol and wherein the distillation is over ascorbic acid.

36. A method of preparing ribociclib or a pharmaceutically acceptable salt thereof with an acid, wherein the method comprises the use of one or more filters which do not comprise a nitrocellulose membrane, optionally wherein none of the filters that are used in the method comprises a nitrocellulose membrane.

37. The method of clause 36, wherein the one or more filters are used to filter a solution of ribociclib and/or a solution of said acid.

38. A method of preparing ribociclib or a pharmaceutically acceptable salt thereof with an acid, wherein the method comprises: (a) obtaining a solution of ribociclib and/or a solution of said acid; and (b) filtering the or each solution with a filter that does not comprise a nitrocellulose membrane.

39. The method of any one of clauses 36 to 38, wherein the or each filter is a metal filter or a stainless steel filter, optionally wherein the mesh size of the filter is 0.5 μm.

40. A method of preparing a pharmaceutically acceptable salt of ribociclib with an acid, wherein the method comprises mixing a first solution of ribociclib with a second solution of said acid, and wherein the method comprises one or more steps of reducing the content of nitrites in said first and/or second solutions, prior to their combination.

41. The method of clause 40, wherein the one or more steps involves passing the first and/or second solutions through an ion exchange resin that is capable of adsorbing nitrites.

42. A method of preparing ribociclib or a pharmaceutically acceptable salt thereof, wherein the method comprises the following deprotection step in which the amount of nitrites in the potassium carbonate is no more than 1 ppm.

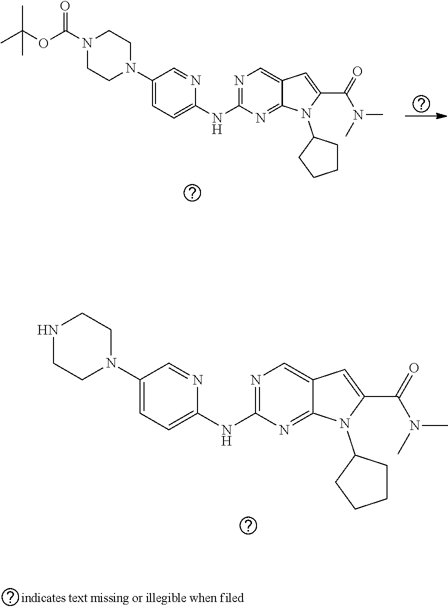 [SMILES: CN(C)C(=O)c1cc2cnc(Nc3ccc(N4CCN(C(=O)OC(C)(C)C)CC4)cn3)nc2n1C1CCCC1 ; CN(C)C(=O)c1cc2cnc(Nc3ccc(N4CCNCC4)cn3)nc2n1C1CCCC1]

43. A method of preparing a pharmaceutical composition comprising mixing ribociclib or a pharmaceutically acceptable salt thereof with one or more pharmaceutically acceptable excipients, wherein each of the one or more excipients has a content of nitrites of no more than 5 ppm, preferably no more than 1 ppm, more preferably no more than 0.1 ppm, relative to the amount of the respective excipient;

optionally wherein, in those embodiments in which the one or more excipients is more than one excipient, the combined excipients have a total content of nitrites of no more than 5 ppm, preferably no more than 1 ppm, more preferably no more than 0.1 ppm, relative to the combined amount of the excipients.

44. The method of clause 43, wherein the or one of the excipients is magnesium stearate.

45. The method of clause 43 or clause 44, wherein the or one of the excipients is microcrystalline cellulose, optionally wherein the content of nitrites in the microcrystalline cellulose is no more than 100 ppb.

46. The method of any one of clauses 43 to 45, wherein the or one of the excipients is crospovidone, optionally wherein the content of nitrites in the crospovidone is no more than 100 ppb.

47. The method of any one of clauses 43 to 46, wherein the or one of the excipients is hydroxypropyl cellulose.

48. A method of preparing a pharmaceutical composition comprising: (i) ribociclib or a pharmaceutically acceptable salt thereof; and (ii) one or more pharmaceutically acceptable excipients, wherein the method comprises: (a) mixing ribociclib or said pharmaceutically acceptable salt thereof with the one or more excipients; and (b) drying the resulting mixture.

49. The method of clause 48, wherein the method further comprises, either before or after the step of drying, a step of compressing the composition into an orally administrable dosage form, optionally a tablet.

50. A method of evaluating a composition comprising or consisting of ribociclib or a pharmaceutically acceptable salt thereof, the method comprising testing the composition for the presence or total amount of N-nitroso-ribociclib in free or salt form.

51. A method of validating a process for the production of a composition comprising or consisting of ribociclib or a pharmaceutically acceptable salt thereof, the method comprising testing a composition produced by said process for the presence or total amount of N-nitroso-ribociclib in free or salt form.

52. A method of obtaining regulatory approval for a pharmaceutical composition which comprises or consists of ribociclib or a pharmaceutically acceptable salt thereof, wherein the method comprises: (i) testing the composition for the presence or total amount of N-nitroso-ribociclib in free or salt form; and (ii) submitting the results of said testing to a regulatory authority.

53. The method of any one of clauses 50 to 52, wherein a sample or batch of the composition is tested.

54. The method of any one of clauses 50 to 53, wherein the composition comprises: (a) ribociclib or a pharmaceutically acceptable salt thereof; and (b) one or more pharmaceutically acceptable excipients.

55. The method of any one of clauses 50 to 54, wherein the method of testing for the presence or total amount of N-nitroso-ribociclib in free or salt form:
(a) includes the Griess test; and/or
(b) includes high performance liquid chromatography (HPLC)-mass spectroscopy and/or gas chromatography (GC)-mass spectroscopy.

56. A process for preparing a pharmaceutical product comprising a composition that comprises: (i) ribociclib or a pharmaceutically acceptable salt thereof; and (ii) one or more pharmaceutically acceptable excipients, the process comprising:
a. obtaining a batch of ribociclib or of a pharmaceutically acceptable salt thereof;
b. determining the total amount of N-nitroso-ribociclib in free or salt form in a sample of said batch; and
c. preparing the pharmaceutical product from the batch only if the sample of the batch is determined to have a total amount of N-nitroso-ribociclib in free or salt form of no more than 1 ppm, preferably no more than 0.7 ppm, more preferably no more than 0.6 ppm, relative to the total amount of ribociclib in free or salt form.

57. A process of distributing a validated batch of a pharmaceutical product comprising a composition that comprises: (i) ribociclib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of N-nitroso-ribociclib in free or salt form in said sample after stability testing; and
d. validating the batch for distribution only if the sample of the batch after stability testing is determined to have a total amount of N-nitroso-ribociclib in free or salt form of no more than 1 ppm, preferably no more than 0.7 ppm, more preferably no more than 0.6 ppm, relative to the total amount of ribociclib in free or salt form.

58. The process of clause 56 or 57, wherein the step of determining the total amount of N-nitroso-ribociclib in free or salt form is performed by a method which includes the Griess test and/or high performance liquid chromatography (HPLC)-mass spectroscopy and/or gas chromatography (GC)-mass spectroscopy.

59. Use of N-nitroso-ribociclib or a salt thereof as a reference standard to detect an impurity in a composition comprising ribociclib or a pharmaceutically acceptable salt thereof.

60. The use of clause 59, wherein the impurity is N-nitroso-ribociclib or a salt thereof.

61. The use of clause 59 or 60, wherein the detection is performed by a method which includes the Griess test and/or high performance liquid chromatography (HPLC)-mass spectroscopy and/or gas chromatography (GC)-mass spectroscopy.

62. The use of any one of clauses 59 to 61, wherein the composition comprises: (i) ribociclib or a pharmaceutically acceptable salt thereof; and (ii) one or more pharmaceutically acceptable excipients.

63. A method of storing a composition comprising or consisting of ribociclib or a pharmaceutically acceptable salt thereof, wherein the method comprises storing the composition in the presence of a desiccant, optionally wherein the method comprises testing the composition for the presence or total amount of N-nitroso-ribociclib in free or salt form.

64. Use of a desiccant for preventing, retarding or reducing the formation of N-nitroso-ribociclib in free or salt form in a composition comprising or consisting of ribociclib or a pharmaceutically acceptable salt thereof, wherein the use comprises storing the composition in the presence of the desiccant.

65. A method of storing a composition comprising or consisting of ribociclib or a pharmaceutically acceptable salt thereof, wherein the method comprises storing the composition under an atmosphere in which the percentage of oxygen is less than 21% by volume, optionally wherein the method comprises testing the composition for the presence or total amount of N-nitroso-ribociclib in free or salt form.

66. Use of an atmosphere in which the percentage of oxygen is less than 21% by volume for preventing, retarding or reducing the formation of N-nitroso-ribociclib in free or salt form in a composition comprising or consisting of ribociclib or a pharmaceutically acceptable salt thereof, wherein the composition is stored under the atmosphere.

67. The method of clause 65, or the use of clause 66, wherein the atmosphere is nitrogen.

68. The method or use of any one of clauses 65 to 67, wherein the composition is stored in the presence of a desiccant.

69. A method of storing a composition comprising or consisting of ribociclib or a pharmaceutically acceptable salt thereof, wherein the method comprises storing the composition at a temperature of 2 to 8° C., optionally wherein the method comprises testing the composition for the presence or total amount of N-nitroso-ribociclib in free or salt form.

70. The method of clause 69, wherein the composition is stored: (a) under an atmosphere in which the percentage of oxygen is less than 21% by volume, optionally wherein the atmosphere is nitrogen; and/or (b) in the presence of a desiccant.

71. The method or use of any one of clauses 63 to 70, wherein the composition comprises: (i) ribociclib or a pharmaceutically acceptable salt thereof; and (ii) one or more pharmaceutically acceptable excipients.

72. The method or use of clause 71, wherein the composition is present in a pharmaceutical product which further comprises a document which provides, either directly or via a link to an electronic database, instructions to a patient as to how to administer the composition.

73. The composition of any one of clauses 1 to 5 or 10 to 15, or the pharmaceutical product of any one of clauses 6 to 15, or the method of any one of clauses 43 to 55, 63, 65, 69 or 70, or the process of any one of clauses 56 to 58, or the use of any one of clauses 59 to 62, 64 or 66, or the method or use of any one of clauses 67, 68, 71 or 72, wherein said composition has a shelf life of at least 1, 2, 3, 4, 5, 6, 7, 8, 9, 10, 11, 12, 13, 14, 15, 16, 17, 18, 24, 30, 36, 42, 48, 54 or 60 months when stored at a temperature of no more than 40° C.

74. The composition or pharmaceutical product according to clause 73, wherein said shelf life is under storage at a temperature of 2 to 8° C.

75. The composition or pharmaceutical product according to clause 73, wherein said shelf life is under storage at a temperature of no more than 25° C., optionally at a temperature of 20 to 25° C.

76. The composition or pharmaceutical product according to any one of clauses 73 to 75, wherein said composition has a shelf life of at least 9 months, preferably at least 10 months.

77. The composition or pharmaceutical product according to any one of clauses 73 to 75, wherein the shelf life is the period of time in which the total amount of N-nitroso-ribociclib in free or salt form in the composition remains no more than 1 ppm, preferably no more than 0.7 ppm, more preferably no more than 0.6 ppm, relative to the total amount of ribociclib in free or salt form.

78. A method of producing N-nitroso-ribociclib or a salt thereof, wherein the method comprises reacting ribociclib with nitrous acid or a derivative thereof so as to produce the said compound.

79. A composition comprising or consisting of ribociclib or a pharmaceutically acceptable salt thereof, wherein said composition is substantially free of nitrites.

80. A composition comprising or consisting of ribociclib or a pharmaceutically acceptable salt thereof, wherein the total amount of nitrites in the composition is no more than 100 ppb.

81. The composition of clause 79 or 80, wherein the composition is a solution of ribociclib or a pharmaceutically acceptable salt thereof in a solvent.

82. The composition according to clause 81, wherein the solvent is isopropyl alcohol.

83. The composition of any one of clauses 1 to 5, 10 to 15, 73 to 77, or 79 to 82, or the pharmaceutical product of any one of clauses 6 to 15 or 73 to 77, or the method of any one of clauses 43 to 55, 63, 65, 69 or 70, or the process of any one of clauses 56 to 58, or the use of any one of clauses 59 to 62, 64 or 66, or the method or use of any one of clauses 67, 68, 71 or 72, wherein the composition is for use in the treatment of breast cancer.

84. The composition, pharmaceutical product, method, process, or use of clause 83, wherein the breast cancer is locally advanced or metastatic breast cancer.

85. The composition, pharmaceutical product, method, process, or use of clause 83, wherein the breast cancer is early breast cancer.

86. The composition, pharmaceutical product, method, process, or use of any one of clauses 83 to 85, wherein the breast cancer is hormone receptor (HR)-positive, human epidermal growth factor receptor 2 (HER2)-negative breast cancer.

87. The composition, pharmaceutical product, method, process, or use of clause 84, wherein the breast cancer is hormone receptor (HR)-positive, human epidermal growth factor receptor 2 (HER2)-negative breast cancer.

88. The composition, pharmaceutical product, method, process, or use of clause 85, wherein the breast cancer is hormone receptor (HR)-positive, human epidermal growth factor receptor 2 (HER2)-negative breast cancer.

89. The composition, pharmaceutical product, method, process, or use of clause 83 or 84, for use in the treatment of a woman with hormone receptor (HR)-positive, human epidermal growth factor receptor 2 (HER2)-negative locally advanced or metastatic breast cancer in combination with an aromatase inhibitor or fulvestrant as initial endocrine-based therapy, or in the treatment of a woman with hormone receptor (HR)-positive, human epidermal growth factor receptor 2 (HER2)-negative locally advanced or metastatic breast cancer who has received prior endocrine therapy.

90. The composition, pharmaceutical product, method, process, or use of clause 89, wherein the woman is pre- or perimenopausal and wherein the initial endocrine-based therapy is combined with a luteinising hormone-releasing hormone (LHRH) agonist.

91. The composition, pharmaceutical product, method, process, or use of clause 89 or 90, wherein the aromatase inhibitor is letrozole, exemestane or anastrozole.

92. The composition, pharmaceutical product, method, process, or use of any one of clauses 83 to 91, wherein the treatment comprises administering a daily amount of ribociclib of 400 mg, or a daily amount of a pharmaceutically acceptable salt of ribociclib which corresponds to 400 mg of ribociclib free base.

93. The composition, pharmaceutical product, method, process, or use of any one of clauses 83 to 92, wherein the patient to be treated receives, as a result of the administration of the composition in accordance with the treatment, a daily total amount of N-nitroso-ribociclib in free or salt form that corresponds to an amount of no more than 400 ng of N-nitroso-ribociclib free base.

94. The composition of any one of clauses 1 to 5, 10 to 15, 73 to 77, or 79 to 82, or the pharmaceutical product of any one of clauses 6 to 15 or 73 to 77, or the method of any one of clauses 16 to 41, 43 to 55, 63, 65, 69 or 70, or the process of any one of clauses 56 to 58, or the use of any one of clauses 59 to 62, 64 or 66, or the method or use of any one of clauses 67, 68, 71 or 72, or the composition, pharmaceutical product, method, process, or use of any one of clauses 83 to 93, wherein the pharmaceutically acceptable salt of ribociclib is ribociclib succinate.

95. The composition, pharmaceutical product, method, process, or use of clause 94, wherein the ribociclib succinate is Form E as defined herein.

96. A method of preparing a composition comprising or consisting of ribociclib succinate, wherein the method comprises: (a) preparing a crystalline form of ribociclib succinate; and (b) washing the crystalline form with a solvent.

97. The method of clause 96, wherein the solvent is isopropyl alcohol.

98. The method of clause 96, wherein the solvent is ethanol.

99. A method of storing over a period of time a composition comprising or consisting of ribociclib or a pharmaceutically acceptable salt thereof, wherein the total amount of N-nitroso-ribociclib in free or salt form in the composition at the start of the period of time is no more than a first threshold amount, and wherein the total amount of N-nitroso-ribociclib in free or salt form in the composition at the end of the period of time is no more than a second threshold amount, wherein the storing of the composition incorporates means to prevent, retard or reduce the formation of N-nitroso-ribociclib in free or salt form.

100. The method of clause 99, wherein the means to prevent, retard or reduce the formation of N-nitroso-ribociclib in free or salt form comprises or consists of one or any combination of the following:
a. storing the composition in a sealed container under an atmosphere in which the percentage of oxygen is less than 21% by volume, optionally wherein the atmosphere is nitrogen;
b. storing the composition in a sealed container which also contains a desiccant, or storing the composition in a sealed pharmaceutical package which also contains a desiccant in a separate container;
c. storing the composition under an atmosphere in which the relative humidity is less than 75%, optionally wherein the relative humidity is less than 60%;
d. storing the composition at a temperature of less than 25° C., optionally wherein the temperature is 2 to 8° C.;
e. incorporating in the composition one or more pharmaceutically acceptable additives which prevent, retard or reduce the formation of N-nitroso-ribociclib in free or salt form, optionally wherein each of the one or more additives is selected from the group consisting of pH adjusters, antioxidants, radical scavenging agents, and peroxide-quenching agents.

101. The method of clause 99 or 100, wherein the period of time is at least 1, 2, 3, 4, 5, 6, 7, 8, 9, 10, 11, 12, 13, 14, 15, 16, 17, 18, 24, 30, 36, 42, 48, 54 or 60 months, preferably wherein the period of time is at least 12 months, more preferably wherein the period of time is at least 18 months, most preferably wherein the period of time is at least 24 months, for example at least 36 months, 48 months, or 60 months.

102. The method of any one of clauses 99 to 101, wherein the first threshold amount is about 0.6 ppm or about 0.7 ppm, relative to the total amount of ribociclib in free or salt form.

103. The method of any one of clauses 99 to 102, wherein the second threshold amount is about 1 ppm, relative to the total amount of ribociclib in free or salt form.

104. The method of any one of clauses 99 to 103, wherein the composition is a pharmaceutical composition, optionally wherein the composition comprises one or more pharmaceutically acceptable excipients.

105. The method of any one of clauses 99 to 104, wherein the composition is part of a pharmaceutical product which further comprises one or more of:
a. a document providing, either directly or via a link to an electronic database, instructions to a patient as to how to administer the composition;
b. a document providing, either directly or via a link to an electronic database, instructions which specify how the composition should be stored;
c. a document which, either directly or via a link to an electronic database, indicates that, provided that the composition is stored in accordance with specified instructions, the total amount of N-nitroso-ribociclib in free or salt form in the composition will be no more than the second threshold amount.

106. A pharmaceutical product comprising: (a) a composition comprising or consisting of ribociclib or a pharmaceutically acceptable salt thereof; and (b) a document providing, either directly or via a link to an electronic database, instructions which specify how the composition should be stored, wherein if the composition is stored in accordance with the instructions, then the composition will be substantially free of N-nitroso-ribociclib in free or salt form.

107. A pharmaceutical product comprising: (a) a composition comprising or consisting of ribociclib or a pharmaceutically acceptable salt thereof; and (b) a document providing, either directly or via a link to an electronic database, instructions which specify how the composition should be stored, wherein if the composition is stored in accordance with the instructions, then the total amount of N-nitroso-ribociclib in free or salt form in the composition will be no more than the maximum amount of nitrosamines that is permitted in such a composition, by a regulatory authority, optionally wherein the total amount of N-nitroso-ribociclib in free or salt form in the composition will be no more than the maximum amount of N-nitroso-ribociclib that is permitted in such a composition, by a regulatory authority.

108. A pharmaceutical product comprising: (a) a composition comprising or consisting of ribociclib or a pharmaceutically acceptable salt thereof; and (b) a document providing instructions which specify how the composition should be stored, wherein if the composition is stored in accordance with the instructions, then the total amount of N-nitroso-ribociclib in free or salt form in the composition will be no more than 1 ppm, relative to the total amount of ribociclib in free or salt form, preferably wherein the total amount of N-nitroso-ribociclib in free or salt form in the composition is or will be no more than 0.7 ppm, more preferably no more than 0.6 ppm, relative to the total amount of ribociclib in free or salt form.

109. The pharmaceutical product of any one of clauses 106 to 108, which further comprises means to prevent, retard or reduce the formation of N-nitroso-ribociclib in free or salt form in the composition, optionally wherein the means are one or any combination of:
a. the composition is present in a sealed container under an atmosphere in which the percentage of oxygen is less than 21% by volume, optionally wherein the atmosphere is nitrogen;
b. the composition is present in a sealed container which also contains a desiccant; or the composition is present in a sealed pharmaceutical package which also contains a desiccant in a separate container;
c. the composition comprises one or more pharmaceutically acceptable additives which prevent, retard or reduce the formation of N-nitroso-ribociclib in free or salt form, optionally wherein each of the one or more additives is selected from the group consisting of pH adjusters, antioxidants, radical scavenging agents, and peroxide-quenching agents.

110. The method of clause 105, or the pharmaceutical product of any one of clauses 106 to 109, wherein the instructions specify a set of storage conditions comprising a storage time of no more than 60 months, optionally no more than 54, 48, 42, 36, 30, 24, 18, 17, 16, 15, 14, 13, 12, 11, 10, 9, 8, 7, 6, 5, 4, 3, 2, or 1 months and a temperature of no more than 25° C., optionally wherein the specified temperature is about 2 to about 8° C.

111. The method or pharmaceutical product of clause 110, wherein the instructions specify: (i) a first set of storage conditions; and (ii) a second set of storage conditions for use after the first set of storage conditions.

112. The method or pharmaceutical product of clause 111, wherein the first set of storage conditions comprises a storage time of no more than 60 months, optionally no more than 54, 48, 42, 36, 30, 24, 18, 17, 16, 15, 14, 13, 12, or 11 months, and a temperature of about 2 to about 8° C.

113. The method or pharmaceutical product of clause 111 or clause 112, wherein the second set of storage conditions comprises a storage time of no more than 10 months, optionally no more than 9, 8, 7, 6, 5, 4, 3, 2, or 1 months, and a temperature of no more than 25° C.

114. The method of any one of clauses 105 or 110 to 113, or the pharmaceutical product of any one of clauses 106 to 113, wherein the pharmaceutical product further comprises a document which provides, either directly or via a link to an electronic database, instructions to a patient as to how to administer the composition.

115. The method of any one of clauses 99 to 105 or 110 to 114, or the pharmaceutical product of any one of clauses 106 to 114, wherein the total amount of ribociclib or pharmaceutically acceptable salt thereof in the composition provides the same amount of ribociclib as 200 mg of ribociclib free base.

116. The method of any one of clauses 99 to 105 or 110 to 115, or the pharmaceutical product of any one of clauses 106 to 115, wherein the composition is an oral dosage form, optionally wherein the oral dosage form is a tablet.

117. The method of any one of clauses 99 to 105 or 110 to 115, or the pharmaceutical product of any one of clauses 106 to 115, wherein the composition comprises or consists of ribociclib or a pharmaceutically acceptable salt thereof in an orally administrable capsule.

118. The method of any one of clauses 99 to 105 or 110 to 117, or the pharmaceutical product of any one of clauses 106 to 117, wherein the pharmaceutically acceptable salt of ribociclib is ribociclib succinate, optionally wherein the ribociclib succinate is Form E as defined herein.

119. The method of any one of clauses 99 to 105 or 110 to 118, or the pharmaceutical product of any one of clauses 106 to 118, wherein the composition comprises one or more pharmaceutically acceptable excipients.

120. The method or pharmaceutical product of clause 119, wherein each of the one or more pharmaceutically acceptable excipients is selected from the group consisting of magnesium stearate, microcrystalline cellulose, crospovidone, and hydroxypropyl cellulose.

121. The method of any one of clauses 99 to 105 or 110 to 120, or the pharmaceutical product of any one of clauses 106 to 120, wherein the composition is for use in the treatment of breast cancer, optionally wherein the breast cancer is (a) locally advanced or metastatic breast cancer; or (b) early breast cancer.

122. The method of any one of clauses 99 to 105 or 110 to 121, wherein the first and second threshold amounts are the same.

123. The method of any one of clauses 99 to 105 or 110 to 121, wherein the second threshold amount is no more than 1.5 ppm greater than the first threshold amount, for example no more than 1, 0.9, 0.8, 0.7, 0.6, 0.5 or 0.4 ppm greater than the first threshold amount.

124. The method of any one of clauses 99 to 105 or 110 to 121, wherein the second threshold amount is no more than 200% of the first threshold amount, for example no more than 190, 180 or 170% of the first threshold amount

125. A plurality of pharmaceutical products according to any one of clauses 6 to 15 or 73 to 77, optionally wherein the pharmaceutical products are packaged together.

126. The plurality of pharmaceutical products of clause 125, wherein the plurality comprises at least 5, 10, 15, 20, 25, 30, 35, 40, 45, 50, 60, 70, 80, 90, 100, 150, 200, 250, 300, 350, 400, 450, 500, 600, 700, 800, 900 or 1000 of said pharmaceutical products.

127. The plurality of pharmaceutical products of clause 125 or clause 126, wherein each of said pharmaceutical products comprises one or any combination of:
a. a document providing, either directly or via a link to an electronic database, instructions to a patient as to how to administer the composition
b. a document providing, either directly or via a link to an electronic database, instructions which specify how the composition should be stored
c. a document which certifies, either directly or via a link to an electronic database, that the total amount of N-nitroso-ribociclib in free or salt form in the composition is no more than 1 ppm, relative to the total amount of ribociclib in free or salt form, preferably wherein the total amount of N-nitroso-ribociclib in free or salt form in the composition is no more than 0.7 ppm, more preferably no more than 0.6 ppm, relative to the total amount of ribociclib in free or salt form.
d. a document which indicates that, provided that the composition is stored in accordance with a set of instructions, the total amount of N-nitroso-ribociclib in free or salt form in the composition will be no more than 1 ppm, relative to the total amount of ribociclib in free or salt form, preferably wherein the total amount of N-nitroso-ribociclib in free or salt form in the composition is or will be no more than 0.7 ppm, more preferably no more than 0.6 ppm, relative to the total amount of ribociclib in free or salt form.

128. The plurality of pharmaceutical products of any one of clauses 125 to 127, wherein each of the said pharmaceutical products is for use in the treatment of breast cancer, optionally wherein the breast cancer is locally advanced or metastatic breast cancer or early breast cancer.

129. The plurality of pharmaceutical products of clause 128, wherein the treatment comprises administering a daily amount of ribociclib of 400 mg, or a daily amount of the pharmaceutically acceptable salt of ribociclib which corresponds to 400 mg of ribociclib free base.

130. The plurality of pharmaceutical products of clause 128 or 129, wherein the patient to be treated receives, as a result of the administration of the composition in accordance with the treatment, a daily total amount of N-nitroso-ribociclib in free or salt form that corresponds to an amount of no more than 400 ng of N-nitroso-ribociclib free base.

131. The plurality of pharmaceutical products of any one of clauses 128 to 130, wherein each of the said pharmaceutical products is suitable to be approved and has been approved by a regulatory authority for its use in the treatment of early breast cancer, optionally wherein each of said pharmaceutical products comprises a document which indicates said approval, either directly or via a link to an electronic database.

132. A pharmaceutical product comprising a plurality of oral dosage forms, wherein each of the oral dosage forms comprises or consists of ribociclib or a pharmaceutically acceptable salt thereof, and wherein each of the oral dosage forms is substantially free of N-nitroso-ribociclib in free or salt form.

133. A pharmaceutical product comprising a plurality of oral dosage forms, wherein each of the oral dosage forms comprises or consists of ribociclib or a pharmaceutically acceptable salt thereof, and wherein the total amount of N-nitroso-ribociclib in free or salt form in each of the oral dosage forms is no more than 1 ppm, relative to the total amount of ribociclib in free or salt form, preferably wherein the total amount of N-nitroso-ribociclib in free or salt form in each of the oral dosage forms is no more than 0.7 ppm, more preferably no more than 0.6 ppm, relative to the total amount of ribociclib in free or salt form.

134. A pharmaceutical product comprising a plurality of oral dosage forms, wherein each of the oral dosage forms comprises or consists of ribociclib or a pharmaceutically acceptable salt thereof, wherein the total amount of N-nitroso-ribociclib in free or salt form in each of the oral dosage forms is no more than the maximum amount of nitrosamines that is permitted in such a composition, by a regulatory authority; optionally wherein the total amount of N-nitroso-ribociclib in free or salt form in each of the oral dosage forms is no more than the maximum amount of N-nitroso-ribociclib that is permitted in such a composition, by a regulatory authority.

135. The pharmaceutical product of any one of clauses 132 to 134, wherein the product comprises at least 5, 10, 15, 20, 25, 30, 35, 40, 45, 50 or 60 of said oral dosage forms, such as 7, 14, 21, 28, 35, 42, 49, 56, 63 or 70 of said oral dosage forms.

136. The pharmaceutical product of any one of clauses 132 to 135, wherein each of the oral dosage forms is a tablet.

137. A process for preparing a pharmaceutical product comprising a composition that comprises: (i) ribociclib or a pharmaceutically acceptable salt thereof; and (ii) one or more pharmaceutically acceptable excipients, the process comprising:
a. preparing a first batch of ribociclib or of a pharmaceutically acceptable salt thereof;
b. determining the total amount of N-nitroso-ribociclib in free or salt form in a sample of said first batch;
c. discarding the first batch if the first batch is determined to have a total amount of N-nitroso-ribociclib in free or salt form of more than 1 ppm, relative to the total amount of ribociclib in free or salt form; and
d. preparing a second batch of ribociclib or of a pharmaceutically acceptable salt thereof; wherein the reagents and/or conditions that are used in step d are adjusted relative to the reagents and/or conditions that are used in step a so as to prevent, retard or reduce the formation of N-nitroso-ribociclib in free or salt form.

138. A process according to clause 137, wherein the first batch is discarded if the first batch is determined to have a total amount of N-nitroso-ribociclib in free or salt form of more than 0.7 ppm, relative to the total amount of ribociclib in free or salt form; preferably wherein the first batch is discarded if the first batch is determined to have a total amount of N-nitroso-ribociclib in free or salt form of more than 0.6 ppm, relative to the total amount of ribociclib in free or salt form.

139. The process according to clause 137 or clause 138, wherein the step of determining the total amount of N-nitroso-ribociclib in free or salt form is performed by a method which includes the Griess test and/or high performance liquid chromatography (HPLC)-spectroscopy and/or gas chromatography (GC)-mass spectroscopy.

140. A pharmaceutical product containing: (a) a pharmaceutical composition comprising ribociclib or a pharmaceutically acceptable salt thereof, and one or more pharmaceutically acceptable excipients; and (b) a document which sets forth, either directly or via a link to an electronic database, that the pharmaceutical composition has (1) a shelf life and (2) the storage condition(s) for the pharmaceutical composition; wherein the total amount of N-nitroso-ribociclib in free or salt form in the pharmaceutical composition is no more than 1 ppm, relative to the total amount of ribociclib in free or salt form, preferably wherein the total amount of N-nitroso-ribociclib in free or salt form in the pharmaceutical composition is no more than 0.7 ppm, relative to the total amount of ribociclib in free or salt form, more preferably wherein the total amount of N-nitroso-ribociclib in free or salt form in the pharmaceutical composition is no more than 0.6 ppm, relative to the total amount of ribociclib in free or salt form; wherein the pharmaceutical composition is at its end of shelf life and has been stored during the shelf life in compliance with the storage condition(s).

141. The pharmaceutical product of clause 140, wherein the shelf life is at least 1, 2, 3, 4, 5, 6, 7, 8, 9, 10, 11, 12, 13, 14, 15, 16, 17, 18, 24, 30, 36, 42, 48, 54 or 60 months.

142. The pharmaceutical product of clause 140 or 141, wherein the storage condition(s) include that the pharmaceutical composition be stored at a temperature of no more than 40° C. during the shelf life.

143. The pharmaceutical product of clause 140 or 141, wherein the storage condition(s) include that the pharmaceutical composition be stored at a temperature of 2° C. to 25° C. during the shelf life.

144. The pharmaceutical product of any one of clauses 140 to 144, wherein the pharmaceutical composition has been stored in its original packaging during the shelf life.

145. The pharmaceutical product of any one of clauses 140 to 144, wherein the pharmaceutical composition was determined to have a total amount of N-nitroso-ribociclib in free or salt form in the pharmaceutical composition, at time of release, of no more than 0.8 ppm, relative to the total amount of ribociclib in free or salt form, preferably of no more than 0.7 ppm, relative to the total amount of ribociclib in free or salt form, more preferably of no more than 0.6 ppm, relative to the total amount of ribociclib in free or salt form.

### Clauses—List C

The invention provides the following numbered clauses:

1. A method of preparing a pharmaceutical product comprising a plurality of oral dosage forms, wherein each of the oral dosage forms is a tablet, and wherein said oral dosage forms comprise a composition comprising: (i) ribociclib or a pharmaceutically acceptable salt thereof; and (ii) one or more pharmaceutically acceptable excipients, wherein the method comprises: (a) providing the composition; (b) compressing the composition into tablets; and (c) drying the tablets until the water activity value of the tablets is less than 0.08.

2. The method of clause 1, wherein the method comprises applying a coating to the tablets before the step of drying the tablets.

3. The method of clause 2, wherein the coating is a film coating, optionally wherein the film coating is a moisture barrier film coating.

4. The method of any one of clauses 1 to 3, wherein the step of drying the tablets is carried out until the water activity value of the tablets is no more than 0.06, and preferably no more than 0.05.

5. The method of any one of clauses 1 to 4, wherein the step of drying the tablets is conducted at a temperature of about 25° C. to about 50° C., preferably at a temperature of about 28° C. to about 32° C., and more preferably at a temperature of about 30° C.

6. The method of clause 5, wherein the step of drying the tablets is conducted at the said temperature for a time period of at least 44 hours, at least 48 hours, at least 52 hours, at least 56 hours, at least 60 hours, at least 64 hours, at least 68 hours, or at least 72 hours, preferably wherein the step of drying the tablets is conducted at the said temperature for a time period of at least 56 hours.

7. The method of any one of clauses 1 to 6, wherein the step of drying the tablets is conducted by flowing an atmosphere over the tablets, wherein the humidity of the atmosphere is no more than 0.5 g/kg, preferably no more than 0.1 g/kg, and more preferably no more than 0.01 g/kg.

8. The method of clause 7, wherein the flow rate of the atmosphere is about 28 to about 30 m³/h.

9. The method of clause 7 or 8, wherein the atmosphere is air.

10. The method of clause 9, wherein prior to being flowed over the tablets, the air is treated to reduce its content of NOx species.

11. The method of any one of clauses 1 to 10, wherein both before and after the step of drying the tablets, the total amount of N-nitroso-ribociclib in free or salt form in each of the tablets is no more than 0.7 ppm, and preferably no more than 0.6 ppm, relative to the total amount of ribociclib in free or salt form.

12. The method of any one of clauses 1 to 11, wherein both before and after the step of drying the tablets, the total amount of nitrosamines in each of the tablets is no more than 0.7 ppm, and preferably no more than 0.6 ppm, relative to the total amount of ribociclib in free or salt form.

13. The method of any one of clauses 1 to 12, wherein after the step of drying the tablets, the tablets are stored and/or packaged under an atmosphere in which the humidity is no more than 2.5 g/kg.

14. The method of any one of clauses 1 to 13, wherein the tablets are packaged in a sealed container, optionally wherein the tablets are packaged into a blister pack such that each tablet is individually packaged within a substantially moisture and/or gas impermeable blister, preferably wherein the blister pack is an alu-alu blister pack, a plastic blister pack, or a laminate blister pack, more preferably wherein the blister pack is an alu-alu blister pack.

15. The method of any one of clauses 1 to 14, wherein the step of providing the composition comprises combining the ribociclib or pharmaceutically acceptable salt thereof with the one or more pharmaceutically acceptable excipients, wherein said one or more pharmaceutically acceptable excipients are selected from the group consisting of magnesium stearate, microcrystalline cellulose, crospovidone, hydroxypropyl cellulose, and silica, optionally wherein the one or more pharmaceutically acceptable excipients comprises one or both of microcrystalline cellulose and crospovidone and (a) the content of nitrites in the microcrystalline cellulose is no more than 100 ppb and/or (b) the content of nitrites in the crospovidone is no more than 100 ppb.

16. The method of any one of clauses 1 to 15, wherein the step of providing the composition comprises a method of preparing ribociclib or pharmaceutically acceptable salt thereof, optionally wherein the method of preparing the ribociclib or pharmaceutically acceptable salt thereof uses one or more solvents and (i) the total amount of nitrites in each of the one or more solvents is no more than 5 ppb, preferably no more than 3.5 ppb, and more preferably no more than 1 ppb, relative to the amount of the respective solvent; and/or (ii) the combined total amount of nitrites in the solvents is no more than 5 ppb, preferably no more than 3.5 ppb, and more preferably no more than 1 ppb, relative to the combined amount of the solvents.

17. The method of any one of clauses 1 to 16, wherein the tablets as comprised within the pharmaceutical product have a shelf life of at least 18 months, and preferably at least 24 months, when the pharmaceutical product is stored at a temperature of about 20° C. to about 25° C.

18. The method of clause 17, wherein the shelf life is the period of time in which the total amount of N-nitroso-ribociclib in free or salt form in each of the tablets remains no more than 1 ppm, preferably no more than 0.7 ppm, and more preferably no more than 0.6 ppm, relative to the total amount of ribociclib in free or salt form.

19. The method of clause 17 or 18, wherein the shelf life is the period of time in which the total amount of nitrosamines in each of the tablets remains no more than 1 ppm, preferably no more than 0.7 ppm, and more preferably no more than 0.6 ppm, relative to the total amount of ribociclib in free or salt form.

20. A method of identifying a process for drying a pharmaceutical product comprising a plurality of oral dosage forms, wherein each of the oral dosage forms is a tablet, wherein said oral dosage forms comprise a composition comprising: (i) ribociclib or a pharmaceutically acceptable salt thereof; and (ii) one or more pharmaceutically acceptable excipients, wherein the process of drying comprises flowing an atmosphere over the tablets, and wherein any one or any combination of the following parameters is adjusted until the process produces a product in which both: (1) the water activity value of the tablets is less than 0.08, preferably no more than 0.06, and more preferably no more than 0.05; and (2) the total amount of nitrosamines in each of the tablets, or at least the total amount of N-nitroso-ribociclib in free or salt form in each of the tablets, is no more than 0.7 ppm, and preferably no more than 0.6 ppm, relative to the total amount of ribociclib in free or salt form, the said parameters being:
a. the temperature at which the drying is conducted, optionally wherein the temperature is adjusted within the range of about 25° C. to about 50° C., and preferably within the range of about 25° C. to about 35° C.;
b. the time period over which the drying is conducted, optionally wherein the time period is adjusted within the range of about 44 hours to about 72 hours, and preferably within the range of about 52 hours to about 64 hours;
c. the humidity of the atmosphere, optionally wherein the humidity is adjusted within the range of no more than 0.5 g/kg;
d. the content of NOx species in the atmosphere; and
e. the flow rate of the atmosphere, optionally wherein the flow rate is adjusted within the range of less than 3000 m³/h.

21. The method of clause 20, wherein the atmosphere is air.

### Clauses—List D

The invention provides the following numbered clauses:

1. A pharmaceutical product comprising a plurality of oral dosage forms, wherein each of the oral dosage forms is a tablet, and wherein the oral dosage forms comprise a composition comprising: (i) ribociclib or a pharmaceutically acceptable salt thereof, and (ii) one or more pharmaceutically acceptable excipients, wherein the total amount of N-nitroso-ribociclib in free or salt form in each of the oral dosage forms is no more than 1 ppm, relative to the total amount of ribociclib in free or salt form.

2. The pharmaceutical product of clause 1, wherein the product comprises at least 5 of the oral dosage forms.

3. The pharmaceutical product of clause 1, wherein each of the one or more pharmaceutically acceptable excipients has a content of nitrites of less than 0.5 ppm, less than 0.4 ppm, less than 0.3 ppm, less than 0.2 ppm, or less than 0.1 ppm relative to the amount of the respective excipient.

4. The pharmaceutical product of clause 1, wherein the one or more pharmaceutically acceptable excipients is more than one excipient, and the combined excipients have a total content of nitrites of less than 0.5 ppm relative to the combined amount of the excipients.

5. The pharmaceutical product of clause 1, wherein the one or more pharmaceutically acceptable excipients are selected from the group consisting of magnesium stearate, microcrystalline cellulose, crospovidone, hydroxypropyl cellulose, and silica; and wherein each of the one or more pharmaceutically acceptable excipients has a content of nitrites of less than 0.2 ppm relative to the amount of the respective excipient, or wherein the one or more pharmaceutically acceptable excipients is more than one excipient and the combined excipients have a total content of nitrites of less than 0.5 ppm relative to the combined amount of the excipients.

6. The pharmaceutical product of clause 1, wherein each of the oral dosage forms comprises ribociclib succinate.

7. The pharmaceutical product of clause 6, wherein the total amount of ribociclib or pharmaceutically acceptable salt thereof in each of the oral dosage forms provides the same amount of ribociclib as 200 mg of ribociclib free base.

8. The pharmaceutical product of clause 7, wherein the water activity value of each of the oral dosage forms is less than 0.08.

9. The pharmaceutical product of clause 7, wherein the water activity value of each of the oral dosage forms is less than 0.06.

10. The pharmaceutical product of clause 8 or 9, wherein the oral dosage forms are each individually or together present in a sealed container.

11. The pharmaceutical product of clause 9, wherein the oral dosage forms have a shelf life of at least 12 months when stored at a temperature of no more than 25° C.

12. The pharmaceutical product of clause 9, wherein the oral dosage forms have a shelf life of at least 18 months when stored at a temperature of no more than 25° C.

13. The pharmaceutical product of clause 1, wherein the pharmaceutical product comprises a document which either directly or via a link to an electronic database specifies how the pharmaceutical product should be stored, wherein if the pharmaceutical product is stored in accordance with the instructions, then the total amount of N-nitroso-ribociclib in free or salt form in each of the oral dosage forms will be no more than 1 ppm, relative to the total amount of ribociclib in free or salt form.

14. A tablet comprising (i) ribociclib or a pharmaceutically acceptable salt thereof; and (ii) one or more pharmaceutically acceptable excipients, wherein: (1) the total amount of nitrosamines in the tablet, or at least the total amount of N-nitroso-ribociclib in free or salt form in the tablet, is no more than 1 ppm (preferably, no more than 0.7 ppm or 0.6 ppm) relative to the total amount of ribociclib in free or salt form; (2) the tablet has a water activity value of less than 0.08; and (3) the total amount of ribociclib or pharmaceutically acceptable salt thereof in the tablet provides the same amount of ribociclib as 200 mg of ribociclib free base.

15. The tablet of clause 14, wherein the tablet has a content of nitrites of no more than 0.5 ppm.

16. The tablet of clause 14, wherein the combined excipients have a total content of nitrites of no more than 0.5 ppm, relative to the combined amount of the excipients.

17. The tablet of clause 16, wherein the one or more pharmaceutically acceptable excipients are selected from the group consisting of magnesium stearate, microcrystalline cellulose, crospovidone, hydroxypropyl cellulose, and silica; and wherein each of the one or more pharmaceutically acceptable excipients has a content of nitrites of less than 0.1 ppm relative to the amount of the respective excipient, or wherein the one or more pharmaceutically acceptable excipients is more than one excipient and the combined excipients have a total content of nitrites of less than 0.5 ppm relative to the combined amount of the excipients.

18. The tablet of clause 17, wherein the tablet is contained within a blister of a blister pack, wherein the blister is substantially moisture and/or gas impermeable.

1. A pharmaceutical product comprising a plurality of oral dosage forms, wherein each of the oral dosage forms is a tablet, and wherein the oral dosage forms comprise a composition comprising: (i) ribociclib or a pharmaceutically acceptable salt thereof, and (ii) one or more pharmaceutically acceptable excipients, wherein the total amount of N-nitroso-ribociclib in free or salt form in each of the oral dosage forms is no more than 1 ppm, relative to the total amount of ribociclib in free or salt form.

2. (canceled)

3. The pharmaceutical product of claim 1, wherein the product comprises at least 5 the oral dosage forms.

4. The pharmaceutical product of claim 1, wherein each of the one or more pharmaceutically acceptable excipients has a content of nitrites of less than 0.5 ppm relative to the amount of the respective excipient.

5. The pharmaceutical product of claim 1, and wherein the one or more pharmaceutically acceptable excipients are selected from the group consisting of magnesium stearate, microcrystalline cellulose, crospovidone, hydroxypropyl cellulose, and silica; and wherein each of the one or more pharmaceutically acceptable excipients has a content of nitrites of less than 0.2 ppm relative to the amount of the respective excipient, or wherein the one or more pharmaceutically acceptable excipients is more than one excipient and the combined excipients have a total content of nitrites of less than 0.5 ppm relative to the combined amount of the excipients.

6. The pharmaceutical product of claim 1, wherein each of the oral dosage forms comprises ribociclib succinate.

7. The pharmaceutical product of claim 6, wherein the total amount of ribociclib or pharmaceutically acceptable salt thereof in each of the oral dosage forms provides the same amount of ribociclib as 200 mg of ribociclib free base.

8. The pharmaceutical product of claim 7, wherein the water activity value of each of the oral dosage forms is less than 0.08.

9. (canceled)

10. The pharmaceutical product of claim 8, wherein the oral dosage forms are each individually or together present in a sealed container.

11. The pharmaceutical product of claim 10, wherein said oral dosage forms have a shelf life of at least 12 months when stored at a temperature of no more than 25° C.

12. The pharmaceutical product claim 1, wherein said pharmaceutical product comprises a document which either directly or via a link to an electronic database specifies how the pharmaceutical product should be stored, wherein if the pharmaceutical product is stored in accordance with the instructions, then the total amount of N-nitroso-ribociclib in free or salt form in each of the oral dosage forms will be no more than 1 ppm, relative to the total amount of ribociclib in free or salt form.

13.-30. (canceled)

31. A tablet comprising (i) ribociclib or a pharmaceutically acceptable salt thereof; and (ii) one or more pharmaceutically acceptable excipients, wherein: (1) the total amount of nitrosamines in the tablet, or at least the total amount of N-nitroso-ribociclib in free or salt form in the tablet, is no more than 1 ppm, relative to the total amount of ribociclib in free or salt form; (2) the tablet has a water activity value of less than 0.08; and (3) the total amount of ribociclib or pharmaceutically acceptable salt thereof in the tablet provides the same amount of ribociclib as 200 mg of ribociclib free base.

32. The tablet of claim 31, wherein the tablet has a content of nitrites of no more than.

33. The tablet of claim 31, wherein the combined excipients have a total content of nitrites of less than 0.5 ppm, relative to the combined amount of the excipients.

34.-35. (canceled)

36. The tablet of claim 31, wherein the tablet is contained within a blister of a blister pack, wherein the blister is substantially moisture and/or gas impermeable.

37.-38. (canceled)
